(19) United States
(12) Patent Application Publication
(10) Pub. No.: US 2011/0312881 A1
(43) Pub. Date: Dec. 22, 2011

(54) BIFUNCTIONAL POLYPEPTIDE COMPOSITIONS AND METHODS FOR TREATMENT OF METABOLIC AND CARDIOVASCULAR DISEASES

(75) Inventors: Joshua Silverman, Sunnyvale, CA (US); Volker Schellenberger, Palo Alto, CA (US); Willem P. Stemmer, Los Gatos, CA (US); Jeffrey L. Cleland, San Carlos, CA (US)

(73) Assignee: AMUNIX, Inc., Mountain View, CA (US)

(21) Appl. No.: 12/975,054

(22) Filed: Dec. 21, 2010

Related U.S. Application Data
(60) Provisional application No. 61/284,527, filed on Dec. 21, 2009.

(51) Int. Cl. A61K 38/16; C12N 1/19; C12N 1/21; C12N 1/15; C12N 15/63; C07H 21/04; A61P 5/50; A61P 3/10; A61P 9/00; A61P 25/28; C07K 14/00; C12N 5/10; A61P 3/04

(52) U.S. Cl. 514/4.8; 435/325; 435/352; 435/353; 435/358; 435/366; 435/252.3; 435/252.31; 435/252.33; 435/252.34; 435/252.35; 435/254.11; 435/254.2; 435/254.21; 435/254.23; 435/254.3; 435/254.4; 435/254.6; 435/254.7; 435/320.1; 514/6.7; 514/6.9; 514/16.4; 514/17.7; 514/21.2; 530/350; 536/23.4

(57) ABSTRACT

The present invention relates to compositions comprising combinations of biologically active proteins linked to extended recombinant polymer, methods of production of the compositions and their use in treatment of metabolic and cardiovascular diseases, disorders and conditions.

### CROSS-REFERENCE TO RELATED APPLICATIONS

This application claims the priority benefit of U.S. Provisional Application Ser. No. 61/284,527, filed Dec. 21, 2009.

### STATEMENT REGARDING FEDERALLY SPONSORED RESEARCH

This invention was made with government support under SBIR grant 2R44GM079873-02 awarded by the National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Feb. 9, 2011, is named 32808201.txt and is 10,726,737 bytes in size.

### BACKGROUND OF THE INVENTION

Metabolic and cardiovascular diseases represent a substantial health care burden in most developed nations, with cardiovascular diseases remaining the number one cause of death and disability in the United States and most European countries. Metabolic diseases and disorders include a large variety of conditions affecting the organs, tissues, and circulatory system of the body. Of particular concern are endocrine and obesity-related diseases and disorders, which have reached epidemic proportions in most developed nations. Chief amongst these is diabetes; one of the leading causes of death in the United States. Diabetes is divided into two major sub-classes-Type I, also known as juvenile diabetes, or Insulin-Dependent Diabetes Mellitus (IDDM), and Type II, also known as adult onset diabetes, or Non-Insulin-Dependent Diabetes Mellitus (NIDDM). Type I Diabetes is a form of autoimmune disease that completely or partially destroys the insulin producing cells of the pancreas in such subjects, and requires use of exogenous insulin during their lifetime. Even in well-managed subjects, episodic complications can occur, some of which are life-threatening.

In Type II diabetics, rising blood glucose levels after meals do not properly stimulate insulin production by the pancreas. Additionally, peripheral tissues are generally resistant to the effects of insulin, and such subjects often have higher than normal plasma insulin levels (hyperinsulinemia) as the body attempts to overcome its insulin resistance. In advanced disease states insulin secretion is also impaired.

Insulin resistance and hyperinsulinemia have also been linked with two other metabolic disorders that pose considerable health risks: impaired glucose tolerance and metabolic obesity. Impaired glucose tolerance is characterized by normal glucose levels before eating, with a tendency toward elevated levels (hyperglycemia) following a meal. These individuals are considered to be at higher risk for diabetes and coronary artery disease. Obesity is also a risk factor for the group of conditions called insulin resistance syndrome, or “Syndrome X,” as is hypertension, coronary artery disease (arteriosclerosis), and lactic acidosis, as well as related disease states. The pathogenesis of obesity is believed to be multifactorial but an underlying problem is that in the obese, nutrient availability and energy expenditure are not in balance until there is excess adipose tissue.

Dyslipidemia is a frequent occurrence among diabetics; typically characterized by elevated plasma triglycerides, low HDL (high density lipoprotein) cholesterol, normal to elevated levels of LDL (low density lipoprotein) cholesterol and increased levels of small dense, LDL particles in the blood. Dyslipidemia is a main contributor to an increased incidence of coronary events and deaths among diabetic subjects.

Cardiovascular disease can be manifest by many disorders involving the heart and vasculature throughout the body, including aneurysms, angina, atherosclerosis, cerebrovascular accident (Stroke), cerebrovascular disease, congestive heart failure, coronary artery disease, myocardial infarction, and peripheral vascular disease, amongst others.

Most metabolic processes and many cardiovascular parameters are regulated by multiple peptides and hormones, and many such peptides and hormones, as well as analogues thereof, have found utility in the treatment of such diseases and disorders. However, the use of single therapeutic peptides and/or hormones, even when augmented by the use of small molecule drugs, has met with limited success in the management of such diseases and disorders. In particular, dose optimization is important for drugs and biologics used in the treatment of metabolic diseases, especially those with a narrow therapeutic window. Hormones in general, and peptides involved in glucose homeostasis often have a narrow therapeutic window. The narrow therapeutic window, coupled with the fact that such hormones and peptides typically have a short half-life, results in difficulties in the management of such patients. Therefore, there remains a need for therapeutics with increased efficacy and safety in the treatment of metabolic and cardiovascular diseases. The present invention addresses this need by providing bifunctional compositions comprising combinations of biologically active proteins fused to extended recombinant polypeptides selected to tailor the pharmacokinetic properties of the compositions, providing controlled and extended exposures within the therapeutic window for the biologics.

### SUMMARY OF THE INVENTION

The present invention is directed to compositions and methods of treatment or prevention of metabolic and/or cardiovascular diseases, disorders or conditions. In particular, the present invention provides compositions comprising biologically active proteins and extended recombinant polypeptides (XTEN), resulting in fusion proteins that are either monomeric fusion proteins with two different biologically active proteins or are compositions of two different monomeric fusion proteins with one biologically active protein each; collectively bifunctional fusion protein compositions (herein after “BFXTEN”). In part, the present disclosure is directed to pharmaceutical compositions comprising the fusion proteins and the uses thereof for treating metabolic- and/or cardiovascular-related diseases, disorders or conditions. The BFXTEN compositions have enhanced pharmacokinetic properties compared to BP not linked to XTEN, which may permit more convenient dosing and improved efficacy. In some embodiments, the BFXTEN compositions of the invention do not have a component selected the group consisting of: polyethylene glycol (PEG), albumin, and an antibody fragment such as an Fc fragment.

In one aspect, the invention provides compositions comprising fusion proteins of BP and XTEN in different configurations and/or in different combinations. In one embodiment, the invention provides compositions of a monomeric fusion protein of formula V:

(XTEN)ᵤ-(S)ᵥ-(BP1)-(S)_{w}-(XTEN)-(S)ₓ-(BP2)-(S)_{y}-(XTEN)_{z} V

wherein independently for each occurrence BP1 is a is a biologically active protein comprising a sequence that exhibiting at least about 90%, or about 95%, or about 96%, or about 97%, or about 98%, or about 99% sequence identity to an amino acid sequence selected from Table 1; BP2 is a is a biologically active protein different from BP1 that exhibits at least about 90%, or about 95%, or about 96%, or about 97%, or about 98%, or about 99% sequence identity to an amino acid sequence selected from Table 1; S is a spacer sequence having between 1 to about 50 amino acid residues that can optionally include a cleavage sequence selected from Table 6 or amino acids compatible with restriction sites selected from Table 5, u is either 0 or 1, v is either 0 or 1, w is either 0 or 1, x is either 0 or 1, y is either 0 or 1, z is either 0 or 1, with the proviso that u+v+w+x+y+z≧1, and XTEN is an extended recombinant polypeptide comprising greater than about 100 to about 3000 amino acids. The XTEN sequence(s) of the fusion protein are characterized in that: the sequence(s) are substantially non-repetitive sequence such that: (1) the sequence contains no three contiguous amino acids that are identical unless the amino acids are serine residues; or (2) at least about 80% of the XTEN sequence consists of non-overlapping sequence motifs, each of the sequence motifs comprising about 9 to about 14 amino acid residues, wherein any two contiguous amino acid residues does not occur more than twice in each of the sequence motifs; or (3) the XTEN has a subsequence score or less than 3 or less than 2; the sum of glycine (G), alanine (A), serine (S), threonine (T), glutamate (E) and proline (P) residues constitutes more than about 80% of the total amino acid sequence of the XTEN; the XTEN sequence lacks a predicted T-cell epitope when analyzed by TEPITOPE algorithm, wherein the TEPITOPE algorithm prediction for epitopes within the XTEN sequence is based on a score of −5, or −6, or −8, or −9 or −10; the XTEN sequence has greater than about 90%, or about 95%, or about 99% random coil formation as determined by GOR algorithm and the XTEN sequence has less than 5%, or less than 4%, or less than 3%, or less than 2% alpha helices and less than 5%, or less than 4%, or less than 3%, or less than 2% beta-sheets as determined by Chou-Fasman algorithm. In one embodiment, the XTEN is further characterized in that the sum of asparagine and glutamine residues is less than 10% of the total amino acid sequence of the XTEN, the sum of methionine and tryptophan residues is less than 2% of the total amino acid sequence of the XTEN, and no one type of amino acid constitutes more than 30% of the XTEN sequence. In one embodiment, the XTEN is further characterized in that the XTEN sequence has less than 10%, or less than 5%, or less than 4%, or less than 3%, or less than 2% amino acid residues with a positive charge. In another embodiment, the XTEN of the composition has at least about 80%, or about 85%, or about 90%, or about 91%, or about 92%, or about 93%, or about 94%, or about 95%, or about 96%, or about 97%, or about 98%, or about 99% of the sequence consisting of non-overlapping sequence motifs, wherein each of the sequence motifs has 12 amino acid residues selected from one or more sequences of Table 3. The motifs of the XTEN sequence can be selected from a single family, i.e., AD, AE, AF, AG, AM, AQ, BC or BD. The XTEN of the composition can be identical or they can be different. In one embodiment, the XTEN of the composition each exhibit at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identity with a sequence selected from Table 4 or a fragment thereof.

In another embodiment, the invention provides compositions of a monomeric fusion protein of formula VI:

(XTEN)ᵥ-(S)_{w}-(BP1)-(S)ₓ-(BP2)-(S)_{y}-(XTEN)_{z} VI

wherein independently for each occurrence BP1 is a is a biologically active protein comprising a sequence that exhibiting at least about 90%, or about 95%, or about 96%, or about 97%, or about 98%, or about 99% sequence identity to an amino acid sequence selected from Table 1; BP2 is a is a biologically active protein different from BP1 that exhibits at least about 90%, or about 95%, or about 96%, or about 97%, or about 98%, or about 99% sequence identity to an amino acid sequence selected from Table 1; S is a spacer sequence having between 1 to about 50 amino acid residues that can optionally include a cleavage sequence selected from Table 6 or amino acids compatible with restriction sites selected from Table 5, v is either 0 or 1, w is either 0 or 1, x is either 0 or 1, y is either 0 or 1, z is either 0 or 1, with the proviso that v+w+x+y+z≧1, and XTEN is an extended recombinant polypeptide comprising greater than about 100 to about 3000 amino acids with the characteristics as described for formula V, above.

In other embodiments, the invention provides BFXTEN compositions comprising a first fusion protein and a second fusion protein, wherein the first fusion protein comprises a first biologically active protein (BP1) comprising a sequence that exhibits at least 90% sequence identity to a sequence from Table 1, wherein the BP1 is linked to one or more extended recombinant polypeptides (XTEN) each comprising greater than about 100 to about 3000 amino acid residues and the second fusion protein comprises a second biologically active protein (BP2) comprising a sequence that exhibits at least 90% sequence identity to a sequence from Table 1 and that is different from the BP1 of (a), wherein the BP2 is linked to one or more extended recombinant polypeptides (XTEN) each comprising greater than about 100 to about 3000 amino acid residues with the characteristics as described for formula V, above. In one embodiment of the foregoing, the first fusion protein is of formula I

(BP1)-(S)ₓ-(XTEN) I

or formula III

(XTEN)-(S)ₓ-(BP1) III

and the second fusion protein is of formula II

(BP2)-(S)_{y}-(XTEN) II

or formula IV

(XTEN)-(S)_{y}-(BP2) IV

wherein independently for each occurrence BP1 is a is a biologically active protein comprising a sequence that exhibiting at least about 80%, or about 90%, or about 95%, or about 96%, or about 97%, or about 98%, or about 99% sequence identity to an amino acid sequence selected from Table 1; BP2 is a is a biologically active protein different from BP1 that exhibits at least about 80%, or about 90%, or about 95%, or about 96%, or about 97%, or about 98%, or about 99% sequence identity to an amino acid sequence selected from Table 1; S is a spacer sequence having between 1 to about 50 amino acid residues that can optionally include a cleavage sequence selected from Table 6 or amino acids compatible with restriction sites selected from Table 5, x is either 0 or 1, y is either 0 or 1, and XTEN is an extended recombinant polypeptide comprising greater than about 100 to about 3000 amino acids with the characteristics as described for formula V, above, and the first and the second fusion protein are at a fixed ratio in the composition of about 1:1 to about 1:1500.

The invention provides BFXTEN compositions comprising two fusion proteins, each with a different BP, wherein each fusion protein has at least about 80%, or about 90%, or about 95%, or about 96%, or about 97%, or about 98%, or about 99% sequence identity to an amino acid sequence selected from Table 33. The invention provides BXTEN compositions of monomeric fusion protein with two different BP, wherein the fusion protein has at least about 80%, or about 90%, or about 95%, or about 96%, or about 97%, or about 98%, or about 99% sequence identity to an amino acid sequence selected from Table 34 or Table 35 or Table 36 or Table 37.

The BFXTEN compositions of the foregoing embodiments have enhanced pharmacokinetic properties when administered to a subject, such as a human, compared to the corresponding BP not linked to XTEN, which may permit more convenient dosing and improved efficacy. The enhanced pharmacokinetic properties include increased terminal half-life, increased area under the curve, volume of distribution, increased time spent within the therapeutic window, increased time between consecutive administrations to maintain the BFXTEN within the therapeutic window, and increased bioavailability. In one embodiment, the BXTEN composition, when administered to a subject, exhibits a terminal half-life at least about two-fold longer, or about three-fold longer, or about four-fold longer, or about five-fold longer, or about 10-fold longer, or about 20-fold longer compared to the corresponding BP1 and/or the BP2 not linked to the XTEN and administered at a comparable dose to a subject. In another embodiment, the BXTEN composition, when administered to a subject, exhibits an increased area under the curve (AUC) of at least about two-fold, or at least about four-fold, or at least about five-fold, or at least about 10-fold, or at least about 15-fold, or at least about 20-fold compared to the corresponding BP1 and/or the BP2 not linked to the XTEN and administered at a comparable dose to a subject. In another embodiment, the BXTEN composition, when administered to a subject, exhibits an increased time within the therapeutic window of at least about two-fold, or at least about four-fold, or at least about five-fold, or at least about 10-fold, or at least about 15-fold, or at least about 20-fold longer compared to the corresponding BP1 and/or the BP2 not linked to the XTEN and administered at a comparable dose to a subject. In another embodiment, the administration of multiple consecutive doses of a BFXTEN using a therapeutically effective dose regimen to a subject in need thereof results in a gain in time of at least two-fold, or at least three-fold, or at least four-fold, or at least five-fold, or at least 10-fold, or at least 20-fold between consecutive Cₘₐₓ peaks and/or Cₘᵢₙ troughs for blood levels of the fusion protein compared to the corresponding BP not linked to the XTEN and administered to a subject at a therapeutically effective dose regimen for the BP.

The invention provides BFXTEN compositions with enhanced pharmacologic properties when administered to a subject, such as a human, compared to the corresponding BP not linked to XTEN, which may permit more convenient dosing and improved efficacy and safety. Administration of multiple consecutive doses using a therapeutically effective dose regimen of the BFXTEN to a subject in need thereof results in an improvement in at least one measured parameter associated with a metabolic or cardiovascular disease or condition using an accumulatively smaller amount of about 5%, or about 10%, or about 20%, or about 40%, or about 50%, or about 60%, or about 70%, or about 80%, or about 90% less moles of fusion protein administered compared to the corresponding BP1 and/or BP2 not linked to the XTEN and administered at a therapeutically effective dose regimen for the BP1 and/or BP2 to a subject. The accumulative amount is measured for a period of at least about one week, or about 14 days, or about 21 days, or about one month. The one measured parameter is selected from the group selected from fasting glucose level, response to oral glucose tolerance test, peak change of postprandial glucose from baseline glucose level, HA_{1c}, level, daily caloric intake, satiety, rate of gastric emptying, insulin secretion in response to glucose challenge, peripheral insulin sensitivity, glucose level in response to insulin challenge, beta cell mass, body weight reduction, left ventricular diastolic function, E/A ratio, left ventricular end diastolic pressure, cardiac output, cardiac contractility, left ventricular mass, left ventricular mass to body weight ratio, left ventricular volume, left atrial volume, left ventricular end diastolic dimension (LVEDD), left ventricular end systolic dimension (LVESD), infarct size, exercise capacity, exercise efficiency, and heart chamber size.

The invention provides BFXTEN wherein a fusion protein of formula I, II, III, IV, V, or VI exhibits a biological activity of at least about 60%, or at least about 70%, or at least about 80%, or at least about 90% for the respective BP1 and BP2 components of the BFXTEN compared to the BP1 and BP2 components not linked to the fusion protein. In another embodiment, the isolated fusion protein of formula I, II, III, IV, V, or VI exhibits binds the target receptor or ligand with about 10%, or at least about 20%, or at least about 30%, or at least about 40%, or at least about 50%, or at least about 60%, or at least about 70%, or at least about 80%, or at least about 90%, or at least about 95%, or at least about 99% or more of the affinity of a native BP not bound to XTEN.

The invention provides a method for increasing the terminal half-life of a BFXTEN by producing a single chain fusion protein construct comprising at least a first biologically active protein and an XTEN sequence in a first N- to C-terminus configuration, wherein the fusion protein in the first configuration of the biologically active protein and XTEN components has reduced receptor-mediated clearance compared to a BFXTEN in a second configuration wherein the biologically active protein and an XTEN components are in a second, different N- to C-terminus configuration. In one embodiment of the method, the configuring of the BFXTEN in the first configuration results in a fusion protein wherein the receptor binding for the receptor of the biologically active protein component in the range of about 2-30%, or about 3-20%, or about 4-15%, or about 5-10% compared to the BFXTEN in the second Configuration. In another embodiment of the method, the configuring of the BFXTEN in the first configuration results in a fusion protein wherein administration of the fusion protein to a subject results in an increase in the terminal half-life of at least about two-fold, or at least three-fold, or at least four-fold, or at least five-fold compared to the half-life of a BFXTEN in the second configuration.

the XTEN sequence has less than 10%, or less than 5%, or less than 4%, or less than 3%, or less than 2% amino acid residues with a positive charge; the XTEN sequence has greater than 80%, or about 85%, or about 90%, or about 91%, or about 92%, or about 93%, or about 94%, or about 95%, or about 96%, or about 97%, or about 98%, or about 99% random coil formation as determined by GOR algorithm; and the XTEN sequence has less than 2% alpha helices and 2% beta-sheets as determined by Chou-Fasman algorithm.

In some cases, the invention provides BFXTEN fusion proteins in which at least about 80%, or about 85%, or about 90%, or about 91%, or about 92%, or about 93%, or about 94%, or about 95%, or about 96%, or about 97%, or about 98%, or about 99% of the XTEN sequence consists of non-overlapping sequence motifs, wherein each of the sequence motifs has 12 amino acid residues. In one embodiment of the foregoing, the sequence motifs are selected from one or more sequences of Table 3.

The invention provides BFXTEN fusion proteins with an increased apparent molecular weight as determined by size exclusion chromatography, compared to the actual molecular weight, wherein the apparent molecular weight is at least about 100 kD, or at least about 150 kD, or at least about 200 kD, or at least about 300 kD, or at least about 400 kD, or at least about 500 kD, or at least about 600 kD, or at least about 700 kD, while the actual molecular weight of each biologically active protein component of the fusion protein is less than about 25 kD. Accordingly, the BFXTEN fusion proteins can have an apparent molecular weight that is about 4-fold greater, or 5-fold greater, or about 6-fold greater, or about 7-fold greater, or about 8-fold, or about 10-fold, or about 5-fold greater than the actual molecular weight of the fusion protein. Accordingly, the BFXTEN fusion proteins have an apparent molecular weight factor under physiologic conditions that is greater than about 4, or about 5, or about 6, or about 7, or about 8, or about 10, or greater than about 15.

The invention provides pharmaceutical compositions comprising a fusion protein of any of the foregoing embodiments at least one pharmaceutically acceptable carrier. In one embodiment, the invention provides pharmaceutical compositions comprising either a monomeric BFXTEN comprising a BP1 and a BP2 or a combination of two BFXTEN fusion proteins each comprising a different biologically active protein and at least one pharmaceutically acceptable carrier. In another embodiment, the invention provides kits, comprising packaging material and at least a first container comprising the pharmaceutical composition of the foregoing embodiment and a label identifying the pharmaceutical composition and storage and handling conditions, and a sheet of instructions for the reconstitution and/or administration of the pharmaceutical compositions to a subject.

In another aspect, the invention provides a method of treating or preventing a metabolic or cardiovascular-related disease, disorder or condition, comprising administering a pharmaceutical composition comprising BFXTEN fusion protein(s) of any of the foregoing embodiments to a subject in need thereof. In one embodiment of the foregoing, the disease, disorder or condition is selected from type 1 diabetes, type 2 diabetes, obesity, hyperglycemia, hyperinsulinemia, decreased insulin production, insulin resistance, syndrome X and retinal neurodegenerative processes. In another embodiment of the foregoing, the disease, disorder or condition is selected from myocardial infarction, cardiac valve disease, stroke, post-surgical catabolic changes, hibernating myocardium or diabetic cardiomyopathy, hypertrophic cardiomyopathy, heart insufficiency, aortic stenosis, valvular regurgitation, and intermittent claudication.

The pharmaceutical composition can be administered subcutaneously (including subcutaneously by infusion pump), intramuscularly, or intravenously. In one embodiment of the method of treatment, the pharmaceutical composition is administered at a therapeutically effective amount. In a feature of the method, the administration of the therapeutically effective amount results in a gain in time spent within a therapeutic window for the fusion protein(s) of the pharmaceutical composition compared to the corresponding biologically active protein component(s) not linked to the fusion protein and administered at a comparable dose to a subject. In one embodiment, the gain in time spent within the therapeutic window is at least three-fold, or at least four-fold, or at least five-fold compared to the corresponding biologically active protein component(s) not linked to the fusion protein and administered at a comparable dose to a subject. The method of treatment includes administration of multiple consecutive doses of the pharmaceutical composition at therapeutically effective doses, thereby establishing a therapeutically effective dose regimen. In one embodiment of the foregoing method of treatment, the therapeutically effective dose regimen results in a gain in time of at least four-fold between at least two consecutive C^{max} peaks and/or Cₘᵢₙ troughs for blood levels of the fusion protein compared to the corresponding glucose regulating peptide(s) of the fusion protein not linked to the fusion protein and administered at a comparable dose regimen to a subject. In another embodiment of the method of treatment, administration of the pharmaceutical composition results in an improvement in at least one measured parameter using a lower dose in moles of the fusion protein(s) of the pharmaceutical composition compared to the corresponding biologically active protein component(s) not linked to the fusion protein and administered at a comparable unit dose or dose regimen to a subject. In one embodiment of the foregoing, the one measured parameter is selected from fasting glucose level, response to oral glucose tolerance test, peak change of postprandial glucose from baseline glucose level, HA1c level, daily caloric intake, satiety, rate of gastric emptying, insulin secretion in response to glucose challenge, peripheral insulin sensitivity, glucose level in response to insulin challenge, beta cell mass, and body weight reduction.

In another aspect, the invention provides an isolated nucleic acid comprising a polynucleotide sequence selected from (a) a polynucleotide encoding the fusion protein of any one of the embodiments hereinabove identified, or (b) the complement of the polynucleotide of (a).

The invention also provides an expression vector comprising a polynucleotide sequence encoding the fusion protein of any one of the embodiments hereinabove identified. In one embodiment, the expression vector further comprises a recombinant regulatory sequence operably linked to the polynucleotide sequence, wherein the regulatory sequence is a promoter. In another embodiment, the regulatory sequence comprises one or more transcriptional regulatory elements that control expression of the polynucleotide sequence. The expression vector can further comprise a polynucleotide sequence fused in frame to a polynucleotide encoding a secretion signal sequence. In one embodiment, the secretion signal sequence is a prokaryotic signal sequence. The secretion signal sequence can be selected from OmpA, DsbA, and PhoA signal sequences. In another embodiment, the secretion signal sequence is a eukaryotic signal sequence. The secretion signal sequence can be selected from yeast, insect, and mammalian signal sequences. The expression vector can further comprise a polynucleotide sequence fused to a leader sequence, separable from the polynucleotide sequence encoding any of the fusion proteins herein identified, by a polynucleotide sequence encoding a cleavage site. The cleavage site can be a chemical cleavage site or a proteolytic site. In one embodiment, the proteolytic site is susceptible to cleavage by a protease selected from FXIa, FXIIa, kallikrein, FVIIa, FIXa, FXa, FIIa (thrombin), Elastase-2, granzyme B, MMP-12, MMP-13, MMP-17 or MMP-20, TEV, enterokinase, rhinovirus 3C protease, and sortase A.

The invention further provides a host cell, comprising any of the expression vectors identified herein. The host cell can be a eukaryotic cell, such as yeast, insect, or mammalian cells. The host cell can be a prokaryotic cell, such as E. coli.

The invention further provides kits, comprising a labeled vial containing any one of the pharmaceutical compositions identified herein and instructions for use.

The invention provides an isolated fusion protein comprising a polypeptide sequence that has at least 80% sequence identity, or 81%, 82%, 83%, 84%, 85%, 86%, 87%, 88%, 89%, 90%, 91%, 92%, 93%, 94%, 95%, 96%, 97%, 98%, 99%, or 100% sequence identity to a sequence selected from Tables 33-38.

The invention provides an isolated nucleic acid comprising a polynucleotide sequence that has at least 80% sequence identity, or 81%, 82%, 83%, 84%, 85%, 86%, 87%, 88%, 89%, 90%, 91%, 92%, 93%, 94%, 95%, 96%, 97%, 98%, 99%, or 100% sequence identity to (a) a polynucleotide sequence that encodes a polypeptide selected from Table 33-38; or (b) the complement of the polynucleotide of (a).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The features and advantages of the invention may be further explained by reference to the following detailed description and accompanying drawings that sets forth illustrative embodiments.

FIG. 1 shows schematic representations of seven exemplary BFXTEN fusion proteins or compositions of BFXTEN fusion proteins (FIGS. 1A-G); all depicted in an N- to C-terminus orientation. FIG. 1A shows a combination BFXTEN composition (100) comprising two fusion proteins; the first of which has an XTEN molecule (102) attached to the C-terminus of a biologically active protein 1 (BP1) (103), and the second of which has an XTEN molecule attached to the N-terminus of a spacer sequence (105), which in turn is attached to the N-terminus of a biologically active protein 2 (BP2) (104). FIG. 1B shows a combination BFXTEN composition (100) comprising two fusion proteins, both of which have an XTEN attached to the C-termini of respective BP1 and BP2. FIG. 1C shows a monomeric BFXTEN fusion protein (101) in which the XTEN is linked to the C-terminus of a BP1 and the N-terminus of a BP2. FIG. 1D shows a monomeric BFXTEN fusion protein (101) in which a BP2 is linked to the C-terminus of a BP1, and an XTEN is linked to the C-terminus of a BP2. FIG. 1D shows a monomeric BFXTEN fusion protein (101) in which a BP2 is linked to the C-terminus of a BP1, and an XTEN is linked to the C-terminus of a BP2. FIG. 1E shows a monomeric BFXTEN fusion protein in the opposite configuration of FIG. 1D in which a BP2 is linked to the C-terminus of a BP1, and an XTEN is linked to the N-terminus of a BP1. FIG. 1F shows a monomeric BFXTEN fusion protein (101) in which a BP1 is linked to the N-terminus of a spacer sequence, which in turn is linked to the N-terminus of an XTEN, the C-terminus of the XTEN is linked to the N-terminus of a second spacer sequence, and the second spacer sequence is linked to the N-terminus of a BP2. FIG. 1G shows a monomeric BFXTEN fusion protein (101) in which a BP1 is linked to the N-terminus of a spacer sequence, the C-terminus of the spacer sequence is linked to the N-terminus of a BP2, and the BP2 is linked to the N-terminus of an XTEN.

FIG. 2 is a schematic illustration of seven representative polynucleotide constructs or combinations of constructs (FIGS. 2A-G) of BPXTEN genes that encode the corresponding BFXTEN polypeptides of FIG. 1; all depicted in a 5′ to 3′ orientation. In these illustrative examples of genes encoding combination BFXTEN (200) or monomeric BFXTEN (201) fusion proteins, the polynucleotide encodes the following components: XTEN (202), BP1 (203); BP2 (204); and spacer amino acids that can include a cleavage sequence (205), with all sequences linked in frame.

FIG. 3 is a schematic illustration of an exemplary monomeric BFXTEN acted upon by an endogenously available protease and the ability of the reaction products to bind to a target receptor on a cell surface, with subsequent cell signaling. FIG. 3A shows a monomeric BFXTEN fusion protein (101) in which a BP1 (103) and a BP2 (104) are each linked to the XTEN (102) by spacer sequences that contain a first (105) and a second (106) cleavable sequence, the latter (106) being susceptible to MMP-13 protease (107). FIG. 3B shows the reaction products of a free BP2 (104) and BP1-Spacer Sequence-XTEN (108), plus unreacted BFXTEN (101). FIG. 3C shows the interaction of the reaction product free BP2 (104) with target receptors (110) to BP2 on a cell surface (109). In this case, optimal binding to the receptor is exhibited when BP2 has a free N-terminus. FIG. 3D shows the interaction of the intact BFXTEN with the BP2 receptor that, in this case, has reduced binding affinity due to lack of a free N-terminus. FIG. 3E shows that the free BP2, with high binding affinity, remains bound to the receptor, which has been internalized into an endosome (112) within the cell (109), illustrating receptor-mediated clearance of the bound BP2 and triggering cell signaling (111), portrayed as stippled cytoplasm. FIG. 3F illustrates that the intact BFXTEN (101), with reduced binding affinity to the receptor (110), is nevertheless able to initiate cell signaling without receptor mediated clearance, with the net result that the BFXTEN remains bioavailable.

FIG. 4 is a schematic flowchart of representative steps in the assembly, production and the evaluation of a XTEN.

FIG. 5 is a schematic flowchart of representative steps in the assembly of an BFXTEN polynucleotide construct encoding a fusion protein. Individual oligonucleotides 501 are annealed into sequence motifs 502 such as a 12 amino acid motif (“12-mer”), which is subsequently ligated with an oligo containing BbsI, and KpnI restriction sites 503. Additional sequence motifs from a library are annealed to the 12-mer until the desired length of the XTEN gene 504 is achieved. The XTEN gene is cloned into a stuffer vector. The vector encodes a glucagon gene 506 followed by BsaI, BbsI, and KpnI sites 507 and an exendin-4 gene 508, resulting in the gene 500 encoding an BFXTEN fusion protein encoding two BP.

FIG. 6 is a schematic flowchart of representative steps in the assembly of a gene encoding fusion protein comprising a biologically active protein (BP) and XTEN, its expression and recovery as a fusion protein, and its evaluation as a candidate BFXTEN component.

FIG. 7 is a schematic representation of the design of Ex4XTEN expression vectors with different processing strategies for use in producing a single fusion protein of a BCXTEN. FIG. 7A shows an exemplary expression vector encoding XTEN fused to the 3′ end of the sequence encoding biologically active protein Ex4. Note that no additional leader sequences are required in this vector. FIG. 7B depicts an expression vector encoding XTEN fused to the 5′ end of the sequence encoding Ex4 with a CBD leader sequence and a TEV protease site. FIG. 7C depicts an expression vector as in FIG. 7B where the CBD and TEV processing site have been replaced with an optimized N-terminal leader sequence (NTS). FIG. 7D depicts an expression vector encoding an NTS sequence, an XTEN, a sequence encoding Ex4, and than a second sequence encoding an XTEN.

FIG. 8 shows results of expression assays for the indicated constructs comprising GFP and XTEN sequences using NTS. The expression cultures were assayed using a fluorescence plate reader (excitation 395 nm, emission 510 nm) to determine the amount of GFP reporter present. The results, graphed as box and whisker plots, indicate that while median expression levels were approximately half of the expression levels compared to the “benchmark” CBD N-terminal helper domain, the best clones from the libraries were much closer to the benchmarks, indicating that further optimization around those sequences was warranted. The results also show that the libraries starting with amino acids MA had better expression levels than those beginning with ME (see Example 14).

FIG. 9 shows three randomized libraries used for the third and fourth codons in the N-terminal sequences of clones from LCW546, LCW547 and LCW552. The libraries were designed with the third and fourth residues modified such that all combinations of allowable XTEN codons were present at these positions, as shown. In order to include all the allowable XTEN codons for each library, nine pairs of oligonucleotides encoding 12 amino acids with codon diversities of third and fourth residues were designed, annealed and ligated into the NdeI/BsaI restriction enzyme digested stuffer vector pCW0551 (Stuffer-XTEN_AM875-GFP), and transformed into E. coli BL21Gold(DE3) competent cells to obtain colonies of the three libraries LCW0569 (SEQ ID NOS 2371-2372), LCW0570 (SEQ ID NOS 2373-2374), and LCW0571 (SEQ ID NOS 2375-2376).

FIG. 10 shows a histogram of a retest of the top 75 clones after the optimization step, as described in Example 15, for GFP fluorescence signal, relative to the benchmark CBD_AM875 construct. The results indicated that several clones were now superior to the benchmark clones, as seen in FIG. 10.

FIG. 11 is a schematic of a combinatorial approach undertaken for the union of codon optimization preferences for two regions of the N-terminus 48 amino acids. The approach created novel 48mers at the N-terminus of the XTEN protein for evaluation of the optimization of expression that resulted in leader sequences that may be a solution for expression of XTEN proteins where the XTEN is N-terminal to the BP.

FIG. 12 shows an SDS-PAGE gel confirming expression of preferred clones obtained from the XTEN N-terminal codon optimization experiments, in comparison to benchmark XTEN clones comprising CBD leader sequences at the N-terminus of the construct sequences.

FIG. 13 shows an SDS-PAGE gel of samples from a stability study of the fusion protein of XTEN_AE864 fused to the N-terminus of GFP (see Example 21). The GFP-XTEN was incubated in cynomolgus plasma and rat kidney lysate for up to 7 days at 37° C. In addition, GFP-XTEN administered to cynomolgus monkeys was also assessed. Samples were withdrawn at 0, 1 and 7 days and analyzed by SDS PAGE followed by detection using Western analysis and detection with antibodies against GFP. The results demonstrate the resistance of fusion proteins comprising XTEN to degradation due to serum proteases; a factor in the enhancement of pharmacokinetic properties of the BFXTEN fusion proteins.

FIG. 14 shows two samples of 2 and 10 mcg of final purified fusion protein of IL-1ra linked to XTEN_AE864 subjected to non-reducing SDS-PAGE, as described in Example 22. The results show that the BFXTEN component fusion protein was recovered by the process, with an approximate MW of about 160 kDa.

FIG. 15 shows the output of a representative size exclusion chromatography analysis performed, as described in Example 23. The calibration standards, shown in the dashed line, include the markers thyroglobulin (670 kDa), bovine gamma-globulin (158 kDa), chicken ovalbumin (44 kDa), equine myoglobuin (17 kDa) and vitamin B12 (1.35 kDa). The BFXTEN component fusion protein of IL-1ra linked to XTEN_AM875 is shown as the solid line. The data show that the apparent molecular weight of the BFXTEN monomeric component is significantly larger than that expected for a globular protein (as shown by comparison to the standard proteins run in the same assay), and has an apparent molecular weight significantly greater than that determined by SDS-PAGE, as shown in FIG. 15, resulting in an apparent molecular weight factor of greater than 9 (see Table 23).

FIG. 16 shows the reverse phase C18 analysis of purified IL-1ra_XTEN_AM875 The output, in absorbance versus time, demonstrates the purity of the final product fusion protein.

FIG. 17 shows the results of the IL-1 receptor binding assay, plotted as a function of IL-1ra-XTEN_AM875 or IL-1ra concentration to produce a binding isotherm. To estimate the binding affinity of each fusion protein for the IL-1 receptor, the binding data was fit to a sigmoidal dose-response curve. From the fit of the data an EC50 (the concentration of IL-1ra or IL-1ra-XTEN at which the signal is half maximal) for each construct was determined, as described in Example 23. The results show that the attachment of IL-1ra to the C-terminus of the XTEN reduces the binding affinity, compared to configuration where IL-1ra is on the N-terminus of the fusion protein. The negative control XTEN_AM875-hGH construct showed no binding under the experimental conditions.

FIG. 18 shows an SDS-PAGE of a thermal stability study comparing IL-1ra to IL-1ra linked to XTEN_AM875, as described in Example 22. Samples of IL-1ra and the IL-1ra linked to XTEN were incubated at 25° C. and 85° C. for 15 min, at which time any insoluble protein was rapidly removed by centrifugation. The soluble fraction was then analyzed by SDS-PAGE as shown in FIG. 18, and shows that only IL-1ra-XTEN remained soluble after heating, while, in contrast, recombinant IL-1ra (without XTEN as a fusion partner) was completely precipitated after heating.

FIG. 19 shows the results of an IL-1ra receptor binding assay performed on the samples shown in FIG. 19. As described in Example 22, the recombinant IL-1ra, which was fully denatured by heat treatment, retained less than 0.1% of its receptor activity following heat treatment. However, IL-1ra linked to XTEN retained approximately 40% of its receptor binding activity.

FIG. 20 shows the pharmacokinetic profile (plasma concentrations) after single subcutaneous doses of three different BPXTEN compositions of IL-1ra linked to different XTEN sequences, separately administered subcutaneously to cynomolgus monkeys, as described in Example 24.

FIG. 21 shows body weight results from a pharmacodynamic and metabolic study using a combination of two fusion proteins emulating a BCXTEN composition; i.e., glucagon linked to Y288 (Gcg-XTEN) and exendin-4 linked to AE864 (Ex4-XTEN) combination efficacy in a diet-induced obesity model in mice (see Example 25 for experimental details). The graph shows change in body weight in Diet-Induced Obese mice over the course of 28 days continuous drug administration. Values shown are the average +/−SEM of 10 animals per group (20 animals in the placebo group).

FIG. 22 shows change in fasting glucose levels from a pharmacodynamic and metabolic study using single and combinations of two fusion proteins emulating a BCXTEN composition; i.e., glucagon linked to Y288 (Gcg-XTEN) and exendin-4 linked to AE864 (Ex4-XTEN) in a diet-induced obesity model in mice (see Example 26 for experimental details). Groups are as follows: Gr. 1 Tris Vehicle; Gr. 2 Ex4-AE576, 10 mg/kg; Gr. 3 Ex4-AE576, 20 mg/kg; Gr. 4 Vehicle, 50% DMSO; Gr. 5 Exenatide, 30 μg/kg/day; Gr. 6 Exenatide, 30 uL/kg/day+Gcg-Y288 20 μg/kg; Gr. 7 Gcg-Y288, 20 mg/kg; Gr. 8 Gcg-Y288, 40 mg/kg; Gr. 9 Ex4-AE576 10 mg/kg+Gcg-Y288 20 μg/kg; Gr. 10 Gcg-Y288 40 μg/kg+Ex4-AE576 20 mg/kg. The graph shows the change in fasting blood glucose levels in Diet-Induced Obese mice over the course of 28 days continuous drug administration. Values shown are the average +/−SEM of 10 animals per group (20 animals in the placebo group).

FIG. 23 shows change in lipid levels from a pharmacodynamic and metabolic study using a combination of two fusion proteins emulating a BCXTEN composition; i.e., glucagon linked to Y288 (Gcg-XTEN) and exendin-4 linked to AE864 (Ex4-XTEN) combination efficacy in a diet-induced obesity model in mice (see Example 25 for experimental details). The graphs show the triglyceride and cholesterol levels in Diet-Induced Obese mice after 28 days continuous drug administration. Values shown are the average +/−SEM of 10 animals per group.

FIG. 24 shows the pharmacokinetic profile (plasma concentrations) in cynomolgus monkeys after single doses of different compositions of GFP linked to unstructured polypeptides of varying length, administered either subcutaneously or intravenously, as described in Example 20. The compositions were GFP-L288, GFP-L576, GPF-XTEN_AF576, GFP-Y576 and XTEN_AD836-GFP. Blood samples were analyzed at various times after injection and the concentration of GFP in plasma was measured by ELISA using a polyclonal antibody against GFP for capture and a biotinylated preparation of the same polyclonal antibody for detection. Results are presented as the plasma concentration versus time (h) after dosing and show, in particular, a considerable increase in half-life for the XTEN_AD836-GFP, the composition with the longest sequence length of XTEN. The construct with the shortest sequence length, the GF-L288 had the shortest half-life.

FIG. 25 shows results of a of a size exclusion chromatography analysis of glucagon-XTEN construct samples measured against protein standards of known molecular weight, with the graph output as absorbance versus retention volume, as described in Example 19. The glucagon-XTEN constructs are 1) glucagon-Y288; 2) glucagonY-144; 3) glucagon-Y72; and 4) glucagon-Y36. The results indicate an increase in apparent molecular weight with increasing length of XTEN moiety.

FIG. 26 shows the near UV circular dichroism spectrum of Ex4-XTEN_AE864, performed as described in Example 27.

FIG. 27 shows the graphic output of subsequence scores per 36-mer blocks across an AE864 XTEN, as described in Example 28.

FIG. 28 shows the graphic output of subsequence scores per 36-mer blocks across an AG864 XTEN, as described in Example 28.

### DETAILED DESCRIPTION OF THE INVENTION

Before the embodiments of the invention are described, it is to be understood that such embodiments are provided by way of example only, and that various alternatives to the embodiments of the invention described herein may be employed in practicing the invention. Numerous variations, changes, and substitutions will now occur to those skilled in the art without departing from the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

### DEFINITIONS

In the context of the present application, the following terms have the meanings ascribed to them unless specified otherwise.

As used in the specification and claims, the singular forms “a”, “an” and “the” include plural references unless the context clearly dictates otherwise. For example, the term “a cell” includes a plurality of cells, including mixtures thereof.

The terms “polypeptide”, “peptide”, and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component.

The term “amino acid” refers to either natural and/or unnatural or synthetic amino acids, including but not limited to both the D or L optical isomers, and amino acid analogs and peptidomimetics. Standard single or three letter codes are used to designate amino acids.

The term “natural L-amino acid” means the L optical isomer forms of glycine (G), proline (P), alanine (A), valine (V), leucine (L), isoleucine (I), methionine (M), cysteine (C), phenylalanine (F), tyrosine (Y), tryptophan (W), histidine (H), lysine (K), arginine (R), glutamine (Q), asparagine (N), glutamic acid (E), aspartic acid (D), serine (S), and threonine (T).

The term “non-naturally occurring,” as applied to sequences and as used herein, means polypeptide or polynucleotide sequences that do not have a counterpart to, are not complementary to, or do not have a high degree of homology with a wild-type or naturally-occurring sequence found in a mammal. As used herein, “non-naturally occurring” is not intended to distinguish recombinant sequences from wild-type sequences. For example, a non-naturally occurring polypeptide may share no more than 99%, 98%, 95%, 90%, 80%, 70%, 60%, 50% or even less amino acid sequence identity as compared to the corresponding natural sequence when suitably aligned.

The terms “hydrophilic” and “hydrophobic” refer to the degree of affinity that a substance has with water. A hydrophilic substance has a strong affinity for water, tending to dissolve in, mix with, or be wetted by water, while a hydrophobic substance substantially lacks affinity for water, tending to repel and not absorb water and tending not to dissolve in or mix with or be wetted by water. Amino acids can be characterized based on their hydrophobicity. A number of scales have been developed. An example is a scale developed by Levitt, M, et al., J Mol Biol (1976) 104:59, which is listed in Hopp, T P, et al., Proc Natl Acad Sci USA (1981) 78:3824. Examples of “hydrophilic amino acids” are arginine, lysine, threonine, alanine, asparagine, and glutamine. Of particular interest are the hydrophilic amino acids aspartate, glutamate, and serine, and glycine. Examples of “hydrophobic amino acids” are tryptophan, tyrosine, phenylalanine, methionine, leucine, isoleucine, and valine.

As applied to biologically active proteins, a “fragment” is a truncated form of a native biologically active protein that retains at least a portion of the therapeutic and/or biological activity. A “variant” is a protein with sequence homology to the native biologically active protein that retains at least a portion of the therapeutic and/or biological activities of the biologically active protein. For example, a variant protein may share at least 70%, 75%, 80%, 85%, 90%, 95%, 96%, 97%, 98% or 99% amino acid sequence identity with the reference biologically active protein. As used herein, the term “biologically active protein moiety” includes proteins modified deliberately, as for example, by site directed mutagenesis, insertions, or accidentally through mutations.

The term “sequence variant” means polypeptides that have been modified compared to their native or original sequence by one or more amino acid insertions, deletions, or substitutions. Insertions may be located at either or both termini of the protein, and/or may be positioned within internal regions of the amino acid sequence. A non-limiting example would be insertion of an XTEN sequence within the sequence of the biologically-active payload protein. In deletion variants, one or more amino acid residues in a polypeptide as described herein are removed. Deletion variants, therefore, include all fragments of a payload polypeptide sequence. In substitution variants, one or more amino acid residues of a polypeptide are removed and replaced with alternative residues. In one aspect, the substitutions are conservative in nature and conservative substitutions of this type are well known in the art.

As used herein, “internal XTEN” refers to XTEN sequences that have been inserted into the sequence of the biologically active protein. Internal XTENs can be constructed by insertion of an XTEN sequence into the sequence of a biologically active protein, either by insertion between two adjacent amino acids or wherein XTEN replaces a partial, internal sequence of the biologically active protein.

As used herein, “terminal XTEN” refers to XTEN sequences that have been fused to or in the N- or C-terminus of the biologically active protein or to a proteolytic cleavage sequence at the N- or C-terminus of the biologically active protein. Terminal XTENs can be fused to the native termini of the biologically active protein. Alternatively, terminal XTENs can replace a terminal sequence of the biologically active protein.

The term “XTEN release site” refers to a cleavage sequence in fusion proteins that can be recognized and cleaved by a mammalian protease, effecting release of an XTEN or a portion of an XTEN from the fusion protein. As used herein, “mammalian protease” means a protease that normally exists in the body fluids, cells or tissues of a mammal. XTEN release sites can be engineered to be cleaved by various mammalian proteases (a.k.a. “XTEN release proteases”) such as FXIa, FXIIa, kallikrein, FVIIIa, FVIIIa, FXa, FIIa (thrombin), Elastase-2, MMP-12, MMP13, MMP-17, MMP-20, or any protease that is present during a clotting event.

A “host cell” includes an individual cell or cell culture which can be or has been a recipient for the subject vectors. Host cells include progeny of a single host cell. The progeny may not necessarily be completely identical (in morphology or in genomic of total DNA complement) to the original parent cell due to natural, accidental, or deliberate mutation. A host cell includes cells transfected in vivo with a vector of this invention.

As used herein, an “antibody” refers to a protein consisting of one or more polypeptides substantially encoded by immunoglobulin genes or fragments of immunoglobulin genes, and includes full-length dimeric antibodies or antibody fragments capable of binding a target antigen of interest. Antibody fragments include CDR regions, single chain antibody molecules (scFv), Fd, and domain antibodies (dAb), The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A “host cell” includes an individual cell or cell culture which can be or has been a recipient for the subject vectors. Host cells include progeny of a single host cell. The progeny may not necessarily be completely identical (in morphology or in genomic of total DNA complement) to the original parent cell due to natural, accidental, or deliberate mutation. A host cell includes cells transfected in vivo with a vector of this invention.

“Isolated,” when used to describe the various polypeptides or fusion proteins disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is generally greater than that of its naturally occurring counterpart. In general, a polypeptide made by recombinant means and expressed in a host cell is considered to be “isolated.”

An “isolated” polynucleotide or polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal or extra-chromosomal location different from that of natural cells.

A “chimeric” protein contains at least one fusion polypeptide comprising regions in a different position in the sequence than that which occurs in nature. The regions may normally exist in separate proteins and are brought together in the fusion polypeptide; or they may normally exist in the same protein but are placed in a new arrangement in the fusion polypeptide. A chimeric protein may be created, for example, by chemical synthesis, or by creating and translating a polynucleotide in which the peptide regions are encoded in the desired relationship.

“Conjugated”, “linked,” “fused,” and “fusion” are used interchangeably herein. These terms refer to the joining together of two more chemical elements or components, by whatever means including chemical conjugation or recombinant means. For example, a promoter or enhancer is operably linked to a coding sequence if it affects the transcription of the sequence. Generally, “operably linked” means that the DNA sequences being linked are contiguous, and in reading phase or in-frame. An “in-frame fusion” refers to the joining of two or more open reading frames (ORFs) to form a continuous longer ORF, in a manner that maintains the correct reading frame of the original ORFs. Thus, the resulting recombinant fusion protein is a single protein containing two ore more segments that correspond to polypeptides encoded by the original ORFs (which segments are not normally so joined in nature).

In the context of polypeptides, a “linear sequence” or a “sequence” is an order of amino acids in a polypeptide in an amino to carboxyl terminus direction in which residues that neighbor each other in the sequence are contiguous in the primary structure of the polypeptide. A “partial sequence” is a linear sequence of part of a polypeptide which is known to comprise additional residues in one or both directions.

“Heterologous” means derived from a genotypically distinct entity from the rest of the entity to which it is being compared. For example, a glycine rich sequence removed from its native coding sequence and operatively linked to a coding sequence other than the native sequence is a heterologous glycine rich sequence. The term “heterologous” as applied to a polynucleotide, a polypeptide, means that the polynucleotide or polypeptide is derived from a genotypically distinct entity from that of the rest of the entity to which it is being compared.

The terms “polynucleotides”, “nucleic acids”, “nucleotides” and “oligonucleotides” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “complement of a polynucleotide” denotes a polynucleotide molecule having a complementary base sequence and reverse orientation as compared to a reference sequence, such that it could hybridize with a reference sequence with complete fidelity.

“Recombinant” as applied to a polynucleotide means that the polynucleotide is the product of various combinations of in vitro cloning, restriction and/or ligation steps, and other procedures that result in a construct that can potentially be expressed in a host cell.

The terms “gene” or “gene fragment” are used interchangeably herein. They refer to a polynucleotide containing at least one open reading frame that is capable of encoding a particular protein after being transcribed and translated. A gene or gene fragment may be genomic or cDNA, as long as the polynucleotide contains at least one open reading frame, which may cover the entire coding region or a segment thereof. A “fusion gene” is a gene composed of at least two heterologous polynucleotides that are linked together.

“Homology” or “homologous” refers to sequence similarity or interchangeability between two or more polynucleotide sequences or two or more polypeptide sequences. When using a program such as BestFit to determine sequence identity, similarity or homology between two different amino acid sequences, the default settings may be used, or an appropriate scoring matrix, such as blosum45 or blosum80, may be selected to optimize identity, similarity or homology scores. Preferably, polynucleotides that are homologous are those which hybridize under stringent conditions as defined herein and have at least 70%, preferably at least 80%, more preferably at least 90%, more preferably 95%, more preferably 97%, more preferably 98%, and even more preferably 99% sequence identity to those sequences.

“Ligation” refers to the process of forming phosphodiester bonds between two nucleic acid fragments or genes, linking them together. To ligate the DNA fragments or genes together, the ends of the DNA must be compatible with each other. In some cases, the ends will be directly compatible after endonuclease digestion. However, it may be necessary to first convert the staggered ends commonly produced after endonuclease digestion to blunt ends to make them compatible for ligation.

The terms “stringent conditions” or “stringent hybridization conditions” includes reference to conditions under which a polynucleotide will hybridize to its target sequence, to a detectably greater degree than other sequences (e.g., at least 2-fold over background). Generally, stringency of hybridization is expressed, in part, with reference to the temperature and salt concentration under which the wash step is carried out. Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short polynucleotides (e.g., 10 to 50 nucleotides) and at least about 60° C. for long polynucleotides (e.g., greater than 50 nucleotides)—for example, “stringent conditions” can include hybridization in 50% formamide, 1 M NaCl, 1% SDS at 37° C., and three washes for 15 min each in 0.1×SSC/1% SDS at 60° C. to 65° C. Alternatively, temperatures of about 65° C., 60° C., 55° C., or 42° C. may be used. SSC concentration may be varied from about 0.1 to 2×SSC, with SDS being present at about 0.1%. Such wash temperatures are typically selected to be about 5° C. to 20° C. lower than the thermal melting point for the specific sequence at a defined ionic strength and pH. The Tm is the temperature (under defined ionic strength and pH) at which 50% of the target sequence hybridizes to a perfectly matched probe. An equation for calculating Tm and conditions for nucleic acid hybridization are well known and can be found in Sambrook, J. et al., “Molecular Cloning: A Laboratory Manual,” 3rd edition, Cold Spring Harbor Laboratory Press, 2001. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity may be measured over the length of an entire defined polynucleotide sequence, or may be measured over a shorter length, for example, over the length of a fragment taken from a larger, defined polynucleotide sequence, for instance, a fragment of at least 45, at least 60, at least 90, at least 120, at least 150, at least 210 or at least 450 contiguous residues. Such lengths are exemplary only, and it is understood that any fragment length supported by the sequences shown herein, in the tables, figures or Sequence Listing, may be used to describe a length over which percentage identity may be measured.

“Percent (%) amino acid sequence identity,” with respect to the polypeptide sequences identified herein, is defined as the percentage of amino acid residues in a query sequence that are identical with the amino acid residues of a second, reference polypeptide sequence or a portion thereof, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The term “non-repetitiveness” as used herein in the context of a polypeptide refers to a lack or limited degree of internal homology in a peptide or polypeptide sequence. The term “substantially non-repetitive” can mean, for example, that there are few or no instances of four contiguous amino acids in the sequence that are identical amino acid types or that the polypeptide has a subsequence score (defined infra) of 3 or less or that there isn't a pattern in the order, from N- to C-terminus, of the sequence motifs that constitute the polypeptide sequence. The term “repetitiveness” as used herein in the context of a polypeptide refers to the degree of internal homology in a peptide or polypeptide sequence. In contrast, a “repetitive” sequence may contain multiple identical copies of short amino acid sequences. For instance, a polypeptide sequence of interest may be divided into n-mer sequences and the number of identical sequences can be counted over the length of the polypeptide or averaged over shorter lengths called “blocks.” Highly repetitive sequences contain a large fraction of identical sequences while non-repetitive sequences contain few identical sequences. In the context of a polypeptide, a sequence can contain multiple copies of shorter sequences of defined or variable length, or motifs, in which the motifs themselves have non-repetitive sequences, rendering the full-length polypeptide substantially non-repetitive. “Repetitiveness” used in the context of polynucleotide sequences refers to the degree of internal homology in the sequence such as, for example, the frequency of identical nucleotide sequences of a given length. Repetitiveness can, for example, be measured by analyzing the frequency of identical sequences.

A “vector” is a nucleic acid molecule, preferably self-replicating in an appropriate host, which transfers an inserted nucleic acid molecule into and/or between host cells. The term includes vectors that function primarily for insertion of DNA or RNA into a cell, replication of vectors that function primarily for the replication of DNA or RNA, and expression vectors that function for transcription and/or translation of the DNA or RNA. Also included are vectors that provide more than one of the above functions. An “expression vector” is a polynucleotide which, when introduced into an appropriate host cell, can be transcribed and translated into a polypeptide(s). An “expression system” usually connotes a suitable host cell comprised of an expression vector that can function to yield a desired expression product.

“Serum degradation resistance,” as applied to a polypeptide, refers to the ability of the polypeptides to withstand degradation in blood or components thereof, which typically involves proteases in the serum or plasma. The serum degradation resistance can be measured by combining the protein with human (or mouse, rat, monkey, as appropriate) serum or plasma, typically for a range of days (e.g. 0.25, 0.5, 1, 2, 4, 8, 16 days), typically at about 37° C. The samples for these time points can be run on a Western blot assay and the protein is detected with an antibody. The antibody can be to a tag in the protein. If the protein shows a single band on the western, where the protein's size is identical to that of the injected protein, then no degradation has occurred. In this exemplary method, the time point where 50% of the protein is degraded, as judged by Western blots or equivalent techniques, is the serum degradation half-life or “serum half-life” of the protein.

The term “t_{1/2}” as used herein means the terminal half-life calculated as ln(2)/Kₑ₁. Kₑ₁ is the terminal elimination rate constant calculated by linear regression of the terminal linear portion of the log concentration vs. time curve. Half-life typically refers to the time required for half the quantity of an administered substance deposited in a living organism to be metabolized or eliminated by normal biological processes. The terms “t_{1/2}”, “terminal half-life”, “elimination half-life” and “circulating half-life” are used interchangeably herein.

“Active clearance” means the mechanisms by which biologically active protein is removed from the circulation other than by filtration or coagulation, and which includes removal from the circulation mediated by cells, receptors, metabolism, or degradation of the biologically active protein.

“Apparent molecular weight factor” and “apparent molecular weight” are related terms referring to a measure of the relative increase or decrease in apparent molecular weight exhibited by a particular amino acid sequence. The apparent molecular weight is determined using size exclusion chromatography (SEC) and similar methods compared to globular protein standards and is measured in “apparent kD” units. The apparent molecular weight factor is the ratio between the apparent molecular weight and the actual molecular weight; the latter predicted by adding, based on amino acid composition, the calculated molecular weight of each type of amino acid in the composition or by estimation from comparison to molecular weight standards in an SDS electrophoresis gel.

The terms “hydrodynamic radius” or “Stokes radius” is the effective radius (Rh in nm) of a molecule in a solution measured by assuming that it is a body moving through the solution and resisted by the solution's viscosity. In the embodiments of the invention, the hydrodynamic radius measurements of the XTEN fusion proteins correlate with the ‘apparent molecular weight factor’, which is a more intuitive measure. The “hydrodynamic radius” of a protein affects its rate of diffusion in aqueous solution as well as its ability to migrate in gels of macromolecules. The hydrodynamic radius of a protein is determined by its molecular weight as well as by its structure, including shape and compactness. Methods for determining the hydrodynamic radius are well known in the art, such as by the use of size exclusion chromatography (SEC), as described in U.S. Pat. Nos. 6,406,632 and 7,294,513. Most proteins have globular structure, which is the most compact three-dimensional structure a protein can have with the smallest hydrodynamic radius. Some proteins adopt a random and open, unstructured, or ‘linear’ conformation and as a result have a much larger hydrodynamic radius compared to typical globular proteins of similar molecular weight.

“Physiological conditions” refer to a set of conditions in a living host as well as in vitro conditions, including temperature, salt concentration, pH, that mimic those conditions of a living subject. A host of physiologically relevant conditions for use in in vitro assays have been established. Generally, a physiological buffer contains a physiological concentration of salt and is adjusted to a neutral pH ranging from about 6.5 to about 7.8, and preferably from about 7.0 to about 7.5. A variety of physiological buffers is listed in Sambrook et al. (1989). Physiologically relevant temperature ranges from about 25° C. to about 38° C., and preferably from about 35° C. to about 37° C.

A “reactive group” is a chemical structure that can be coupled to a second reactive group. Examples for reactive groups are amino groups, carboxyl groups, sulfhydryl groups, hydroxyl groups, aldehyde groups, azide groups. Some reactive groups can be activated to facilitate coupling with a second reactive group. Examples for activation are the reaction of a carboxyl group with carbodiimide, the conversion of a carboxyl group into an activated ester, or the conversion of a carboxyl group into an azide function.

“Controlled release agent”, “slow release agent”, “depot formulation” or “sustained release agent” are used interchangeably to refer to an agent capable of extending the duration of release of a polypeptide of the invention relative to the duration of release when the polypeptide is administered in the absence of agent. Different embodiments of the present invention may have different release rates, resulting in different therapeutic amounts.

The terms “antigen”, “target antigen” or “immunogen” are used interchangeably herein to refer to the structure or binding determinant that an antibody fragment or an antibody fragment-based therapeutic binds to or has specificity against.

The term “payload” as used herein refers to a protein or peptide sequence that has biological or therapeutic activity; the counterpart to the pharmacophore of small molecules. Examples of payloads include, but are not limited to, cytokines, enzymes, hormones and blood and growth factors. Payloads can further comprise genetically fused or chemically conjugated moieties such as chemotherapeutic agents, antiviral compounds, toxins, or contrast agents. These conjugated moieties can be joined to the rest of the polypeptide via a linker which may be cleavable or non-cleavable.

The term “antagonist”, as used herein, includes any molecule that partially or fully blocks, inhibits, or neutralizes a biological activity of a native polypeptide disclosed herein. Methods for identifying antagonists of a polypeptide may comprise contacting a native polypeptide with a candidate antagonist molecule and measuring a detectable change in one or more biological activities normally associated with the native polypeptide. In the context of the present invention, antagonists may include proteins, nucleic acids, carbohydrates, antibodies or any other molecules that decrease the effect of a biologically active protein.

The term “agonist” is used in the broadest sense and includes any molecule that mimics a biological activity of a native polypeptide disclosed herein. Suitable agonist molecules specifically include agonist antibodies or antibody fragments, fragments or amino acid sequence variants of native polypeptides, peptides, small organic molecules, etc. Methods for identifying agonists of a native polypeptide may comprise contacting a native polypeptide with a candidate agonist molecule and measuring a detectable change in one or more biological activities normally associated with the native polypeptide.

“Activity” for the purposes herein refers to an action or effect of a component of a fusion protein consistent with that of the corresponding native biologically active protein, wherein “biological” activity” refers to an in vitro or in vivo biological function or effect, including but not limited to receptor binding, agonist activity, or a cellular or physiologic response.

As used herein, “treat” or “treating,” or “palliating” or “ameliorating” are used interchangeably and mean administering a drug or a biologic to achieve a therapeutic benefit, to cure or reduce the severity of an existing disease, disorder or condition, or to achieve a prophylactic benefit, prevent or reduce the likelihood of onset or severity the occurrence of a disease, disorder or condition. By therapeutic benefit is meant eradication or amelioration of the underlying disorder being treated or one or more of the physiological symptoms associated with the underlying disorder such that an improvement is observed in the subject, notwithstanding that the subject may still be afflicted with the underlying disorder.

A “therapeutic effect” or “therapeutic benefit,” as used herein, refers to a physiologic effect, including but not limited to the cure, mitigation, amelioration, or prevention of disease in humans or other animals, or to otherwise enhance physical or mental wellbeing of humans or animals, caused by a fusion polypeptide of the invention other than the ability to induce the production of an antibody against an antigenic epitope possessed by the biologically active protein. For prophylactic benefit, the compositions may be administered to a subject at risk of developing a particular disease, or to a subject reporting one or more of the physiological symptoms of a disease, even though a diagnosis of this disease may not have been made.

The terms “therapeutically effective amount” and “therapeutically effective dose”, as used herein, refer to an amount of a drug or a biologically active protein, either alone or as a part of a fusion protein composition, that is capable of having any detectable, beneficial effect on any symptom, aspect, measured parameter or characteristics of a disease state or condition when administered in one or repeated doses to a subject. Such effect need not be absolute to be beneficial. Determination of a therapeutically effective amount is well within the capability of those skilled in the art, especially in light of the detailed disclosure provided herein.

The term “therapeutically effective dose regimen”, as used herein, refers to a schedule for consecutively administered multiple doses (i.e., at least two or more) of a biologically active protein, either alone or as a part of a fusion protein composition, wherein the doses are given in therapeutically effective amounts to result in sustained beneficial effect on any symptom, aspect, measured parameter or characteristics of a disease state or condition.

The terms “prevention”, “prevent”, “preventing”, “suppression”, “suppress”, “suppressing”, “inhibit” and “inhibition” as used herein refer to a course of action, including administering a compound or composition initiated in a manner (e.g., prior to the onset of a clinical symptom of a disease state or condition) so as to prevent, suppress or reduce, either temporarily or permanently, the onset of a clinical manifestation or physiologic parameter of the disease state or condition. Such preventing, suppressing or reducing need not be absolute to be useful.

### I). General Techniques

The practice of the present invention employs, unless otherwise indicated, conventional techniques of immunology, biochemistry, chemistry, molecular biology, microbiology, cell biology, genomics and recombinant DNA, which are within the skill of the art. See Sambrook, J. et al., “Molecular Cloning: A Laboratory Manual,” 3^{rd} edition, Cold Spring Harbor Laboratory Press, 2001; “Current protocols in molecular biology”, F. M. Ausubel, et al. eds., 1987; the series “Methods in Enzymology,” Academic Press, San Diego, Calif.; “PCR 2: a practical approach”, M. J. MacPherson, B. D. Hames and G. R. Taylor eds., Oxford University Press, 1995; “Antibodies, a laboratory manual” Harlow, E. and Lane, D. eds., Cold Spring Harbor Laboratory, 1988; “Goodman & Gilman's The Pharmacological Basis of Therapeutics,” 11^{th} Edition, McGraw-Hill, 2005; and Freshney, R. I., “Culture of Animal Cells: A Manual of Basic Technique,” 4^{th} edition, John Wiley & Sons, Somerset, N.J., 2000, the contents of which are incorporated in their entirety herein by reference.

### II). Bifunctional Fusion Protein Compositions

The present invention relates in part to fusion protein compositions and methods of use of fusion proteins for treatment or prevention of metabolic and/or cardiovascular diseases, disorders or conditions.

In one aspect, the invention provides combinations of a first biologically active protein (hereinafter “BP”) and a second BP covalently linked to one or more extended recombinant polypeptides (hereinafter “XTEN), resulting in a chimeric bifunctional monomeric XTEN fusion protein composition (hereinafter “BMXTEN”). In another aspect, the invention provides fixed compositions of at least two individual fusion proteins, each with a different payload BP linked to one or more XTEN, resulting in a chimeric bifunctional combination XTEN fusion protein composition (hereinafter “BCXTEN”). Collectively, the BMXTEN and BCXTEN are bifunctional XTEN fusion proteins and it is intended that the term “BFXTEN” encompass both forms unless specifically indicated otherwise. Thus, BFXTEN are chimeric polypeptides that comprise one or two payload regions, each comprising a biologically active protein (BP) that mediates one or more biological or therapeutic activities and at least one other region comprising an XTEN polypeptide that is not a biologically active protein and that has an extended, non-repetitive, non-naturally occurring sequence with unstructured characteristics, amongst other properties as described herein.

(a) Biologically Active Proteins (BP)

The bifunctional BFXTEN compositions of the invention comprise a first BP and a second BP that is not identical to the first BP. The BP for inclusion in the bifunctional BFXTEN of the invention can include any protein of biologic, therapeutic, or prophylactic interest or function that is useful for preventing, treating, mediating, or ameliorating a metabolic and/or cardiovascular disease, disorder or condition or can prolong the survival of the subject being treated. In one embodiment, the BP incorporated into the subject compositions can be a recombinant polypeptide with a sequence corresponding to a protein found in nature. In another embodiment, the BP can be sequence variants, fragments, homologs, and mimetics of a natural sequence that retain at least a portion of the biological activity of the native BP. It is specifically contemplated that the term “biologically active protein” or “BP” encompasses antibodies and fragments and variants thereof. Table 1 provides a non-limiting list of biologically active proteins that are encompassed by the BFXTEN fusion proteins of the invention. In one embodiment, a BFXTEN fusion protein comprises a first biologically active protein that exhibits at least about 80% sequence identity, or alternatively 81%, 82%, 83%, 84%, 85%, 86%, 87%, 88%, 89%, 90%, 91%, 92%, 93%, 94%, 95%, 96%, 97%, 98% or 99% sequence identity to a protein sequence selected from Table 1, linked to an XTEN (as described more fully below). In another embodiment, the BFXTEN comprises the first biologically active protein of the foregoing embodiment and a second biologically active protein that exhibits at least about 80% sequence identity, or alternatively 81%, 82%, 83%, 84%, 85%, 86%, 87%, 88%, 89%, 90%, 91%, 92%, 93%, 94%, 95%, 96%, 97%, 98% or 99% sequence identity to a protein sequence selected from Table 1, wherein the second biologically active protein is different from the first biologically active protein, resulting in a monomeric fusion protein comprising the two BP linked to one or more XTEN (as described more fully below). In another embodiment, a BFTXEN composition comprises two fusion proteins; a first fusion protein comprising a first BP linked to at least a first XTEN and a second fusion protein comprising a second BP different from the first BP linked to an XTEN that may be identical or may be different from the first XTEN.

In general, BP will exhibit a binding specificity to a given target or another desired biological characteristic when used in vivo or when utilized in an in vitro assay. For example, the BP can be an agonist, a receptor, a ligand, an antagonist, a hormone, or an antibody or antibody fragment. Of particular interest are BP used or known to be useful for a metabolic and/or cardiovascular disease or disorder wherein the native BP have a relatively short terminal half-life and for which an enhancement of a pharmacokinetic parameter or the combination with a second BP would permit less frequent dosing or an enhanced pharmacologic effect. Also of interest are BP that have a narrow therapeutic window between the minimum effective dose or blood concentration (Cₘᵢₙ) and the maximum tolerated dose or blood concentration (Cₘₐₓ).

In another embodiment, the invention provides bifunctional BFXTEN compositions wherein one BP can be an antigen binding moiety, such as an antibody or antibody fragment. Many forms of antibody fragments are known in the art and encompassed herein. Antibody fragments comprise only a portion of an intact antibody, generally including at least a portion of an antigen binding site of the intact antibody that retains the ability to bind antigen. Examples of monomeric antibody fragments encompassed by the present definition include: (i) isolated CDR regions, with or without framework regions; (ii) single chain antibody molecules (scFv) comprising the V_{H} and V_{L} domains of an antibody wherein these domains are present in a single polypeptide chain (Bird et al., Science 242:423-426 (1988), and Huston et al., PNAS (USA) 85:5879-5883 (1988)); (iii) Fd (a fragment consisting of the VH and CH1 domains); and (iv) domain antibodies (dAb), consisting of a V_{H} or V_{L} domain (as described in WO 2007/087673). The methods to make such antibody fragments are well-known in the art, and antigen-binding sequences can be derived from natural or synthetic sources. A library of V_{H} and V_{L} region domains to be screened for binding activity can be a naturally occurring repertoire of immunoglobulin sequences or a synthetic repertoire. A naturally occurring repertoire can be prepared, for example, from immunoglobulin-expressing cells harvested from a mammalian source. Synthetic repertoires of single immunoglobulin variable domains can be prepared by artificially introducing sequence diversity into a cloned variable domain. A library repertoire of V_{H} and V_{L} domains can be screened for desired binding specificity to a specific target by, for example, phage display. Methods for the construction of bacteriophage display libraries and lambda phage expression libraries are well known in the art. In one embodiment, the antigen-binding moiety can have the binding portions of the variable regions of an antibody tight chain and the binding portion of the variable region of an antibody heavy chain. In another embodiment, the antigen-binding moiety can have the binding portions of a first a second variable region of antibody light chains. In another embodiment, the antigen-binding moiety can have the binding portions of the variable region of a first and a second antibody heavy chain. In another embodiment, the antigen-binding moiety is a multimer of antigen-binding fragments, each linked by intervening XTEN sequences of 100-300 amino acid residues. In the foregoing embodiments hereinabove described in this paragraph, the antigen-binding moiety of the BFXTEN composition can be a pharmacologic effector moiety wherein the binding results in an agonist, antagonist, or immune clearance effect, or can be a targeting moiety Wherein the second BP of the BFXTEN composition can be a therapeutic protein, and wherein the targeting by the antigen-binding moiety results in enhanced delivery of the therapeutic protein component of the BFXTEN to a target cell, tissue or organ. In one embodiment, the BFXTEN comprises a BP wherein the BP is a targeting moiety with binding affinity to a cell surface receptor. In one embodiment, the antigen-binding moiety of the BFXTEN fusion protein binds CD3, such as, but not limited to, an anti-CD3 antibody or binding fragment(s) as described in U.S. Pat. Nos. 5,885,573 and 6,491,916.

TABLE 1
Biologically active proteins and corresponding amino acid sequences

| Name of Protein | | SEQ ID |
|---|---|---|
| (Synonym) | Sequence | NO: |
| adrenomedullin | YRQSMNNFQGLRSFGCRFGTCTVQKLAHQIYQFTDKDKDNVAPRSKISPQ | 1 |
| (ADM) | GY | |
| Amylin, rat | KCNTATCATQRLANFLVRSSNNLGPVLPPTNVGSNTY | 2 |
| Amylin, human | KCNTATCATQRLANFLVHSSNNFGAILSSTNVGSNTY | 3 |
| Anti-CD3 | See U.S. Pat Nos. 5,885,573 and 6,491,916 | |
| IL1ra (kineret) | MRPSGRKSSKMQAFRIWDVNQKTFYLRNNQLVAGYLQGPNVNLEEKIDV | 4 |
| | VPIEPHALFLGIHGGKMCLSCVKSGDETRLQLEAVNITDLSENRKQDKRFA | |
| | FIRSDSGPTTSFESAACPGWFLCTAMEADQPVSLTNMPDEGVMVTKFYFQ | |
| | EDE | |
| Calcitonin (hCT) | CGNLSTCMLGTYTQDFNKFHTFPQTAIGVGAP | 5 |
| Calcitonin, salmon | CSNLSTCVLGKLSQELHKLQTYPRTNTGSTP | 6 |
| calcitonin gene related | ACDTATCVTHRLAGLLSRSGGVVKNMVPTNVGSKAF | 7 |
| peptide (h-CGRP α) | | |
| calcitonin gene related | ACNTATCVTHRLAGLLSRSGGMVKSNFVPTNVGSKAF | 8 |
| peptide (h-CGRP β) | | |
| FGF-19 | MRSGCVVVHVWILAGLWLAVAGRPLAFSDAGPHVHYGWGDPIRLRHLY | 9 |
| | TSGPHGLSSCFLRIRADGVVDCARGQSAHSLLEIKAVALRTVAIKGVHSVR | |
| | YLCMGADGKMQGLLQYSEEDCAFEEEIRPDGYNVYRSEKHRLPVSLSSA | |
| | KQRQLYKNRGFLPLSHFLPMLPMVPEEPEDLRGHLESDMFSSPLETDSMD | |
| | PFGLVTGLEAVRSPSFEK | |
| FGF-21 | MDSDETGFEHSGLWVSVLAGLLLGACQAHPIPDSSPLLQFGGQVRQRYLY | 10 |
| | TDDAQQTEAHLEIREDGTVGGAADQSPESLLQLKALKPGVIQILGVKTSRF | |
| | LCQRPDGALYGSLHFDPEACSFRELLLEDGYNVYQSEAHGLPLHLPGNKS | |
| | PHRDPAPRGPARFLPLPGLPPALPEPPGILAPQPPDVGSSDPLSMVGPSQGR | |
| | SPSYAS | |
| Gastrin | QLGPQGPPHLVADPSKKQGPWLEEEEEAYGWMDF | 11 |
| Gastric inhibitory | YAEGTFISDYSIAMDKIHQ QDFVNWLLAQKGKKNDWKHNITQ | 12 |
| polypeptide (GIP) | | |
| Ghrelin | GSSFLSPEHQR VQQRKESKKPPAKLQPR | 13 |
| IGF-1 | GPETLCGAELVDALQFVCGDRGFYFNKPTGYGSSSRRAPQTG | 14 |
| | IVDECCFRSCDLRRLEMYCAPLKPAKSA | |
| IGF-2 | AYRPSETLCGGELVDTLQFVCGDRGFYFSRPASRVSRRSRGIVEECCFRSC | 15 |
| | DLALLETYCATPAKSE | |
| INGAP peptide | EESQKKLPSSRITCPQGSVAYGSYCYSLILIPQTWSNAELSCQMHFSGHLAF | 16 |
| (islet neogenesis- | LLSTGEITFVSSLVKNSLTAYQYIWIGLHDPSHGTLPNGSGWKWSSSNVLT | |
| associated protein) | FYNWERNPSIAADRGYCAVLSQKSGFQKWRDFNCENELPYICKFKV | |
| Pramlintide | KCNTATCATNRLANFLVHSSNNFGPILPPTNVGSNTY-H2 | 17 |
| α-natriuretic peptide | SLRRSSCFGGRMDRIGAQSGLGCNSFRY | 18 |
| (ANP) | | |
| β-natriuretic peptide, | SPKMVQGSGGFGRKMDRISSSSGLGCKVLRRH | 19 |
| human (BNP human) | | |
| Brain natriuretic | NSKMAHSSSCFGQKIDRIGAVSRLGCDGLRLF | 20 |
| peptide, Rat: | | |
| (BNP Rat) | | |
| C-type natriuretic | GLSKGCFGLKLDRIGSMSGLGC | 21 |
| peptide (CNP porcine) | | |
| cholecystokinin | MNSGVCLCVLMAVLAAGALTQPVPPADPAGSGLQRAEEAPRRQLRVSQR | 22 |
| (CCK) | TDGESRAHLGALLARYIQQARKAPSGRMSIVKNLQNLDPSHRISDRDYMG | |
| | WMDFGRRSAEEYEYPS | |
| CCK-58 | VSQRTDGESRAHLGALLARYIQQARKAPSGRMSIVKNLQNLDPSHRISDR | 23 |
| | DYMGWMDF | |
| CCK-33 | KAPSGRMSIVKNLQNLDPSHRISDRDYMGWMDF | 24 |
| CCK-8 | DYMGWMDF | 25 |
| CCK-7 | YMGWMDF | 26 |
| CCK-8- | DY(SO3)MGWMDF | 27 |
| Sulfated | | |
| CCK-5 | GWMDF | 28 |
| Exendin-3 | HSDGTFTSDLSKQMEEEAVRLFIEWLKNGGPSSGAPPPS | 29 |
| Exendin-4 | HGEGTFTSDLSKQMEEEAVR LFIEWLKNGGPSSGAPPPS | 30 |
| Gastrin-17 | DPSKKQGPWLEEEEEAYGWMDF | 31 |
| Glucagon | HSQGTFTSDYSKYLDSRRAQDFVQWLMNT | 32 |
| Glucagon-like peptide- | HDEFERHAEGTFTSDVSSTLEGQAALEFIAWLVKGRG | 33 |
| 1 (hGLP-1) (GLP-1; | | |
| 1-37) | | |
| h-GLP-1 (7-36) | HAEGTFTSDVSSYLEGQAALEFIAWLVKGR | 34 |
| h-GLP-1 (7-37) | HAEGTFTSDVSSTLEGQAALEFIAWLVKGRG | 35 |
| GLP-1, frog | HAEGTYTNDVTEYLEEKAAKEFIEWLIKGKPKKIRYS | 36 |
| glucagon-like peptide | HADGSFSDEMNTILDNLAARDFINWLIETKITD | 37 |
| 2 (hGLP-2) | | |
| GLP-2, frog | HAEGTFTNDMTNYLEEKAAKEFVGWLIKGRP-OH | 38 |
| Intermedin (AFP-6) | TQAQULRVGCVLGTCQVQNLSHRLWQLMGPAGRQDSAPVDPSSPHSY | 39 |
| h-Leptin | VPIQKVQDDTKTLIKTIVTRINDISHTQSVSSKQKVTGLDFIPGLHPILTLSK | 40 |
| | MDQTLAVYQQILTSMPSRNVIQISNDLENLRDLLHVLAFSKSCHLPWASG | |
| | LETLDSLGGVLEASGYSTEVVALSRLQGSLQDMLWQLDLSPGC | |
| Neuromedin | YFLFRPRN | 41 |
| (U-8, porcine) | | |
| Neuromedin (U-9) | GYFLFRPRN | 42 |
| neuromedin (U25, | FRVDEEFQSPFASQSRGYFLFRPRN | 43 |
| human) | | |
| Neuromedin (U25, | FKVDEEFQGPIVSQNRRYFLFRPRN | 44 |
| pig) | | |
| Neuromedin S, human | ILQRGSGTAAVDFTKKDHTATWGRPFFLFRPRN | 45 |
| Neuromedin U, rat | YKVNEYQGPVAPSGGFFLFRPRN | 46 |
| oxyntomodulin OXM) | HSQGTFTSDYSKYLDSRRAQDFVQWLMNTKRNRNNIA | 47 |
| peptide YY (PYY) | YPIKPEAPGEDASPEELNRYYASLRHYLNLVTRQRY | 48 |
| urodilatin | TAPRSLRRSSCFGGRMDRIGAQSGLGCNSFRY | 49 |
| Urocortin (Ucn-1) | DNPSLSIDLTFHLLRTLLELARTQSQRERAEQNRIIFDSV | 50 |
| Urocortin (Ucn-2) | IVLSLDVPIGLLQILLEQARARAAREQATTNARILARVGHC | 51 |
| Urocortin (Ucn-3) | FTLSLDVPTNIMNLLFNIAKAKNLRAQAAANAHLMAQI | 52 |

“Adrenomedullin” or “ADM” means the human adrenomedulin peptide hormone and species and non-natural sequence variants having at least a portion of the biological activity of mature ADM. ADM is generated from a 185 amino acid preprohormone through consecutive enzymatic cleavage and amidation, resulting in a 52 amino acid bioactive peptide with a measured plasma half-life of 22 min. ADM-containing fusion proteins of the invention may find particular use in diabetes for stimulatory effects on insulin secretion from islet cells for glucose regulation or in subjects with sustained hypotension. The complete genomic infrastructure for human AM has been reported (Ishimitsu, et al., Biochem. Biophys. Res. Commun 203:631-639 (1994)), and analogs of ADM peptides have been cloned, as described in U.S. Pat. No. 6,320,022.

“Amylin” means the human peptide hormone referred to as amylin, pramlintide, species variations thereof, as described in U.S. Pat. No. 5,234,906, and non-natural sequence variants having at least a portion of the biological activity of mature amylin. Amylin is a 37-amino acid polypeptide hormone co-secreted with insulin by pancreatic beta cells in response to nutrient intake (Koda et al., Lancet 339:1179-1180. 1992), and has been reported to modulate several key pathways of carbohydrate metabolism, including incorporation of glucose into glycogen. Amylin-containing fusion proteins of the invention may find particular use in diabetes and obesity for regulating gastric emptying, suppressing glucagon secretion and food intake, thereby affecting the rate of glucose appearance in the circulation. Thus, the fusion proteins may complement the action of insulin, which regulates the at of glucose disappearance from the circulation and its uptake by peripheral tissues. Amylin analogues have been cloned, as described in U.S. Pat. Nos. 5,686,411 and 7,271,238. Amylin mimetics can be created that retain biologic activity. For example, pramlintide has the sequence KCNTATCATNRLANFLVHSSNNFGPILPPTNVGSNTY (SEQ ID NO: 53), wherein amino acids from the rat amylin sequence are substituted for amino acids in the human amylin sequence. In one embodiment, the invention contemplates fusion proteins comprising amylin mimetics of formula

KCNTATCATXRLANFLVHSSNNFGZILZZTNVGSNTY (SEQ ID NO: 54)

wherein X is independently N or Q and Z is independently S, P or G. In one embodiment, the amylin mimetic incorporated into a BFXTEN has the sequence KCNTATCATNRLANFLVHSSNNFGGILGGTNVGSNTY (SEQ ID NO: 55). In another embodiment, wherein the amylin mimetic is used at the C-terminus of the BFXTEN, the mimetic has the sequence KCNTATCATNRLANFLVHSSNNFGGILGGTNVGSNTY(NH2) (SEQ ID NO: 56)

“Anti-CD3” means the monoclonal antibody against the T cell surface protein CD3, species and sequence variants, and fragments thereof, including OKT3 (also called muromonab) and humanized anti-CD3 monoclonal antibody (hOKT31(Ala-Ala))(K C Herold et al., New England Journal of Medicine 346:1692-1698. 2002) Anti-CD3 prevents T-cell activation and proliferation by binding the T-cell receptor complex present on all differentiated T cells. Anti-CD3-containing fusion proteins of the invention may find particular use to slow new-onset Type 1 diabetes, including use of the anti-CD3 as a therapeutic effector as well as a targeting moiety for a second therapeutic BP in the BFXTEN composition. The sequences for the variable region and the creation of anti-CD3 have been described in U.S. Pat. Nos. 5,885,573 and 6,491,916.

“Calcitonin” (CT) means the human calcitonin protein, species variants thereof, including salmon calcitonin (“sCT”), and non-natural sequence variants having at least a portion of the biological activity of mature CT. CT is a 32 amino acid peptide cleaved from a larger prohormone of the thyroid that appears to function in the nervous and vascular systems, but has also been reported to be a potent hormonal mediator of the satiety reflex. CT is named for its secretion in response to induced hypercalcemia and its rapid hypocalcemic effect. It is produced in and secreted from neuroendocrine cells in the thyroid termed C cells. CT has effects on the osteoclast, and the inhibition of osteoclast functions by CT results in a decrease in bone resorption. In vitro effects of CT include the rapid loss of ruffled borders and decreased release of lysosomal enzymes. A major function of CT(1-32) is to combat acute hypercalcemia in emergency situations and/or protect the skeleton during periods of “calcium stress” such as growth, pregnancy, and lactation. (Reviewed in Becker, JCEM, 89(4): 1512-1525 (2004) and Sexton, Current Medicinal Chemistry 6: 1067-1093 (1999)). Calcitonin-containing fusion proteins of the invention may find particular use for the treatment of osteoporosis and as a therapy for Paget's disease of bone. Synthetic calcitonin peptides have been created, as described in U.S. Pat. Nos. 5,175,146 and 5,364,840.

“Calcitonin gene related peptide” or “CGRP” means the human CGRP peptide and species and non-natural sequence variants having at least a portion of the biological activity of mature CGRP. Calcitonin gene related peptide is a member of the calcitonin family of peptides, which in humans exists in two forms, α-CGRP (a 37 amino acid peptide) and β-CGRP. CGRP has 43-46% sequence identity with human amylin. CGRP-containing fusion proteins of the invention may find particular use in decreasing morbidity associated with diabetes, ameliorating hyperglycemia and insulin deficiency, inhibition of lymphocyte infiltration into the islets, and protection of beta cells against autoimmune destruction. Methods for making synthetic and recombinant CGRP are described in U.S. Pat. No. 5,374,618.

“Cholecystokinin” or “CCK” means the human CCK peptide and species and non-natural sequence variants having at least a portion of the biological activity of mature CCK. CCK-58 is the mature sequence, while the CCK-33 amino acid sequence first identified in humans is the major circulating form of the peptide. The CCK family also includes an 8-amino acid in vivo C-terminal fragment (“CCK-8”), pentagastrin or CCK-5 being the C-terminal peptide CCK(29-33), and CCK-4 being the C-terminal tetrapeptide CCK(30-33). CCK is a peptide hormone of the gastrointestinal system responsible for stimulating the digestion of fat and protein. CCK-33 and CCK-8-containing fusion proteins of the invention may find particular use in reducing the increase in circulating glucose after meal ingestion and potentiating the increase in circulating insulin. Analogues of CCK-8 have been prepared, as described in U.S. Pat. No. 5,631,230.

“Exendin-3” means a glucose regulating peptide isolated from Heloderma horridum and non-natural sequence variants having at least a portion of the biological activity of mature exendin-3. Exendin-3 amide is a specific exendin receptor antagonist from that mediates an increase in pancreatic cAMP, and release of insulin and amylase. Exendin-3-containing fusion proteins of the invention may find particular use in the treatment of diabetes and insulin resistance disorders. The sequence and methods for its assay are described in U.S. Pat. No. 5,424,286.

Exendin-4″ means a glucose regulating peptide found in the saliva of the Gila-monster Heloderma suspectum, as well as species and sequence variants thereof, and includes the native 39 amino acid sequence His-Gly-Gly-Gly-Pro-Ser-Ser-Gly-Ala-Pro-Pro-Pro-Ser (SEQ ID NO: 57) and homologous sequences and peptide mimetics, including GLP-1 and variants thereof; natural sequences, such as from primates and non-natural sequence variants having at least a portion of the biological activity of mature exendin 4. Exendin-4 is an incretin polypeptide hormone that decreases blood glucose, promotes insulin secretion, slows gastric emptying and improves satiety, providing a marked improvement in postprandial hyperglycemia. The exendins have some sequence similarity to members of the glucagon-like peptide family, with the highest identity being to GLP-1 (Goke, et al., J. Biol. Chem., 268:19650-55 (1993)). Exendin-4 binds at GLP-1 receptors on insulin-secreting βTC1 cells, and also stimulates somatostatin release and inhibits gastrin release in isolated stomachs (Goke, et al., J. Biol. Chem. 268:19650-55, 1993). As a mimetic of GLP-1, exendin-4 displays a similar broad range of biological activities, yet has a longer half-life than GLP-1, with a mean terminal half-life of 2.4 h. Exenatide is a synthetic version of exendin-4, marketed as Byetta. However, due to its short half-life, exenatide is currently dosed twice daily, limiting its utility. Exendin-4-containing fusion proteins of the invention may find particular use in the treatment of diabetes and insulin resistance disorders.

‘Fibroblast growth factor 21’, or “FGF21” means the human protein encoded by the FGF21 gene, or species and non-natural sequence variants having at least a portion of the biological activity of mature FGF21. FGF21 stimulates glucose uptake in adipocytes but not in other cell types; the effect is additive to the activity of insulin. FGF21 injection in ob/ob mice results in an increase in Glut1 in adipose tissue. FGF21 also protects animals from diet-induced obesity when over expressed in transgenic mice and lowers blood glucose and triglyceride levels when administered to diabetic rodents (Kharitonenkov A, et al., (2005). “FGF-21 as a novel metabolic regulator”. J. Clin. Invest. 115: 1627-35). FGF21-containing fusion proteins of the invention may find particular use in treatment of diabetes, including causing increased energy expenditure, fat utilization and lipid excretion. FGF21 has been cloned, as disclosed in U.S. Pat. No. 6,716,626.

“FGF-19”, or “fibroblast growth factor 19” means the human protein encoded by the FGF19 gene, or species and non-natural sequence variants having at least a portion of the biological activity of mature FGF-19. FGF-19 is a protein member of the fibroblast growth factor (FGF) family. FGF family members possess broad mitogenic and cell survival activities, and are involved in a variety of biological processes. FGF-19 increases liver expression of the leptin receptor, metabolic rate, stimulates glucose uptake in adipocytes, and leads to loss of weight in an obese mouse model (Fu, L, et al. FGF-19-containing fusion proteins of the invention may find particular use in increasing metabolic rate and reversal of dietary and leptin-deficient diabetes. FGF-19 has been cloned and expressed, as described in US Patent Application No. 20020042367.

“Gastrin” means the human gastrin peptide, truncated versions, and species and non-natural sequence variants having at least a portion of the biological activity of mature gastrin. Gastrin is a linear peptide hormone produced by G cells of the duodenum and in the pyloric antrum of the stomach and is secreted into the bloodstream. Gastrin is found primarily in three forms: gastrin-34 (“big gastrin”); gastrin-17 (“little gastrin”); and gastrin-14 (“minigastrin”). It shares sequence homology with CCK. Gastrin-containing fusion proteins of the invention may find particular use in the treatment of obesity and diabetes for glucose regulation. Gastrin has been synthesized, as described in U.S. Pat. No. 5,843,446.

“Ghrelin” means the human hormone that induces satiation, or species and non-natural sequence variants having at least a portion of the biological activity of mature ghrelin, including the native, processed 27 or 28 amino acid sequence and homologous sequences. Ghrelin is produced mainly by P/D1 cells lining the fundus of the human stomach and epsilon cells of the pancreas that stimulates hunger, and is considered the counterpart hormone to leptin. Ghrelin levels increase before meals and decrease after meals, and can result in increased food intake and increase fat mass by an action exerted at the level of the hypothalamus. Ghrelin also stimulates the release of growth hormone. Ghrelin is acylated at a serine residue by n-octanoic acid; this acylation is essential for binding to the GHS1a receptor and for the agonist activity and the GH-releasing capacity of ghrelin. Ghrelin-containing fusion proteins of the invention may find particular use as agonists; e.g., to selectively stimulate motility of the GI tract in gastrointestinal motility disorder, to accelerate gastric emptying, or to stimulate the release of growth hormone. The invention also contemplates unacylated forms and sequence variants of ghrelin, which act as antagonists. Ghrelin analogs with sequence substitutions or truncated variants, such as described in U.S. Pat. No. 7,385,026, may find particular use as fusion partners to XTEN for use as antagonists for improved glucose homeostasis, treatment of insulin resistance and treatment of obesity. The isolation and characterization of ghrelin has been reported (Kojima M, et al., Ghrelin is a growth-hormone-releasing acylated peptide from stomach. Nature. 1999; 402(6762):656-660) and synthetic analogs have been prepared by peptide synthesis, as described in U.S. Pat. No. 6,967,237.

“Glucagon” means the human glucagon glucose regulating peptide, or species and sequence variants thereof, including the native 29 amino acid sequence and homologous sequences; natural, such as from primates, and non-natural sequence variants having at least a portion of the biological activity of mature glucagon. The term “glucagon” as used herein also includes peptide mimetics of glucagon. Native glucagon is produced by the pancreas, released when blood glucose levels start to fall too low, causing the liver to convert stored glycogen into glucose and release it into the bloodstream. While the action of glucagon is opposite that of insulin, which signals the body's cells to take in glucose from the blood, glucagon also stimulates the release of insulin, so that newly-available glucose in the bloodstream can be taken up and used by insulin-dependent tissues. Glucagon-containing fusion proteins of the invention may find particular use in increasing blood glucose levels in individuals with extant hepatic glycogen stores and maintaining glucose homeostasis in diabetes. Glucagon has been cloned, as disclosed in U.S. Pat. No. 4,826,763.

“GLP-1” means human glucagon like peptide-1 and non-natural sequence variants having at least a portion of the biological activity of mature GLP-1. The term “GLP-1” includes human GLP-1(1-37), GLP-1(7-37), GLP-1(7-36)amide, and the GLP-1 analogs of Table 39. GLP-1 stimulates insulin secretion, but only during periods of hyperglycemia. The safety of GLP-1 compared to insulin is enhanced by this property and by the observation that the amount of insulin secreted is proportional to the magnitude of the hyperglycemia. The biological half-life of GLP-1(7-37)OH is a mere 3 to 5 minutes (U.S. Pat. No. 5,118,666). GLP-1-containing fusion proteins of the invention may find particular use in the treatment of diabetes and insulin-resistance disorders for glucose regulation, as well as cardiovascular disorders such as prevention of cardiac remodeling. GLP-1 has been cloned and derivatives prepared, as described in U.S. Pat. No. 5,118,666.

“GLP-2” means human glucagon like peptide-2 and non-natural sequence variants having at least a portion of the biological activity of mature GLP-2. More particularly, GLP-2 is a 33 amino acid peptide, co-secreted along with GLP-1 from intestinal endocrine cells in the small and large intestine.

“IGF-1” or “Insulin-like growth factor 1” means the human IGF-1 protein and species and non-natural sequence variants having, at least a portion of the biological activity of mature IGF-1. IGF-1, which was once called somatomedin C, is a polypeptide protein anabolic hormone similar in molecular structure to insulin, and that modulates the action of growth hormone. IGF-1 consists of 70 amino acids and is produced primarily by the liver as an endocrine hormone as well as in target tissues in a paracrine/autocrine fashion. IGF-1-containing fusion proteins of the invention may find particular use in the treatment of diabetes and insulin-resistance disorders for glucose regulation. IGF-1 has been cloned and expressed in E. coli and yeast, as described in U.S. Pat. No. 5,324,639.

“IGF-2” or “Insulin-like growth factor 2” means the human IGF-2 protein and species and non-natural sequence variants having at least a portion of the biological activity of mature IGF-2. IGF-2 is a polypeptide protein hormone similar in molecular structure to insulin, with a primary role as a growth-promoting hormone during gestation. IGF-2 has been cloned, as described in Bell G I, et al. Isolation of the human insulin-like growth factor genes: insulin-like growth factor II and insulin genes are contiguous. Proc Natl Acad Sci USA. 1985. 82(19):6450-4.

“IL-1ra” means the human IL-1 receptor antagonist protein and species and sequence variants thereof, including the sequence variant anakinra (Kineret®), having at least a portion of the biological activity of mature IL-1ra. IL-1ra is a protein that acts as a natural inhibitor or antagonist of interleukin-1 by binding to the IL-1 receptor (IL-1R). IL-1ra-containing fusion proteins of the invention may find particular use in the treatment of type 2 diabetes for glucose regulation or chronic inflammatory disorders. IL-1ra has been cloned, as described in U.S. Pat. Nos. 5,075,222 and 6,858,409.

“INGAP”, or “islet neogenesis-associated protein”, or “pancreatic beta cell growth factor” means the human INGAP peptide and species and non-natural sequence variants having at least a portion of the biological activity of mature INGAP. INGAP is capable of initiating duct cell proliferation, a prerequisite for islet neogenesis. INGAP-containing fusion proteins of the invention may find particular use in the treatment or prevention of diabetes and insulin-resistance disorders. INGAP has been cloned and expressed, as described in R Rafaeloff R, et al., Cloning and sequencing of the pancreatic islet neogenesis associated protein (INGAP) gene and its expression in islet neogenesis in hamsters. J Clin Invest. 1997. 99(9): 2100-2109.

“Intermedin” or “AFP-6” means the human intermedin peptide and species and sequence variants thereof having at least a portion of the biological activity of mature intermedin. Intermedin is a ligand for the calcitonin receptor-like receptor. Intermedin treatment leads, to blood pressure reduction both in normal and hypertensive subjects, as well as the suppression of gastric emptying activity, and is implicated in glucose homeostasis. Intermedin-containing fusion proteins of the invention may find particular use in the treatment of diabetes, insulin-resistance disorders, and obesity. Intermedin peptides and variants have been cloned, as described in U.S. Pat. No. 6,965,013.

“Leptin” means the naturally occurring leptin from any species, as well as biologically active D-isoforms, or fragments and non-natural sequence variants having at least a portion of the biological activity of mature leptin. Leptin plays a key role in regulating energy intake and energy expenditure, including appetite and metabolism. Leptin-containing fusion proteins of the invention may find particular use in the treatment of diabetes for glucose regulation, insulin-resistance disorders, and obesity. Leptin is the polypeptide product of the ob gene as described in the International Patent Pub. No. WO 96/05309. Leptin has been cloned, as described in U.S. Pat. No. 7,112,659, and leptin analogs and fragments in U.S. Pat. No. 5,521,283, U.S. Pat. No. 5,532,336, PCT/US96/22308 and PCT/US96/01471.

“Natriuretic peptides” means atrial natriuretic peptide (ANP), brain natriuretic peptide (BNP or B-type natriuretic peptide) and C-type natriuretic peptide (CNP); both human and non-human species and sequence variants thereof baying at least a portion of the biological activity of the mature counterpart natriuretic peptides. Alpha atrial natriuretic peptide (aANP) or (ANP) and brain natriuretic peptide (BNP) and type C natriuretic peptide (CNP) are homologous polypeptide hormones involved in the regulation of fluid and electrolyte homeostasis. Sequences of useful forms of natriuretic peptides are disclosed in U.S. Patent Publication 20010027181. Examples of ANPs include human ANP (Kangawa et al., BBRC 118:131 (1984)) or that from various species, including pig and rat ANP (Kangawa et al., BBRC 121:585 (1984)). Sequence analysis reveals that preproBNP consists of 134 residues and is cleaved to a 108-amino acid ProBNP. Cleavage of a 32-amino acid sequence from the C-terminal end of ProBNP results in human BNP (77-108), which is the circulating, physiologically active form. The 32-amino acid human BNP involves the formation of a disulfide bond (Sudoh et al., BBRC 159:1420 (1989)) and U.S. Pat. Nos. 5,114,923, 5,674,710, 5,674,710, and 5,948,761. BFXTEN-containing one or more natriuretic functions may be useful in treating hypertension, diuresis inducement, natriuresis inducement, vascular conduct dilatation or relaxation, natriuretic peptide receptors (such as NPR-A) binding, renin secretion suppression from the kidney, aldostrerone secretion suppression from the adrenal gland, treatment of cardiovascular diseases and disorders, reducing, stopping or reversing cardiac remodeling after a cardiac event or as a result of congestive heart failure, treatment of renal diseases and disorders; treatment or prevention of ischemic stroke, and treatment of asthma.

“Neuromedin” means the neuromedin family of peptides including neuromedin U and S peptides, and non-natural sequence variants having at least a portion of the biological activity of mature neuromedin. The native active human neuromedin U peptide hormone is neuromedin-U25, particularly its amide form. Of particular interest are their processed active peptide hormones and analogs, derivatives and fragments thereof. Included in the neuromedin U family are various truncated or splice variants, e.g., FLFHYSKTQKLGKSNVVEELQSPFASQSRGYFLFRPRN (SEQ ID NO: 58). Exemplary of the neuromedin S family is human neuromedin S with the sequence ILQRGSGTAAVDFTKKDHTATWGRPFFLFRPRN (SEQ ID NO: 59), particularly its amide form. Neuromedin fusion proteins of the invention may find particular use in treating obesity, diabetes, reducing food intake, and other related conditions and disorders as described herein. Of particular interest are neuromedin modules combined with an amylin family peptide, an exendin peptide family or a GLP I peptide family module.

“Oxyntomodulin”, or “OXM” means human oxyntomodulin and species and sequence variants thereof having at least a portion of the biological activity of mature OXM. OXM is a 37 amino acid peptide produced in the colon that contains the 29 amino acid sequence of glucagon followed by an 8 amino acid carboxyterminal extension. OXM has been found to suppress appetite. OXM-containing fusion proteins of the invention may find particular use in the treatment of diabetes for glucose regulation, insulin-resistance disorders, obesity, and can be used as a weight loss treatment.

“PYY” means human peptide YY polypeptide and species and non-natural sequence variants having at least a portion of the biological activity of that we PYY. “PYY” includes both the human full length, 36 amino acid peptide, PYY₁₋₃₆ and PYY₃₋₃₆ which have the PP fold structural motif. PYY inhibits gastric motility and increases water and electrolyte absorption in the colon. PYY may also suppress pancreatic secretion. PPY-containing fusion proteins of the invention may find particular use in the treatment of diabetes for glucose regulation, insulin-resistance disorders, and obesity. Analogs of PYY have been prepared, as described in U.S. Pat. Nos. 5,604,203, 5,574,010 and 7,166,575.

“Urocortin” means a human urocortin peptide hormone and non-natural sequence variants having at least a portion of the biological activity of mature urocortin. There are three human urocortins: Ucn-1, Ucn-2 and Ucn-3. Further urocortins and analogs have been described in U.S. Pat. No. 6,214,797. Urocortins Ucn-2 and Ucn-3 have food-intake suppression, antihypertensive, cardioprotective, and inotropic properties. Ucn-2 and Ucn-3 have the ability to suppress the chronic HPA activation following a stressful stimulus such as dieting/fasting, and are specific for the CRF type 2 receptor and do not activate CRF-R1 which mediates ACTH release. BFXTEN comprising urocortin, e.g., Ucn-2 or Ucn-3, may be useful for vasodilation and thus for cardiovascular uses such as chronic heart failure. Urocortin-containing fusion proteins of the invention may also find particular use in treating or preventing conditions associated with stimulating ACTH release, hypertension due to vasodilatory effects, inflammation mediated via other than ACTH elevation, hyperthermia, appetite disorder, congestive heart failure, stress, anxiety, and psoriasis. Urocortin-containing fusion proteins may also be combined with a natriuretic peptide module, amylin family, and exendin family, or a GLP1 family module to provide an enhanced cardiovascular benefit, e.g. treating CHF, as by providing a beneficial vasodilation effect.

“Urodilatin” means the C-terminal 32 amino acids of the protein gamma-hANaP and non-natural sequence variants having at least a portion of the biological activity of mature urodilatin. Urodilatin originates from precursor proteins formed in the kidneys by post-translator processing, Urodilantin-containing fusion proteins of the invention may find particular use for vasodilation and treating hypertension. The isolation and synthesis of urodilantin has been described in U.S. Pat. No. 5,665,861.

The BP of the subject compositions, particularly those disclosed in Table 1 together with their corresponding nucleic acid and amino acid sequences, are well known in the art and descriptions and sequences are available in public databases such as Chemical Abstracts Services Databases (e.g., the CAS Registry), GenBank, The Universal Protein Resource (UniProt) and subscription provided databases such as GenSeq (e.g., Derwent). Polynucleotide sequences may be a wild type polynucleotide sequence encoding a given BP (e.g., either full length or mature), or in some instances the sequence may be a variant of the wild type polynucleotide sequence (e.g., a polynucleotide which encodes the wild type biologically active protein, wherein the DNA sequence of the polynucleotide has been optimized, for example, for expression in a particular species; or a polynucleotide encoding a variant of the wild type protein, such as a site directed mutant or an allelic variant. It is well within the ability of the skilled artisan to use a wild-type or consensus cDNA sequence or a codon-optimized variant of a BP to create BFXTEN constructs contemplated by the invention using methods known in the art and/or in conjunction with the guidance and methods provided herein, and described more fully in the Examples.

The BP of the subject compositions are not limited to native, full-length polypeptides, but also include recombinant versions as well as biologically and/or pharmacologically active variants or fragments and non-natural sequence variants having at least a portion of the biological activity of the mature BP. For example, it will be appreciated that various amino acid substitutions can be made in the BP to create variants without departing from the spirit of the invention with respect to the biological activity or pharmacologic properties of the BP. Examples of conservative substitutions for amino acids in polypeptide sequences are shown in Table 2. However, the invention contemplates substitution of any of the other 19 natural L-amino acids for a given amino acid residue of the native BP, which may be at any position within the sequence of the BP, including adjacent amino acid residues. If any one substitution results in an undesirable change in biological activity, then one of the alternative amino acids can be employed and the construct evaluated by the methods described herein, or using any of the techniques and guidelines for conservative and non-conservative mutations set forth, for instance, in U.S. Pat. No. 5,364,934, the contents of which is incorporated by reference in its entirety, or using methods generally known to those of skill in the art. In addition, variants can also include, for instance, polypeptides wherein one or more amino acid residues are added or deleted at the N- or C-terminus of the full-length native amino acid sequence of a BP that retains at least a portion of the biological activity of the native peptide. Sequence variants of BP, whether exhibiting substantially the same or better bioactivity than wild-type BP, or, alternatively, exhibiting substantially modified or reduced bioactivity relative to wild-type BP, include, without limitation, polypeptides having an amino acid sequence that differs from the sequence of wild-type BP by insertion, deletion, or substitution of one or more amino acids.

TABLE 2
Exemplary conservative amino acid substitutions

| Original Residue | Exemplary Substitutions |
|---|---|
| Ala (A) | val; leu; ile |
| Arg (R) | lys; gin; asn |
| Asn (N) | gin; his; Iys; arg |
| Asp (D) | Glu |
| Cys (C) | ser |
| Gln (Q) | asn |
| Glu (E) | asp |
| Gly (G) | pro |
| His (H) | asn: gin: Iys: arg |
| Ile (I) | leu; val; met; ala; phe: norleucine |
| Leu (L) | norleucine: ile: val; met; ala: phe |
| Lys (K) | arg: gin: asn |
| Met (M) | leu; phe; ile |
| Phe (F) | leu: val: ile; ala |
| Pro (P) | gly |
| Ser (S) | thr |
| Thr (T) | ser |
| Trp (W) | tyr |
| Tyr (Y) | trp: phe: thr: ser |
| Val (V) | ile; leu; met; phe; ala; norleucine |

(b) Extended Recombinant Polypeptides (XTEN)

In one aspect, the invention provides XTEN polypeptide compositions that are useful as fusion protein partner(s) to which BP are linked, resulting in BFXTEN fusion proteins. XTEN are generally extended length polypeptides with non-naturally occurring, substantially non-repetitive sequences that are composed mainly of small hydrophilic amino acids, with the sequence having a low degree or no secondary or tertiary structure under physiologic conditions.

XTENs have utility as a fusion protein partners in that they serve in various roles, conferring certain desirable pharmacokinetic, physicochemical and pharmaceutical properties when linked to a BP protein to a create a fusion protein. Such desirable properties include but are not limited to enhanced pharmacokinetic parameters and solubility characteristics of the compositions, or can serve as linkers between or within domains of the functional protein, amongst other properties described herein. Such fusion protein compositions have utility to treat certain metabolic or cardiovascular diseases, disorders or conditions, as described herein. As used herein, “XTEN” specifically excludes whole antibodies or antibody fragments (e.g. single-chain antibodies and Fc fragments), albumin, and polypeptides with highly repetitive sequences.

In some embodiments the XTEN serves as a carrier that is a long polypeptide having greater than about 100 to about 3000 amino acid residues as a single polypeptide or cumulatively when more than one XTEN unit is used in a single fusion protein. In other embodiments, when XTEN is used as a linker between BP fusion protein components or insertion sites internal to BP, an XTEN sequence or a fragment of an XTEN sequence shorter than a carrier can be used, such as about 288 amino acid residues, or about 144, or about 100, or about 96, or about 84, or about 72, or about 60, or about 48, or about 42, or about 36, or about 12, or about 6 amino acid residues incorporated at one or more locations into the BFXTEN fusion protein composition.

The selection criteria for the XTEN to be linked to the biologically active proteins used to create the inventive fusion proteins compositions generally relate to attributes of physicochemical properties and conformational structure of the XTEN that is, in turn, used to confer enhanced pharmaceutical and pharmacokinetic properties to the fusion proteins compositions. The XTEN of the present invention exhibits one or more of the following advantageous properties: conformational flexibility, enhanced aqueous solubility, high degree of protease resistance, low immunogenicity, low binding to mammalian receptors, and increased hydrodynamic (or Stokes) radii; properties that make them particularly useful as fusion protein partners. Non-limiting examples of the properties of the fusion proteins comprising BP that are enhanced by XTEN include increases in the overall solubility and/or metabolic stability, reduced susceptibility to proteolysis, reduced immunogenicity, reduced rate of absorption when administered subcutaneously or intramuscularly, and enhanced pharmacokinetic properties such as longer terminal half-life and increased area under the curve (AUC), lower volume of distribution, slower absorption after subcutaneous or intramuscular injection (compared to BP not linked to XTEN and administered by a similar route) such that the Cmax is lower, which, in turn, results in reductions in adverse effects of the BP that, collectively, results in an increased period of time that a fusion protein of a BFXTEN composition administered to a subject retains therapeutic activity. As a result of these enhanced properties, it is contemplated that BFXTEN compositions for subcutaneous or intramuscular administration will provide enhanced bioavailability and permit less frequent dosing compared to BP not linked to XTEN and administered in a comparable fashion.

A variety of methods and assays are known in the art for determining the physical/chemical properties of proteins such as XTEN or BFXTEN fusion protein compositions comprising the inventive XTEN; properties such as secondary or tertiary structure, solubility, protein aggregation, melting properties, contamination and water content. Such methods include analytical centrifugation, EPR, HPLC-ion exchange, HPLC-size exclusion, HPLC-reverse phase, light scattering, capillary electrophoresis, circular dichroism, differential scanning calorimetry, fluorescence, HPLC-ion exchange, HPLC-size exclusion, IR, NMR, Raman spectroscopy, refractometry, and UV/Visible spectroscopy. Additional methods are disclosed in Arnau et al, Prot Expr and Purif (2006) 48, 1-13.

In one embodiment, XTEN is designed to behave like denatured peptide sequence under physiological conditions, despite the extended length of the polymer. “Denatured” describes the state of a peptide in solution that is characterized by a large conformational freedom of the peptide backbone. Most peptides and proteins adopt a denatured conformation in the presence of high concentrations of denaturants or at elevated temperature. Peptides in denatured conformation have, for example, characteristic circular dichroism (CD) spectra and are characterized by a lack of long-range interactions as determined by NMR. “Denatured conformation” and “unstructured conformation” are used synonymously herein. In some embodiments, the invention provides XTEN sequences that, under physiologic conditions, resemble denatured sequences that are largely devoid in secondary structure. The XTEN sequences of the BFXTEN compositions of the invention are substantially devoid of secondary structure under physiologic conditions. “Largely devoid,” as used in this context, means that less than 50% of the XTEN amino acid residues of the XTEN sequence contribute to secondary structure as measured or determined by the means described herein. “Substantially devoid,” as used in this context, means that at least about 60%, or about 70%, or about 80%, or about 90%, or about 95%, or at least about 99% of the XTEN amino acid residues of the XTEN sequence do not contribute to secondary structure, as measured or determined by the methods described herein.

A variety of methods have been established in the art to discern the presence or absence of secondary and tertiary structures in a given polypeptide. In particular, secondary structure can be measured spectrophotometrically, e.g., by circular dichroism spectroscopy in the “far-UV” spectral region (190-250 nm). Secondary structure elements, such as alpha-helix and beta-sheet, each give rise to a characteristic shape and magnitude of CD spectra. Secondary structure can also be predicted for a polypeptide sequence via certain computer programs or algorithms, such as the well-known Chou-Fasman algorithm (Chou, P. Y., et al. (1974) Biochemistry, 13: 222-45) and the Garnier-Osguthorpe-Robson (“GOR”) algorithm (Garnier J, Gibrat J F, Robson B. (1996), GOR method for predicting protein secondary structure from amino acid sequence. Methods Enzymol 266:540-553), as described in US Patent Application Publication No. 20030228309A1. For a given sequence, the algorithms can predict whether there exists some or no secondary structure at all, expressed as the total and/or percentage of residues of the sequence that form, for example, alpha-helices or beta-sheets or the percentage of residues of the sequence predicted to result in random coil formation (which lacks secondary structure).

The XTEN sequences used in the subject fusion protein compositions have an alpha-helix percentage ranging from 0% to less than about 5% as determined by the Chou-Fasman algorithm. In some embodiments, the XTEN sequences of the fusion protein compositions have an alpha-helix percentage less than about 2% and a beta-sheet percentage less than about 2%. The XTEN sequences of the BFXTEN fusion protein compositions have a high degree of random coil percentage, as determined by the GOR algorithm. In some embodiments, an XTEN sequence have at least about 80%, more preferably at least about 90%, more preferably at least about 91%, more preferably at least about 92%, more preferably at least about 93%, more preferably at least about 94%, more preferably at least about 95%, more preferably at least about 96%, more preferably at least about 97%, more preferably at least about 98%, and most preferably at least about 99% random coil, as determined by the GOR algorithm.

1. Non-Repetitive Sequences

It is specifically contemplated that the XTEN sequences of the BFXTEN compositions are substantially non-repetitive. In general, repetitive amino acid sequences have a tendency to aggregate or form higher order structures, as exemplified by natural repetitive sequences such as collagens and leucine zippers. These repetitive amino acids may also tend to form contacts resulting in crystalline or pseudocrystalline structures. In contrast, the low tendency of non-repetitive sequences to aggregate enables the design of long-sequence XTENs with a relatively low frequency of charged amino acids that would otherwise be likely to aggregate if the sequences were repetitive. In one embodiment, the XTEN sequences have greater than about 36 to about 1000 amino acid residues, or about 100 to about 3000 amino acid residues in which no three contiguous amino acids in the sequence are identical amino acid types unless the amino acid is serine, in which case no more than three contiguous amino acids are serine residues. In the foregoing embodiment, the XTEN sequence is “substantially non-repetitive.” In another embodiment, as described more fully below, the XTEN sequences of the compositions comprise non-overlapping sequence motifs of 9 to 14 amino acid residues wherein the motifs consist of 4 to 6 types of amino acids selected from glycine (G), alanine (A), serine (S), threonine (T), glutamate (E) and proline (P), and wherein the sequence of any two contiguous amino acid residues in any one motif is not repeated more than twice in the sequence motif. In the foregoing embodiment, the XTEN sequence is “substantially non-repetitive.”

The degree of repetitiveness of a polypeptide or a gene can be measured by computer programs or algorithms or by other means known in the art. In one non-limiting example, repetitiveness in a polypeptide sequence can be assessed by determining the number of times shorter specific sequences of a given length occur within the polypeptide. For example, a polypeptide of 200 amino acid residues length has a total of 165 overlapping 36-amino acid “blocks” (or “36-mers”) and 198 3-mer “subsequences”, but the number of unique 3-mer subsequences (meaning a unique specific amino acid sequence of the 3-mer) found within the 200 amino acid sequence will depend on the amount of repetitiveness within the sequence; a polypeptide with a higher degree of repetitiveness within the blocks of the polypeptide will have fewer unique 3-mer subsequences and more repeat occurrences of 3-mer subsequences compared to a polypeptide with a lower degree of repetitiveness. A score can be generated (hereinafter “subsequence score”) that is reflective of the degree of repetitiveness for a polypeptide of any length. In one embodiment, the subsequence score is determined for a polypeptide of a given length by determining the average of the cumulative number of occurrences (the “count”) of each unique subsequence (the sequence of a fixed, short peptide length) per each overlapping block (defined as a fixed, intermediate peptide length) of the polypeptide of interest. The subsequence score can be determined by applying the following equation to the polypeptide of interest:

$$Subsequence score = \frac{{\sum }_{i = 1}^{n} ( \frac{{Count}_{i}}{m} )}{n}$$

where: n=(amino acid length of polypeptide)−(amino acid length of block)+1;
   m=(amino acid length of block)−(amino acid length of subsequence)+1; and
   Countᵢ=cumulative number of occurrences of each unique subsequence within blockᵢ
      While the invention contemplates that the equation variable “subsequence” can be a peptide length of 3 to about 10 amino acid residues and that the variable “block” can be a peptide length of about 20 to about 200 amino acid residues, as used herein, “subsequence score” for a polypeptide is determined by application of the foregoing equation to a polypeptide sequence wherein the block length is set at 36 amino acids and the subsequence length is set at 3 amino acids. Examples of subsequence scores derived using the equation with a block length of 36 and frame length of 3 applied to polypeptides of varying composition and sequence, including XTEN sequences of varying length, are presented in Example 28. In one embodiment, the present invention provides BFXTEN comprising one XTEN in which the XTEN has a subsequence score of 3 or less, and more preferably less than 2. In another embodiment, the invention provides BFXTEN comprising two or more XTEN in which at least one XTEN has a subsequence score of 3 or less, and more preferably less than 2. In yet another embodiment, the invention provides BFXTEN comprising multiple XTEN in which each individual XTEN has a subsequence score of 3 or less, and more preferably less than 2. In the embodiments of the BFXTEN fusion protein compositions described herein, an XTEN component of a fusion protein with a subsequence score of 3 or less is “substantially non-repetitive.”

It is believed that the non-repetitive characteristic of XTEN of the present invention contributes to many of the enhanced physicochemical and biological properties of the BFXTEN fusion proteins; either solely or in conjunction with the choice of the particular types of amino acids that predominate in the XTEN of the compositions disclosed herein. These properties include a higher degree of expression of the fusion protein in the host cell, greater genetic stability of the gene encoding XTEN, and a greater degree of solubility and less tendency to aggregate of the resulting BFXTEN compared to fusion proteins comprising polypeptides having repetitive sequences. These properties permit more efficient manufacturing, lower cost of goods, and facilitate the formulation of XTEN-comprising pharmaceutical preparations containing extremely high drug concentrations, in some cases exceeding 100 mg/ml. Furthermore, the XTEN polypeptide sequences of the embodiments are designed to have a low degree of internal repetitiveness in order to reduce or substantially eliminate immunogenicity when administered to a mammal. Polypeptide sequences composed of short, repeated motifs largely limited to only three amino acids, such as glycine, serine and glutamate, may result in relatively high antibody titers when administered to a mammal despite the absence of predicted T-cell epitopes in these sequences. This may be caused by the repetitive nature of polypeptides, as it has been shown that immunogens with repeated epitopes, including protein aggregates, cross-linked immunogens, and repetitive carbohydrates are highly immunogenic and can, for example, result in the cross-linking of B-cell receptors causing B-cell activation. (Johansson, J., et al. (2007) Vaccine, 25:1676-82; Yankai, Z., et al. (2006) Biochem Biophys Res Commun, 345:1365-71; Hsu, C. T., et al. (2000) Cancer Res, 60:3701-5); Bachmann M F, et al. Eur J. Immunol. (1995) 25(12):3445-3451).

2. Exemplary Sequence Motifs

The present invention encompasses XTEN used as fusion partners that comprise multiple units of shorter sequences, or motifs, in which the amino acid sequences of the motifs are non-repetitive. The non-repetitive criterion can be met despite the use of a “building block” approach using a library of sequence motifs that are multimerized to create the XTEN sequences. Thus, while an XTEN sequence may consist of multiple units of as few as four different types of sequence motifs, because the motifs themselves generally consist of non-repetitive amino acid sequences, the overall XTEN sequence is rendered substantially non-repetitive.

In one embodiment, XTEN have a non-repetitive sequence of greater than about 36 to about 3000 amino acid residues wherein at least about 80%, or at least about 85%, or at least about 90%, or at least about 95%, or at least about 97%, or about 100% of the XTEN sequence consists of non-overlapping sequence motifs, wherein each of the motifs has about 9 to 36 amino acid residues. In other embodiments, at least about 80%, or at least about 85%, or at least about 90%, or at least about 95%, or at least about 97%, or about 100% of the XTEN sequence consists of non-overlapping sequence motifs wherein each of the motifs has 9 to 14 amino acid residues. In still other embodiments, at least about 80%, or at least about 85%, or at least about 90%, or at least about 95%, or at least about 97%, or about 100% of the XTEN sequence component consists of non-overlapping sequence motifs wherein each of the motifs has 12 amino acid residues. In these embodiments, it is preferred that the sequence motifs be composed mainly of small hydrophilic amino acids, such that the overall sequence has an unstructured, flexible characteristic. Examples of amino acids that are included in XTEN are, e.g., arginine, lysine, threonine, alanine, asparagine, glutamine, aspartate, glutamate, serine, and glycine. As a result of testing variables such as codon optimization, assembly polynucleotides encoding sequence motifs, expression of protein, charge distribution and solubility of expressed protein, and secondary and tertiary structure, it was discovered that XTEN compositions with enhanced characteristics mainly include glycine (G), alanine (A), serine (S), threonine (T), glutamate (E) and proline (P) residues wherein the sequences are designed to be substantially non-repetitive. XTEN sequences have at least 80% of the sequence consisting of four to six types of amino acids selected from glycine (G), alanine (A), serine (S), threonine (T), glutamate (E) or proline (P) that are arranged in a substantially non-repetitive sequence that is greater than about 36 to about 3000 amino acid residues in length. In some embodiments, XTEN have sequences of greater than about 36 to about 3000 amino acid residues wherein at least about 80% of the sequence consists of non-overlapping sequence motifs wherein each of the motifs has 9 to 36 amino acid residues wherein each of the motifs consists of 4 to 6 types of amino acids selected from glycine (G), alanine (A), serine (S), threonine (T), glutamate (E) and proline (P), and wherein the content of any one amino acid type in the full-length XTEN does not exceed 30%. In other embodiments, at least about 90% of the XTEN sequence consists of non-overlapping sequence motifs wherein each of the motifs has 9 to 36 amino acid residues wherein the motifs consist of 4 to 6 types of amino acids selected from glycine (G), alanine (A), serine (S), threonine (T), glutamate (E) and proline (P), and wherein the content of any one amino acid type in the full-length XTEN does not exceed 30%. In other embodiments, at least about 90% of the XTEN sequence consists of non-overlapping sequence motifs wherein each of the motifs has 12 amino acid residues consisting of 4 to 6 types of amino acids selected from glycine (G), alanine (A), serine (S), threonine (T), glutamate (E) and proline (P), and wherein the content of any one amino acid type in the full-length XTEN does not exceed 30%. In yet other embodiments, at least about 80%, or about 90%, or about 91%, or about 92%, or about 93%, or about 94%, or about 95%, or about 96%, or about 97%, or about 98%, or about 99%, to about 100% of the XTEN sequence consists of non-overlapping sequence motifs wherein each of the motifs has 12 amino acid residues consisting of glycine (G), alanine (A), serine (S), threonine (T), glutamate (E) and proline (P), and wherein the content of any one amino acid type in the full-length XTEN does not exceed 30%.

In still other embodiments, XTENs comprise non-repetitive sequences of greater than about 36 to about 3000 amino acid residues wherein at least about 80%, or at least about 90%, or about 91%, or about 92%, or about 93%, or about 94%, or about 95%, or about 96%, or about 97%, or about 98%, or about 99% of the sequence consists of non-overlapping sequence motifs of 9 to 14 amino acid residues wherein the motifs consist of 4 to 6 types of amino acids selected from glycine (G), alanine (A), serine (S), threonine (T), glutamate (E) and proline (P), and wherein the sequence of any two contiguous amino acid residues in any one motif is not repeated more than twice in the sequence motif. In other embodiments, at least about 80%, or about 90%, or about 91%, or about 92%, or about 93%, or about 94%, or about 95%, or about 96%, or about 97%, or about 98%, or about 99% of an XTEN sequence consists of non-overlapping sequence motifs of 12 amino acid residues wherein the motifs consist of four to six types of amino acids selected from glycine (G), alanine (A), serine (S), threonine (T), glutamate (E) and proline (P), and wherein the sequence of any two contiguous amino acid residues in any one sequence motif is not repeated more than twice in the sequence motif. In other embodiments, at least about 80%, or about 90%, or about 91%, or about 92%, or about 93%, or about 94%, or about 95%, or about 96%, or about 97%, or about 98%, or about 99% of an XTEN sequence consists of non-overlapping sequence motifs of 12 amino acid residues wherein the motifs consist of glycine (G), alanine (A), serine (S), threonine (T), glutamate (E) and proline (P), and wherein the sequence of any two contiguous amino acid residues in any one sequence motif is not repeated more than twice in the sequence motif. In yet other embodiments, XTENs consist of 12 amino acid sequence motifs wherein the amino acids are selected from glycine (G), alanine (A), serine (S), threonine (T), glutamate (E) and proline (P), and wherein the sequence of any two contiguous amino acid residues in any one sequence motif is not repeated more than twice in the sequence motif, and wherein the content of any one amino acid type in the full-length XTEN does not exceed 30%. In the foregoing embodiments hereinabove described in this paragraph, the XTEN sequences is “substantially non-repetitive.”.

In some embodiments, the BFXTEN compositions comprise one or more non-repetitive XTEN sequences of greater than about 100 to about 3000 amino acid residues, or greater than 400 to about 3000 residues, wherein at least about 80%, or at least about 90%, or about 91%, or about 92%, or about 93%, or about 94%, or about 95%, or about 96%, or about 97%, or about 98%, or about 99% to about 100% of the sequence consists of multiple units of two or more non-overlapping sequence motifs selected from the amino acid sequences of Table 3 wherein the overall sequence is substantially non-repetitive. In some embodiments, the XTEN comprises non-overlapping sequence motifs in which about 80%, or at least about 85%, or at least about 90%, or about 91%, or about 92%, or about 93%, or about 94%, or about 95%, or about 96%, or about 97%, or about 98%, or about 99% or about 100% of the sequence consists of multiple units of two or more non-overlapping sequences selected from a single motif family selected from Table 3, resulting in a family sequence. As used herein, “family” means that the XTEN has motifs selected only from a single motif category from Table 3; i.e., AD, AE, AF, AG, AM, AQ, BC, or BD XTEN, and that any other amino acids in the XTEN not from a family motif are selected to achieve a needed property, such as to permit incorporation of a restriction site by the encoding nucleotides, incorporation of a cleavage sequence, or to achieve a better linkage to a BP component. Accordingly, in the embodiments of XTEN families, an XTEN sequence comprises multiple units of non-overlapping sequence motifs of the AD motif family, or an XTEN sequence comprises multiple units of non-overlapping sequence motifs of the AE motif family, or an XTEN sequence comprises multiple units of non-overlapping sequence motifs of the AF motif family, or an XTEN sequence comprises multiple units of non-overlapping sequence motifs of the AG motif family, or an XTEN sequence comprises multiple units of non-overlapping sequence motifs of the AM motif family, or an XTEN sequence comprises multiple units of non-overlapping sequence motifs of the AQ motif family, or an XTEN sequence comprises multiple units of non-overlapping sequence motifs of the BC family, or an XTEN sequence comprises multiple units of non-overlapping sequence motifs of the BD family. In other embodiments, the XTEN comprises multiple units of motif sequences from two or more of the motif families of Table 3, selected to achieve desired physicochemical characteristics, including such properties as net charge, lack of secondary structure, or lack of repetitiveness that may be conferred by the amino acid composition of the motifs, described more fully below. In the embodiments hereinabove described in this paragraph, the motifs of Table 3 incorporated into the XTEN can be selected and assembled using the methods described herein to achieve an XTEN of about 36 to about 3000 amino acid residues.

TABLE 3
XTEN Sequence Motifs of 12 Amino Acids and
Motif Families

| Motif | MOTIF | SEQ ID |
|---|---|---|
| Family* | SEQUENCE | NO: |
| AD | GESPGGSSGSES | 60 |
| AD | GSEGSSGPGESS | 61 |
| AD | GSSESGSSEGGP | 62 |
| AD | GSGGEPSESGSS | 63 |
| AE, AM | GSPAGSPTSTEE | 64 |
| AE, AM, AQ | GSEPATSGSETP | 65 |
| AE, AM, AQ | GTSESATPESGP | 66 |
| AE, AM, AQ | GTSTEPSEGSAP | 67 |
| AF, AM | GSTSESPSGTAP | 68 |
| AF, AM | GTSTPESGSASP | 69 |
| AF, AM | GTSPSGESSTAP | 70 |
| AF, AM | GSTSSTAESPGP | 71 |
| AG, AM | GTPGSGTASSSP | 72 |
| AG, AM | GSSTPSGATGSP | 73 |
| AG, AM | GSSPSASTGTGP | 74 |
| AG, AM | GASPGTSSTGSP | 75 |
| AQ | GEPAGSPTSTSE | 76 |
| AQ | GTGEPSSTPASE | 77 |
| AQ | GSGPSTESAPTE | 78 |
| AQ | GSETPSGPSETA | 79 |
| AQ | GPSETSTSEPGA | 80 |
| AQ | GSPSEPTEGTSA | 81 |
| BC | GSGASEPTSTEP | 82 |
| BC | GSEPATSGTEPS | 83 |
| BC | GTSEPSTSEPGA | 84 |
| BC | GTSTEPSEPGSA | 85 |
| BD | GSTAGSETSTEA | 86 |
| BD | GSETATSGSETA | 87 |
| BD | GTSESATSESGA | 88 |
| BD | GTSTEASEGSAS | 89 |

*Denotes individual motif sequences that, when used together in various permutations, results in a “family sequence”

In other embodiments, the BFXTEN composition comprises one or more non-repetitive XTEN sequences of about 36 to about 3000 amino acid residues, wherein at least about 80%, or at least about 90%, or about 91%, or about 92%, or about 93%, or about 94%, or about 95%, or about 96%, or about 97%, or about 98%, or about 99% to about 100% of the sequence consists of non-overlapping 36 amino acid sequence motifs selected from one or more of the polypeptide sequences of Tables 9-12, either as a family sequence, or where motifs are selected from two or more families of motifs.

In those embodiments wherein the XTEN component of the BFXTEN fusion protein has less than 100% of its amino acids consisting of four to six amino acid selected from glycine (G), alanine (A), serine (S), threonine (T), glutamate (E) and proline (P), or less than 100% of the sequence consisting of the sequence motifs of Table 3 or the sequences of Tables 9-12, or less than 100% sequence identity compared with an XTEN from Table 4, the other amino acid residues are selected from any other of the 14 natural L-amino acids, but are preferentially selected from hydrophilic amino acids such that the XTEN sequence contains at least about 90%, or at least about 91%, or at least about 92%, or at least about 93%, or at least about 94%, or at least about 95%, or at least about 96%, or at least about 97%, or at least about 98%, or at least about 99% hydrophilic amino acids. The XTEN amino acids that are not glycine (G), alanine (A), serine (S), threonine (T), glutamate (E) and proline (P) are interspersed throughout the XTEN sequence, are located within or between the sequence motifs, or are concentrated in one or more short stretches of the XTEN sequence. In such cases where the XTEN component of the BFXTEN comprises amino acids other than glycine (G), alanine (A), serine (S), threonine (T), glutamate (E) and proline (P), it is preferred that the amino acids not be hydrophobic residues and should not substantially confer secondary structure of the XTEN component. Hydrophobic residues that are less favored in construction of XTEN include tryptophan, phenylalanine, tyrosine, leucine, isoleucine, valine, and methionine. Additionally, one can design the XTEN sequences to contain less than 5% or less than 4% or less than 3% or less than 2% or less than 1% or none of the following amino acids: cysteine (to avoid disulfide formation and oxidation), methionine (to avoid oxidation), asparagine and glutamine (to avoid desamidation). Thus, in some embodiments, the XTEN component of the BFXTEN fusion protein comprising other amino acids in addition to glycine (G), alanine (A), serine (S), threonine (T), glutamate (E) and proline (P) would have a sequence with less than 5% of the residues contributing to alpha-helices and beta-sheets as measured by the Chou-Fasman algorithm and have at least 90%, or at least about 95% or more random coil formation as measured by the GOR algorithm.

3. Length of Sequence

In another aspect, the invention encompasses BFXTEN compositions comprising one or more XTEN polypeptides wherein the length of the XTEN sequences is selected based on the property or function to be achieved. In one embodiment, XTEN or fragments of XTEN are incorporated into the BFXTEN as a linker, with lengths of about 6 to about 150 amino acids joining components such as two BP or between a cleavage sequence and a BP and/or an XTEN. In another embodiment, one or more XTEN are incorporated into the BFXTEN as a carrier that can be inserted between two BP and/or can be inserted at the terminus of the BFXTEN fusion protein. When XTEN is used as a carrier, the embodiment takes advantage of the discovery that increasing the length of the non-repetitive, unstructured polypeptides enhances the unstructured nature of the XTENs and correspondingly enhances the biological and pharmacokinetic properties of fusion proteins comprising the XTEN carrier. As described more fully in the Examples, proportional increases in the length of the XTEN, even if created by a repeated order of single family sequence motifs (e.g., the four AE motifs of Table 3), result in a sequence with a higher percentage of random coil formation, as determined by GOR algorithm, or a low percentage of alpha-helices or beta-sheets, as determined by Chou-Fasman algorithm, compared to shorter XTEN lengths. In general, increasing the length of the unstructured polypeptide fusion partner, as described in the Examples, results in a fusion protein with a disproportionate increase in terminal half-life compared to fusion proteins with unstructured polypeptide partners with shorter sequence lengths. Depending on the intended function, XTEN or fragments of XTEN incorporated into BFXTEN can be about 6, or about 12, or about 36, or about 40, or about 100, or about 144, or about 288, or about 401, or about 500, or about 600, or about 700, or about 800, or about 900, or about 1000, or about 1500, or about 2000, or about 2500, or up to about 3000 amino acid residues in length. In other cases, the XTEN sequences can be about 6 to about 50, or about 100 to 150, about 150 to 250, about 250 to 400, about 400 to about 500, about 500 to 900, about 900 to 1500, about 1500 to 2000, or about 2000 to about 3000 amino acid residues in length. Non-limiting examples of XTEN contemplated for inclusion in the BFXTEN of the invention are presented in Tables 4 and 9-12, below. In the embodiments hereinabove described in this paragraph, the one or more XTEN sequences incorporated into BXTEN individually exhibit at least about 80% sequence identity, or alternatively 81%, 82%, 83%, 84%, 85%, 86%, 87%, 88%, 89%, 90%, 91%, 92%, 93%, 94%, 95%, 96%, 97%, 98%, 99%, or 100% sequence identity compared to an XTEN selected from Table 4 or Table 9 or Table 10 or Table 11 or Table 12, or a fragment thereof with comparable length. In one non-limiting example, the AG864 sequence of 864 amino acid residues can be truncated to yield an AG144 with 144 residues, an AG288 with 288 residues, an AG576 with 576 residues, or other intermediate lengths. It is specifically contemplated that such an approach can be utilized with any of the XTEN embodiments described herein or with any of the sequences listed in Tables 4 or 9-13 to result in XTEN of an intermediate length. Alternatively, in other embodiments, the BFXTEN comprise one or more XTEN wherein the individual XTEN are created by the linking together of sequence motifs selected from Table 3 and/or the 36-amino acid sequences of Tables 9-12 using the methods described herein. In one embodiment of the foregoing, the 12-amino acid motifs of Table 3 or the 36-amino acid sequences of Tables 9-12 would be selected from a single family of XTEN; e.g., AD, AE, AF, AG, AM, AQ, BC or BD. The invention also encompasses XTEN created by selecting sequences from two or more different XTEN families of the 12-amino acid motifs of Table 3 or the 36-amino acid sequences of Tables 9-12.

In other embodiments, the BFXTEN fusion protein comprises a first and a second XTEN sequence, wherein the cumulative total of the residues in the XTEN sequences is greater than about 400 to about 3000 amino acid residues and the XTEN can be identical or they can be different in sequence. As used herein, “cumulative length” is intended to encompass the total length, in amino acid residues, when more than one XTEN is used in the fusion protein. In embodiments of the foregoing, the BFXTEN fusion protein comprises a first and a second XTEN sequence wherein the sequences each exhibit at least about 80% sequence identity, or alternatively 81%, 82%, 83%, 84%, 85%, 86%, 87%, 88%, 89%, 90%, 91%, 92%, 93%, 94%, 95%, 96%, 97%, 98%, 99%, or 100% sequence identity compared to at least a first or additionally a second XTEN selected from Table 4. Examples where more than one XTEN is used in a BFXTEN composition include, but are not limited to constructs with an XTEN linked to both the N- and C-termini of at least one BP.

As described more fully below, methods are disclosed in which the BFXTEN is designed by selecting the length of the XTEN to confer a target half-life or other physicochemical property on a fusion protein administered to a subject. In general, XTEN cumulative lengths longer that about 400 residues incorporated into the BFXTEN compositions result in longer half-life compared to shorter cumulative lengths; e.g., shorter than about 280 residues. Thus, BFXTEN fusion proteins designs are contemplated that comprise a single XTEN with a long sequence length of at least about 288, or at least about 400, or at least about 600, or at least about 800, or at least about 1000 or more amino acids, or, in the alternative, multiple XTEN are incorporated into the fusion protein to achieve long cumulative lengths of at least about 288, or at least about 400, or at least about 600, or at least about 800, or at least about 1000 or more amino acids; either of which are designed to confer slower rates of systemic absorption, increased bioavailability, and increased half-life after subcutaneous or intramuscular administration to a subject compared to shorter XTEN lengths. In such embodiments, the Cₘₐₓ is reduced in comparison to a comparable dose of a BP not linked to XTEN, thereby contributing to the ability to keep the BFXTEN within the therapeutic window for the composition. Thus, the XTEN confers the property of a depot to the administered BFXTEN, in addition to the other physical/chemical properties described herein.

TABLE 4
XTEN Polypeptides

| XTEN | | SEQ ID |
|---|---|---|
| Name | Amino Acid Sequence | NO: |
| AE42_1 | TEPSEGSAPGSPAGSPTSTEEGTSESATPESGPGSEPATSGS | 90 |
| AE42_2 | PAGSPTSTEEGTSTEPSEGSAPGTSESATPESGPGSEPATSG | 91 |
| AE42_3 | SEPATSGSETPGTSESATPESGPGTSTEPSEGSAPGSPAGSP | 92 |
| AE43_4 | GSPGGSPAGSPTSTEEGTSESATPESGPGSEPATSGSETPGT | 93 |
| AE42_5 | GAPGSPAGSPTSTEEGTSESATPESGPGSEPATSGSETPGPA | 94 |
| AG42_1 | GAPSPSASTGTGPGTPGSGTASSSPGSSTPSGATGSPGPSGP | 95 |
| AG42_2 | GPGTPGSGTASSSPGSSTPSGATGSPGSSPSASTGTGPGASP | 96 |
| AG42_3 | SPSASTGTGPGASPGTSSTGSPGTPGSGTASSSPGSSTPSGA | 97 |
| AG42_4 | SASTGTGPGASPGTSSTGSPGTPGSGTASSSPGSSTPSGATG | 98 |
| AG42_5 | GAPGASPGTSSTGSPGSSPSASTGTGPGTPGSGTASSSPGPA | 99 |
| AG42_6 | GSPGGASPGTSSTGSPGSSPSASTGTGPGTPGSGTASSSPTG | 100 |
| AE48 | MAEPAGSPTSTEEGTPGSGTASSSPGSSTPSGATGSPGASPGTSSTGS | 101 |
| AM48 | MAEPAGSPTSTEEGASPGTSSTGSPGSSTPSGATGSPGSSTPSGATGS | 102 |
| AE144 | GSEPATSGSETPGTSESATPESGPGSEPATSGSETPGSPAGSPTSTEEGTSTEPSEGSAPG | 103 |
| | SEPATSGSETPGSEPATSGSETPGSEPATSGSETPGTSTEPSEGSAPGTSESATPESGPGS | |
| | EPATSGSETPGTSTEPSEGSAP | |
| AF144 | GTSTPESGSASPGTSPSGESSTAPGTSPSGESSTAPGSTSSTAESPGPGSTSESPSGTAPGS | 104 |
| | TSSTAESPGPGTSPSGESSTAPGTSTPESGSASPGSTSSTAESPGPGTSPSGESSTAPGTSP | |
| | SGESSTAPGTSPSGESSTAP | |
| AG144_ | PGSSPSASTGTGPGSSPSASTGTGPGTPGSGTASSSPGSSTPSGATGSPGSSPSASTGTGP | 105 |
| 1 | GASPGTSSTGSPGTPGSGTASSSPGSSTPSGATGSPGTPGSGTASSSPGASPGTSSTGSPG | |
| | ASPGTSSTGSPGTPGSGTASSS | |
| AG144_ | SGTASSSPGSSTPSGATGSPGTPGSGTASSSPGSSTPSGATGSPGSSTPSGATGSPGSSPS | 106 |
| 2 | ASTGTGPGSSPSASTGTGPGASPGTSSTGSPGTPGSGTASSSPGSSTPSGATGSPGSSPSA | |
| | STGTGPGSSPSASTGTGPGASP | |
| AG144_ | GTPGSGTASSSPGSSTPSGATGSPGSSTPSGATGSPGSSPSASTGTGPGSSPSASTGTGPG | 107 |
| 3 | ASPGTSSTGSPGTPGSGTASSSPGSSTPSGATGSPGSSPSASTGTGPGSSPSASTGTGPGA | |
| | SPGTSSTGSPGASPGTSSTGSP | |
| AG144_ | GTPGSGTASSSPGSSTPSGATGSPGSSPSASTGTGPGSSPSASTGTGPGASPGTSSTGSPG | 108 |
| 4 | ASPGTSSTGSPGSSTPSGATGSPGSSPSASTGTGPGASPGTSSTGSPGSSPSASTGTGPGT | |
| | PGSGTASSSPGSSTPSGATGSP | |
| AE288 | GTSESATPESGPGSEPATSGSETPGTSESATPESGPGSEPATSGSETPGTSESATPESGPG | 109 |
| | TSTEPSEGSAPGSPAGSPTSTEEGTSESATPESGPGSEPATSGSETPGTSESATPESGPGS | |
| | PAGSPTSTEEGSPAGSPTSTEEGTSTEPSEGSAPGTSESATPESGPGTSESATPESGPGTS | |
| | ESATPESGPGSEPATSGSETPGSEPATSGSETPGSPAGSPTSTEEGTSTEPSEGSAPGTST | |
| | EPSEGSAPGSEPATSGSETPGTSESATPESGPGTSTEPSEGSAP | |
| AG288_ | PGASPGTSSTGSPGASPGTSSTGSPGTPGSGTASSSPGSSTPSGATGSPGTPGSGTASSSP | 110 |
| 1 | GSSTPSGATGSPGTPGSGTASSSPGSSTPSGATGSPGSSTPSGATGSPGSSPSASTGTGPG | |
| | SSPSASTGTGPGASPGTSSTGSPGTPGSGTASSSPGSSTPSGATGSPGSSPSASTGTGPGS | |
| | SPSASTGTGPGASPGTSSTGSPGASPGTSSTGSPGSSTPSGATGSPGSSPSASTGTGPGAS | |
| | PGTSSTGSPGSSPSASTGTGPGTPGSGTASSSPGSSTPSGATGS | |
| AG288_ | GSSPSASTGTGPGSSPSASTGTGPGTPGSGTASSSPGSSTPSGATGSPGSSPSASTGTGPG | 111 |
| 2 | ASPGTSSTGSPGTPGSGTASSSPGSSTPSGATGSPGTPGSGTASSSPGASPGTSSTGSPGA | |
| | SPGTSSTGSPGTPGSGTASSSPGSSTPSGATGSPGASPGTSSTGSPGTPGSGTASSSPGSS | |
| | TPSGATGSPGSSPSASTGTGPGSSPSASTGTGPGSSTPSGATGSPGSSTPSGATGSPGASP | |
| | GTSSTGSPGASPGTSSTGSPGASPGTSSTGSPGTPGSGTASSSP | |
| AF504 | GASPGTSSTGSPGSSPSASTGTGPGSSPSASTGTGPGTPGSGTASSSPGSSTPSGATGSPG | 112 |
| | SXPSASTGTGPGASPGTSSTGSPGTPGSGTASSSPGSSTPSGATGSPGTPGSGTASSSPG | |
| | ASPGTSSTGSPGASPGTSSTGSPGTPGSGTASSSPGSSTPSGATGSPGASPGTSSTGSPGT | |
| | PGSGTASSSPGSSTPSGATGSPGSXPSASTGTGPGSSPSASTGTGPGSSTPSGATGSPGSS | |
| | TPSGATGSPGASPGTSSTGSPGASPGTSSTGSPGASPGTSSTGSPGTPGSGTASSSPGASP | |
| | GTSSTGSPGASPGTSSTGSPGASPGTSSTGSPGSSPSASTGTGPGTPGSGTASSSPGASPG | |
| | TSSTGSPGASPGTSSTGSPGASPGTSSTGSPGSSTPSGATGSPGSSTPSGATGSPGASPGT | |
| | SSTGSPGTPGSGTASSSPGSSTPSGATGSPGSSTPSGATGSPGSSTPSGATGSPGSSPSAS | |
| | TGTGPGASPGTSSTGSP | |
| AF540 | GSTSSTAESPGPGSTSSTAESPGPGSTSESPSGTAPGSTSSTAESPGPGSTSSTAESPGPG | 113 |
| | TSTPESGSASPGSTSESPSGTAPGTSPSGESSTAPGSTSESPSGTAPGSTSESPSGTAPGTS | |
| | PSGESSTAPGSTSESPSGTAPGSTSESPSGTAPGTSPSGESSTAPGSTSESPSGTAPGSTSE | |
| | SPSGTAPGSTSESPSGTAPGTSTPESGSASPGSTSESPSGTAPGTSTPESGSASPGSTSSTA | |
| | ESPGPGSTSSTAESPGPGTSTPESGSASPGTSTPESGSASPGSTSESPSGTAPGTSTPESGS | |
| | ASPGTSTPESGSASPGSTSESPSGTAPGSTSESPSGTAPGSTSESPSGTAPGSTSSTAESPG | |
| | PGTSTPESGSASPGTSTPESGSASPGSTSESPSGTAPGSTSESPSGTAPGTSTPESGSASPG | |
| | STSESPSGTAPGSTSESPSGTAPGTSTPESGSASPGTSPSGESSTAPGSTSSTAESPGPGTS | |
| | PSGESSTAPGSTSSTAESPGPGTSTPESGSASPGSTSESPSGTAP | |
| AD576 | GSSESGSSEGGPGSGGEPSESGSSGSSESGSSEGGPGSSESGSSEGGPGSSESGSSEGGPG | 114 |
| | SSESGSSEGGPGSSESGSSEGGPGESPGGSSGSESGSEGSSGPGESSGSSESGSSEGGPGS | |
| | SESGSSEGGPGSSESGSSEGGPGSGGEPSESGSSGESPGGSSGSESGESPGGSSGSESGSG | |
| | GEPSESGSSGSSESGSSEGGPGSGGEPSESGSSGSGGEPSESGSSGSEGSSGPGESSGESP | |
| | GGSSGSESGSGGEPSESGSSGSGGEPSESGSSGSGGEPSESGSSGSSESGSSEGGPGESPG | |
| | GSSGSESGESPGGSSGSESGESPGGSSGSESGESPGGSSGSESGESPGGSSGSESGSSESG | |
| | SSEGGPGSGGEPSESGSSGSEGSSGPGESSGSSESGSSEGGPGSGGEPSESGSSGSSESGS | |
| | SEGGPGSGGEPSESGSSGESPGGSSGSESGESPGGSSGSESGSSESGSSEGGPGSGGEPS | |
| | ESGSSGSSESGSSEGGPGSGGEPSESGSSGSGGEPSESGSSGESPGGSSGSESGSEGSSGP | |
| | GESSGSSESGSSEGGPGSEGSSGPGESS | |
| AE576 | GSPAGSPTSTEEGTSESATPESGPGTSTEPSEGSAPGSPAGSPTSTEEGTSTEPSEGSAPG | 115 |
| | TSTEPSEGSAPGTSESATPESGPGSEPATSGSETPGSEPATSGSETPGSPAGSPTSTEEGT | |
| | SESATPESGPGTSTEPSEGSAPGTSTEPSEGSAPGSPAGSPTSTEEGTSTEPSEGSAPGTS | |
| | TEPSEGSAPGTSESATPESGPGTSTEPSEGSAPGTSESATPESGPGSEPATSGSETPGTST | |
| | EPSEGSAPGTSTEPSEGSAPGTSESATPESGPGTSESATPESGPGSPAGSPTSTEEGTSES | |
| | ATPESGPGSEPATSGSETPGTSESATPESGPGTSTEPSEGSAPGTSTEPSEGSAPGTSTEP | |
| | SEGSAPGTSTEPSEGSAPGTSTEPSEGSAPGTSTEPSEGSAPGSPAGSPTSTEEGTSTEPS | |
| | EGSAPGTSESATPESGPGSEPATSGSETPGTSESATPESGPGSEPATSGSETPGTSESATP | |
| | ESGPGTSTEPSEGSAPGTSESATPESGPGSPAGSPTSTEEGSPAGSPTSTEEGSPAGSPTS | |
| | TEEGTSESATPESGPGTSTEPSEGSAP | |
| AF576 | GSTSSTAESPGPGSTSSTAESPGPGSTSESPSGTAPGSTSSTAESPGPGSTSSTAESPGPG | 116 |
| | TSTPESGSASPGSTSESPSGTAPGTSPSGESSTAPGSTSESPSGTAPGSTSESPSGTAPGTS | |
| | PSGESSTAPGSTSESPSGTAPGSTSESPSGTAPGTSPSGESSTAPGSTSESPSGTAPGSTSE | |
| | SPSGTAPGSTSESPSGTAPGTSTPESGSASPGSTSESPSGTAPGTSTPESGSASPGSTSSTA | |
| | ESPGPGSTSSTAESPGPGTSTPESGSASPGTSTPESGSASPGSTSESPSGTAPGTSTPESGS | |
| | ASPGTSTPESGSASPGSTSESPSGTAPGSTSESPSGTAPGSTSESPSGTAPGSTSSTAESPG | |
| | PGTSTPESGSASPGTSTPESGSASPGSTSESPSGTAPGSTSESPSGTAPGTSTPESGSASPG | |
| | STSESPSGTAPGSTSESPSGTAPGTSTPESGSASPGTSPSGESSTAPGSTSSTAESPGPGTS | |
| | PSGESSTAPGSTSSTAESPGPGTSTPESGSASPGSTSESPSGTAPGSTSSTAESPGPGTSTP | |
| | ESGSASPGTSTPESGSASP | |
| AG576 | PGTPGSGTASSSPGSSTPSGATGSPGSSPSASTGTGPGSSPSASTGTGPGSSTPSGATGSP | 117 |
| | GSSTPSGATGSPGASPGTSSTGSPGASPGTSSTGSPGASPGTSSTGSPGTPGSGTASSSPG | |
| | ASPGTSSTGSPGASPGTSSTGSPGASPGTSSTGSPGSSPSASTGTGPGTPGSGTASSSPGA | |
| | SPGTSSTGSPGASPGTSSTGSPGASPGTSSTGSPGSSTPSGATGSPGSSTPSGATGSPGAS | |
| | PGTSSTGSPGTPGSGTASSSPGSSTPSGATGSPGSSTPSGATGSPGSSTPSGATGSPGSSP | |
| | SASTGTGPGASPGTSSTGSPGASPGTSSTGSPGTPGSGTASSSPGASPGTSSTGSPGASP | |
| | GTSSTGSPGASPGTSSTGSPGASPGTSSTGSPGTPGSGTASSSPGSSTPSGATGSPGTPGS | |
| | GTASSSPGSSTPSGATGSPGTPGSGTASSSPGSSTPSGATGSPGSSTPSGATGSPGSSPSA | |
| | STGTGPGSSPSASTGTGPGASPGTSSTGSPGTPGSGTASSSPGSSTPSGATGSPGSSPSAS | |
| | TGTGPGSSPSASTGTGPGASPGTSSTGS | |
| AE624 | MAEPAGSPTSTEEGTPGSGTASSSPGSSTPSGATGSPGASPGTSSTGSPGSPAGSPTSTE | 118 |
| | EGTSESATPESGPGTSTEPSEGSAPGSPAGSPTSTEEGTSTEPSEGSAPGTSTEPSEGSAP | |
| | GTSESATPESGPGSEPATSGSETPGSEPATSGSETPGSPAGSPTSTEEGTSESATPESGPG | |
| | TSTEPSEGSAPGTSTEPSEGSAPGSPAGSPTSTEEGTSTEPSEGSAPGTSTEPSEGSAPGT | |
| | SESATPESGPGTSTEPSEGSAPGTSESATPESGPGSEPATSGSETPGTSTEPSEGSAPGTS | |
| | TEPSEGSAPGTSESATPESGPGTSESATPESGPGSPAGSPTSTEEGTSESATPESGPGSEP | |
| | ATSGSETPGTSESATPESGPGTSTEPSEGSAPGTSTEPSEGSAPGTSTEPSEGSAPGTSTE | |
| | PSEGSAPGTSTEPSEGSAPGTSTEPSEGSAPGSPAGSPTSTEEGTSTEPSEGSAPGTSESA | |
| | TPESGPGSEPATSGSETPGTSESATPESGPGSEPATSGSETPGTSESATPESGPGTSTEPS | |
| | EGSAPGTSESATPESGPGSPAGSPTSTEEGSPAGSPTSTEEGSPAGSPTSTEEGTSESATP | |
| | ESGPGTSTEPSEGSAP | |
| AD836 | GSSESGSSEGGPGSSESGSSEGGPGESPGGSSGSESGSGGEPSESGSSGESPGGSSGSESG | 119 |
| | ESPGGSSGSESGSSESGSSEGGPGSSESGSSEGGPGSSESGSSEGGPGESPGGSSGSESGE | |
| | SPGGSSGSESGESPGGSSGSESGSSESGSSEGGPGSSESGSSEGGPGSSESGSSEGGPGSS | |
| | ESGSSEGGPGSSESGSSEGGPGSSESGSSEGGPGSGGEPSESGSSGESPGGSSGSESGESP | |
| | GGSSGSESGSGGEPSESGSSGSEGSSGPGESSGSSESGSSEGGPGSGGEPSESGSSGSEGS | |
| | SGPGESSGSSESGSSEGGPGSGGEPSESGSSGESPGGSSGSESGSGGEPSESGSSGSGGEP | |
| | SESGSSGSSESGSSEGGPGSGGEPSESGSSGSGGEPSESGSSGSEGSSGPGESSGESPGGS | |
| | SGSESGSEGSSGPGESSGSEGSSGPGESSGSGGEPSESGSSGSSESGSSEGGPGSSESGSS | |
| | EGGPGESPGGSSGSESGSGGEPSESGSSGSEGSSGPGESSGESPGGSSGSESGSEGSSGP | |
| | GSSESGSSEGGPGSGGEPSESGSSGSEGSSGPGESSGSEGSSGPGESSGSEGSSGPGESSG | |
| | SGGEPSESGSSGSGGEPSESGSSGESPGGSSGSESGESPGGSSGSESGSGGEPSESGSSGS | |
| | EGSSGPGESSGESPGGSSGSESGSSESGSSEGGPGSSESGSSEGGPGSSESGSSEGGPGSG | |
| | GEPSESGSSGSSESGSSEGGPGESPGGSSGSESGSGGEPSESGSSGSSESGSSEGGPGESP | |
| | GGSSGSESGSGGEPSESGSSGESPGGSSGSESGSGGEPSESGSS | |
| AE864 | GSPAGSPTSTEEGTSESATPESGPGTSTEPSEGSAPGSPAGSPTSTEEGTSTEPSEGSAPG | 120 |
| | TSTEPSEGSAPGTSESATPESGPGSEPATSGSETPGSEPATSGSETPGSPAGSPTSTEEGT | |
| | SESATPESGPGTSTEPSEGSAPGTSTEPSEGSAPGSPAGSPTSTEEGTSTEPSEGSAPGTS | |
| | TEPSEGSAPGTSESATPESGPGTSTEPSEGSAPGTSESATPESGPGSEPATSGSETPGTST | |
| | EPSEGSAPGTSTEPSEGSAPGTSESATPESGPGTSESATPESGPGSPAGSPTSTEEGTSES | |
| | ATPESGPGSEPATSGSETPGTSESATPESGPGTSTEPSEGSAPGTSTEPSEGSAPGTSTEP | |
| | SEGSAPGTSTEPSEGSAPGTSTEPSEGSAPGTSTEPSEGSAPGSPAGSPTSTEEGTSTEPS | |
| | EGSAPGTSESATPESGPGSEPATSGSETPGTSESATPESGPGSEPATSGSETPGTSESATP | |
| | ESGPGTSTEPSEGSAPGTSESATPESGPGSPAGSPTSTEEGSPAGSPTSTEEGSPAGSPTS | |
| | TEEGTSESATPESGPGTSTEPSEGSAPGTSESATPESGPGSEPATSGSETPGTSESATPES | |
| | GPGSEPATSGSETPGTSESATPESGPGTSTEPSEGSAPGSPAGSPTSTEEGTSESATPESG | |
| | PGSEPATSGSETPGTSESATPESGPGSPAGSPTSTEEGSPAGSPTSTEEGTSTEPSEGSAP | |
| | GTSESATPESGPGTSESATPESGPGTSESATPESGPGSEPATSGSETPGSEPATSGSETPG | |
| | SPAGSPTSTEEGTSTEPSEGSAPGTSTEPSEGSAPGSEPATSGSETPGTSESATPESGPGT | |
| | STEPSEGSAP | |
| AF864 | GSTSESPSGTAPGTSPSGESSTAPGSTSESPSGTAPGSTSESPSGTAPGTSTPESGSASPG | 121 |
| | TSTPESGSASPGSTSESPSGTAPGSTSESPSGTAPGTSPSGESSTAPGSTSESPSGTAPGTS | |
| | PSGESSTAPGTSPSGESSTAPGSTSSTAESPGPGTSPSGESSTAPGTSPSGESSTAPGSTSS | |
| | TAESPGPGTSTPESGSASPGTSTPESGSASPGSTSESPSGTAPGSTSESPSGTAPGTSTPES | |
| | GSASPGSTSSTAESPGPGTSTPESGSASPGSTSESPSGTAPGTSPSGESSTAPGSTSSTAES | |
| | PGPGTSPSGESSTAPGTSTPESGSASPGSTSSTAESPGPGSTSSTAESPGPGSTSSTAESPG | |
| | PGSTSSTAESPGPGTSPSGESSTAPGSTSESPSGTAPGSTSESPSGTAPGTSTPESGPXXX | |
| | GASASGAPSTXXXXSESPSGTAPGSTSESPSGTAPGSTSESPSGTAPGSTSESPSGTAPG | |
| | STSESPSGTAPGSTSESPSGTAPGTSTPESGSASPGTSPSGESSTAPGTSPSGESSTAPGST | |
| | SSTAESPGPGTSPSGESSTAPGTSTPESGSASPGSTSESPSGTAPGSTSESPSGTAPGTSPS | |
| | GESSTAPGSTSESPSGTAPGTSTPESGSASPGTSTPESGSASPGSTSESPSGTAPGTSTPES | |
| | GSASPGSTSSTAESPGPGSTSESPSGTAPGSTSESPSGTAPGTSPSGESSTAPGSTSSTAES | |
| | PGPGTSPSGESSTAPGTSTPESGSASPGTSPSGESSTAPGTSPSGESSTAPGTSPSGESSTA | |
| | PGSTSSTAESPGPGSTSSTAESPGPGTSPSGESSTAPGSSPSASTGTGPGSSTPSGATGSP | |
| | GSSTPSGATGSP | |
| AG864 | GASPGTSSTGSPGSSPSASTGTGPGSSPSASTGTGPGTPGSGTASSSPGSSTPSGATGSPG | 122 |
| | SSPSASTGTGPGASPGTSSTGSPGTPGSGTASSSPGSSTPSGATGSPGTPGSGTASSSPGA | |
| | SPGTSSTGSPGASPGTSSTGSPGTPGSGTASSSPGSSTPSGATGSPGASPGTSSTGSPGTP | |
| | GSGTASSSPGSSTPSGATGSPGSSPSASTGTGPGSSPSASTGTGPGSSTPSGATGSPGSST | |
| | PSGATGSPGASPGTSSTGSPGASPGTSSTGSPGASPGTSSTGSPGTPGSGTASSSPGASP | |
| | GTSSTGSPGASPGTSSTGSPGASPGTSSTGSPGSSPSASTGTGPGTPGSGTASSSPGASPG | |
| | TSSTGSPGASPGTSSTGSPGASPGTSSTGSPGSSTPSGATGSPGSSTPSGATGSPGASPGT | |
| | SSTGSPGTPGSGTASSSPGSSTPSGATGSPGSSTPSGATGSPGSSTPSGATGSPGSSPSAS | |
| | TGTGPGASPGTSSTGSPGASPGTSSTGSPGTPGSGTASSSPGASPGTSSTGSPGASPGTS | |
| | STGSPGASPGTSSTGSPGASPGTSSTGSPGTPGSGTASSSPGSSTPSGATGSPGTPGSGT | |
| | ASSSPGSSTPSGATGSPGTPGSGTASSSPGSSTPSGATGSPGSSTPSGATGSPGSSPSAST | |
| | GTGPGSSPSASTGTGPGASPGTSSTGSPGTPGSGTASSSPGSSTPSGATGSPGSSPSASTG | |
| | TGPGSSPSASTGTGPGASPGTSSTGSPGASPGTSSTGSPGSSTPSGATGSPGSSPSASTGT | |
| | GPGASPGTSSTGSPGSSPSASTGTGPGTPGSGTASSSPGSSTPSGATGSPGSSTPSGATGS | |
| | PGASPGTSSTGSP | |
| AM875 | GTSTEPSEGSAPGSEPATSGSETPGSPAGSPTSTEEGSTSSTAESPGPGTSTPESGSASPG | 123 |
| | STSESPSGTAPGSTSESPSGTAPGTSTPESGSASPGTSTPESGSASPGSEPATSGSETPGTS | |
| | ESATPESGPGSPAGSPTSTEEGTSTEPSEGSAPGTSESATPESGPGTSTEPSEGSAPGTST | |
| | EPSEGSAPGSPAGSPTSTEEGTSTEPSEGSAPGTSTEPSEGSAPGTSESATPESGPGTSES | |
| | ATPESGPGTSTEPSEGSAPGTSTEPSEGSAPGTSESATPESGPGTSTEPSEGSAPGSEPAT | |
| | SGSETPGSPAGSPTSTEEGSSTPSGATGSPGTPGSGTASSSPGSSTPSGATGSPGTSTEPS | |
| | EGSAPGTSTEPSEGSAPGSEPATSGSETPGSPAGSPTSTEEGSPAGSPTSTEEGTSTEPSE | |
| | GSAPGASASGAPSTGGTSESATPESGPGSPAGSPTSTEEGSPAGSPTSTEEGSTSSTAESP | |
| | GPGSTSESPSGTAPGTSPSGESSTAPGTPGSGTASSSPGSSTPSGATGSPGSSPSASTGTG | |
| | PGSEPATSGSETPGTSESATPESGPGSEPATSGSETPGSTSSTAESPGPGSTSSTAESPGP | |
| | GTSPSGESSTAPGSEPATSGSETPGSEPATSGSETPGTSTEPSEGSAPGSTSSTAESPGPG | |
| | TSTPESGSASPGSTSESPSGTAPGTSTEPSEGSAPGTSTEPSEGSAPGTSTEPSEGSAPGSS | |
| | TPSGATGSPGSSPSASTGTGPGASPGTSSTGSPGSEPATSGSETPGTSESATPESGPGSPA | |
| | GSPTSTEEGSSTPSGATGSPGSSPSASTGTGPGASPGTSSTGSPGTSESATPESGPGTSTE | |
| | PSEGSAPGTSTEPSEGSAP | |
| AE912 | MAEPAGSPTSTEEGTPGSGTASSSPGSSTPSGATGSPGASPGTSSTGSPGSPAGSPTSTE | 124 |
| | EGTSESATPESGPGTSTEPSEGSAPGSPAGSPTSTEEGTSTEPSEGSAPGTSTEPSEGSAP | |
| | GTSESATPESGPGSEPATSGSETPGSEPATSGSETPGSPAGSPTSTEEGTSESATPESGPG | |
| | TSTEPSEGSAPGTSTEPSEGSAPGSPAGSPTSTEEGTSTEPSEGSAPGTSTEPSEGSAPGT | |
| | SESATPESGPGTSTEPSEGSAPGTSESATPESGPGSEPATSGSETPGTSTEPSEGSAPGTS | |
| | TEPSEGSAPGTSESATPESGPGTSESATPESGPGSPAGSPTSTEEGTSESATPESGPGSEP | |
| | ATSGSETPGTSESATPESGPGTSTEPSEGSAPGTSTEPSEGSAPGTSTEPSEGSAPGTSTE | |
| | PSEGSAPGTSTEPSEGSAPGTSTEPSEGSAPGSPAGSPTSTEEGTSTEPSEGSAPGTSESA | |
| | TPESGPGSEPATSGSETPGTSESATPESGPGSEPATSGSETPGTSESATPESGPGTSTEPS | |
| | EGSAPGTSESATPESGPGSPAGSPTSTEEGSPAGSPTSTEEGSPAGSPTSTEEGTSESATP | |
| | ESGPGTSTEPSEGSAPGTSESATPESGPGSEPATSGSETPGTSESATPESGPGSEPATSGS | |
| | ETPGTSESATPESGPGTSTEPSEGSAPGSPAGSPTSTEEGTSESATPESGPGSEPATSGSE | |
| | TPGTSESATPESGPGSPAGSPTSTEEGSPAGSPTSTEEGTSTEPSEGSAPGTSESATPESG | |
| | PGTSESATPESGPGTSESATPESGPGSEPATSGSETPGSEPATSGSETPGSPAGSPTSTEE | |
| | GTSTEPSEGSAPGTSTEPSEGSAPGSEPATSGSETPGTSESATPESGPGTSTEPSEGSAP | |
| AM923 | MAEPAGSPTSTEEGASPGTSSTGSPGSSTPSGATGSPGSSTPSGATGSPGTSTEPSEGSA | 125 |
| | PGSEPATSGSETPGSPAGSPTSTEEGSTSSTAESPGPGTSTPESGSASPGSTSESPSGTAP | |
| | GSTSESPSGTAPGTSTPESGSASPGTSTPESGSASPGSEPATSGSETPGTSESATPESGPG | |
| | SPAGSPTSTEEGTSTEPSEGSAPGTSESATPESGPGTSTEPSEGSAPGTSTEPSEGSAPGS | |
| | PAGSPTSTEEGTSTEPSEGSAPGTSTEPSEGSAPGTSESATPESGPGTSESATPESGPGTS | |
| | TEPSEGSAPGTSTEPSEGSAPGTSESATPESGPGTSTEPSEGSAPGSEPATSGSETPGSPA | |
| | GSPTSTEEGSSTPSGATGSPGTPGSGTASSSPGSSTPSGATGSPGTSTEPSEGSAPGTSTE | |
| | PSEGSAPGSEPATSGSETPGSPAGSPTSTEEGSPAGSPTSTEEGTSTEPSEGSAPGASASG | |
| | APSTGGTSESATPESGPGSPAGSPTSTEEGSPAGSPTSTEEGSTSSTAESPGPGSTSESPS | |
| | GTAPGTSPSGESSTAPGTPGSGTASSSPGSSTPSGATGSPGSSPSASTGTGPGSEPATSGS | |
| | ETPGTSESATPESGPGSEPATSGSETPGSTSSTAESPGPGSTSSTAESPGPGTSPSGESST | |
| | APGSEPATSGSETPGSEPATSGSETPGTSTEPSEGSAPGSTSSTAESPGPGTSTPESGSAS | |
| | PGSTSESPSGTAPGTSTEPSEGSAPGTSTEPSEGSAPGTSTEPSEGSAPGSSTPSGATGSP | |
| | GSSPSASTGTGPGASPGTSSTGSPGSEPATSGSETPGTSESATPESGPGSPAGSPTSTEEG | |
| | SSTPSGATGSPGSSPSASTGTGPGASPGTSSTGSPGTSESATPESGPGTSTEPSEGSAPGT | |
| | STEPSEGSAP | |
| AM1318 | GTSTEPSEGSAPGSEPATSGSETPGSPAGSPTSTEEGSTSSTAESPGPGTSTPESGSASPG | 126 |
| | STSESPSGTAPGSTSESPSGTAPGTSTPESGSASPGTSTPESGSASPGSEPATSGSETPGTS | |
| | ESATPESGPGSPAGSPTSTEEGTSTEPSEGSAPGTSESATPESGPGTSTEPSEGSAPGTST | |
| | EPSEGSAPGSPAGSPTSTEEGTSTEPSEGSAPGTSTEPSEGSAPGTSESATPESGPGTSES | |
| | ATPESGPGTSTEPSEGSAPGTSTEPSEGSAPGTSESATPESGPGTSTEPSEGSAPGSEPAT | |
| | SGSETPGSPAGSPTSTEEGSSTPSGATGSPGTPGSGTASSSPGSSTPSGATGSPGTSTEPS | |
| | EGSAPGTSTEPSEGSAPGSEPATSGSETPGSPAGSPTSTEEGSPAGSPTSTEEGTSTEPSE | |
| | GSAPGPEPTGPAPSGGSEPATSGSETPGTSESATPESGPGSPAGSPTSTEEGTSESATPES | |
| | GPGSPAGSPTSTEEGSPAGSPTSTEEGTSESATPESGPGSPAGSPTSTEEGSPAGSPTSTE | |
| | EGSTSSTAESPGPGSTSESPSGTAPGTSPSGESSTAPGSTSESPSGTAPGSTSESPSGTAP | |
| | GTSPSGESSTAPGTSTEPSEGSAPGTSESATPESGPGTSESATPESGPGSEPATSGSETPG | |
| | TSESATPESGPGTSESATPESGPGTSTEPSEGSAPGTSESATPESGPGTSTEPSEGSAPGT | |
| | SPSGESSTAPGTSPSGESSTAPGTSPSGESSTAPGTSTEPSEGSAPGSPAGSPTSTEEGTST | |
| | EPSEGSAPGSSPSASTGTGPGSSTPSGATGSPGSSTPSGATGSPGSSTPSGATGSPGSSTP | |
| | SGATGSPGASPGTSSTGSPGASASGAPSTGGTSPSGESSTAPGSTSSTAESPGPGTSPSG | |
| | ESSTAPGTSESATPESGPGTSTEPSEGSAPGTSTEPSEGSAPGSSPSASTGTGPGSSTPSG | |
| | ATGSPGASPGTSSTGSPGTSTPESGSASPGTSPSGESSTAPGTSPSGESSTAPGTSESATP | |
| | ESGPGSEPATSGSETPGTSTEPSEGSAPGSTSESPSGTAPGSTSESPSGTAPGTSTPESGS | |
| | ASPGSPAGSPTSTEEGTSESATPESGPGTSTEPSEGSAPGSPAGSPTSTEEGTSESATPES | |
| | GPGSEPATSGSETPGSSTPSGATGSPGASPGTSSTGSPGSSTPSGATGSPGSTSESPSGTA | |
| | PGTSPSGESSTAPGSTSSTAESPGPGSSTPSGATGSPGASPGTSSTGSPGTPGSGTASSSP | |
| | GSPAGSPTSTEEGSPAGSPTSTEEGTSTEPSEGSAP | |
| BC 864 | GTSTEPSEPGSAGTSTEPSEPGSAGSEPATSGTEPSGSGASEPTSTEPGSEPATSGTEPSG | 127 |
| | SEPATSGTEPSGSEPATSGTEPSGSGASEPTSTEPGTSTEPSEPGSAGSEPATSGTEPSGT | |
| | STEPSEPGSAGSEPATSGTEPSGSEPATSGTEPSGTSTEPSEPGSAGTSTEPSEPGSAGSE | |
| | PATSGTEPSGSEPATSGTEPSGTSEPSTSEPGAGSGASEPTSTEPGTSEPSTSEPGAGSEP | |
| | ATSGTEPSGSEPATSGTEPSGTSTEPSEPGSAGTSTEPSEPGSAGSGASEPTSTEPGSEPA | |
| | TSGTEPSGSEPATSGTEPSGSEPATSGTEPSGSEPATSGTEPSGTSTEPSEPGSAGSEPAT | |
| | SGTEPSGSGASEPTSTEPGTSTEPSEPGSAGSEPATSGTEPSGSGASEPTSTEPGTSTEPS | |
| | EPGSAGSGASEPTSTEPGSEPATSGTEPSGSGASEPTSTEPGSEPATSGTEPSGSGASEPT | |
| | STEPGTSTEPSEPGSAGSEPATSGTEPSGSGASEPTSTEPGTSTEPSEPGSAGSEPATSGT | |
| | EPSGTSTEPSEPGSAGSEPATSGTEPSGTSTEPSEPGSAGTSTEPSEPGSAGTSTEPSEPGS | |
| | AGTSTEPSEPGSAGTSTEPSEPGSAGTSTEPSEPGSAGTSEPSTSEPGAGSGASEPTSTEP | |
| | GTSTEPSEPGSAGTSTEPSEPGSAGTSTEPSEPGSAGSEPATSGTEPSGSGASEPTSTEPG | |
| | SEPATSGTEPSGSEPATSGTEPSGSEPATSGTEPSGSEPATSGTEPSGTSEPSTSEPGAGS | |
| | EPATSGTEPSGSGASEPTSTEPGTSTEPSEPGSAGSEPATSGTEPSGSGASEPTSTEPGTS | |
| | TEPSEPGSA | |
| BD864 | GSETATSGSETAGTSESATSESGAGSTAGSETSTEAGTSESATSESGAGSETATSGSETA | 128 |
| | GSETATSGSETAGTSTEASEGSASGTSTEASEGSASGTSESATSESGAGSETATSGSETA | |
| | GTSTEASEGSASGSTAGSETSTEAGTSESATSESGAGTSESATSESGAGSETATSGSETA | |
| | GTSESATSESGAGTSTEASEGSASGSETATSGSETAGSETATSGSETAGTSTEASEGSAS | |
| | GSTAGSETSTEAGTSESATSESGAGTSTEASEGSASGSETATSGSETAGSTAGSETSTEA | |
| | GSTAGSETSTEAGSETATSGSETAGTSESATSESGAGTSESATSESGAGSETATSGSETA | |
| | GTSESATSESGAGTSESATSESGAGSETATSGSETAGSETATSGSETAGTSTEASEGSAS | |
| | GSTAGSETSTEAGSETATSGSETAGTSESATSESGAGSTAGSETSTEAGSTAGSETSTE | |
| | AGSTAGSETSTEAGTSTEASEGSASGSTAGSETSTEAGSTAGSETSTEAGTSTEASEGS | |
| | ASGSTAGSETSTEAGSETATSGSETAGTSTEASEGSASGTSESATSESGAGSETATSGSE | |
| | TAGTSESATSESGAGTSESATSESGAGSETATSGSETAGTSESATSESGAGSETATSGSE | |
| | TAGTSTEASEGSASGTSTEASEGSASGSTAGSETSTEAGSTAGSETSTEAGSETATSGSE | |
| | TAGTSESATSESGAGTSESATSESGAGSETATSGSETAGSETATSGSETAGSETATSGSE | |
| | TAGTSTEASEGSASGTSESATSESGAGSETATSGSETAGSETATSGSETAGTSESATSES | |
| | GAGTSESATSESGAGSETATSGSETA | |
| Y288 | GEGSGEGSEGEGSEGSGEGEGSEGSGEGEGGSEGSEGEGGSEGSEGEGGSEGSEGEGS | 129 |
| | GEGSEGEGGSEGSEGEGSGEGSEGEGSEGGSEGEGGSEGSEGEGSGEGSEGEGGEGGS | |
| | EGEGSEGSGEGEGSGEGSEGEGSEGSGEGEGSGEGSEGEGSEGSGEGEGSEGSGEGEG | |
| | GSEGSEGEGSEGSGEGEGGEGSGEGEGSGEGSEGEGGGEGSEGEGSGEGGEGEGSEG | |
| | GSEGEGGSEGGEGEGSEGSGEGEGSEGGSEGEGSEGGSEGEGSEGSGEGEGSEGSGE | |
| Y576 | GEGSGEGSEGEGSEGSGEGEGSEGSGEGEGGSEGSEGEGSEGSGEGEGGEGSGEGEGS | 130 |
| | GEGSEGEGGGEGSEGEGSGEGGEGEGSEGGSEGEGGSEGGEGEGSEGSGEGEGSEGG | |
| | SEGEGSEGGSEGEGSEGSGEGEGSEGSGEGEGSEGSGEGEGSEGSGEGEGSEGGSEGE | |
| | GGSEGSEGEGSGEGSEGEGGSEGSEGEGGGEGSEGEGSGEGSEGEGGSEGSEGEGGSE | |
| | GSEGEGGEGSGEGEGSEGSGEGEGSGEGSEGEGSEGSGEGEGSEGSGEGEGGSEGSEG | |
| | EGSGEGSEGEGSEGSGEGEGSEGSGEGEGGSEGSEGEGGSEGSEGEGGSEGSEGEGGE | |
| | GSGEGEGSEGSGEGEGSGEGSEGEGSEGSGEGEGSEGSGEGEGGSEGSEGEGSEGSGE | |
| | GEGGEGSGEGEGSGEGSEGEGGGEGSEGEGSEGSGEGEGSEGSGEGEGSEGGSEGEG | |
| | GSEGSEGEGSEGGSEGEGSEGGSEGEGSEGSGEGEGSEGSGEGEGSGEGSEGEGGSEG | |
| | GEGEGSEGGSEGEGSEGGSEGEGGEGSGEGEGGGEGSEGEGSEGSGEGEGSGEGSE | |

4. N-Terminal XTEN Expression-Enhancing Sequences

In some embodiments, the invention provides a short-length XTEN sequence incorporated as the N-terminal portion of the BFXTEN fusion protein. It has been discovered that the expression of the fusion protein is enhanced in a host cell transformed with a suitable expression vector comprising an optimized N-terminal leader polynucleotide sequence (that encodes the N-terminal XTEN) incorporated into the polynucleotide encoding the binding fusion protein. As described in Examples 14-17, a host cell transformed with such an expression vector comprising an optimized N-terminal leader sequence (NTS) in the binding fusion protein gene results in greatly-enhanced expression of the fusion protein compared to the expression of a corresponding fusion protein from a polynucleotide not comprising the NTS, and obviates the need for incorporation of a non-XTEN leader sequence used to enhance expression. In one embodiment, the invention provides BFXTEN fusion proteins comprising an NTS wherein the expression of the binding fusion protein from the encoding gene in a host cell is enhanced about 50%, or about 75%, or about 100%, or about 150%, or about 200%, or about 400% compared to expression of a BFXTEN fusion protein not comprising the N-terminal XTEN sequence (where the encoding gene lacks the NTS).

In one embodiment, the N-terminal XTEN polypeptide of the BFXTEN comprises a sequence that exhibits at least about 80%, more preferably at least about 90%, more preferably at least about 91%, more preferably at least about 92%, more preferably at least about 93%, more preferably at least about 94%, more preferably at least about 95%, more preferably at least about 96%, more preferably at least about 97%, more preferably at least about 98%, more preferably at least 99%, or exhibits 100% sequence identity compared to the amino acid sequence of AE48 or AM48, the respective amino acid sequences of which are as follows:

(SEQ ID NO: 131)
AE48:
MAEPAGSPTSTEEGTPGSGTASSSPGSSTPSGATGSPGASPGTSSTGS
(SEQ ID NO: 132)
AM48:
MAEPAGSPTSTEEGASPGTSSTGSPGSSTPSGATGSPGSSTPSGATGS

In another embodiment, the short-length N-terminal XTEN is linked to an XTEN of longer length to form the N-terminal region of the BFXTEN fusion protein, wherein the polynucleotide sequence encoding the short-length N-terminal XTEN confers the property of enhanced expression in the host cell, and wherein the long length of the expressed XTEN contributes to the enhanced properties of the XTEN carrier in the fusion protein, as described above. In the foregoing, the short-length XTEN is linked to any of the XTEN disclosed herein (e.g., an XTEN of Table 4) and the resulting XTEN, in turn, is linked to the N-terminal of any of the BP disclosed herein (e.g., a BP of Table 1 or a sequence variant or fragment thereof) as a component of the fusion protein. Alternatively, polynucleotides encoding the short-length XTEN (or its complement) is linked to polynucleotides encoding any of the XTEN (or its complement) disclosed herein and the resulting gene encoding the N-terminal XTEN, in turn, is linked to the 5′ end of polynucleotides encoding any of the BP (or to the 3′ end of its complement) disclosed herein. In some embodiments, the N-terminal XTEN polypeptide with long length exhibits at least about 80%, or at least about 90%, or at least about 91%, or at least about 92%, or at least about 93%, or at least about 94%, or at least about 95%, or at least about 96%, or at least about 97%, or at least about 98%, or at least 99%, or exhibits 100% sequence identity compared to an amino acid sequence selected from the group consisting of the sequences AE624, AE912, and AM923.

In any of the foregoing N-terminal XTEN embodiments described above, the N-terminal XTEN can have from about one to about six additional amino acid residues, preferably selected from GESTPA, to accommodate the endonuclease restriction sites that is employed to join the nucleotides encoding the N-terminal XTEN to the gene encoding the targeting moiety of the fusion protein. Non-limiting examples of amino acids compatible with the restrictions sites and the preferred amino acids are listed in Table 5, below. The methods for the generation of the N-terminal sequences and incorporation into the fusion proteins of the invention are described more fully in the Examples.

5. Net Charge

In other embodiments, the XTEN polypeptides have an unstructured characteristic imparted by incorporation of amino acid residues with a net charge and containing a low proportion or no hydrophobic amino acids in the XTEN sequence. The overall net charge and net charge density is controlled by modifying the content of charged amino acids in the XTEN sequences, either positive or negative, with the net charge typically represented as the percentage of amino acids in the polypeptide contributing to a charged state beyond those residues that are cancelled by a residue with an opposing charge. In some embodiments, the net charge density of the XTEN of the compositions may be above +0.1 or below −0.1 charges/residue. By “net charge density” of a protein or peptide herein is meant the net charge divided by the total number of amino acids in the protein or propeptide. In other embodiments, the net charge of an XTEN can be about 0%, about 1%, about 2%, about 3%, about 4%, about 5%, about 6%, about 7%, about 8%, about 9%, about 10% about 11%, about 12%, about 13%, about 14%, about 15%, about 16%, about 17%, about 18%, about 19%, or about 20% or more. In some embodiments, the XTEN sequence comprises charged residues separated by other residues such as serine or glycine, which leads to better expression or purification behavior. Based on the net charge, some XTENs have an isoelectric point (pI) of 1.0, 1.5, 2.0, 2.5, 3.0, 3.5, 4.0, 4.5, 5.0, 5.5, 6.0, or even 6.5. In preferred embodiments, the XTEN will have an isoelectric point between 1.5 and 4.5 and carry a net negative charge under physiologic conditions.

Not to be bound by a particular theory, it is believed that the XTEN can adopt open conformations due to electrostatic repulsion between individual amino acids of the XTEN polypeptide that individually carry a net negative charge and that are distributed across the sequence of the XTEN polypeptide. Such a distribution of net negative charge in the extended sequence lengths of XTEN can lead to an unstructured conformation that, in turn, can result in an effective increase in hydrodynamic radius. Since most tissues and surfaces in a human or animal have a net negative charge, in some embodiments, the XTEN sequences are designed to have a net negative charge to minimize non-specific interactions between the XTEN containing compositions and various surfaces such as blood vessels, healthy tissues, or various receptors, which would further contribute to reduced active clearance of the fusion protein comprising XTEN with a net negative charge.

In preferred embodiments, the negative charge of the subject XTEN is conferred by incorporation of glutamic acid residues. For example, where an XTEN with a negative charge is desired, the XTEN can be selected solely from an AE family sequence, which has approximately a 17% net charge due to incorporated glutamic acid, or can include varying proportions of glutamic acid-containing motifs of Table 3 to provide the desired degree of net charge. Non-limiting examples of AE XTEN include, but are not limited to the AE36, AE42, AE48, AE144, AE288, AE576, AE624, AE864, and AE912 polypeptide sequences of Tables 4 and 10, or fragments thereof. In one embodiment, an XTEN sequence of Tables 4 or 9-13 can be modified to include additional glutamic acid residues to achieve the desired net negative charge. Accordingly, in one embodiment the invention provides XTEN in which the XTEN sequences contain about 1%, 2%, 4%, 8%, 10%, 15%, 17%, 20%, 25%, or even about 30% glutamic acid. Generally, the glutamic residues are spaced uniformly across the XTEN sequence. In some cases, the XTEN can contain about 10-80, or about 15-60, or about 20-50 glutamic residues per 20 kDa of XTEN that can result in an XTEN with charged residues that would have very similar pKa, which can increase the charge homogeneity of the product and sharpen its isoelectric point, enhance the physicochemical properties of the resulting BFXTEN fusion protein for, and hence, simplifying purification procedures. In one embodiment, the invention contemplates incorporation of aspartic acid residues into XTEN in addition to glutamic acid in order to achieve a net negative charge.

In other embodiments, where no net charge is desired, the XTEN can be selected from, for example, AG XTEN components, such as the AG motifs of Table 3, or those AM motifs of Table 3 that have no net charge. Non-limiting examples of AG XTEN include, but are not limited to AG42, AG144, AG288, AG576, and AG864 polypeptide sequences of Tables 4 and 11, or fragments thereof. In another embodiment, the XTEN can comprise varying proportions of AE and AG motifs in order to have a net charge that is deemed optimal for a given use or to maintain a given physicochemical property.

The XTEN of the compositions of the present invention generally have no or a low content of positively charged amino acids. In some embodiments, the XTEN may have less than about 10% amino acid residues with a positive charge, or less than about 7%, or less than about 5%, or less than about 2%, or less than about 1% amino acid residues with a positive charge. However, the invention contemplates constructs where a limited number of amino acids with a positive charge, such as lysine, are incorporated into XTEN to permit conjugation between the epsilon amine of the lysine and a reactive group on a peptide, a linker bridge, or a reactive group on a drug or small molecule to be conjugated to the XTEN backbone. In one embodiment of the foregoing, the XTEN has between about 1 to about 100 lysine residues, or about 1 to about 70 lysine residues, or about 1 to about 50 lysine residues, or about 1 to about 30 lysine residues, or about 1 to about 20 lysine residues, or about 1 to about 10 lysine residues, or about 1 to about 5 lysine residues, or alternatively only a single lysine residue. Using the foregoing lysine-containing XTEN, fusion proteins are constructed that comprises XTEN, a BP, plus a chemotherapeutic agent useful in the treatment of growth-related diseases or disorders linked to the lysine, wherein the maximum number of molecules of the agent incorporated into the XTEN component is determined by the numbers of lysines or other amino acids with reactive side chains (e.g., cysteine) incorporated into the XTEN.

As hydrophobic amino acids can impart structure to a polypeptide, the invention provides that the content of hydrophobic amino acids in the XTEN will typically be less than 5%, or less than 2%, or less than 1% hydrophobic amino acid content. In one embodiment, the amino acid content of methionine and tryptophan in the XTEN component of a BFXTEN fusion protein is less than 5%, or less than 2%, and most preferably less than 1%. In another embodiment, the XTEN will have a sequence that has less than 10% amino acid residues with a positive charge, the sum of methionine and tryptophan residues will be less than 2%, and the sum of asparagine and glutamine residues will be less than 10% of the total XTEN sequence.

6. Low Immunogenicity

In another aspect, the invention provides BFXTEN in which the XTEN sequences have a low degree of immunogenicity or are substantially non-immunogenic. Several factors can contribute to the low immunogenicity of XTEN, including but not limited to the non-repetitive sequence, the unstructured conformation, the high degree of solubility, the low degree or lack of self-aggregation, the low degree or lack of proteolytic sites within the sequence, and the low degree or lack of epitopes in the XTEN sequence.

Conformational epitopes are formed by regions of the protein surface that are composed of multiple discontinuous amino acid sequences of the protein antigen. The precise folding of the protein brings these sequences into a well-defined, stable spatial configurations, or epitopes, that can be recognized as “foreign” by the host humoral immune system, resulting in the production of antibodies to the protein or triggering a cell-mediated immune response. In the latter case, the immune response to a protein in an individual is heavily influenced by T-cell epitope recognition that is a function of the peptide binding specificity of that individual's HLA-DR allotype. Engagement of a MHC Class II peptide complex by a cognate T-cell receptor on the surface of the T-cell, together with the cross-binding of certain other co-receptors such as the CD4 molecule, can induce an activated state within the T-cell. Activation leads to the release of cytokines further activating other lymphocytes such as B cells to produce antibodies or activating T killer cells as a full cellular immune response.

The ability of a peptide to bind a given MHC Class II molecule for presentation on the surface of an APC (antigen presenting cell) is dependent on a number of factors; most notably its primary sequence. In one embodiment, a lower degree of immunogenicity may be achieved by designing XTEN sequences that resist antigen processing in antigen presenting cells, and/or choosing sequences that do not bind MHC receptors well. The invention provides BFXTEN with substantially non-repetitive XTEN polypeptides designed to reduce binding with MHC II receptors, as well as avoiding formation of epitopes for T-cell receptor or antibody binding, resulting in a low degree of immunogenicity. Avoidance of immunogenicity is, in part, a direct result of the conformational flexibility of XTEN sequences; i.e., the lack of secondary structure due to the selection and order of amino acid residues. For example, of particular interest are sequences having a low tendency to adapt compactly folded conformations in aqueous solution or under physiologic conditions that could result in conformational epitopes. The administration of fusion proteins comprising XTEN, using conventional therapeutic practices and dosing, would generally not result in the formation of neutralizing antibodies to the XTEN sequence, and may also reduce the immunogenicity of the BP fusion partner in the BFXTEN compositions.

In one embodiment, the XTEN sequences utilized in the subject fusion proteins can be substantially free of epitopes recognized by human T cells. The elimination of such epitopes for the purpose of generating less immunogenic proteins has been disclosed previously; see for example WO 98/52976, WO 02/079232, and WO 00/3317 which are incorporated by reference herein. Assays for human T cell epitopes have been described (Stickler, M., et al. (2003) J Immunol Methods, 281: 95-108). Of particular interest are peptide sequences that can be oligomerized without generating T cell epitopes or non-human sequences. This is achieved by testing direct repeats of these sequences for the presence of T-cell epitopes and for the occurrence of 6 to 15-mer and, in particular, 9-mer sequences that are not human, and then altering the design of the XTEN sequence to eliminate or disrupt the epitope sequence. In some embodiments, the XTEN sequences are substantially non-immunogenic by the restriction of the numbers of epitopes of the XTEN predicted to bind MHC receptors. With a reduction in the numbers of epitopes capable of binding to MHC receptors, there is a concomitant reduction in the potential for T cell activation as well as T cell helper function, reduced B cell activation or upregulation and reduced antibody production. The low degree of predicted T-cell epitopes can be determined by epitope prediction algorithms such as, e.g., TEPITOPE (Sturniolo, T., et al. (1999) Nat Biotechnol, 17: 555-61), as shown in Example 36. The TEPITOPE score of a given peptide frame within a protein is the log of the K_{d} (dissociation constant, affinity, off-rate) of the binding of that peptide frame to multiple of the most common human MHC alleles, as disclosed in Sturniolo, T. et al. (1999) Nature Biotechnology 17:555). The score ranges over at least 20 logs, from about 10 to about −10 (corresponding to binding constraints of 10e¹⁰ K_{d} to 10e⁻¹⁰ K_{d}), and can be reduced by avoiding hydrophobic amino acids that serve as anchor residues during peptide display on MHC, such as M, I, L, V, F. In some embodiments, an XTEN component incorporated into a BFXTEN does not have a predicted T-cell epitope at a TEPITOPE threshold score of about −5, or −6, or −7, or −8, or −9, or at a TEPITOPE score of −10. As used herein, a score of “−9” would be a more stringent TEPITOPE threshold than a score of −5.

In another embodiment, the XTEN sequence of the subject BFXTEN fusion proteins can be rendered substantially non-immunogenic by the restriction of known proteolytic sites from the sequence of the XTEN, reducing the processing of XTEN into small peptides that can bind to MHC II receptors. In another embodiment, the XTEN sequence can be rendered substantially non-immunogenic by the use a sequence that is substantially devoid of secondary structure, conferring resistance to many proteases due to the high entropy of the structure. Accordingly, the reduced TEPITOPE score and elimination of known proteolytic sites from the XTEN may render the XTEN of the BFXTEN fusion proteins substantially unable to be bound by mammalian receptors, including those of the immune system. In one embodiment, an XTEN of a BFXTEN fusion protein can have >100 nM K_{d} binding to a mammalian receptor, or greater than 500 nM K_{d}, or greater than 1 μM K_{d} towards a mammalian cell surface or circulating polypeptide receptor.

Additionally, the non-repetitive sequence and corresponding lack of epitopes of XTEN can limit the ability of B cells to bind to or be activated by XTEN. A repetitive sequence is recognized and can form multivalent contacts with even a few B cells and, as a consequence of the cross-linking of multiple T-cell independent receptors, can stimulate B cell proliferation and antibody production. In contrast, while a XTEN can make contacts with many different B cells over its extended sequence, each individual B cell may only make one or a small number of contacts with an individual XTEN due to the lack of repetitiveness of the sequence. As a result, XTENs typically may have a much lower tendency to stimulate proliferation of B cells and thus an immune response. In one embodiment, the BFXTEN may have reduced immunogenicity as compared to the corresponding BP that is not fused. In one embodiment, the administration of up to three parenteral doses of a BFXTEN to a mammal may result in detectable anti-BFXTEN IgG at a serum dilution of 1:100 but not at a dilution of 1:1000. In another embodiment, the administration of up to three parenteral doses of an BFXTEN to a mammal may result in detectable anti-BP IgG at a serum dilution of 1:100 but not at a dilution of 1:1000. In another embodiment, the administration of up to three parenteral doses of an BFXTEN to a mammal may result in detectable anti-XTEN IgG at a serum dilution of 1:100 but not at a dilution of 1:1000. In the foregoing embodiments, the mammal can be a mouse, a rat, a rabbit, or a cynomolgus monkey.

An additional feature of XTENs with non-repetitive sequences relative to sequences with a high degree of repetitiveness can be that non-repetitive XTENs form weaker contacts with antibodies. Antibodies are multivalent molecules. For instance, IgGs have two identical binding sites and IgMs contain 10 identical binding sites. Thus antibodies against repetitive sequences can form multivalent contacts with such repetitive sequences with high avidity, which can affect the potency and/or elimination of such repetitive sequences. In contrast, antibodies against non-repetitive XTENs may yield monovalent interactions, resulting in less likelihood of immune clearance such that the BFXTEN compositions can remain in circulation for an increased period of time.

7. Increased Hydrodynamic Radius

In another aspect, the present invention provides BFXTEN in which the XTEN sequences can have a high hydrodynamic radius that confers a corresponding increased apparent molecular weight to the BFXTEN fusion protein. As detailed in Example 19, the linking of XTEN to BP sequences can result in BFXTEN compositions that can have increased hydrodynamic radii, increased apparent molecular weight, and increased apparent molecular weight factor compared to a BP not linked to an XTEN. For example, in therapeutic applications in which prolonged half-life is desired, compositions in which a XTEN with a high hydrodynamic radius is incorporated into a fusion protein comprising one or more BP can effectively enlarge the hydrodynamic radius of the composition beyond the glomerular pore size of approximately 3-5 nm (corresponding to an apparent molecular weight of about 70 kDA) (Caliceti. 2003. Pharmacokinetic and biodistribution properties of poly(ethylene glycol)-protein conjugates. Adv Drug Deliv Rev 55:1261-1277), resulting in reduced renal clearance of circulating proteins. The hydrodynamic radius of a protein is determined by its molecular weight as well as by its structure, including shape and compactness. Not to be bound by a particular theory, the XTEN can adopt open conformations due to electrostatic repulsion between individual charges of the peptide or the inherent flexibility imparted by the particular amino acids in the sequence that lack potential to confer secondary structure. The open, extended and unstructured conformation of the XTEN polypeptide can have a greater proportional hydrodynamic radius compared to polypeptides of a comparable sequence length and/or molecular weight that have secondary and/or tertiary structure, such as typical globular proteins. Methods for determining the hydrodynamic radius are well known in the art, such as by the use of size exclusion chromatography (SEC), as described in U.S. Pat. Nos. 6,406,632 and 7,294,513. As the results of Example 19 demonstrate, the addition of increasing lengths of XTEN results in proportional increases in the parameters of hydrodynamic radius, apparent molecular weight, and apparent molecular weight factor, permitting the tailoring of BXTEN to desired characteristic cut-offs. Accordingly, in certain embodiments, the BFXTEN fusion protein can be configured to have a hydrodynamic radius of at least about 5 nm, or at least about 8 nm, or at least about 10 nm, or 12 nm, or at least about 15 nm. In the foregoing embodiments, the large hydrodynamic radius conferred by the XTEN in an BFXTEN fusion protein can lead to reduced renal clearance of the resulting fusion protein, leading to a corresponding increase in terminal half-life, an increase in mean residence time, and/or a decrease in renal clearance rate.

In another embodiment, the invention provides BFXTEN wherein the length of the XTEN is chosen and selectively linked to a BP to create a fusion protein that has, under physiologic conditions, an apparent molecular weight of at least about 150 kDa, or at least about 300 kDa, or at least about 400 kDa, or at least about 500 kDA, or at least about 600 kDa, or at least about 700 kDA, or at least about 800 kDa, or at least about 900 kDa, or at least about 1000 kDa, or at least about 1200 kDa, or at least about 1500 kDa, or at least about 1800 kDa, or at least about 2000 kDa, or at least about 2300 kDa or more. In another embodiment, an XTEN of a chosen length and is linked to a BP to result in a BFXTEN fusion protein that has, under physiologic conditions, an apparent molecular weight factor of at least three, alternatively of at least four, alternatively of at least five, alternatively of at least six, alternatively of at least eight, alternatively of at least 10, alternatively of at least 15, or an apparent molecular weight factor of at least 20 or greater. In another embodiment, the BFXTEN fusion protein has, under physiologic conditions, an apparent molecular weight factor that is about 4 to about 20, or is about 6 to about 15, or is about 8 to about 12, or is about 9 to about 10.

### III). Bifunctional Fusion Protein Composition Configurations

The invention provides BFXTEN fusion protein compositions with the BP and XTEN components linked in specific N- to C-terminus configurations. In some embodiments, the composition is a monomeric BMXTEN fusion protein with two different BP linked to one or more XTEN polypeptides. In other embodiments, the bifunctional combination BCXTEN composition can include a first fusion protein comprising a first BP linked to one or more XTEN polypeptides and a second fusion protein comprising a second BP different from the first BP that is linked to one or more XTEN polypeptides. It is specifically intended that BFXTEN encompasses both BMXTEN and BCXTEN forms of the compositions. The invention contemplates BFXTEN comprising, but not limited to BP selected from Table 1 or fragments or sequence variants thereof, and XTEN selected from Tables 4 or 9-12 or sequence variants or fragments thereof. In one embodiment, the BP incorporated into BFXTEN fusion protein each have a sequence that exhibits at least about 80% sequence identity to sequences from Table 1, alternatively at least about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99% sequence identity as compared with sequences from Table 1, and one or more XTEN that each exhibit at least about 80% sequence identity to a sequence from Table 1, alternatively at least about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99% sequence identity as compared with a sequence from Tables 4 or 9-12.

In one embodiment, the invention provides compositions of two monomeric fusion proteins comprising a first fusion protein comprising a first biologically active protein (BP1) linked to an XTEN and a second fusion protein comprising a second biologically active protein (BP2) different from BP1, each linked to an XTEN that can be identical or can be different. In one embodiment of the bispecifiic combination BFXTEN composition, wherein both BP require a free N-terminus for optimal biological activity, the invention provides compositions of a fusion protein of formula I:

(BP1)-(S)ₓ-(XTEN) I

and a second fusion protein, wherein the fusion protein is of formula II:

(BP2)-(S)_{y}-(XTEN) II

wherein independently for each occurrence, BP1 is a is a biologically active protein (BP) as described hereinabove; BP2 is a is a biologically active protein different from BP1; S is a spacer sequence having between 1 to about 50 amino acid residues that can optionally include a cleavage sequence or amino acids compatible with restriction sites (as described more fully below); x is either 0 or 1; y is either 0 or 1; and XTEN is an extended recombinant polypeptide as described hereinabove.

In another embodiment of the combination BFXTEN composition, wherein both BP require a free C-terminus for optimal biological activity, the invention provides a fusion protein of formula III:

(XTEN)-(S)ₓ-(BP1) III

and a second fusion protein, wherein the fusion protein is of formula IV:

(XTEN)-(S)_{y}-(BP2) IV

wherein independently for each occurrence, BP1 is a is a biologically active protein (BP) as described hereinabove; BP2 is a is a biologically active protein different from BP1; BP2 is a is a biologically active protein different from BP1; S is a spacer sequence having between 1 to about 50 amino acid residues that can optionally include a cleavage sequence or amino acids compatible with restriction sites (as described more fully below); x is either 0 or 1; y is either 0 or 1; and XTEN is an extended recombinant polypeptide as described hereinabove.

In another embodiment, the invention provides bispecifiic combination BFXTEN compositions comprising a fusion protein of formula I and formula IV. In another embodiment, the invention provides bispecifiic combination BFXTEN compositions comprising a fusion protein of formula II and formula IIII.

Thus, the invention encompasses combination BFXTEN comprising two fusion proteins in at least the following permutations of configurations, each listed in an N- to C-terminus orientation: BP1-XTEN+BP2-XTEN; BP1-XTEN+XTEN-BP2; XTEN-BP1+XTEN-BP2; XTEN-BP1+BP2-XTEN; BP1-S-XTEN+BP2-XTEN; BP1-XTEN+BP2-S-XTEN; BP1-S-XTEN+BP2-S-XTEN; BP1-S-XTEN+XTEN-BP2: BP1-XTEN+XTEN-S-BP2: BP1-S-XTEN+XTEN-S-BP2; XTEN-S-BP1+XTEN-BP2; XTEN-BP1+XTEN-S-BP2; XTEN-S-BP1+XTEN-S-BP2; XTEN-S-BP1+BP2-XTEN; XTEN-BP1+BP2-S-XTEN; or XTEN-S-BP1+BP2-S-XTEN.

In another embodiment, the invention provides an isolated fusion protein, wherein the fusion protein is of formula V:

(XTEN)ᵤ-(S)ᵥ-(BP1)-(S)_{w}-(XTEN)-(S)ₓ-(BP2)-(S)_{y}-(XTEN)_{z} V

wherein independently for each occurrence, BP1 is a is a biologically active protein (BP) as described hereinabove; BP2 is a is a biologically active protein different from BP1; S is a spacer sequence having between 1 to about 50 amino acid residues that can optionally include a cleavage sequence (as described more fully below); u is either 0 or 1; v is either 0 or 1; w is either 0 or 1; x is either 0 or 1; y is either 0 or 1; z is either 0 or 1, with the proviso that u+v+w+x+y+z≧1; and XTEN is an extended recombinant polypeptide as described hereinabove.

In another embodiment, the invention provides an isolated fusion protein, wherein the fusion protein is of formula VI:

(XTEN)ᵥ-(S)_{w}-(BP1)-(S)ₓ-(BP2)-(S)_{y}-(XTEN)_{z} VI

wherein independently for each occurrence, BP1 is a is a biologically active protein (BP) as described hereinabove; BP2 is a is a biologically active protein different from BP1; S is a spacer sequence having between 1 to about 50 amino acid residues that can optionally include a cleavage sequence (as described more fully below); v is either 0 or 1; w is either 0 or 1; x is either 0 or 1; y is either 0 or 1; z is either 0 or 1, with the proviso that v+w+x+y+z≧1; and XTEN is an extended recombinant polypeptide as described hereinabove.

The embodiments of formulae I-VI provide configurations wherein the XTEN are optionally linked to BP via spacer sequences that are designed to incorporate or enhance a functionality or property to the composition. For spacers and methods of identifying desirable spacers, see, for example, George, et al. (2003) Protein Engineering 15:871-879, specifically incorporated by reference herein. In one embodiment, the spacer comprises one or more peptide sequences that are between 1-50 amino acid residues in length, or about 1-25 residues, or about 1-10 residues in length. Spacer sequences, exclusive of cleavage sites, can comprise any of the 20 natural L amino acids, and will preferably have XTEN-like properties in that: 1) they comprise hydrophilic amino acids that are sterically unhindered such as, but not limited to, glycine (G), alanine (A), serine (S), threonine (T), glutamate (E), proline (P) and aspartate (D); and 2) they are substantially non-repetitive. In some cases, the spacer can be polyglycines or polyalanines, or is predominately a mixture of combinations of glycine, serine and alanine residues. The spacer polypeptide exclusive of a cleavage sequence is largely to substantially devoid of secondary structure; e.g., less than about 10%, or less than about 5% as determined by the Chou-Fasman and/or GOR algorithms or, in the case of short spacer sequences, would not substantially contribute to the secondary structure of the attached XTEN.

In one embodiment the spacer comprises amino acids compatible with restrictions sites; e.g., one or two sequences selected from Table 5, to facilitate incorporation of the XTEN encoding sequence into a polynucleotide encoding a BFXTEN construct. For XTEN that are incorporated internal to the BP or BFXTEN sequence, each XTEN would generally be flanked by two spacer sequences comprising amino acids compatible with restriction sites, while XTEN attached to the N- or C-termini would only require a single spacer sequence at the junction of the two components and another at the opposite end for incorporation into the vector. As would be apparent to one of ordinary skill in the art, the spacer sequences comprising amino acids compatible with restriction sites that are internal to BP could be dispensed with when an entire BFXTEN gene is synthetically generated.

TABLE 5
Space Sequences Compatible with
Restriction Sites

| Spacer | Restriction |
|---|---|
| Sequence | Enzyme |
| GSPG | BsaI |
| (SEQ ID NO. 133) | |
| ETET | BsaI |
| (SEQ ID NO: 134) | |
| PGSSS | BbsI |
| (SEQ ID NO: 135) | |
| GAP | AscI |
| GPA | FseI |
| GPSGP | SfiI |
| (SEQ ID NO: 136) | |
| TG | AgeI |
| GT | KpnI |

In one embodiment, one or more spacer sequences in a BFXTEN fusion protein composition may each further contain a cleavage sequence, which may be identical or may be different, wherein the cleavage sequence may be acted on by a protease appropriate for the cleavage sequence to release the BP from the fusion protein. In some cases, the incorporation of the cleavage sequence into the BFXTEN is designed to permit release of a BP that becomes active or more active upon its release from the XTEN. In one embodiment, the BP that is released from the fusion protein by cleavage of the cleavage sequence exhibits at least about a two-fold, or at least about a three-fold, or at least about a four-fold, or at least about a five-fold, or at least about a six-fold, or at least about a eight-fold, or at least about a ten-fold, or at least about a 20-fold increase in biological activity compared to the intact BFXTEN fusion protein; e.g., binding to a receptor or ligand or an increase or decrease of a biochemical parameter described herein or those known in the art to be associated with metabolic or cardiovascular disorders. The cleavage sequences are located sufficiently close to the BP sequences, generally within 18, or within 12, or within 6, or within 2 amino acids of the BP sequence terminus, such that any remaining residues attached to the BP after cleavage do not appreciably interfere with the activity of the BP, yet provide sufficient access to the protease to be able to effect cleavage of the cleavage sequence. In some embodiments, the cleavage site is a sequence that can be cleaved by a protease endogenous to the mammalian subject such alai the BFXTEN can be cleaved after administration to a subject. In such cases, the BFXTEN can serve as a prodrug or a circulating depot for the BP. Examples of cleavage sequences contemplated by the invention include, but are not limited to, a polypeptide sequence cleavable by a mammalian endogenous protease selected from FXIa, FXIIa, kallikrein, FVIIa, FIXa, FXa, FIIa (thrombin), Elastase-2, granzyme B, MMP-12, MMP-13, MMP-17 or MMP-20, or by non-mammalian proteases such as TEV, enterokinase, PreScission™ protease (rhinovirus 3C protease), and sortase A. Sequences known to be cleaved by the foregoing proteases are known in the art. Exemplary cleavage sequences and cut sites within the sequences are presented in Table 6. For example, thrombin (activated clotting factor II) acts on the sequence LTPR↓SLLV (SEQ ID NO: 144) [Rawlings N. D., et al. (2008) Nucleic Acids Res., 36: D320], which would be cut after the arginine at position 4 in the sequence. Active FIIa is produced by cleavage of FII by FXa in the presence of phospholipids and calcium and is down stream from factor IX in the coagulation pathway. Once activated its natural role in coagulation is to cleave fibrinogen, which then in turn, begins clot formation. FIIa activity is tightly controlled and only occurs when coagulation is necessary for proper hemostasis. However, as coagulation is an on-going process in mammals, by incorporation of the LTPRSLLV (SEQ ID NO: 144) sequence into the BFXTEN between the BP and the XTEN, the XTEN domain would be removed from the adjoining BP concurrent with activation of either the extrinsic or intrinsic coagulation pathways when coagulation is required physiologically, thereby releasing BP over time Similarly, incorporation of other sequences into BFXTEN that are acted upon by endogenous proteases would provide for sustained release of BP that may, in certain cases, provide a higher degree of activity for the BP from the “prodrug” form of the BFXTEN.

In some cases, only the two or three amino acids flanking both sides of the cut site (four to six amino acids total) would be incorporated into the cleavage sequence. In other cases, the known cleavage sequence can have one or more deletions or insertions or one or two or three amino acid substitutions for any one or two or three amino acids in the known sequence, wherein the deletions, insertions or substitutions result in reduced or enhanced susceptibility but not an absence of susceptibility to the protease, resulting in an ability to tailor the rate of release of the BP from the XTEN. Exemplary substitutions are shown in Table 6.

TABLE 6
Protease Cleavage Sequences

| | Exemplary | | | |
|---|---|---|---|---|
| Protease Acting | Cleavage | SEQ ID | | SEQ ID |
| Upon Sequence | Sequence | NO: | Minimal Cut Site* | NO: |
| FXIa | KLTR↓AET | 137 | KD/FL/T/R↓VA/VE/GT/GV | |
| FXIa | DFTR↓VVG | 138 | KD/FL/T/R↓VA/VE/GT/GV | |
| FXIIa | TMTR↓IVGG | 139 | NA | |
| Kallikrein | SPFR↓STGG | 140 | -/-/FL/RY↓SR/RT/-/- | |
| FVIIa | LQVR↓IVGG | 141 | NA | |
| FIXa | PLGR↓IVGG | 142 | -/-/G/R↓-/-/-/- | |
| FXa | IEGR↓TVGG | 143 | IA/E/GFP/R↓STI/VFS/-/G | |
| FIIa (thrombin) | LTPR↓SLLV | 144 | -/-/PLA/R↓SAG/-/-/- | |
| Elastase-2 | LGPV↓SGVP | 145 | -/-/-/VIAT↓-/-/-/- | |
| Granzyme-B | VAGD↓SLEE | 146 | V/-/-/D↓-/-/-/- | |
| MMP-12 | GPAG↓LGGA | 147 | G/PA/-/G↓L/-/G/- | 148 |
| MMP-13 | GPAG↓LRGA | 149 | G/P/-/G↓L/-/GA/- | 150 |
| MMP-17 | APLG↓LRLR | 151 | -/PS/-/-↓LQ/-/LT/- | |
| MMP-20 | PALP↓LVAQ | 152 | NA | |
| TEV | ENLYFQ↓G | 153 | ENLYFQ↓G/S | 154 |
| Enterokinase | DDDK↓IVGG | 155 | DDDK↓IVGG | 156 |
| Protease 3C | LEVLFQ↓GP | 157 | LEVLFQ↓GP | 158 |
| (PreScission ™) | | | | |
| Sortase A | LPKT↓GSES | 159 | L/P/KEAD/T↓G/-/EKS/S | 160 |

↓indicates cleavage site
NA: not applicable
*the listing of multiple amino acids before, between, or after a slash indicate alternative amino acids that can be substituted at the position; “-” indicates that any amino acid may be substituted for the corresponding amino acid indicated in the middle column

In some embodiments of the BFXTEN compositions, at least a portion of the biological activity of the respective BP is retained by the intact BFXTEN. In other cases, the BP component either becomes biologically active or has an increase in activity upon its release from the XTEN by cleavage of an optional cleavage sequence(s) incorporated within spacer sequences into the BFXTEN, described above. The BP for inclusion into the subject BFXTEN can be evaluated for activity using assays or measured or determined parameters as described herein (e.g., the assays of the Examples or Table 32), and those sequences that retain at least about 40%, or about 50%, or about 55%, or about 60%, or about 70%, or about 80%, or about 90%, or about 95% or more activity compared to the corresponding native BP sequence would be considered suitable for inclusion in the subject BFXTEN. In one embodiment, a single BP found to retain a suitable level of activity can be linked to one or more XTEN polypeptides having at least about 80% sequence identity to a sequence from Table 4, alternatively at least about 81%, 82%, 83%, 84%, 85%, 86%, 87%, 88%, 89%, 90%, 91%, 92%, 93%, 94%, 95%, 96%, 97%, 98% or 99% sequence identity as compared with a sequence of Table 4, resulting in a chimeric fusion protein. In another embodiment, two BP, different from each other (e.g., BP1 and BP2 as described above) and found to retain suitable levels of activity can be linked to one or more XTEN polypeptides having at least about 80% sequence identity to a sequence from Table 4, alternatively at least about 81%, 82%, 83%, 84%, 85%, 86%, 87%, 88%, 89%, 90%, 91%, 92%, 93%, 94%, 95%, 96%, 97%, 98% or 99% sequence identity as compared with a sequence from Table 4, resulting in a chimeric, monomeric BFXTEN fusion protein.

Non-limiting examples of sequences of fusion proteins containing a single BP linked to a single XTEN are presented in Table 33. In one embodiment, a combination BFXTEN composition would comprise a first fusion protein having at least about 80% sequence identity to a sequence from Table 33, alternatively at least about 81%, 82%, 83%, 84%, 85%, 86%, 87%, 88%, 89%, 90%, 91%, 92%, 93%, 94%, 95%, 96%, 97%, 98% or 99% sequence identity as compared with a sequence from Table 33, and a second fusion protein with at least about 80% sequence identity to a sequence from Table 33, alternatively at least about 81%, 82%, 83%, 84%, 85%, 86%, 87%, 88%, 89%, 90%, 91%, 92%, 93%, 94%, 95%, 96%, 97%, 98% or 99% sequence identity as compared with a sequence from Table 33, wherein the BP component of the second fusion protein is different from the BP component of the first fusion protein. Non-limiting examples of sequences of monomeric BFXTEN fusion proteins comprising two BP linked to a single XTEN that can be used in the treatment of metabolic and/or cardiovascular diseases, disorders or conditions are presented in Table 34. In one embodiment, a BFXTEN composition would comprise a sequence with at least about 80% sequence identity to a sequence from Table 34, alternatively at least about 81%, 82%, 83%, 84%, 85%, 86%, 87%, 88%, 89%, 90%, 91%, 92%, 93%, 94%, 95%, 96%, 97%, 98% or about 99% sequence identity as compared with a sequence from Table 34. Non-limiting examples of sequences of monomeric BFXTEN fusion proteins containing two BP linked to a single XTEN that can be used in the treatment of cardiovascular diseases, disorders or conditions are presented in Table 35. In one embodiment, a BFXTEN composition would comprise a sequence with at least about 80% sequence identity to a sequence from Table 35, alternatively at least about 81%, 82%, 83%, 84%, 85%, 86%, 87%, 88%, 89%, 90%, 91%, 92%, 93%, 94%, 95%, 96%, 97%, 98% or about 99% sequence identity as compared with a sequence from Table 35. Non-limiting examples of sequences of monomeric BFXTEN fusion proteins containing two BP in which BP1 is linked to the N-terminus of BP2 and BP2 is linked to the N-terminus of an XTEN are presented in Table 36. In one embodiment, a BFXTEN composition would comprise a sequence with at least about 80% sequence identity to a sequence from Table 36, alternatively at least about 81%, 82%, 83%, 84%, 85%, 86%, 87%, 88%, 89%, 90%, 91%, 92%, 93%, 94%, 95%, 96%, 97%, 98% or about 99% sequence identity as compared with a sequence from Table 36. Non-limiting examples of sequences of monomeric BFXTEN fusion proteins containing two BP and two XTEN in an N- to C-terminus configuration of BP1-XTEN1-BP2-XTEN2 are presented in Table 37. In one embodiment, a BFXTEN composition would comprise a sequence with at least about 80% sequence identity to a sequence from Table 37, alternatively at least about 81%, 82%, 83%, 84%, 85%, 86%, 87%, 88%, 89%, 90%, 91%, 92%, 93%, 94%, 95%, 96%, 97%, 98% or about 99% sequence identity as compared with a sequence from Table 37. In the foregoing embodiments of the paragraph, the invention contemplates substitution of a different BP sequence from Table 1 for the sequence of either BP1 or BP2 of any BP sequence of the Tables, and a different XTEN sequence from Table 4 (or a fragment or sequence variant thereof) substituted for either of the XTEN of that sequence. In the foregoing embodiments hereinabove described in this paragraph, the BFXTEN fusion protein can further comprise one or more spacer sequences from Tables 5 and/or 6; the sequences being located between the BP1 and/or BP2 and the XTEN. Non-limiting examples of BFXTEN comprising a BP1, BP2, XTEN, cleavage sequence(s) and spacer amino acids are presented in Table 38.

### IV). Properties of the BFXTEN Compositions of the Invention

(a) Pharmacokinetic Properties of BFXTEN

The invention provides BFXTEN fusion protein compositions comprising a first and a second BP linked to XTEN with enhanced pharmacokinetics compared to the first or second BP not linked to XTEN. The pharmacokinetic properties of a BP that can be enhanced by linking a given XTEN to the BP to create a BFXTEN fusion protein (which includes the BMXTEN chimeric bifunctional monomeric XTEN fusion protein compositions with two BP, as well as the BCXTEN chimeric bifunctional combination compositions of two individual fusion proteins, each with a different payload BP linked to one or more XTEN) include, but are not limited to, terminal half-life, area under the curve (AUC), Cmax, volume of distribution, maintaining the biologically active BFXTEN within the therapeutic window above the minimum effective blood concentration for a longer period of time compared to the BP not linked to XTEN, and bioavailability. The half-life and other pharmacokinetic parameters of a BFXTEN can be determined by standard methods involving dosing, the taking of blood samples at times intervals, and the assaying of the protein using ELISA, HPLC, radioassay, or other methods known in the art or as described herein, followed by standard calculations of the data to derive the half-life and other PK parameters. It is intended that “BFXTEN” encompasses both BMXTEN and BCXTEN compositions in the pharmacokinetic embodiments that follow. It is further intended that “BP” encompasses either of the single BP or both, unless indicated otherwise (e.g., BP1 or BP2).

As a result of the enhanced pharmacokinetic properties conferred by XTEN, the BFXTEN, when used at the dose and dose regimen determined to be appropriate for the composition by the methods described herein, can achieve a circulating concentration resulting in a desired pharmacologic or clinical effect for an extended period of time compared to a comparable dose of the BP not linked to XTEN; properties that permits less frequent dosing or an enhanced pharmacologic effect, resulting in enhanced utility in the treatment of metabolic or cardiovascular disorders, diseases and related conditions. As used herein, a “comparable dose” means a dose with an equivalent moles/kg for the active BP pharmacophore that is administered to a subject in a comparable fashion. It will be understood in the art that a “comparable dosage” of BFXTEN fusion protein would represent a greater weight of agent but would have essentially the same mole-equivalents of BP in the dose of the fusion protein administered.

When used at the appropriate dose determined for the composition by the methods described herein, the BFXTEN can achieve a circulating concentration resulting in a pharmacologic effect, yet stay within the safety range for either active component of the composition for an extended period of time compared to the BP not linked to XTEN; the BFXTEN remains within the therapeutic window for both the first and second BP components of the fusion protein composition. In some embodiments, a monomeric BFXTEN fusion protein comprising two different BP can result in an additive or synergistic effect when administered to a subject in treatment of the target disease or disorder such that the therapeutic window may be attained at a lower dose compared to an equivalent or comparable dose of one or the other of the BPs not linked to the XTEN.

As described more fully in the Examples pertaining to pharmacokinetic characteristics of fusion proteins comprising XTEN, it was surprisingly discovered that increasing the length of the XTEN sequence could confer a disproportionate increase in the terminal half-life of a fusion protein comprising the XTEN and a payload portion, such as a BP. Accordingly, the invention provides BFXTEN fusion proteins comprising XTEN wherein the XTEN is selected to provide a targeted half-life for the BFXTEN composition administered to a subject. In some embodiments, the invention provides monomeric BFXTEN fusion proteins comprising XTEN wherein the XTEN is selected to confer an increase in the terminal half-life for the administered BFXTEN, compared to the corresponding BP not linked to XTEN, of at least about 8 h, or at least about 16 h, or at least about 24 h, or at least about 48 h, or at least about 72 h, or at least about 96 h, or at least about 120 h, or at least about 200 h, or at least about 300 h, or at least about 400 h, or an increase in terminal half-life of at least about 500 h. In another embodiment, the invention provides monomeric BFXTEN fusion proteins comprising XTEN wherein the XTEN is selected to confer an increase in the terminal half-life for the administered BFXTEN, compared to the corresponding BP not linked to XTEN and administered at a comparable dose, wherein the increase in terminal half-life is at least about two-fold longer, or at least about three-fold, or at least about four-fold, or at least about five-fold, or at least about six-fold, or at least about seven-fold, or at least about eight-fold, or at least about nine-fold, or at least about ten-fold, or at least about 15-fold, or at least a 20-fold, or at least a 40-fold or greater increase in terminal half-life compared to the BP not linked to XTEN. In another embodiment, administration of a therapeutically effective dose of a BFXTEN fusion protein to a subject in need thereof can result in a gain in time between consecutive doses necessary to maintain a therapeutically effective blood level of the fusion protein of at least 48 h, or at least 72 h, or at least about 96 h, or at least about 120 h, or at least about 7 days, or at least about 14 days, or at least about 21 days between consecutive doses compared to a BP not linked to XTEN and administered at a comparable dose. It will be understood in the art that the time between consecutive doses to maintain a “therapeutically effective blood level” will vary greatly depending on the physiologic state of the subject.

In one embodiment, the BFXTEN fusion proteins exhibit an increase in AUC of at least about 50%, or at least about 60%, or at least about 70%, or at least about 80%, or at least about 90%, or at least about a 100%, or at least about 150%, or at least about 200%, or at least about 300%, or at least about 500%, or at least about 1000%, or at least about a 2000% compared to the corresponding BP not linked to the XTEN and administered to a subject at a comparable dose. The pharmacokinetic parameters of a BFXTEN can be determined by standard methods involving dosing, the taking of blood samples at times intervals, and the assaying of the protein using ELISA, HPLC, radioassay, or other methods known in the art or as described herein, followed by standard calculations of the data to derive the half-life and other PK parameters.

The invention further provides combination BXTEN of a first and a second fusion protein in which the first and the second XTEN sequences of the first and the second fusion protein may each be selected to confer substantially the same terminal half-life on the respective fusion proteins of the combination BFXTEN composition when administered to a subject. In one embodiment, the terminal half-life of each fusion protein is within at least about 25% of each other, or more preferably within at least about 20%, or more preferably within at least about 15%, and most preferably within at least about 10%. In the foregoing embodiment, the XTEN of the first and the second fusion protein can have an identical or different sequence, and will each exhibit at least about 80% sequence identity, or at least about 90%, or at least about 95%, or at least about 97% or greater sequence identity to each other or to a sequence selected from Table 4 or a fragment thereof.

The invention also provides combination BCXTEN compositions comprising a first and a second fusion protein in which the XTEN sequences of the first and the second fusion protein may each be selected to confer a different terminal half-life on the respective fusion proteins of the combination BCXTEN composition. In one embodiment, the XTEN is selected to confer a terminal half-life on the first fusion protein that is at least about 25% longer than the terminal half-life of the second fusion protein, alternatively at least about 50% longer, or at least about 75% longer, or at least about 100% longer, or at least about 150% longer, or at least about 200% longer, or at least about 300% longer, or at least about 400% longer, or at least 500% longer than the terminal half-life of the second fusion protein of the combination BFXTEN composition. In the foregoing embodiment, the XTEN sequence of the first fusion protein of the combination composition is longer than the XTEN sequence of the second fusion protein, and has at least about 72 more amino acids, alternatively at least about 96 more amino acids, alternatively at least about 96 more amino acids, alternatively at least about 120 more amino acids, alternatively at least about 144 more amino acids, alternatively at least about 200 more amino acids, alternatively at least about 250 more amino acids, alternatively at least about 300 more amino acids, alternatively at least about 350 more amino acids, alternatively at least about 400 more amino acids, alternatively at least about 450 more amino acids, alternatively at least about 450 more amino acids, alternatively at least about 500 more amino acids, alternatively at least about 750 more amino acids, or at least about 1000 more amino acids than the XTEN sequence of the second fusion protein. In the embodiments hereinabove described in this paragraph, the XTEN of the first and second fusion proteins of the BCXTEN compositions can each exhibit at least about 90%, or about 91%, or about 92%, or about 93%, or about 94%, or about 95%, or about 96%, or about 97%, or about 98%, or about 99%, to about 100% sequence identity to a first and a second sequence of comparable length selected from Table 4, or a fragment thereof.

The enhanced PK parameters of the subject BFXTEN compositions allow for reduced amounts of the compositions to be administered to a subject in need thereof, compared to BP not linked to XTEN, particularly for those subjects receiving repeated doses of a biologic for an extended period of time. In one embodiment, about two-fold less, or about three-fold less, or about four-fold less, or about five-fold less, or about six-fold less, or about eight-fold less, or about 10-fold less of moles of the fusion protein is administered to a subject under a dose regimen to maintain a given physiologic effect or biochemical parameter (e.g., glucose homeostasis, change in body weight, maintain cardiac function, etc.), compared to the corresponding BP not linked to the XTEN. In another embodiment, a smaller amount of moles of about two-fold less, or about three-fold less, or about four-fold less, or about five-fold less, or about six-fold less, or about eight-fold less, or about 10-fold less or greater of moles of fusion protein is administered in comparison to the corresponding BP not linked to the XTEN under a dose regimen needed to maintain or achieve a given physiologic effect or biochemical parameter, and the fusion protein achieves a comparable area under the curve as the corresponding equivalent amount of moles of the BP not linked to the XTEN. In another embodiment, the BFXTEN fusion protein requires less frequent administration for routine treatment of a subject with diabetes, insulin resistance, or a cardiovascular disorder, wherein the dose is administered about every four days, about every seven days, about every 10 days, about every 14 days, about every 21 days, or about monthly of the fusion protein administered to a subject, and the fusion protein achieves a comparable area under the curve as the corresponding BP not linked to the XTEN. In another embodiment, an accumulative smaller amount of about 5%, or about 10%, or about 20%, or about 40%, or about 50%, or about 60%, or about 70%, or about 80%, or about 90% less of the moles of fusion protein are administered to a subject in comparison to the corresponding equivalent amount of moles of the BP not linked to the XTEN under a dose regimen needed to maintain or achieve the physiologic effect, yet the fusion protein achieves at least a comparable area under the curve as the corresponding BP not linked to the XTEN. The accumulative smaller amount is measure for a period of at least about one week, or about 14 days, or about 21 days, or about one month.

(b) Pharmacology and Pharmaceutical Properties of BFXTEN

The present invention provides BFXTEN compositions comprising BP covalently linked to XTEN that can have enhanced pharmacologic or pharmaceutical properties compared to BP not linked to XTEN, as well as methods to enhance the therapeutic and/or biologic activity or effect of the respective two BP components of the compositions. In addition, the invention provides BFXTEN compositions with enhanced properties compared to those art-known fusion proteins containing immunoglobulin polypeptide partners, polypeptides of shorter length and/or polypeptide partners with repetitive sequences. In addition, BFXTEN fusion proteins provide significant advantages over chemical conjugates, such as pegylated constructs, notably the fact that recombinant BFXTEN fusion proteins can be made in bacterial cell expression systems, which can reduce time and cost at both the research and development and manufacturing stages of a product, as well as result in a more homogeneous, defined product with less toxicity for both the product and metabolites of the BFXTEN compared to pegylated conjugates.

As therapeutic agents, the BFXTEN may possess a number of advantages over therapeutics not comprising XTEN including, for example, increased solubility, increased thermal stability, reduced immunogenicity, increased apparent molecular weight, reduced renal clearance, reduced proteolysis, reduced metabolism, enhanced therapeutic efficiency, a lower effective therapeutic dose, increased bioavailability, increased time between dosages to maintain blood levels within the therapeutic window for the BP, a “tailored” rate of absorption, enhanced lyophilization stability, enhanced serum/plasma stability, increased terminal half-life, increased solubility in blood stream, decreased binding by neutralizing antibodies, decreased receptor-mediated clearance, reduced side effects, retention of receptor/ligand binding affinity or receptor/ligand activation, stability to degradation, stability to freeze-thaw, stability to proteases, stability to ubiquitination, ease of administration, compatibility with other pharmaceutical excipients or carriers, persistence in the subject, increased stability in storage (e.g., increased shelf-life), reduced toxicity in an organism or environment and the like. The net effect of the enhanced properties is that the BFXTEN may result in enhanced therapeutic and/or pharmacologic effect when administered to a subject with a metabolic and/or cardiovascular disease or disorder.

In other cases where, for example, the pharmaceutical or physicochemical properties of the first and the second BP are different (such as the degree of aqueous solubility or stability), the length and/or the motif family composition of the first and the second XTEN sequences of the first and the second fusion protein may each be selected to confer a different degree of solubility and/or stability on the respective fusion proteins such that the overall pharmaceutical properties of the two fusion proteins of the combination BFXTEN composition are similar. The respective first and second fusion proteins can be constructed and assayed, using methods described herein, to confirm their physicochemical properties and the XTEN length or family composition adjusted, as needed, to result in the desired properties. In such cases, the combination BFXTEN could be formulated with the first and the second fusion proteins such that the overall composition can have uniform properties. In one embodiment, the XTEN sequence of the respective first and second fusion proteins of the combination BFXTEN are selected such that each fusion protein has a aqueous solubility that is within at least about 25% of the other fusion protein, or at least about 20%, or at least about 15%, or at least about 10%, or at least about 9%, or at least about 8%, or at least about 7%, or at least about 6%, or at least about within 5% of the solubility of the other fusion protein. In the embodiments hereinabove described in this paragraph, the XTEN of the first and second fusion proteins can each exhibit at least about 80%, or about 90%, or about 91%, or about 92%, or about 93%, or about 94%, or about 95%, or about 96%, or about 97%, or about 98%, or about 99%, to about 100% sequence identity to a sequence selected from Table 4, or a fragment thereof. Specific assays and methods for measuring the physical and structural properties of expressed proteins are known in the art, including methods for determining properties such as protein aggregation, solubility, secondary and tertiary structure, melting properties, contamination and water content, etc. Such methods include analytical centrifugation, EPR, HPLC-ion exchange, HPLC-size exclusion, HPLC-reverse phase, light scattering, capillary electrophoresis, circular dichroism, differential scanning calorimetry, fluorescence, HPLC-ion exchange, HPLC-size exclusion, IR, NMR, Raman spectroscopy, refractometry, and UV/Visible spectroscopy. Additional methods are disclosed in Arnau et al., Prot Expr and Purif (2006) 48, 1-13. Application of these methods to the invention would be within the grasp of a person skilled in the art.

The invention provides BFXTEN compositions that can maintain each BP component within a therapeutic window for a greater period of time compared to comparable dosages of the respective BP not linked to XTEN. It will be understood in the art that a “comparable dosage” of BFXTEN fusion protein would represent a greater weight of agent but would have the same approximate mole-equivalents of BP in the dose of the fusion protein and/or would have the same approximate molar concentration relative to the BP. The invention also provides methods to select the XTEN appropriate for conjugation to provide the desired pharmacokinetic properties that, when matched with the selection of dose, enable enhanced efficacy of the administered composition by maintaining the circulating concentrations of each BP within the therapeutic window for an extended period of time. As used herein, “therapeutic window” means that amount of drug or biologic as a blood or plasma concentration range, that provides efficacy or a desired pharmacologic effect over time for the disease or condition without unacceptable toxicity; the range of the circulating blood concentrations between the minimal amount to achieve a positive therapeutic effect and the maximum amount which results in a response that is the response immediately before toxicity to the subject (at a higher dose or concentration). Additionally, therapeutic window generally encompasses an aspect of time; the blood concentration that results in a desired pharmacologic effect over time that does not result in unacceptable toxicity or adverse events. A dosed composition that stays within the therapeutic window for the subject could also be said to be within the “safety range.”

Dose optimization is important for many biologics, especially for those with a narrow therapeutic window. For example, many peptides involved in glucose homeostasis have a narrow therapeutic window; e.g., insulin or glucagon. For a BP with a narrow therapeutic window, such as glucagon or a glucagon analog, a standardized single dose for all patients presenting with a variety of symptoms may not always be effective. Since two different biologically active proteins are being used together in the compositions of the present invention, the potency of each of the BPs and the interactive effects achieved by combining and dosing them together is taken into account in order to achieve safe and effective BFXTEN compositions. One such combination is exenatide and glucagon, detailed in Example 25, where two fusion proteins of different length were used together in a model of diabetes to result in multiple beneficial effects without evidence of overt toxicity. A consideration of these factors is well within the purview of the ordinarily skilled clinician or pharmacologist for the purpose of determining the therapeutically or pharmacologically effective amount of the BFXTEN, versus that amount that would result in unacceptable toxicity and place it outside of the safety range.

In many cases, the therapeutic window for the BP components of the subject compositions have been established and are available in published literature or are stated on the drug label for approved products containing the BP. In other cases, and in particular where two BPs are being used together, the therapeutic window can be established. The methods for establishing the therapeutic window for a given composition are known to those of skill in the art (see, e.g., Goodman & Gilman's The Pharmacological Basis of Therapeutics, 11^{th} Edition, McGraw-Hill (2005)). For example, by using dose-escalation studies in subjects with the target disease or disorder to determine efficacy or a desirable pharmacologic effect, appearance of adverse events, and determination of circulating blood levels, the therapeutic window for a given subject or population of subjects can be determined for a given drug or biologic, or combinations of biologics or drugs. The dose escalation studies can evaluate the activity of a BFXTEN through metabolic studies in a subject or group of subjects that monitor physiological or biochemical parameters, as known in the art or as described herein for one or more parameters associated with the metabolic and/or cardiovascular disease or disorder, or clinical parameters associated with a beneficial outcome for the particular indication, together with observations and/or measured parameters to determine the no effect dose, adverse events, maximum tolerated dose and the like, together with measurement of pharmacokinetic parameters that establish the determined or derived circulating blood levels. The results can then be correlated with the dose administered and the blood concentrations of the therapeutic that are coincident with the foregoing determined parameters or effect levels. By these methods, a range of doses and blood concentrations can be correlated to the minimum effective dose as well as the maximum dose and blood concentration at which a desired effect occurs and above which toxicity occurs, thereby establishing the therapeutic window for the administered BFXTEN. Blood concentrations of the BXTEN fusion protein (or as measured by the BP component) above the maximum would be considered outside the therapeutic window or safety range. Thus, by the foregoing methods, a Cₘᵢₙ blood level would be established, below which the BFXTEN fusion protein would not have the desired pharmacologic effect, and a Cₘₐₓ blood level would be established that would represent the highest circulating concentration before reaching a concentration that would elicit unacceptable side effects, toxicity or adverse events, placing it outside the safety range for the BFXTEN. With such concentrations established, the frequency of dosing and the dosage amount can be further refined by measurement of the Cₘₐₓ and Cₘᵢₙ to provide the appropriate dose amount and dose frequency to keep the fusion protein(s) within the therapeutic window. By the method, one of skill in the art can, by the means disclosed herein or by other methods known in the art, confirm that the administered BFXTEN remains in the therapeutic window for the desired interval or requires adjustment in dose or length or sequence of XTEN. Further, the determination of the appropriate dose and dose frequency to keep the BFXTEN within the therapeutic window establishes the therapeutically effective dose regimen; the schedule for administration of multiple consecutive doses using a therapeutically effective dose regimen of the fusion protein to a subject in need thereof resulting in consecutive Cₘₐₓ peaks and/or Cₘᵢₙ troughs that remain within the therapeutic window and result in an improvement in at least one measured parameter relevant for the metabolic and/or cardiovascular disease, disorder or condition.

The activity of the BFXTEN compositions of the invention, including functional characteristics or biologic and pharmacologic activity and parameters that result, may be determined by any suitable screening assay known to the art for measuring the desired characteristic. The activity of the BFXTEN polypeptides comprising BP components and their effects on biochemical of physiological parameters may be measured by assays described herein; e.g., one or more assays selected from Table 32, assays of the Examples, or by methods known in the art to ascertain the degree of solubility, structure and retention of biologic activity. Specific in vivo and ex vivo biological assays may also be used to assess the activity of each BFXTEN and/or BP component to be incorporated into BFXTEN. For example, the increase of insulin secretion and/or transcription from the pancreatic beta cells can be measured by methods described in Table 32 or assays known in the art. Glucose uptake by tissues can also be assessed by methods such as the glucose clamp assay and the like. Other in vivo and ex vivo parameters suitable to assess the activity of administered BFXTEN fusion proteins in treatment of metabolic diseases and disorders include fasting glucose level, peak change of postprandial glucose level compared to baseline, glucose homeostasis, response to oral glucose tolerance test, response to insulin challenge, HA_{1c}, level, daily caloric intake, satiety, rate of gastric emptying, pancreatic secretion, insulin secretion in response to glucose challenge, peripheral tissue insulin sensitivity, beta cell mass, beta cell destruction, blood lipid levels or profiles, cholesterol level, body mass index, or body weight reduction.

For cardiovascular diseases and disorders, a number of markers and/or parameters can be used to assess the biological activity of each BFXTEN and/or the BP component. Such markers parameters include, but are not limited to left ventricular diastolic function, E/A ratio, left ventricular end diastolic pressure, cardiac output, cardiac contractility, left ventricular mass, left ventricular mass to body weight ratio, left ventricular volume, left atrial volume, left ventricular end diastolic dimension (LVEDD), left ventricular end systolic dimension (LVESD), infarct size, exercise capacity, exercise efficiency, and heart chamber size.

In some cases, the BP component of the BFXTEN fusion proteins of the invention retain at least about 25%, preferably about 30%, 40%, 50%, 60%, 70%, 80%, 90%, 95%, 98%, or 99% percent of the biological activity of a native BP with regard to an in vitro biologic activity or pharmacologic effect known or associated with the use of the native BP in the treatment and prevention of metabolic and/or cardiovascular conditions and disorders. In some cases of the foregoing embodiment, the activity of the BP component may be manifest by the intact BFXTEN fusion protein, while in other cases the activity of the BP component would be primarily manifested upon cleavage and release of the BP from the fusion protein by action of a protease that acts on a cleavage sequence incorporated into the BFXTEN fusion protein.

Assays can be conducted that allow determination of binding characteristics of the BFXTEN for BP receptors or a ligand, including binding constant (K_{d}), EC₅₀ values, as well as their half-life of dissociation of the ligand-receptor complex (T_{1/2}). Binding affinity can be measured, for example, by a competition-type binding assay that detects changes in the ability to specifically bind to a receptor or ligand. Additionally, techniques such as flow cytometry or surface plasmon resonance can be used to detect binding events. The assays may comprise soluble receptor molecules, or may determine the binding to cell-expressed receptors. Such assays may include cell-based assays, including assays for proliferation, cell death, apoptosis and cell migration. Other assays may determine receptor binding of expressed polypeptides, wherein the assay may comprise soluble receptor molecules, or may determine the binding to cell-expressed receptors. The binding affinity of a BFXTEN for the receptors or ligands specific to the BP can be assayed using binding or competitive binding assays, such as Biacore assays with chip-bound receptors or binding proteins or ELISA assays, as described in U.S. Pat. No. 5,534,617, or other assays known in the art. In addition, BP sequence variants (assayed as single components or as BFXTEN fusion proteins) can be compared to the native BP using a competitive ELISA binding assay to determine whether they have the same binding specificity and affinity as the native BP, or some fraction thereof such that they are suitable for inclusion in BFXTEN. The binding affinity for receptors or ligands of the BFXTEN of the invention can be at least about 10%, or at least about 20%, or at least about 30%, or at least about 40%, or at least about 50%, or at least about 60%, or at least about 70%, or at least about 80%, or at least about 90%, or at least about 95%, or at least about 99% or more of the affinity of a native BP not bound to XTEN. In one embodiment, the binding affinity K_{d} between the subject BFXTEN and a native receptor or ligand of the BFXTEN is at least about 10⁻⁴ M, alternatively at least about 10⁻⁵M, alternatively at least about 10⁻⁶M, or at least about 10⁻⁷M, or at least about 10⁻⁸M, or at least about 10⁻⁹M. In another embodiment, the BFXTEN are designed to reduce the binding affinity of the BP component when linked to the XTEN to, for example, increase the terminal half-life of BFXTEN administered to a subject by reducing receptor-mediated clearance.

In another embodiment, the invention provides BFXTEN designed to provide reduced binding affinity of a BP component for the receptor or ligand when linked to the XTEN but have a higher degree of affinity restored when the BP is released from XTEN through the cleavage of cleavage sequence(s) incorporated into the BFXTEN sequence, as described more fully above.

In some cases, the invention provides combination BCXTEN compositions in which the composition can be formulated as a fixed ratio of the two individual fusion proteins, each comprising a different BP. In one embodiment, the fixed ratio of the respective fusion proteins can maintain the individual BP components of the combination within the respective therapeutic windows for each fusion protein for a greater period of time compared to a comparable dose of one or both of the respective BP not linked to XTEN and achieve an enhanced physiologic effect due to a positive interaction of the combination of the two different BP. The use of a fixed ratio is a reflection of differences in the efficacy potency or the potential for eliciting adverse events at a given concentration or dose between the two BPs of the combination BCXTEN composition. For example, therapeutic use of glucagon to overcome hypoglycemia has long been known to result in hyperglycemia episodes (D R Owens, et al., “The metabolic response to glucagon and glucagon-(1-21)-peptide in normal subjects and non insulin dependent diabetics.” Br J Clin Pharmacol. 1986; 22(3): 325-329). To reduce the potential for side effects that would place a BP component outside the therapeutic window, the ratio of the first fusion protein to the second fusion protein in the combination BCXTEN composition can be varied. In some embodiments, the ratio (as moles:moles or molecule:molecule) of the first fusion protein to the second fusion protein in the combination BFXTEN is fixed at 1:1, while in other embodiments the ratio will be about 1:2, or about 1:4, or about 1:8, or about 1:10, or about 1:12, or about 1:15, or about 1:20, or about 1:25, or about 1:30, or about 1:40, or about 1:50, or about 1:75, or about 1:100, or about 1:150, or about 1:200, or about 1:300, or about 1:400, or about 1:500, or about 1:750, or about 1:1000, or about 1:1500 or more; the ratio of the two component fusion proteins of the combination compositions being fixed by and in consideration of the determination of the appropriate dose for the therapeutic window for each individual fusion protein. Once established, the fixed ratio combination BCXTEN composition permits administration of a single composition containing two fusion proteins, each with a different BP, to a subject that may result in safe, additive or synergistic effects against the target disease or disorder such that the therapeutic window may be achieved at a lower dose or with less frequent dosing compared to a comparable dose of one or both of the BPs not linked to the XTEN. Additionally, the fixed ratio combination of the two component BCXTENs can result in enhanced pharmacokinetics such that, when used at an appropriate dose for the composition, circulating concentrations resulting in a pharmacologic effect stay within the safety range for either active component of the composition for an extended period of time compared to the BPs not linked to XTEN; i.e., the BCXTEN remains within the therapeutic window for both the first and second BP components of the fusion protein composition for an extended period of time. In one embodiment, administration administered of an effective dose the BCXTEN to a subject may result in blood concentrations of one or both of the fusion proteins that remain within the therapeutic window at least about 100% longer compared to the corresponding BP not linked to XTEN and administered at a comparable dose; alternatively at least about 200% longer; alternatively at least about 300% longer; alternatively at least about 400% longer; alternatively at least about 500% longer; alternatively at least about 1000% longer; alternatively at least about 1500% longer; or at least about 2000% longer compared to the corresponding BP not linked to XTEN and administered at a comparable dose. As used herein, an “appropriate dose” means a dose of a drug or biologic that, when administered to a subject, would result in a desirable therapeutic or pharmacologic effect and a blood concentration within the therapeutic window.

Where the toxicological no-effect dose or the blood concentration of a first BP not linked to an XTEN that would elicit an undesirable side effect is considerably lower than that of a second BP (meaning that the native peptide has a higher potency to result in side effects), the invention provides monomeric fusion proteins with two different BP or combinations of fusion proteins, each with a different BP, in which the fusion protein is configured to reduce the biologic potency of the first BP. In some embodiments, the invention provides monomeric BFXTEN fusion proteins comprising two BPs (BP1 and BP2, in which at least the BP1 component requires a free N-terminus for full potency) configured, N- to C-terminus, as BP2-XTEN-BP1, or alternatively BP2-BP1-XTEN, or alternatively BP2-XTEN-BP1-XTEN. In another embodiment, the invention provides a monomeric fusion protein comprising a single BP (wherein the BP component requires a free N-terminus for full potency) configured, N- to C-terminus, as XTEN-BP, in combination with a second monomeric fusion protein with a second different BP linked to an XTEN. The invention takes advantage of the finding that while some biologically active proteins require a free N-terminus in order to remain fully potent, they retain at least a portion of their biologic activity when linked to the C-terminus of another polypeptide, and their incorporation into BFXTEN of the foregoing configurations results in a composition that, when administered to a subject at an appropriate dose, results in efficacy mediated by the BP1 component yet remains within the therapeutic window for that dose. In another embodiment, wherein the BP1 requires a free C-terminus for full potency, the invention provides a monomeric BFXTEN fusion protein configured BP1-XTEN-BP2, or alternatively BP1-BP2-XTEN, or alternatively BP1-XTEN-BP2-XTEN. In another embodiment, wherein a BP requires a free C-terminus for full potency, the invention provides a monomeric BFXTEN fusion protein configured BP-XTEN used in combination with a second fusion protein comprising the second BP. In the foregoing embodiments described in the paragraph, the fusion proteins can optionally further comprise a spacer sequence with a cleavage site. As will be apparent to those of skill in the art, other permutations or multimers of the foregoing BFXTEN are possible to achieve the desired outcome described above and are contemplated by the present invention.

In another aspect, the invention provides BFXTEN fusion protein compositions configured to increase the terminal half-life of the administered BFXTEN wherein at least a portion of the increased half-life can be due to reduced receptor-mediated clearance (RMC). For many ligands, RMC can occur where activation of the target cell receptor by a bound ligand results in the internalization of the receptor-bound polypeptide ligand with subsequent lysosomal degradation of the ligand. In other cases, where the binding of a polypeptide to its receptor does not lead to activation or where the ligand initiates activation but has an increased off-rate from the receptor, the binding of the polypeptide ligand may not lead to RMC because the ligand-receptor complex is not internalized.

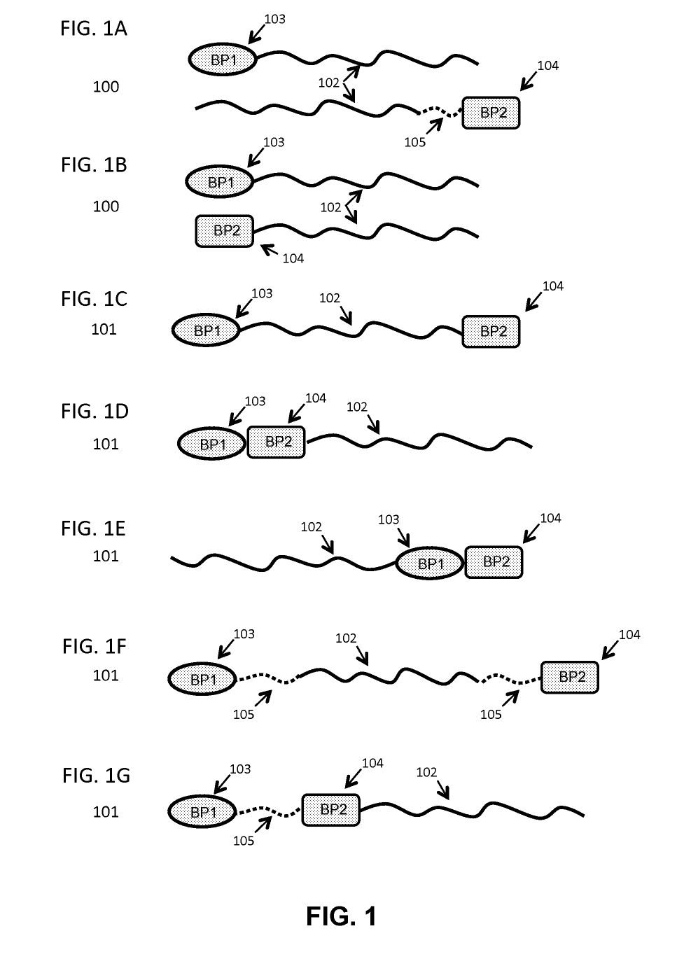

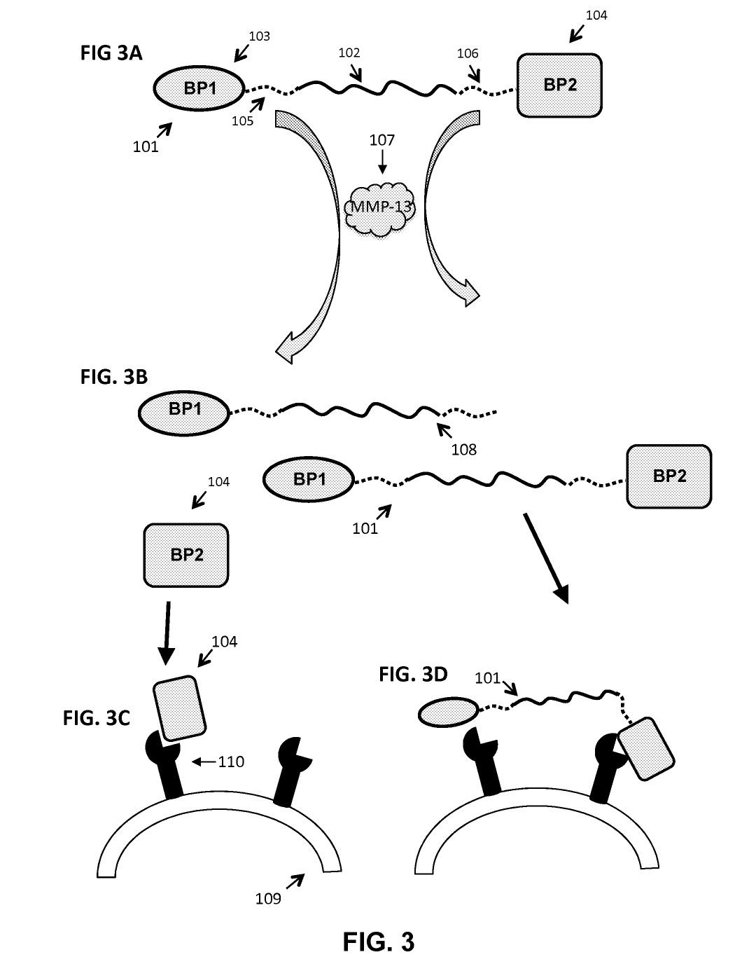

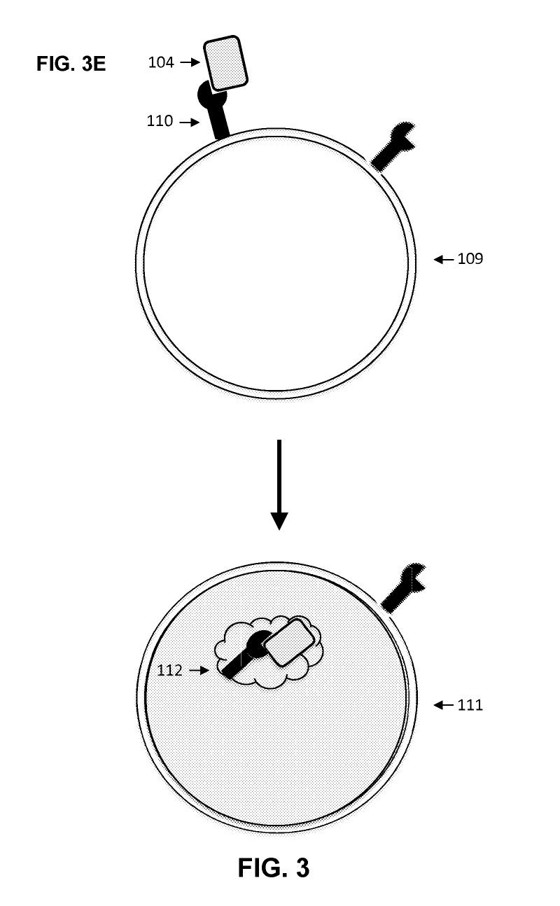

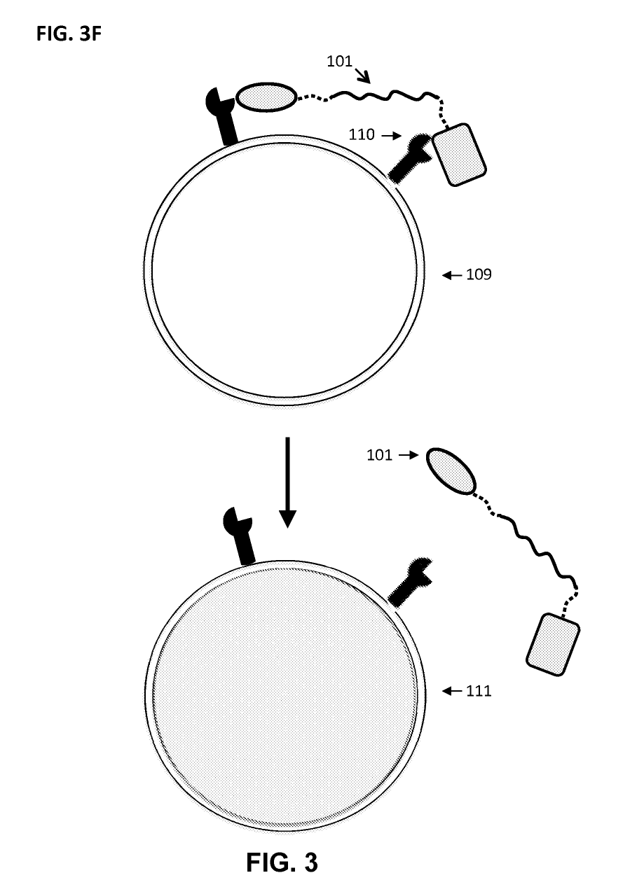

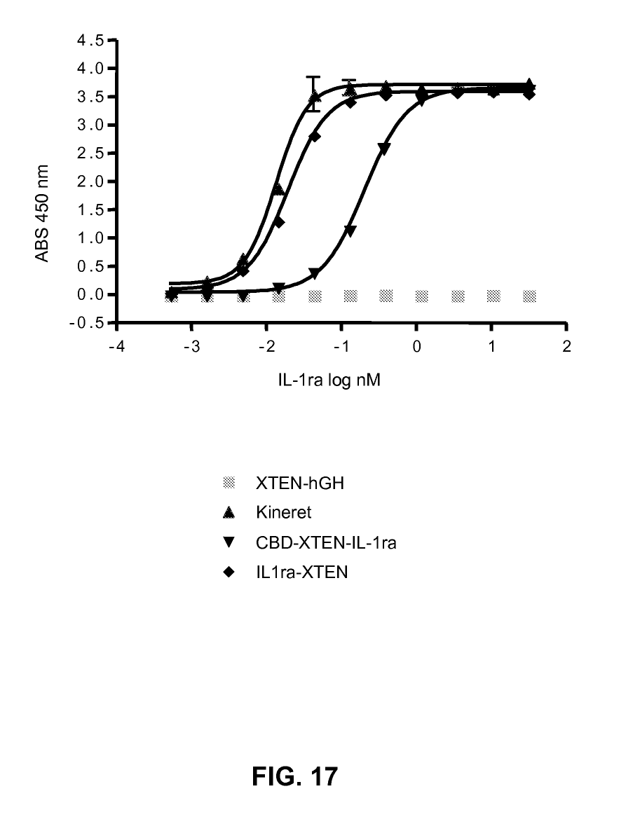

It is believed that configuring a BFXTEN with at least a first BP component with a substantially reduced binding affinity (expressed as Kd) that retains a degree of, but reduced bioactivity compared to the BP not linked to XTEN, is advantageous in terms of having a composition that displays both a long terminal half-life and retains a sufficient degree of bioactivity. The invention takes advantage of BP ligands wherein reduced binding affinity to a receptor, either as a result of a decreased on-rate or an increased off-rate, may be effected by the obstruction of either the N- or C-terminus, and using that terminus as the linkage to another polypeptide of the composition, whether another BP, an XTEN, or a spacer sequence, as illustrated in FIG. 3. The choice of the particular configuration of the BFXTEN fusion protein can reduce the degree of binding affinity to the receptor such that a reduced rate of receptor-mediated clearance can be achieved. For example, it has been found that while linking of IL-1ra to the N-terminus of an XTEN molecule does not substantially interfere with the binding to its native receptor, the addition of a IL-1ra to the C-terminus of the same XTEN molecule significantly reduced the affinity of the molecule to the receptor, as shown in FIG. 17 and detailed in Example 23. As will be appreciated by those skilled in the art, the ability to reduce binding affinity of the BP to its target receptor may be dependent on the requirement to have a free N- or C-terminus for the particular BP. Thus, depending on the therapeutic goals to be attained by the composition, BFXTEN can be configured with a first BP (BP1) linked to the fusion protein wherein the BP1 retains its binding affinity for a target receptor, and a second BP (BP2) linked to the fusion protein wherein the BP2 has reduced binding affinity for a target receptor compared to the BP2 not linked to the fusion protein. Accordingly, the invention contemplates that BFXTEN are constructed in various configurations, listed in an N- to C-terminus orientation (exclusive of spacer sequences), that can include, but are not limited to BP-XTEN; XTEN-BP; BP1-XTEN-BP2; XTEN1-BP-XTEN2; BP1-BP2-XTEN1; BP2-BP1-XTEN; BP2-XTEN-BP1; BP1-XTEN1-BP2-XTEN2; XTEN1-BP1-XTEN2-BP2 (wherein “1”, “2”, and “3” represent different molecules of the respective BP and XTEN portions of the fusion proteins), the configurations of one of formulae I-VI above, or the configurations of FIG. 1, and are then evaluated for receptor binding affinity, biologic activity, and pharmacokinetic properties in order to select the BFXTEN configuration with the desired characteristics of retained biologic activity, reduced RMC and increased terminal half-life. Exemplary construct sequences of BFXTEN encompassed by the invention can be found, for example, in Tables 34 and 35. Thus, in one embodiment, the invention provides a BFXTEN composition configured such that the binding affinity of the BFXTEN for a target receptor is reduced by at least about 60%, or at least about 70%, or at least about 80%, or at least about 90%, or at least about 95%, or at least about 99%, or at least about 99.99% as compared to the binding affinity of a corresponding BFXTEN in a configuration wherein the binding affinity of the BP component to the target receptor is not reduced or compared to the BP not linked to the fusion protein, determined under comparable conditions. Expressed differently, the BP component of the configured BFXTEN composition has a binding affinity that is about 0.01%, or at least about 0.1%, or at least about 1%, or at least about 2%, or at least about 3%, or at least about 4%, or at least about 5%, or at least about 10%, or at least about 20%, or at least about 30%, or at least 40% that of the corresponding BP component of a BFXTEN in a configuration wherein the binding affinity of the BP component is not reduced. In the foregoing embodiments, the binding affinity of the configured BFXTEN for the target receptor are “substantially reduced” compared to a corresponding native BP or a BFXTEN with a configuration in which the binding affinity of the corresponding BP component is not reduced. Accordingly, the present invention provides compositions and methods to produce compositions with reduced RMC by configuring the BFXTEN so as to be able to bind and activate a sufficient number of receptors to obtain a desired in vivo biological response yet avoid activation of more receptors than is required for obtaining such response. The increased half-life of the configured compositions permits higher dosages and/or reduced frequency of dosing compared to BP not linked to XTEN or compared to BFXTEN configurations and the BP components retain sufficient biological or pharmacological activity to result in a composition with clinical efficacy maintained despite reduced dosing frequency. In cases where a reduction in binding affinity is desired in order to reduce receptor-mediated clearance, it will be clear that sufficient binding affinity to obtain the desired receptor activation must nevertheless be maintained. Accordingly, the present invention provides compositions with reduced RMC by configuring the BFXTEN so as to be able to bind and activate a sufficient number of receptors to obtain a desired in vivo biological response yet avoid activation of more receptors than is required for obtaining such response. In the foregoing embodiments hereinabove described in this paragraph, the subject BFXTEN with a reduced binding affinity for the target receptor can still retain or elicit at least about 5% biological activity, or at least about 10%, or at least about 15%, or at least about 20%, or at least about 30%, or at least about 40%, or at least about 50% of the biological activity compared to at least one of the corresponding BP not linked to XTEN.

The assays used to assess the activity of the BFXTEN can be those of Table 32, or others known in the art to be useful for assessing the activity or pharmacologic response of a given biological protein. The receptor-polypeptide binding affinity may be determined by any suitable method known in the art, including, for example, a suitably configured Biacore assay described herein. The in vitro RMC may also be determined by a radio-receptor assay wherein the BFXTEN is labeled (e.g. radioactive or fluorescent labeling), cells with the target receptor to the BP component of the BFXTEN are exposed to the labeled BFXTEN, thereby stimulating cells comprising the receptor for the BP, washing the cells, and measuring label activity remaining on the cells. Alternatively, the BFXTEN may be exposed to cells expressing the relevant receptor. After an appropriate incubation time the supernatant is removed and transferred to a well containing similar cells and the biological response of these cells to the supernatant is determined relative to a non-conjugated BP used as a control to determine the extent of the reduced RMC.

The invention provides that the configuration of the BFXTEN can be designed to tailor the magnitude of the biological activity or the pharmacologic response of a first BP component when the BFXTEN composition is administered to a subject, where the first BP has high potential for unacceptable side effects or toxicity or reduced tolerability of the dose compared to the second BP of the composition. In one embodiment, the invention provides a BFXTEN configured such that the binding affinity of at least one BP component of the BFXTEN for a target receptor is in the range of about 2%, or at least about 3%, or at least about 4%, or at least about 5%, or at least about 10%, or at least about 20%, or at least about 30%, or at least about 40% of that of the corresponding BP component not linked to XTEN. The binding affinity of the configured BXTEN is thus preferably reduced by at least about 60%, or at least about 65%, or at least about 70%, or at least about 75%, or at least about 80%, or at least about 85%, or at least about 90%, or at least about 95%, or at least about 98% as compared to the binding affinity of a corresponding BFXTEN in a configuration wherein the binding affinity of the BP component to the target receptor is not reduced or compared to the BP not linked to the fusion protein, determined under comparable conditions. In the foregoing embodiments hereinabove described in this paragraph, the binding affinity of the configured BFXTEN for the target receptor would be “substantially reduced” compared to a corresponding native BP or a BFXTEN with a configuration in which the binding affinity of the corresponding BP component is not reduced. In one embodiment, the invention provides a BFXTEN in a first configuration comprising at least a first BP linked to the N-terminus of an XTEN wherein the linking results in at least about a two-fold, or at least about a three-fold, or at least about a four-fold, or at least about a five-fold reduction in binding affinity of the BP to the target receptor compared to a BFXTEN in a second configuration in which the first BP is linked to the C-terminus of the XTEN and wherein the half-life of the BFXTEN is increased at least about 50%, or at least about 75%, or at least about 100%, or at least about 150%, or at least about 200%, at least about 300%, at least 400%, or at least 500% compared to the BP component not linked to XTEN. In another embodiment, the invention provides a BFXTEN in a first configuration comprising at least a first BP linked to the C-terminus of an XTEN wherein the linking results in at least about a two-fold, or at least about a three-fold, or at least about a four-fold, or at least about a five-fold reduction in binding affinity of the BP to the target receptor compared to a BFXTEN in a second configuration in which the first BP is linked to the N-terminus of the XTEN, and wherein the half-life of the BFXTEN is increased at least about 50%, or at least about 75%, or at least about 100%, or at least about 150%, or at least about 200%, or at least about 300%, or at least about 400%, or at least about 500% compared to the BP component not linked to XTEN. In the foregoing embodiments hereinabove described in this paragraph, the increased half-life permits higher dosages and reduced frequency of dosing of the BFXTEN compared to BP not linked to XTEN or compared to BFXTEN configurations wherein the BP component retains a binding affinity to the receptor comparable to the native BP.

In another embodiment, the invention provides a method for increasing the terminal half-life of a BFXTEN by producing a fusion protein construct with a specific N- to C-terminus configuration of the BP and XTEN components. In the method, the half-life of the BFXTEN is increased by designing the configuration to have reduced receptor-mediated clearance (RMC) compared to a BFXTEN in a second, different N- to C-terminus configuration.

In general, the steps in the design and production of the fusion proteins of the inventive compositions to increase terminal half-life include: (1) the selection of BPs (e.g., native protein sequences of Table 1 of sequence variants or fragments thereof) to treat the particular disease, disorder or condition; (2) selecting the XTEN that will confer the desired PK and physicochemical characteristics on the resulting BFXTEN (e.g., the sequences of Table 4 or sequence variants or fragments thereof); (3) establishing a desired N- to C-terminus configuration of the BFXTEN to achieve the desired efficacy or PK parameters; (4) establishing the design of the expression vector encoding the configured BFXTEN; (5) transforming a suitable host with the expression vector; and (6) expression and recovery of the resultant BFXTEN fusion protein. The method of increasing the terminal half-life provides that the BP and XTEN components can be configured and produced as compositions in an N- to C-terminus orientation (exclusive of spacer sequences), that include, but are not limited to BP-XTEN; XTEN-BP; BP1-XTEN-BP2; XTEN1-BP-XTEN2; BP1-BP2-XTEN1; BP2-BP1-XTEN; BP2-XTEN-BP1; BP1-XTEN1-BP2-XTEN2; XTEN1-BP1-XTEN2-BP2 (wherein “1”, “2”, and “3” represent different molecules of the respective BP and XTEN portions of the fusion proteins), one of the configurations of formulae I-VI above, or the configurations of FIG. 1, and the compositions are subsequently produced and evaluated for receptor binding affinity for the respective BP1 or BP2 components, and those exhibiting reduced binding affinity are evaluated for a concomitant RMC and increased terminal half-life compared to one of the alternative configurations. In some embodiments, the foregoing method provides configured BFXTEN compositions that have an increase in the terminal half-life of at least about 30%, or about 50%, or about 75%, or about 100%, or about 150%, or about 200%, or about 300%, or about 400%, or about 500% or more compared to the half-life of a BFXTEN in a second configuration where receptor binding of at least one BP is not reduced, or compared to the corresponding BP not linked to XTEN, yet still retain at least a portion of the biological activity of the corresponding BP. The method takes advantage of the fact that certain ligands with reduced binding affinity to a receptor, either as a result of a decreased on-rate or an increased off-rate, may be effected by the obstruction of either the N- or C-terminus (as shown in FIG. 3), and using that terminus as the linkage to another polypeptide of the composition, whether another molecule of a BP, an XTEN, or a spacer sequence results in the reduced binding affinity. The choice of the particular configuration of the BFXTEN fusion protein reduces the degree of binding affinity to the receptor such that a reduced rate of receptor-mediated clearance is achieved. Generally, activation of the receptor is coupled to RMC such that binding of a polypeptide to its receptor without activation does not lead to RMC, while activation of the receptor leads to RMC. However, in some cases, particularly where the ligand has an increased off rate, the ligand may nevertheless be able to bind sufficiently to initiate cell signaling without triggering receptor mediated clearance, with the net result that the BFXTEN remains bioavailable. In such cases, the configured BFXTEN has an increased half-life compared to those configurations that lead to a higher degree of RMC.

Accordingly, in some embodiments, the method provides that the half-life of the BFXTEN can be increased by designing the BFXTEN to have an N- to C-terminus configuration wherein the terminal half-life is increased at least about 50%, or at least about 75%, or at least about 100%, or at least about 150%, or at least about 200%, at least about 300% wherein the BFXTEN has reduced binding affinity of at least one BP component for the target receptor by at least about two-fold, or at least about three-fold, or at least about four-fold, or at least about five-fold compared to a BFXTEN configured wherein the binding affinity of the BP component is not reduced.

### V). Methods of Use: Treatment Applications of BFXTEN and Methods of Enhancing Biologically Active Proteins

In another aspect, the invention provides a method of for achieving a beneficial effect in a metabolic and/or cardiovascular disease, disorder or condition mediated by BP. The present invention addresses disadvantages and/or limitations of the use of single BP or combinations of BP that have a relatively short terminal half-life and/or a narrow therapeutic window between the minimum effective dose and the maximum tolerated dose.

In one embodiment, the invention provides a method for achieving a beneficial affect in a subject, such as a human with a metabolic and/or cardiovascular disease, disorder or condition, comprising the step of administering to the subject an effective amount of a BFXTEN wherein the administered BXTEN results in an improvement in at least one physiological parameter or clinical symptom associated with the disease, disorder or condition. The effective amount produces a beneficial effect in helping to treat (e.g., cure or reduce the severity) or prevent (e.g., reduce the likelihood of onset or severity) a disease or disorder in a subject suffering from or at risk of developing a metabolic- or cardiovascular-related disease, disorder or condition, including, but not limited to, one or more selected from Table 7. Other examples of other diseases or clinical disorders that may benefit from treatment with the BFXTEN compositions of the present invention include, but are not limited to, the “honeymoon period” of Type I diabetes, excessive appetite, insufficient satiety, metabolic disorder, glucagonomas, secretory disorders of the airway, arthritis, osteoporosis, central nervous system disease, restenosis, neurodegenerative disease, renal failure, congestive heart failure, cardiac hypertrophy, nephrotic syndrome, cirrhosis, pulmonary edema, hypertension, disorders wherein the reduction of food intake is desired, a disease or disorder of the central nervous system, irritable bowel syndrome, myocardial infarction, cardiac valve disease, stroke, post-surgical catabolic changes, hibernating myocardium or diabetic cardiomyopathy, hypertrophic cardiomyopathy, heart insufficiency, aortic stenosis, valvular regurgitation, intermittent claudication, insufficient urinary sodium excretion, excessive urinary potassium concentration, conditions or disorders associated with toxic hypervolemia, polycystic ovary syndrome, respiratory distress, chronic skin ulcers, nephropathy, and left ventricular systolic dysfunction.

TABLE 7
Metabolic and Cardiovascular Diseases
Metabolic and/or Cardiovascular Diseases
Diabetes
Type 1 diabetes
Type 2 diabetes
Syndrome X
Insulin resistance
Hyperinsulinemia
Atherosclerosis
Cardiovascular disease
Congestive heart failure
Diabetic neuropathy
Dyslipidemia
Eating disorders
Gestational diabetes
Hypercholesterolemia
Hypertension
Insufficient pancreatic beta cell mass
Myocardial ischemia
Myocardial reperfusion
Obesity
Pulmonary hypertension
Retinal neurodegenerative processes
Stroke

The invention contemplates use of BFXTEN that incorporate specific combinations of BP selected from Table 1 (or sequence variants thereof) that mediate or result in pharmacologic effects that are complementary, additive in effect, or synergistic in effect on one or more of the clinical, biochemical, or physiologic parameters disclosed herein for a metabolic and/or cardiovascular disease or disorder. In the case of glucose or insulin resistance disorders, such parameters include, but are not limited to HbA1c concentrations, insulin concentrations, stimulated C peptide, fasting plasma glucose (FPG), serum cytokine levels, CRP levels, insulin secretion and Insulin-sensitivity index derived from an oral glucose tolerance test (OGTT), body weight, triglyceride levels, cholesterol, body weight, and food consumption.

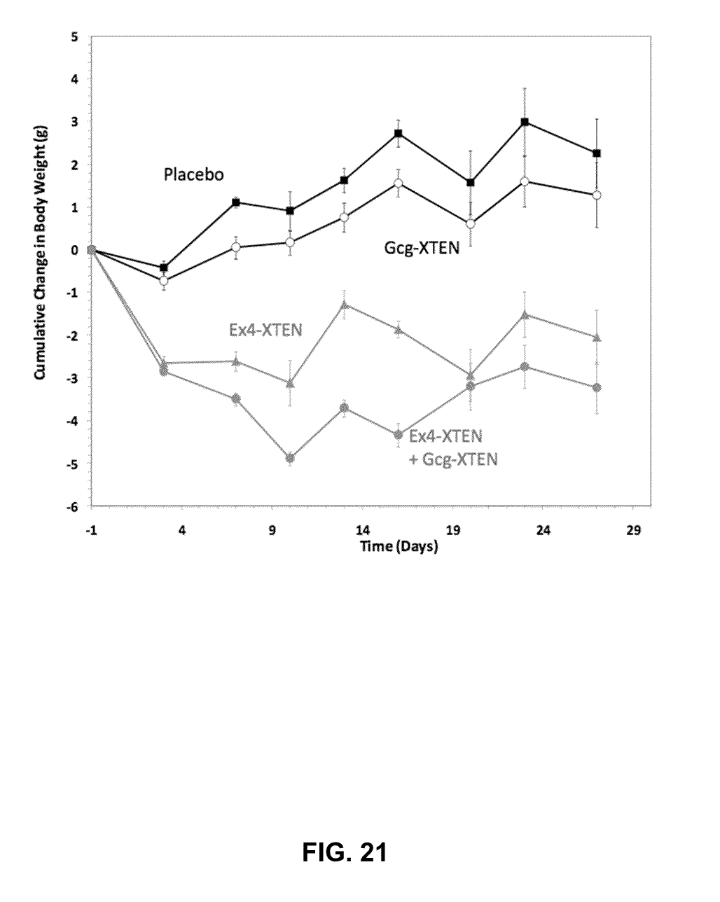

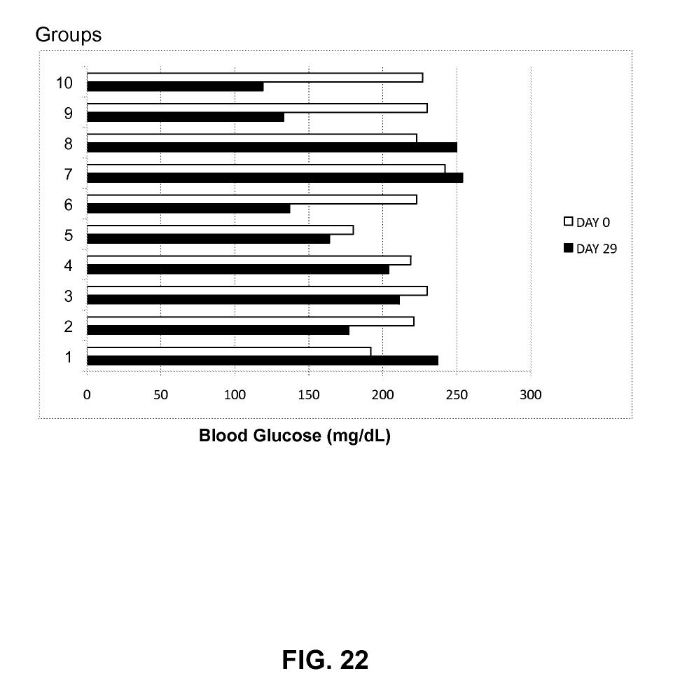

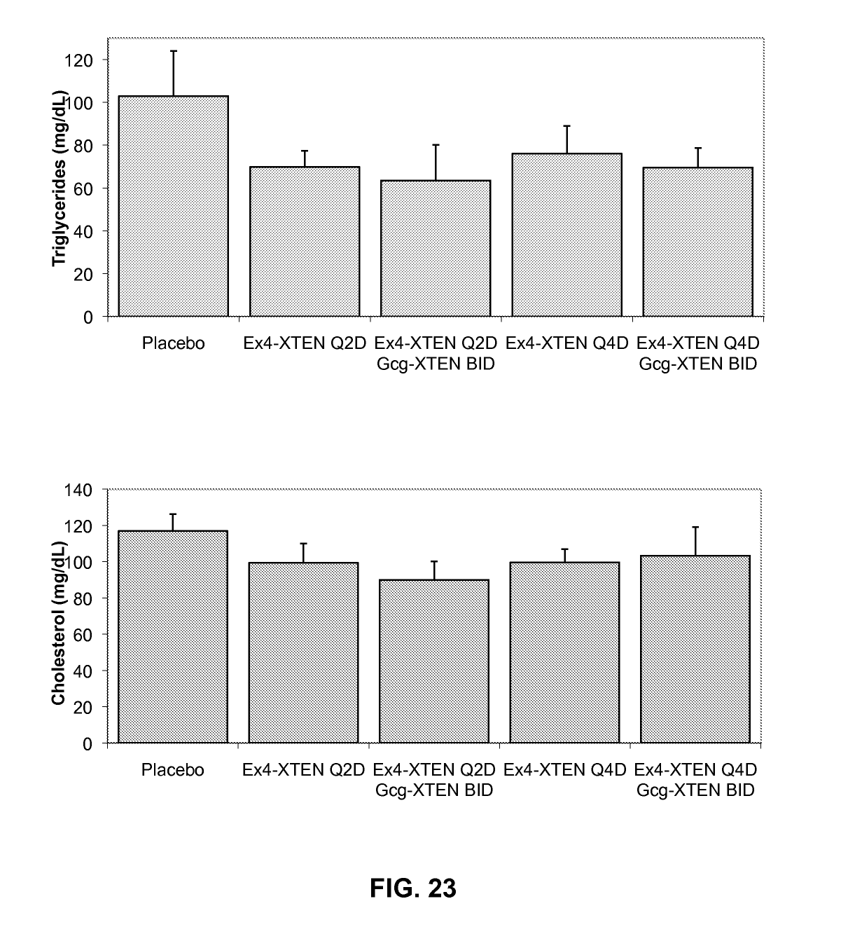

In one embodiment, the method comprises administering a therapeutically-effective amount of a pharmaceutical composition comprising a monomeric BFXTEN fusion protein or a combination BFXTEN fusion protein composition comprising a first BP and a second BP selected from Table 1 (or fragments or sequence variants thereof) linked to XTEN sequence(s) and at least one pharmaceutically acceptable carrier to a subject in need thereof that results in greater improvement or a change of greater magnitude in at least one parameter, physiologic condition, or clinical outcome mediated by the first and/or the second BP component(s) compared to the effect mediated by administration of a pharmaceutical composition comprising just one of the BP. In another embodiment, the administration of a BFXTEN may result in improvement of at least one additional bio-activity, that may result from the inclusion of the second component BP or may be a result of an additive or synergistic effect of the combination of the first and the second BPs. In one example of the foregoing embodiments, the method of treatment comprises administration of a BFXTEN comprising BP using a therapeutically effective dose regimen to effect improvements in one or more parameters associated with diabetes or insulin resistance. The improvements may be assessed by a primary efficacy or clinical endpoint, for example an improvement in hemoglobin A1c (HbA1c, see for example Reynolds et al., BMJ, 333(7568):586-589, 2006). Improvements in HbA1c that are indicative of therapeutic efficacy may vary depending on the initial baseline measurement in a patient, with a larger decrease often corresponding to a higher initial baseline and a smaller decrease often corresponding to a lower initial baseline. In one embodiments, the method results in an HbA1c decrease of at least about 0.2%, or alternatively at least about 0.5%, or alternatively at least about 1%, or alternatively at least about 1.5%, or alternatively at least about 2%, or alternatively at least about 2.5%, or alternatively at least about 3%, or alternatively at least about 3.5%, or at least about 4% or more compared with pre-dose levels. In another embodiment, the method of treatment results in reductions in fasting blood sugar (e.g., glucose) levels to <140 mg/dL, alternatively <130 mg/dL, alternatively <125 mg/dL, alternatively <120 mg/dL, alternatively <115 mg/dL, alternatively <110 mg/dL, alternatively <105 mg/dL, or fasting blood sugar levels <100 mg/dL. In other embodiments, the method can result in 120 minute oral glucose tolerance test (OGTT) glucose levels of <200 mg/dL, more preferably <190 mg/dL, more preferably <180 mg/dL, more preferably <170 mg/dL, more preferably <160 mg/dL, more preferably <150 mg/dL, and most preferably <140 mg/dL. In one embodiment, wherein a BFXTEN comprising two BP associated with glucose homeostasis is administered to a subject in need thereof, the administration results in an improvement in fasting blood glucose of <140 mg/dL, alternatively <130 mg/dL, alternatively <125 mg/dL, alternatively <120 mg/dL, alternatively <115 mg/dL, alternatively <110 mg/dL, alternatively <105 mg/dL, or fasting blood sugar levels <100 mg/dL, and further results in an improvement in HbA1c of at least about 0.2%, or at least about 0.5%, or at least about 1%, or at least about 2%, or at least 3%, or at least about 4% or more. In other embodiments of the method, the administration of a BFXTEN may result in improvements of any two parameters selected from insulin concentration, stimulated C peptide, serum cytokine levels, CRP levels, insulin secretion and Insulin-sensitivity index in response to an oral glucose tolerance test (OGTT), body weight, triglyceride levels, cholesterol, body weight, and food consumption. In another embodiment, administration of the BFXTEN to a subject in need thereof can result in an improvement in one or more of the clinical or biochemical or physiologic parameters that is of longer duration or greater magnitude than the that of one of the single BP components not linked to XTEN and administered at a comparable dose, determined using that same assay or based on a measured clinical parameter. Data supporting such beneficial combinations are presented in Example 25 and FIGS. 21-23, where exenatide and glucagon were prepared as two fusion proteins of different length and were used together in a model of diabetes to result in multiple beneficial effects, including reductions in body weight and fasting blood glucose, without evidence of overt toxicity.

As a result of the enhanced PK of the BFXTEN, as described herein, the method provides that the BFXTEN may be administered using longer intervals between doses compared to the corresponding BP not linked to XTEN to prevent, alleviate, reverse or ameliorate symptoms of the metabolic and/or cardiovascular disease, disorder or condition or prolong the survival of the subject being treated. The method of treatment may include administration of consecutive doses of a therapeutically effective amount of the BFXTEN for a period of time sufficient to achieve and/or maintain the desired physiological parameter or biological effect, and such consecutive doses of a therapeutically effective amount establishes the therapeutically effective dose regimen for the BFXTEN; i.e., the schedule for consecutively administered doses of the fusion protein composition, wherein the doses are given in therapeutically effective amounts to result in a sustained beneficial effect on or improvement in any clinical sign or symptom, aspect, measured physiological parameter or characteristic of a metabolic and/or cardiovascular disease state or condition, including, but not limited to, those described herein. A therapeutically effective amount of the BFXTEN may vary according to factors such as the disease state, age, sex, and weight of the individual, and the ability of the antibody or antibody portion to elicit a desired response in the individual. A therapeutically effective amount is also one in which any toxic or detrimental effects of the BFXTEN are outweighed by the therapeutically beneficial effects. A prophylactically effective amount refers to an amount of BFXTEN required for the period of time necessary to achieve the desired prophylactic result.

For the methods of treatment, longer acting BFXTEN compositions are preferred, so as to improve patient convenience and to increase the interval between doses and to reduce the amount of drug required to achieve a sustained effect. In one embodiment of the method of treatment, the administration of an effective amount of a BFXTEN to a subject in need thereof results in a gain in time spent within a therapeutic window established for the fusion protein of the composition compared to the corresponding BP component(s) not linked to the fusion protein and administered at a comparable dose to a subject. In the embodiment, the gain in time spent within the therapeutic window is at least about three-fold, or at least about four-fold, or at least about five-fold, or at least about six-fold, or at least about eight-fold, or at least about 10-fold, or at least about 20-fold, or at least about 40-fold compared to the corresponding BP component(s) not linked to the fusion protein and administered at a comparable dose to a subject. In another embodiment, the method of treatment provides that administration of multiple consecutive doses of a BFXTEN administered using a therapeutically effective dose regimen to a subject in need thereof results in a gain in time between consecutive Cₘₐₓ peaks and/or Cₘᵢₙ troughs for blood levels of the fusion protein compared to the corresponding BP(s) not linked to the fusion protein and administered using a dose regimen established for that BP. In the foregoing embodiment, the gain in time spent between consecutive Cₘₐₓ peaks and/or Cₘᵢₙ troughs can be at least about three-fold, or at least about four-fold, or at least about five-fold, or at least about six-fold, or at least about eight-fold, or at least about 10-fold, or at least about 20-fold, or at least about 40-fold compared to the corresponding BP component(s) not linked to the fusion protein and administered using a dose regimen established for that BP. In the embodiments hereinabove described in this paragraph the administration of the fusion protein can result in an improvement in at least one of the parameters disclosed herein as being related to metabolic or cardiovascular diseases using a lower unit dose in moles of fusion protein compared to the corresponding BP component(s) not linked to the fusion protein and administered at a comparable unit dose or dose regimen to a subject.

In some embodiments of the method of treatment, (i) a smaller molar amount of (e.g. of about two-fold less, or about three-fold less, or about four-fold less, or about five-fold less, or about six-fold less, or about eight-fold less, or about 10-fold-less or greater) the BXTEN fusion protein composition is administered in comparison to the corresponding BPs not linked to the XTEN under an otherwise same dose regimen, and the fusion protein achieves a comparable therapeutic effect as the corresponding BPs not linked to the XTEN; (ii) the fusion protein is administered less frequently (e.g., an increase of at least 2 days, or about 4 days, or about 7 days, or about 10 days, or about 14 days, or about 21 days longer between consecutive doses) in comparison to the corresponding BPs not linked to the XTEN under an otherwise same dose amount, and the fusion protein achieves a comparable therapeutic effect as the corresponding BPs not linked to the XTEN; or (iii) an accumulative smaller molar amount (e.g. about 5%, or about 10%, or about 20%, or about 40%, or about 50%, or about 60%, or about 70%, or about 80%, or about 90% less) of the fusion protein is administered in comparison to the corresponding BPs not linked to the XTEN under the otherwise same dose regimen the fusion protein achieves a comparable therapeutic effect as the corresponding BPs not linked to the XTEN. The accumulative smaller molar amount is measure for a period of at least about one week, or about 14 days, or about 21 days, or about one month. The therapeutic effect can be determined by any of the measured parameters or clinical endpoints described herein.

The invention further contemplates that BFXTEN used in accordance with the methods provided herein may be administered in conjunction with other treatment methods and pharmaceutical compositions. Such compositions, may include for example, DPP-IV inhibitors, insulin, insulin analogues, PPAR gamma agonists, dual-acting PPAR agonists, GLP-1 agonists or analogues, PTP1B inhibitors, SGLT inhibitors, insulin secretagogues, RXR agonists, glycogen synthase kinase-3 inhibitors, insulin sensitizers, immune modulators, beta-3 adrenergic receptor agonists, Pan-PPAR agonists, 11beta-HSD1 inhibitors, amylin analogues, biguanides, alpha-glucosidase inhibitors, meglitinides, thiazolidinediones, sulfonylureas and other diabetes medicants known in the art.

The foregoing notwithstanding, in certain embodiments, the BFXTEN used in accordance with the methods of the present invention may prevent or delay the need for additional treatment methods or use of drugs or other pharmaceutical compositions in subjects with metabolic and/or cardiovascular diseases or disorders. In other embodiments, the BFXTEN may reduce the amount, frequency or duration of additional treatment methods or drugs or other pharmaceutical compositions required to treat the underlying metabolic and/or cardiovascular disease, disorder or condition.

In another aspect, the invention provides a method of designing the bifunctional BXTEN compositions with desired pharmacologic or pharmaceutical properties. The bifunctional BMXTEN and BCXTEN fusion proteins are designed and prepared with various objectives in mind, including improving the therapeutic efficacy over the single bioactive compounds in the treatment of metabolic and/or cardiovascular diseases or disorders, enhancing the pharmacokinetic characteristics of the BP components of one or both of the fusion proteins, lowering the dose of one or both of the BP components required to achieve a pharmacologic effect, and to enhance the ability of the BP components to remain within the therapeutic window for an extended period of time. The design criteria for the fusion proteins may include, but not be limited to: (a) desired in vivo efficacy for a single parameter of the metabolic and/or cardiovascular disease, such as an additive or a synergistic effect that may be achieved with a lower dose or less frequent dosing compared to a use of a single BP; (b) desired in vivo efficacy for two parameters of the therapeutic or prophylactic indication, each mediated by one of the different BPs that collectively result in an enhanced effect; and (c) optional dual action of the paired BPs for multiple therapeutic or prophylactic indications.

The steps in the design of the fusion proteins and the inventive compositions generally involve: (1) the identification, selection and pairing of BPs (e.g., native proteins, peptide hormones, peptide analogs or derivatives with activity, peptide fragments, such as those of Table 1) to treat the particular metabolic and/or cardiovascular disease, disorder or condition; (2) selecting the XTEN that will confer the desired PK and physicochemical characteristics on the respective BP (e.g., the XTEN of Table 4 or sequence variants or fragments thereof; (3) establishing the optimal N- to C-termini configuration of the BFXTEN to achieve the desired efficacy (e.g., the configurations of formulae I-VI); (4) the covalent linking of BPs either directly or via a spacer to an XTEN selected for its particular pharmaceutical properties; (5) expression and recovery of the resultant fusion protein(s); and in the case of combination BFXTEN comprising two fusion proteins; (6) establishing the fixed ratio of the two fusion proteins in the BFXTEN composition, wherein the administration of the composition to a subject results in the fusion protein(s) being maintained within the therapeutic window for a greater period compared to BPs not linked to XTEN.

In another aspect, the invention provides methods of making BFXTEN compositions to improve ease of manufacture, result in increased stability, increased water solubility, and/or ease of formulation, as compared to the native BPs. In one embodiment, the invention includes a method of increasing the water solubility of a BP comprising the step of linking the BP to one or more XTEN such that a higher concentration in soluble form of the resulting BFXTEN can be achieved, under physiologic conditions, compared to the BP in an un-fused state. Factors that contribute to the property of XTEN to confer increased water solubility of BPs when incorporated into a fusion protein include the high solubility of the XTEN fusion partner and the low degree of self-aggregation between molecules of XTEN in solution. In some embodiments, the method results in a BFXTEN fusion protein wherein the water solubility is at least about 50%, alternatively 60%, alternatively 70%, alternatively 80%, alternatively 90%, alternatively 100%, alternatively 150%, or at least about 200% greater, or at least about 400% greater, or at least about 600% greater, or at least about 800% greater, or at least about 1000% greater, or at least about 2000% greater, or at least about 4000% greater, or at least about 6000% greater under physiologic conditions, compared to the un-fused BP.

In another embodiment, the invention includes a method of enhancing the shelf-life of a BP comprising the step of linking the BP with one or more XTEN selected such that the shelf-life of the resulting BFXTEN is extended compared to the BP in an un-fused state. As used herein, shelf-life refers to the period of time over which the functional activity of a BP or BFXTEN that is in solution or in some other storage formulation remains stable without undue loss of activity. As used herein, “functional activity” refers to a pharmacologic effect or biological activity, such as the ability to bind a receptor or ligand, or an enzymatic activity, or to display one or more known functional activities associated with a BP, as known in the art. A BP that degrades or aggregates generally has reduced functional activity or reduced bioavailability compared to one that remains in solution. Factors that contribute to the ability of the method to extend the shelf life of BPs when incorporated into a fusion protein include the increased water solubility, reduced self-aggregation in solution, and increased heat stability of the XTEN fusion partner. In particular, the low tendency of XTEN to aggregate facilitates methods of formulating pharmaceutical preparations containing higher drug concentrations of BPs, and the heat-stability of XTEN contributes to the property of BFXTEN fusion proteins to remain soluble and functionally active for extended periods. In one embodiment, the method results in BFXTEN fusion proteins with “prolonged” or “extended” shelf-life that exhibit greater activity relative to a standard that has been subjected to the same storage and handling conditions. The standard may be the un-fused full-length BP. In one embodiment, the method includes the step of formulating the isolated BFXTEN with one or more pharmaceutically acceptable excipients that enhance the ability of the XTEN to retain its unstructured conformation and for the BFXTEN to remain soluble in the formulation for a time that is greater than that of the corresponding un-fused BP. In one embodiment, the step of linking a BP to an XTEN to create a BFXTEN fusion protein results in a solution that retains greater than about 100% of the functional activity, or greater than about 105%, 110%, 120%, 130%, 150% or 200% of the functional activity of a standard when subjected to the same storage and handling conditions as the standard when compared at a given time point, thereby enhancing its shelf-life.

Shelf-life may also be assessed in terms of functional activity remaining after storage, normalized to functional activity when storage began. BFXTEN fusion proteins of the invention with prolonged or extended shelf-life as exhibited by prolonged or extended functional activity may retain about 50% more functional activity, or about 60%, 70%, 80%, or 90% more of the functional activity of the equivalent BP not linked to XTEN when subjected to the same conditions for the same period of time. For example, a BFXTEN fusion protein of the invention comprising exendin-4 or glucagon fused to a XTEN sequence may retain about 80% or more of its original activity in solution for periods of up to 5 weeks or more under various temperature conditions. In some embodiments, the BFXTEN retains at least about 50%, or about 60%, or at least about 70%, or at least about 80%, and most preferably at least about 90% or more of its original activity in solution when heated at 80° C. for 10 min In other embodiments, the BFXTEN retains at least about 50%, preferably at least about 60%, or at least about 70%, or at least about 80%, or alternatively at least about 90% or more of its original activity in solution when heated or maintained at 37° C. for about 7 days. In another embodiment, BFXTEN fusion protein retains at least about 80% or more of its functional activity after exposure to a temperature of about 30° C. to about 70° C. over a period of time of about one hour to about 18 hours.

### VI). The DNA Sequences of the Invention

The present invention provides isolated polynucleic acids encoding BFXTEN chimeric polypeptides and sequences complementary to polynucleic acid molecules encoding BFXTEN chimeric polypeptides, including homologous variants. In another aspect, the invention encompasses methods to produce polynucleic acids encoding BFXTEN chimeric polypeptides and sequences complementary to polynucleic acid molecules encoding BFXTEN chimeric polypeptides, including homologous variants. In general, the methods of producing biologically active BFXTEN comprise providing a polynucleotide sequence coding for a fusion protein comprising BP linked with one or more XTEN tails, and causing the fusion protein to be expressed in a transformed host cell, thereby producing the biologically-active BFXTEN polypeptide. Standard recombinant techniques in molecular biology can be used to make the polynucleotides of the present invention.

In accordance with the invention, nucleic acid sequences that encode BFXTEN may be used to generate recombinant DNA molecules that direct the expression of BFXTEN fusion proteins in appropriate host cells. Several cloning strategies are envisioned to be suitable for performing the present invention, many of which can be used to generate a construct that comprises a gene coding for a fusion protein of the BFXTEN composition of the present invention, or its complement. In one embodiment, the cloning strategy would be used to create a gene that encodes a monomeric BFXTEN that comprises two BP and at least a first XTEN polypeptide, or its complement. In another embodiment, the cloning strategy would be used to create a first gene that encodes a monomeric BFXTEN that comprises a first BP and at least a first XTEN (or its complement), and a second gene that encodes a monomeric BFXTEN that comprises a second BP and at least a first XTEN (or its complement) that would be used to transform separate host cells for expression of fusion proteins used to formulate a combination BFXTEN composition.

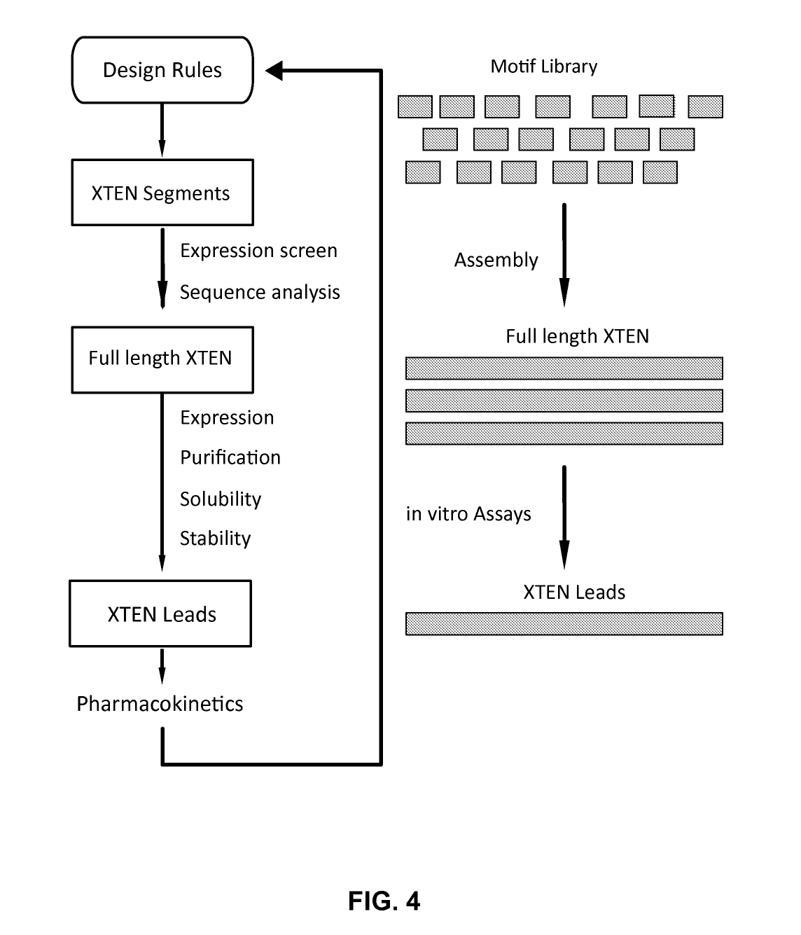

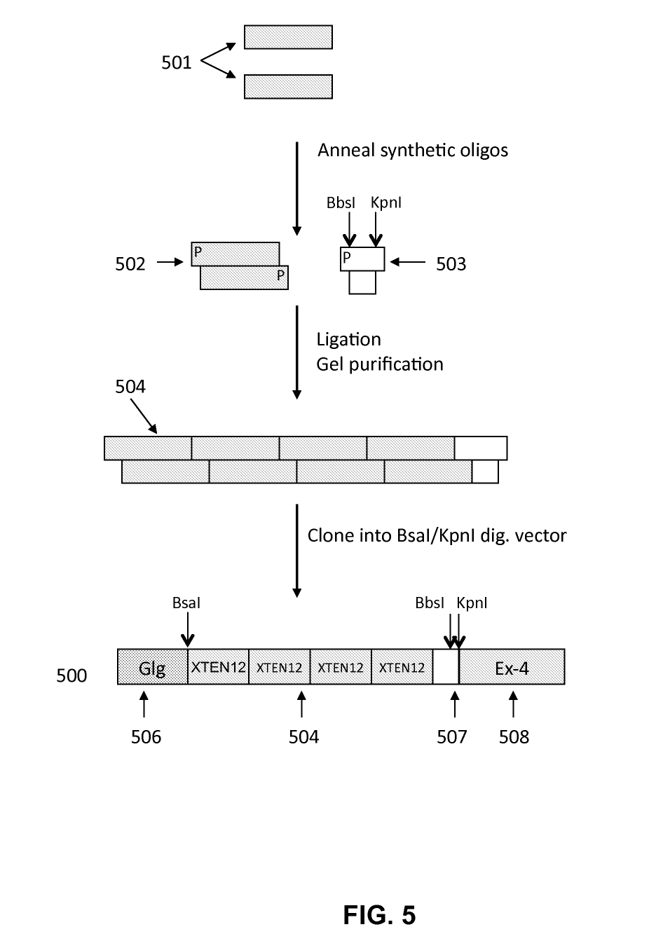

In designing optimal XTEN sequences, it was discovered that the non-repetitive nature of the XTEN of the inventive compositions can be achieved despite use of a “building block” molecular approach in the creation of the XTEN-encoding sequences. This was achieved by the use of a library of polynucleotides encoding sequence motifs that are then multimerized to create the genes encoding the XTEN sequences (see FIGS. 4 and 5). Thus, while the expressed XTEN may consist of multiple units of as few as four different sequence motifs, because the motifs themselves consist of non-repetitive amino acid sequences, the overall XTEN sequence is rendered non-repetitive. Accordingly, in one embodiment, the XTEN-encoding polynucleotides comprise multiple polynucleotides that encode non-repetitive sequences, or motifs, operably linked in frame and in which the resulting expressed XTEN amino acid sequences are non-repetitive.

In one approach, a construct is first prepared containing the DNA sequence corresponding to BFXTEN fusion protein. DNA encoding the respective BP of the bifunctional compositions may be obtained from a cDNA library prepared using standard methods from tissue or isolated cells believed to possess BP mRNA and to express it at a detectable level. If necessary, the coding sequence can be obtained using conventional primer extension procedures as described in Sambrook, et al., supra, to detect precursors and processing intermediates of mRNA that may not have been reverse-transcribed into cDNA. Accordingly, DNA can be conveniently obtained from a cDNA library prepared from such sources. The BP encoding gene(s) may also be obtained from a genomic library or created by standard synthetic procedures known in the art (e.g., automated nucleic acid synthesis) using DNA sequences obtained from publicly available databases, patents, or literature references. Such procedures are well known in the art and well described in the scientific and patent literature. For example, sequences can be obtained from Chemical Abstracts Services (CAS) Registry Numbers (published by the American Chemical Society) and/or GenBank Accession Numbers (e.g., Locus ID, NP_XXXXX, and XP_XXXXX) Model Protein identifiers available through the National Center for Biotechnology Information (NCBI) webpage, available on the world wide web at ncbi.nlm.nih.gov that correspond to entries in the CAS Registry or GenBank database that contain an amino acid sequence of the BAP or of a fragment or variant of the BAP. For such sequence identifiers provided herein, the summary pages associated with each of these CAS and GenBank and GenSeq Accession Numbers as well as the cited journal publications (e.g., PubMed ID number (PMID)) are each incorporated by reference in their entireties, particularly with respect to the amino acid sequences described therein. In one embodiment, the BP encoding gene encodes a protein of Table 1, or a fragment or variant thereof.

A gene or polynucleotide encoding the BP portion of the subject BFXTEN protein, in the case of an expressed fusion protein that will comprise a single BP, and a second gene or polynucleotide encoding a second BP in the case of an expressed monomeric fusion protein that will comprise two BP, can be then be cloned into a construct, which can be a plasmid or other vector under control of appropriate transcription and translation sequences for high level protein expression in a biological system. In a later step, a second gene or polynucleotide coding for the XTEN is genetically fused to the nucleotides encoding the N- and/or C-terminus of the BP gene by cloning it into the construct adjacent and in frame with the gene(s) coding for the BP. This second step can occur through a ligation or multimerization step. In the foregoing embodiments hereinabove described in this paragraph, it is to be understood that the gene constructs that are created can alternatively be the complement of the respective genes that encode the respective fusion proteins.

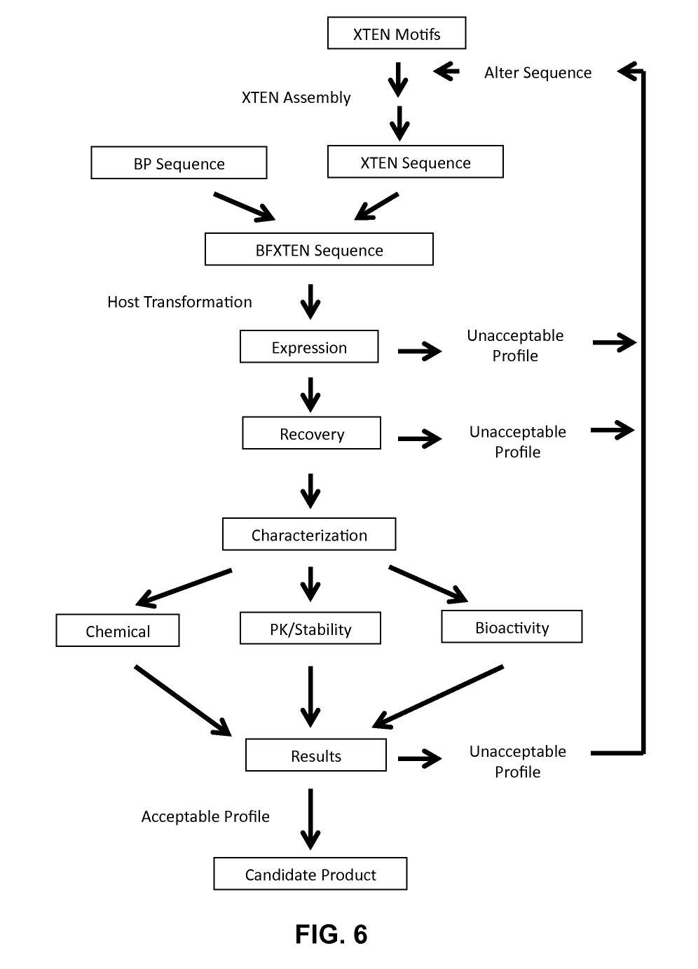

The gene encoding for the XTEN can be made in one or more steps, either fully synthetically or by synthesis combined with enzymatic processes, such as restriction enzyme-mediated cloning, PCR and overlap extension. XTEN polypeptides can be constructed such that the XTEN-encoding gene has low repetitiveness while the encoded amino acid sequence has a degree of repetitiveness. Genes encoding XTEN with non-repetitive sequences can be assembled from oligonucleotides using standard techniques of gene synthesis. The gene design can be performed using algorithms that optimize codon usage and amino acid composition. In one method of the invention, a library of relatively short XTEN-encoding polynucleotide constructs is created and then assembled, as illustrated in FIGS. 5 and 6. This can be a pure codon library such that each library member has the same amino acid sequence but many different coding sequences are possible. Such libraries can be assembled from partially randomized oligonucleotides and used to generate large libraries of XTEN segments comprising the sequence motifs. The randomization scheme can be optimized to control amino acid choices for each position as well as codon usage.

Polynucleotide Libraries

In another aspect, the invention provides libraries of polynucleotides that encode XTEN sequences that can be used to assemble genes that encode XTEN of a desired length and sequence.

In certain embodiments, the XTEN-encoding library constructs comprise polynucleotides that encode polypeptide segments of a fixed length. As an initial step, a library of oligonucleotides that encode motifs of 9-14 amino acid residues can be assembled. In a preferred embodiment, libraries of oligonucleotides that encode motifs of 12 amino acids are assembled.

The XTEN-encoding sequence segments can be dimerized or multimerized into longer encoding sequences. Dimerization or multimerization can be performed by ligation, overlap extension, PCR assembly or similar cloning techniques known in the art. This process of can be repeated multiple times until the resulting XTEN-encoding sequences have reached the organization of sequence and desired length, providing the XTEN-encoding genes. As will be appreciated, a library of polynucleotides that encodes 12 amino acids can be dimerized into a library of polynucleotides that encode 36 amino acids. In turn, the library of polynucleotides that encode 36 amino acids can be serially dimerized into a library containing successively longer lengths of polynucleotides that encode XTEN sequences. In some embodiments, libraries can be assembled of polynucleotides that encode amino acids that are limited to specific sequence XTEN families; e.g., AD, AE, AF, AG, AM, or AQ sequences of Table 3. In other embodiments, libraries can comprises sequences that encode two or more of the motif family sequences from Table 3. Representative polynucleotide sequences of libraries that encode 36mers are presented in Tables 9-12, the design and making of which are described more fully in the Examples. The libraries can be used, in turn, for serial dimerization or ligation to achieve polynucleotide sequence libraries that encode XTEN sequences, for example, of 72, 144, 288, 576, 864, 1296 amino acids, up to a total length of about 3000 amino acids, as well as for the production of intermediate lengths that represent fragments of the XTEN polypeptide sequences of Table 4. In some cases, the polynucleotide library sequences may also include additional bases used as “sequencing islands,” described more fully below.

FIG. 6 is a schematic flowchart of representative, non-limiting steps in the assembly of a XTEN polynucleotide construct and a BFXTEN polynucleotide construct in the embodiments of the invention. Individual oligonucleotides 501 can be annealed into sequence motifs 502 such as a 12 amino acid motif (“12-mer”), which is subsequently ligated with an oligo containing BbsI, and KpnI restriction sites 503. Additional sequence motifs from a library are annealed to the 12-mer until the desired length of the XTEN gene 504 is achieved. The XTEN gene is cloned into a stuffer vector. The vector encodes a glucagon gene 506 followed by a stuffer sequence that is flanked by BsaI, BbsI, and KpnI sites 507 and a gene encoding exendin-4 508, resulting in the gene encoding a BFXTEN comprising a two BP 500. A non-exhaustive list of polynucleotides encoding XTEN and precursor sequences is provided in Table 8.

TABLE 8
DNA sequences of XTEN and precursor sequences

| XTEN | | SEQ ID |
|---|---|---|
| Name | DNA Sequence | NO: |
| AE144 | GGTAGCGAACCGGCAACTTCCGGCTCTGAAACCCCAGGTACTTCTGAAAGCGCT | 161 |
| | ACTCCTGAGTCTGGCCCAGGTAGCGAACCTGCTACCTCTGGCTCTGAAACCCCA | |
| | GGTAGCCCGGCAGGCTCTCCGACTTCCACCGAGGAAGGTACCTCTACTGAACCT | |
| | TCTGAGGGTAGCGCTCCAGGTAGCGAACCGGCAACCTCTGGCTCTGAAACCCCA | |
| | GGTAGCGAACCTGCTACCTCCGGCTCTGAAACTCCAGGTAGCGAACCGGCTACT | |
| | TCCGGTTCTGAAACTCCAGGTACCTCTACCGAACCTTCCGAAGGCAGCGCACCA | |
| | GGTACTTCTGAAAGCGCAACCCCTGAATCCGGTCCAGGTAGCGAACCGGCTACT | |
| | TCTGGCTCTGAGACTCCAGGTACTTCTACCGAACCGTCCGAAGGTAGCGCACCA | |
| AF144 | GGTACTTCTACTCCGGAAAGCGGTTCCGCATCTCCAGGTACTTCTCCTAGCGGTG | 162 |
| | AATCTTCTACTGCTCCAGGTACCTCTCCTAGCGGCGAATCTTCTACTGCTCCAGG | |
| | TTCTACCAGCTCTACCGCTGAATCTCCTGGCCCAGGTTCTACCAGCGAATCCCCG | |
| | TCTGGCACCGCACCAGGTTCTACTAGCTCTACCGCAGAATCTCCGGGTCCAGGT | |
| | ACTTCCCCTAGCGGTGAATCTTCTACTGCTCCAGGTACCTCTACTCCGGAAAGCG | |
| | GCTCCGCATCTCCAGGTTCTACTAGCTCTACTGCTGAATCTCCTGGTCCAGGTAC | |
| | CTCCCCTAGCGGCGAATCTTCTACTGCTCCAGGTACCTCTCCTAGCGGCGAATCT | |
| | TCTACCGCTCCAGGTACCTCCCCTAGCGGTGAATCTTCTACCGCACCA | |
| AE288 | GGTACCTCTGAAAGCGCAACTCCTGAGTCTGGCCCAGGTAGCGAACCTGCTACC | 163 |
| | TCCGGCTCTGAGACTCCAGGTACCTCTGAAAGCGCAACCCCGGAATCTGGTCCA | |
| | GGTAGCGAACCTGCAACCTCTGGCTCTGAAACCCCAGGTACCTCTGAAAGCGCT | |
| | ACTCCTGAATCTGGCCCAGGTACTTCTACTGAACCGTCCGAGGGCAGCGCACCA | |
| | GGTAGCCCTGCTGGCTCTCCAACCTCCACCGAAGAAGGTACCTCTGAAAGCGCA | |
| | ACCCCTGAATCCGGCCCAGGTAGCGAACCGGCAACCTCCGGTTCTGAAACCCCA | |
| | GGTACTTCTGAAAGCGCTACTCCTGAGTCCGGCCCAGGTAGCCCGGCTGGCTCT | |
| | CCGACTTCCACCGAGGAAGGTAGCCCGGCTGGCTCTCCAACTTCTACTGAAGAA | |
| | GGTACTTCTACCGAACCTTCCGAGGGCAGCGCACCAGGTACTTCTGAAAGCGCT | |
| | ACCCCTGAGTCCGGCCCAGGTACTTCTGAAAGCGCTACTCCTGAATCCGGTCCA | |
| | GGTACTTCTGAAAGCGCTACCCCGGAATCTGGCCCAGGTAGCGAACCGGCTACT | |
| | TCTGGTTCTGAAACCCCAGGTAGCGAACCGGCTACCTCCGGTTCTGAAACTCCA | |
| | GGTAGCCCAGCAGGCTCTCCGACTTCCACTGAGGAAGGTACTTCTACTGAACCT | |
| | TCCGAAGGCAGCGCACCAGGTACCTCTACTGAACCTTCTGAGGGCAGCGCTCCA | |
| | GGTAGCGAACCTGCAACCTCTGGCTCTGAAACCCCAGGTACCTCTGAAAGCGCT | |
| | ACTCCTGAATCTGGCCCAGGTACTTCTACTGAACCGTCCGAGGGCAGCGCACCA | |
| AE576 | GGTAGCCCGGCTGGCTCTCCTACCTCTACTGAGGAAGGTACTTCTGAAAGCGCT | 164 |
| | ACTCCTGAGTCTGGTCCAGGTACCTCTACTGAACCGTCCGAAGGTAGCGCTCCA | |
| | GGTAGCCCAGCAGGCTCTCCGACTTCCACTGAGGAAGGTACTTCTACTGAACCT | |
| | TCCGAAGGCAGCGCACCAGGTACCTCTACTGAACCTTCTGAGGGCAGCGCTCCA | |
| | GGTACTTCTGAAAGCGCTACCCCGGAATCTGGCCCAGGTAGCGAACCGGCTACT | |
| | TCTGGTTCTGAAACCCCAGGTAGCGAACCGGCTACCTCCGGTTCTGAAACTCCA | |
| | GGTAGCCCGGCAGGCTCTCCGACCTCTACTGAGGAAGGTACTTCTGAAAGCGCA | |
| | ACCCCGGAGTCCGGCCCAGGTACCTCTACCGAACCGTCTGAGGGCAGCGCACCA | |
| | GGTACTTCTACCGAACCGTCCGAGGGTAGCGCACCAGGTAGCCCAGCAGGTTCT | |
| | CCTACCTCCACCGAGGAAGGTACTTCTACCGAACCGTCCGAGGGTAGCGCACCA | |
| | GGTACCTCTACTGAACCTTCTGAGGGCAGCGCTCCAGGTACTTCTGAAAGCGCT | |
| | ACCCCGGAGTCCGGTCCAGGTACTTCTACTGAACCGTCCGAAGGTAGCGCACCA | |
| | GGTACTTCTGAAAGCGCAACCCCTGAATCCGGTCCAGGTAGCGAACCGGCTACT | |
| | TCTGGCTCTGAGACTCCAGGTACTTCTACCGAACCGTCCGAAGGTAGCGCACCA | |
| | GGTACTTCTACTGAACCGTCTGAAGGTAGCGCACCAGGTACTTCTGAAAGCGCA | |
| | ACCCCGGAATCCGGCCCAGGTACCTCTGAAAGCGCAACCCCGGAGTCCGGCCC | |
| | AGGTAGCCCTGCTGGCTCTCCAACCTCCACCGAAGAAGGTACCTCTGAAAGCGC | |
| | AACCCCTGAATCCGGCCCAGGTAGCGAACCGGCAACCTCCGGTTCTGAAACCCC | |
| | AGGTACCTCTGAAAGCGCTACTCCGGAGTCTGGCCCAGGTACCTCTACTGAACC | |
| | GTCTGAGGGTAGCGCTCCAGGTACTTCTACTGAACCGTCCGAAGGTAGCGCACC | |
| | AGGTACTTCTACCGAACCGTCCGAAGGCAGCGCTCCAGGTACCTCTACTGAACC | |
| | TTCCGAGGGCAGCGCTCCAGGTACCTCTACCGAACCTTCTGAAGGTAGCGCACC | |
| | AGGTACTTCTACCGAACCGTCCGAGGGTAGCGCACCAGGTAGCCCAGCAGGTTC | |
| | TCCTACCTCCACCGAGGAAGGTACTTCTACCGAACCGTCCGAGGGTAGCGCACC | |
| | AGGTACCTCTGAAAGCGCAACTCCTGAGTCTGGCCCAGGTAGCGAACCTGCTAC | |
| | CTCCGGCTCTGAGACTCCAGGTACCTCTGAAAGCGCAACCCCGGAATCTGGTCC | |
| | AGGTAGCGAACCTGCAACCTCTGGCTCTGAAACCCCAGGTACCTCTGAAAGCGC | |
| | TACTCCTGAATCTGGCCCAGGTACTTCTACTGAACCGTCCGAGGGCAGCGCACC | |
| | AGGTACTTCTGAAAGCGCTACTCCTGAGTCCGGCCCAGGTAGCCCGGCTGGCTC | |
| | TCCGACTTCCACCGAGGAAGGTAGCCCGGCTGGCTCTCCAACTTCTACTGAAGA | |
| | AGGTAGCCCGGCAGGCTCTCCGACCTCTACTGAGGAAGGTACTTCTGAAAGCGC | |
| | AACCCCGGAGTCCGGCCCAGGTACCTCTACCGAACCGTCTGAGGGCAGCGCACC | |
| | A | |
| AF576 | GGTTCTACTAGCTCTACCGCTGAATCTCCTGGCCCAGGTTCCACTAGCTCTACCG | 165 |
| | CAGAATCTCCGGGCCCAGGTTCTACTAGCGAATCCCCTTCTGGTACCGCTCCAG | |
| | GTTCTACTAGCTCTACCGCTGAATCTCCGGGTCCAGGTTCTACCAGCTCTACTGC | |
| | AGAATCTCCTGGCCCAGGTACTTCTACTCCGGAAAGCGGTTCCGCTTCTCCAGGT | |
| | TCTACCAGCGAATCTCCTTCTGGCACCGCTCCAGGTACCTCTCCTAGCGGCGAAT | |
| | CTTCTACCGCTCCAGGTTCTACTAGCGAATCTCCTTCTGGCACTGCACCAGGTTC | |
| | TACCAGCGAATCTCCTTCTGGCACCGCTCCAGGTACCTCTCCTAGCGGCGAATCT | |
| | TCTACCGCTCCAGGTTCTACTAGCGAATCTCCTTCTGGCACTGCACCAGGTTCTA | |
| | CCAGCGAATCTCCTTCTGGCACCGCTCCAGGTACCTCTCCTAGCGGCGAATCTTC | |
| | TACCGCTCCAGGTTCTACTAGCGAATCTCCTTCTGGCACTGCACCAGGTTCTACT | |
| | AGCGAATCTCCTTCTGGCACTGCACCAGGTTCTACCAGCGAATCTCCGTCTGGC | |
| | ACTGCACCAGGTACCTCTACCCCTGAAAGCGGTTCCGCTTCTCCAGGTTCTACTA | |
| | GCGAATCTCCTTCTGGTACCGCTCCAGGTACTTCTACCCCTGAAAGCGGCTCCGC | |
| | TTCTCCAGGTTCCACTAGCTCTACCGCTGAATCTCCGGGTCCAGGTTCTACTAGC | |
| | TCTACTGCAGAATCTCCTGGCCCAGGTACCTCTACTCCGGAAAGCGGCTCTGCA | |
| | TCTCCAGGTACTTCTACCCCTGAAAGCGGTTCTGCATCTCCAGGTTCTACTAGCG | |
| | AATCCCCGTCTGGTACCGCACCAGGTACTTCTACCCCGGAAAGCGGCTCTGCTT | |
| | CTCCAGGTACTTCTACCCCGGAAAGCGGCTCCGCATCTCCAGGTTCTACTAGCG | |
| | AATCTCCTTCTGGTACCGCTCCAGGTTCTACCAGCGAATCCCCGTCTGGTACTGC | |
| | TCCAGGTTCTACCAGCGAATCTCCTTCTGGTACTGCACCAGGTTCTACTAGCTCT | |
| | ACTGCAGAATCTCCTGGCCCAGGTACCTCTACTCCGGAAAGCGGCTCTGCATCT | |
| | CCAGGTACTTCTACCCCTGAAAGCGGTTCTGCATCTCCAGGTTCTACTAGCGAAT | |
| | CTCCTTCTGGCACTGCACCAGGTTCTACCAGCGAATCTCCGTCTGGCACTGCACC | |
| | AGGTACCTCTACCCCTGAAAGCGGTTCCGCTTCTCCAGGTTCTACTAGCGAATCT | |
| | CCTTCTGGCACTGCACCAGGTTCTACCAGCGAATCTCCGTCTGGCACTGCACCA | |
| | GGTACCTCTACCCCTGAAAGCGGTTCCGCTTCTCCAGGTACTTCTCCGAGCGGTG | |
| | AATCTTCTACCGCACCAGGTTCTACTAGCTCTACCGCTGAATCTCCGGGCCCAGG | |
| | TACTTCTCCGAGCGGTGAATCTTCTACTGCTCCAGGTTCCACTAGCTCTACTGCT | |
| | GAATCTCCTGGCCCAGGTACTTCTACTCCGGAAAGCGGTTCCGCTTCTCCAGGTT | |
| | CTACTAGCGAATCTCCGTCTGGCACCGCACCAGGTTCTACTAGCTCTACTGCAG | |
| | AATCTCCTGGCCCAGGTACCTCTACTCCGGAAAGCGGCTCTGCATCTCCAGGTA | |
| | CTTCTACCCCTGAAAGCGGTTCTGCATCTCCA | |
| AM875 | GGTACTTCTACTGAACCGTCTGAAGGCAGCGCACCAGGTAGCGAACCGGCTACT | 166 |
| | TCCGGTTCTGAAACCCCAGGTAGCCCAGCAGGTTCTCCAACTTCTACTGAAGAA | |
| | GGTTCTACCAGCTCTACCGCAGAATCTCCTGGTCCAGGTACCTCTACTCCGGAA | |
| | AGCGGCTCTGCATCTCCAGGTTCTACTAGCGAATCTCCTTCTGGCACTGCACCAG | |
| | GTTCTACTAGCGAATCCCCGTCTGGTACTGCTCCAGGTACTTCTACTCCTGAAAG | |
| | CGGTTCCGCTTCTCCAGGTACCTCTACTCCGGAAAGCGGTTCTGCATCTCCAGGT | |
| | AGCGAACCGGCAACCTCCGGCTCTGAAACCCCAGGTACCTCTGAAAGCGCTACT | |
| | CCTGAATCCGGCCCAGGTAGCCCGGCAGGTTCTCCGACTTCCACTGAGGAAGGT | |
| | ACCTCTACTGAACCTTCTGAGGGCAGCGCTCCAGGTACTTCTGAAAGCGCTACC | |
| | CCGGAGTCCGGTCCAGGTACTTCTACTGAACCGTCCGAAGGTAGCGCACCAGGT | |
| | ACTTCTACCGAACCGTCCGAGGGTAGCGCACCAGGTAGCCCAGCAGGTTCTCCT | |
| | ACCTCCACCGAGGAAGGTACTTCTACCGAACCGTCCGAGGGTAGCGCACCAGGT | |
| | ACTTCTACCGAACCTTCCGAGGGCAGCGCACCAGGTACTTCTGAAAGCGCTACC | |
| | CCTGAGTCCGGCCCAGGTACTTCTGAAAGCGCTACTCCTGAATCCGGTCCAGGT | |
| | ACCTCTACTGAACCTTCCGAAGGCAGCGCTCCAGGTACCTCTACCGAACCGTCC | |
| | GAGGGCAGCGCACCAGGTACTTCTGAAAGCGCAACCCCTGAATCCGGTCCAGGT | |
| | ACTTCTACTGAACCTTCCGAAGGTAGCGCTCCAGGTAGCGAACCTGCTACTTCT | |
| | GGTTCTGAAACCCCAGGTAGCCCGGCTGGCTCTCCGACCTCCACCGAGGAAGGT | |
| | AGCTCTACCCCGTCTGGTGCTACTGGTTCTCCAGGTACTCCGGGCAGCGGTACTG | |
| | CTTCTTCCTCTCCAGGTAGCTCTACCCCTTCTGGTGCTACTGGCTCTCCAGGTAC | |
| | CTCTACCGAACCGTCCGAGGGTAGCGCACCAGGTACCTCTACTGAACCGTCTGA | |
| | GGGTAGCGCTCCAGGTAGCGAACCGGCAACCTCCGGTTCTGAAACTCCAGGTAG | |
| | CCCTGCTGGCTCTCCGACTTCTACTGAGGAAGGTAGCCCGGCTGGTTCTCCGACT | |
| | TCTACTGAGGAAGGTACTTCTACCGAACCTTCCGAAGGTAGCGCTCCAGGTGCA | |
| | AGCGCAAGCGGCGCGCCAAGCACGGGAGGTACTTCTGAAAGCGCTACTCCTGA | |
| | GTCCGGCCCAGGTAGCCCGGCTGGCTCTCCGACTTCCACCGAGGAAGGTAGCCC | |
| | GGCTGGCTCTCCAACTTCTACTGAAGAAGGTTCTACCAGCTCTACCGCTGAATCT | |
| | CCTGGCCCAGGTTCTACTAGCGAATCTCCGTCTGGCACCGCACCAGGTACTTCCC | |
| | CTAGCGGTGAATCTTCTACTGCACCAGGTACCCCTGGCAGCGGTACCGCTTCTTC | |
| | CTCTCCAGGTAGCTCTACCCCGTCTGGTGCTACTGGCTCTCCAGGTTCTAGCCCG | |
| | TCTGCATCTACCGGTACCGGCCCAGGTAGCGAACCGGCAACCTCCGGCTCTGAA | |
| | ACTCCAGGTACTTCTGAAAGCGCTACTCCGGAATCCGGCCCAGGTAGCGAACCG | |
| | GCTACTTCCGGCTCTGAAACCCCAGGTTCCACCAGCTCTACTGCAGAATCTCCG | |
| | GGCCCAGGTTCTACTAGCTCTACTGCAGAATCTCCGGGTCCAGGTACTTCTCCTA | |
| | GCGGCGAATCTTCTACCGCTCCAGGTAGCGAACCGGCAACCTCTGGCTCTGAAA | |
| | CTCCAGGTAGCGAACCTGCAACCTCCGGCTCTGAAACCCCAGGTACTTCTACTG | |
| | AACCTTCTGAGGGCAGCGCACCAGGTTCTACCAGCTCTACCGCAGAATCTCCTG | |
| | GTCCAGGTACCTCTACTCCGGAAAGCGGCTCTGCATCTCCAGGTTCTACTAGCG | |
| | AATCTCCTTCTGGCACTGCACCAGGTACTTCTACCGAACCGTCCGAAGGCAGCG | |
| | CTCCAGGTACCTCTACTGAACCTTCCGAGGGCAGCGCTCCAGGTACCTCTACCG | |
| | AACCTTCTGAAGGTAGCGCACCAGGTAGCTCTACTCCGTCTGGTGCAACCGGCT | |
| | CCCCAGGTTCTAGCCCGTCTGCTTCCACTGGTACTGGCCCAGGTGCTTCCCCGGG | |
| | CACCAGCTCTACTGGTTCTCCAGGTAGCGAACCTGCTACCTCCGGTTCTGAAACC | |
| | CCAGGTACCTCTGAAAGCGCAACTCCGGAGTCTGGTCCAGGTAGCCCTGCAGGT | |
| | TCTCCTACCTCCACTGAGGAAGGTAGCTCTACTCCGTCTGGTGCAACCGGCTCCC | |
| | CAGGTTCTAGCCCGTCTGCTTCCACTGGTACTGGCCCAGGTGCTTCCCCGGGCAC | |
| | CAGCTCTACTGGTTCTCCAGGTACCTCTGAAAGCGCTACTCCGGAGTCTGGCCC | |
| | AGGTACCTCTACTGAACCGTCTGAGGGTAGCGCTCCAGGTACTTCTACTGAACC | |
| | GTCCGAAGGTAGCGCACCA | |
| AE864 | GGTAGCCCGGCTGGCTCTCCTACCTCTACTGAGGAAGGTACTTCTGAAAGCGCT | 167 |
| | ACTCCTGAGTCTGGTCCAGGTACCTCTACTGAACCGTCCGAAGGTAGCGCTCCA | |
| | GGTAGCCCAGCAGGCTCTCCGACTTCCACTGAGGAAGGTACTTCTACTGAACCT | |
| | TCCGAAGGCAGCGCACCAGGTACCTCTACTGAACCTTCTGAGGGCAGCGCTCCA | |
| | GGTACTTCTGAAAGCGCTACCCCGGAATCTGGCCCAGGTAGCGAACCGGCTACT | |
| | TCTGGTTCTGAAACCCCAGGTAGCGAACCGGCTACCTCCGGTTCTGAAACTCCA | |
| | GGTAGCCCGGCAGGCTCTCCGACCTCTACTGAGGAAGGTACTTCTGAAAGCGCA | |
| | ACCCCGGAGTCCGGCCCAGGTACCTCTACCGAACCGTCTGAGGGCAGCGCACCA | |
| | GGTACTTCTACCGAACCGTCCGAGGGTAGCGCACCAGGTAGCCCAGCAGGTTCT | |
| | CCTACCTCCACCGAGGAAGGTACTTCTACCGAACCGTCCGAGGGTAGCGCACCA | |
| | GGTACCTCTACTGAACCTTCTGAGGGCAGCGCTCCAGGTACTTCTGAAAGCGCT | |
| | ACCCCGGAGTCCGGTCCAGGTACTTCTACTGAACCGTCCGAAGGTAGCGCACCA | |
| | GGTACTTCTGAAAGCGCAACCCCTGAATCCGGTCCAGGTAGCGAACCGGCTACT | |
| | TCTGGCTCTGAGACTCCAGGTACTTCTACCGAACCGTCCGAAGGTAGCGCACCA | |
| | GGTACTTCTACTGAACCGTCTGAAGGTAGCGCACCAGGTACTTCTGAAAGCGCA | |
| | ACCCCGGAATCCGGCCCAGGTACCTCTGAAAGCGCAACCCCGGAGTCCGGCCC | |
| | AGGTAGCCCTGCTGGCTCTCCAACCTCCACCGAAGAAGGTACCTCTGAAAGCGC | |
| | AACCCCTGAATCCGGCCCAGGTAGCGAACCGGCAACCTCCGGTTCTGAAACCCC | |
| | AGGTACCTCTGAAAGCGCTACTCCGGAGTCTGGCCCAGGTACCTCTACTGAACC | |
| | GTCTGAGGGTAGCGCTCCAGGTACTTCTACTGAACCGTCCGAAGGTAGCGCACC | |
| | AGGTACTTCTACCGAACCGTCCGAAGGCAGCGCTCCAGGTACCTCTACTGAACC | |
| | TTCCGAGGGCAGCGCTCCAGGTACCTCTACCGAACCTTCTGAAGGTAGCGCACC | |
| | AGGTACTTCTACCGAACCGTCCGAGGGTAGCGCACCAGGTAGCCCAGCAGGTTC | |
| | TCCTACCTCCACCGAGGAAGGTACTTCTACCGAACCGTCCGAGGGTAGCGCACC | |
| | AGGTACCTCTGAAAGCGCAACTCCTGAGTCTGGCCCAGGTAGCGAACCTGCTAC | |
| | CTCCGGCTCTGAGACTCCAGGTACCTCTGAAAGCGCAACCCCGGAATCTGGTCC | |
| | AGGTAGCGAACCTGCAACCTCTGGCTCTGAAACCCCAGGTACCTCTGAAAGCGC | |
| | TACTCCTGAATCTGGCCCAGGTACTTCTACTGAACCGTCCGAGGGCAGCGCACC | |
| | AGGTACTTCTGAAAGCGCTACTCCTGAGTCCGGCCCAGGTAGCCCGGCTGGCTC | |
| | TCCGACTTCCACCGAGGAAGGTAGCCCGGCTGGCTCTCCAACTTCTACTGAAGA | |
| | AGGTAGCCCGGCAGGCTCTCCGACCTCTACTGAGGAAGGTACTTCTGAAAGCGC | |
| | AACCCCGGAGTCCGGCCCAGGTACCTCTACCGAACCGTCTGAGGGCAGCGCACC | |
| | AGGTACCTCTGAAAGCGCAACTCCTGAGTCTGGCCCAGGTAGCGAACCTGCTAC | |
| | CTCCGGCTCTGAGACTCCAGGTACCTCTGAAAGCGCAACCCCGGAATCTGGTCC | |
| | AGGTAGCGAACCTGCAACCTCTGGCTCTGAAACCCCAGGTACCTCTGAAAGCGC | |
| | TACTCCTGAATCTGGCCCAGGTACTTCTACTGAACCGTCCGAGGGCAGCGCACC | |
| | AGGTAGCCCTGCTGGCTCTCCAACCTCCACCGAAGAAGGTACCTCTGAAAGCGC | |
| | AACCCCTGAATCCGGCCCAGGTAGCGAACCGGCAACCTCCGGTTCTGAAACCCC | |
| | AGGTACTTCTGAAAGCGCTACTCCTGAGTCCGGCCCAGGTAGCCCGGCTGGCTC | |
| | TCCGACTTCCACCGAGGAAGGTAGCCCGGCTGGCTCTCCAACTTCTACTGAAGA | |
| | AGGTACTTCTACCGAACCTTCCGAGGGCAGCGCACCAGGTACTTCTGAAAGCGC | |
| | TACCCCTGAGTCCGGCCCAGGTACTTCTGAAAGCGCTACTCCTGAATCCGGTCC | |
| | AGGTACTTCTGAAAGCGCTACCCCGGAATCTGGCCCAGGTAGCGAACCGGCTAC | |
| | TTCTGGTTCTGAAACCCCAGGTAGCGAACCGGCTACCTCCGGTTCTGAAACTCC | |
| | AGGTAGCCCAGCAGGCTCTCCGACTTCCACTGAGGAAGGTACTTCTACTGAACC | |
| | TTCCGAAGGCAGCGCACCAGGTACCTCTACTGAACCTTCTGAGGGCAGCGCTCC | |
| | AGGTAGCGAACCTGCAACCTCTGGCTCTGAAACCCCAGGTACCTCTGAAAGCGC | |
| | TACTCCTGAATCTGGCCCAGGTACTTCTACTGAACCGTCCGAGGGCAGCGCACC | |
| | A | |
| AF864 | GGTTCTACCAGCGAATCTCCTTCTGGCACCGCTCCAGGTACCTCTCCTAGCGGCG | 168 |
| | AATCTTCTACCGCTCCAGGTTCTACTAGCGAATCTCCTTCTGGCACTGCACCAGG | |
| | TTCTACTAGCGAATCCCCGTCTGGTACTGCTCCAGGTACTTCTACTCCTGAAAGC | |
| | GGTTCCGCTTCTCCAGGTACCTCTACTCCGGAAAGCGGTTCTGCATCTCCAGGTT | |
| | CTACCAGCGAATCTCCTTCTGGCACCGCTCCAGGTTCTACTAGCGAATCCCCGTC | |
| | TGGTACCGCACCAGGTACTTCTCCTAGCGGCGAATCTTCTACCGCACCAGGTTCT | |
| | ACTAGCGAATCTCCGTCTGGCACTGCTCCAGGTACTTCTCCTAGCGGTGAATCTT | |
| | CTACCGCTCCAGGTACTTCCCCTAGCGGCGAATCTTCTACCGCTCCAGGTTCTAC | |
| | TAGCTCTACTGCAGAATCTCCGGGCCCAGGTACCTCTCCTAGCGGTGAATCTTCT | |
| | ACCGCTCCAGGTACTTCTCCGAGCGGTGAATCTTCTACCGCTCCAGGTTCTACTA | |
| | GCTCTACTGCAGAATCTCCTGGCCCAGGTACCTCTACTCCGGAAAGCGGCTCTG | |
| | CATCTCCAGGTACTTCTACCCCTGAAAGCGGTTCTGCATCTCCAGGTTCTACTAG | |
| | CGAATCTCCTTCTGGCACTGCACCAGGTTCTACCAGCGAATCTCCGTCTGGCACT | |
| | GCACCAGGTACCTCTACCCCTGAAAGCGGTTCCGCTTCTCCAGGTTCTACCAGCT | |
| | CTACCGCAGAATCTCCTGGTCCAGGTACCTCTACTCCGGAAAGCGGCTCTGCAT | |
| | CTCCAGGTTCTACTAGCGAATCTCCTTCTGGCACTGCACCAGGTACTTCTCCGAG | |
| | CGGTGAATCTTCTACCGCACCAGGTTCTACTAGCTCTACCGCTGAATCTCCGGGC | |
| | CCAGGTACTTCTCCGAGCGGTGAATCTTCTACTGCTCCAGGTACCTCTACTCCTG | |
| | AAAGCGGTTCTGCATCTCCAGGTTCCACTAGCTCTACCGCAGAATCTCCGGGCC | |
| | CAGGTTCTACTAGCTCTACTGCTGAATCTCCTGGCCCAGGTTCTACTAGCTCTAC | |
| | TGCTGAATCTCCGGGTCCAGGTTCTACCAGCTCTACTGCTGAATCTCCTGGTCCA | |
| | GGTACCTCCCCGAGCGGTGAATCTTCTACTGCACCAGGTTCTACTAGCGAATCTC | |
| | CTTCTGGCACTGCACCAGGTTCTACCAGCGAATCTCCGTCTGGCACTGCACCAG | |
| | GTACCTCTACCCCTGAAAGCGGTCCXXXXXXXXXXXXTGCAAGCGCAAGCGGC | |
| | GCGCCAAGCACGGGAXXXXXXXXTAGCGAATCTCCTTCTGGTACCGCTCCAGGT | |
| | TCTACCAGCGAATCCCCGTCTGGTACTGCTCCAGGTTCTACCAGCGAATCTCCTT | |
| | CTGGTACTGCACCAGGTTCTACTAGCGAATCTCCTTCTGGTACCGCTCCAGGTTC | |
| | TACCAGCGAATCCCCGTCTGGTACTGCTCCAGGTTCTACCAGCGAATCTCCTTCT | |
| | GGTACTGCACCAGGTACTTCTACTCCGGAAAGCGGTTCCGCATCTCCAGGTACT | |
| | TCTCCTAGCGGTGAATCTTCTACTGCTCCAGGTACCTCTCCTAGCGGCGAATCTT | |
| | CTACTGCTCCAGGTTCTACCAGCTCTACTGCTGAATCTCCGGGTCCAGGTACTTC | |
| | CCCGAGCGGTGAATCTTCTACTGCACCAGGTACTTCTACTCCGGAAAGCGGTTC | |
| | CGCTTCTCCAGGTTCTACCAGCGAATCTCCTTCTGGCACCGCTCCAGGTTCTACT | |
| | AGCGAATCCCCGTCTGGTACCGCACCAGGTACTTCTCCTAGCGGCGAATCTTCT | |
| | ACCGCACCAGGTTCTACTAGCGAATCCCCGTCTGGTACCGCACCAGGTACTTCT | |
| | ACCCCGGAAAGCGGCTCTGCTTCTCCAGGTACTTCTACCCCGGAAAGCGGCTCC | |
| | GCATCTCCAGGTTCTACTAGCGAATCTCCTTCTGGTACCGCTCCAGGTACTTCTA | |
| | CCCCTGAAAGCGGCTCCGCTTCTCCAGGTTCCACTAGCTCTACCGCTGAATCTCC | |
| | GGGTCCAGGTTCTACCAGCGAATCTCCTTCTGGCACCGCTCCAGGTTCTACTAGC | |
| | GAATCCCCGTCTGGTACCGCACCAGGTACTTCTCCTAGCGGCGAATCTTCTACCG | |
| | CACCAGGTTCTACCAGCTCTACTGCTGAATCTCCGGGTCCAGGTACTTCCCCGAG | |
| | CGGTGAATCTTCTACTGCACCAGGTACTTCTACTCCGGAAAGCGGTTCCGCTTCT | |
| | CCAGGTACCTCCCCTAGCGGCGAATCTTCTACTGCTCCAGGTACCTCTCCTAGCG | |
| | GCGAATCTTCTACCGCTCCAGGTACCTCCCCTAGCGGTGAATCTTCTACCGCACC | |
| | AGGTTCTACTAGCTCTACTGCTGAATCTCCGGGTCCAGGTTCTACCAGCTCTACT | |
| | GCTGAATCTCCTGGTCCAGGTACCTCCCCGAGCGGTGAATCTTCTACTGCACCA | |
| | GGTTCTAGCCCTTCTGCTTCCACCGGTACCGGCCCAGGTAGCTCTACTCCGTCTG | |
| | GTGCAACTGGCTCTCCAGGTAGCTCTACTCCGTCTGGTGCAACCGGCTCCCCA | |
| | XXXX was inserted in two areas where no sequence | |
| | information is available. | |
| AG864 | GGTGCTTCCCCGGGCACCAGCTCTACTGGTTCTCCAGGTTCTAGCCCGTCTGCTT | 169 |
| | CTACTGGTACTGGTCCAGGTTCTAGCCCTTCTGCTTCCACTGGTACTGGTCCAGG | |
| | TACCCCGGGTAGCGGTACCGCTTCTTCTTCTCCAGGTAGCTCTACTCCGTCTGGT | |
| | GCTACCGGCTCTCCAGGTTCTAACCCTTCTGCATCCACCGGTACCGGCCCAGGTG | |
| | CTTCTCCGGGCACCAGCTCTACTGGTTCTCCAGGTACCCCGGGCAGCGGTACCG | |
| | CATCTTCTTCTCCAGGTAGCTCTACTCCTTCTGGTGCAACTGGTTCTCCAGGTAC | |
| | TCCTGGCAGCGGTACCGCTTCTTCTTCTCCAGGTGCTTCTCCTGGTACTAGCTCT | |
| | ACTGGTTCTCCAGGTGCTTCTCCGGGCACTAGCTCTACTGGTTCTCCAGGTACCC | |
| | CGGGTAGCGGTACTGCTTCTTCCTCTCCAGGTAGCTCTACCCCTTCTGGTGCAAC | |
| | CGGCTCTCCAGGTGCTTCTCCGGGCACCAGCTCTACCGGTTCTCCAGGTACCCCG | |
| | GGTAGCGGTACCGCTTCTTCTTCTCCAGGTAGCTCTACTCCGTCTGGTGCTACCG | |
| | GCTCTCCAGGTTCTAACCCTTCTGCATCCACCGGTACCGGCCCAGGTTCTAGCCC | |
| | TTCTGCTTCCACCGGTACTGGCCCAGGTAGCTCTACCCCTTCTGGTGCTACCGGC | |
| | TCCCCAGGTAGCTCTACTCCTTCTGGTGCAACTGGCTCTCCAGGTGCATCTCCGG | |
| | GCACTAGCTCTACTGGTTCTCCAGGTGCATCCCCTGGCACTAGCTCTACTGGTTC | |
| | TCCAGGTGCTTCTCCTGGTACCAGCTCTACTGGTTCTCCAGGTACTCCTGGCAGC | |
| | GGTACCGCTTCTTCTTCTCCAGGTGCTTCTCCTGGTACTAGCTCTACTGGTTCTCC | |
| | AGGTGCTTCTCCGGGCACTAGCTCTACTGGTTCTCCAGGTGCTTCCCCGGGCACT | |
| | AGCTCTACCGGTTCTCCAGGTTCTAGCCCTTCTGCATCTACTGGTACTGGCCCAG | |
| | GTACTCCGGGCAGCGGTACTGCTTCTTCCTCTCCAGGTGCATCTCCGGGCACTAG | |
| | CTCTACTGGTTCTCCAGGTGCATCCCCTGGCACTAGCTCTACTGGTTCTCCAGGT | |
| | GCTTCTCCTGGTACCAGCTCTACTGGTTCTCCAGGTAGCTCTACTCCGTCTGGTG | |
| | CAACCGGTTCCCCAGGTAGCTCTACTCCTTCTGGTGCTACTGGCTCCCCAGGTGC | |
| | ATCCCCTGGCACCAGCTCTACCGGTTCTCCAGGTACCCCGGGCAGCGGTACCGC | |
| | ATCTTCCTCTCCAGGTAGCTCTACCCCGTCTGGTGCTACCGGTTCCCCAGGTAGC | |
| | TCTACCCCGTCTGGTGCAACCGGCTCCCCAGGTAGCTCTACTCCGTCTGGTGCAA | |
| | CCGGCTCCCCAGGTTCTAGCCCGTCTGCTTCCACTGGTACTGGCCCAGGTGCTTC | |
| | CCCGGGCACCAGCTCTACTGGTTCTCCAGGTGCATCCCCGGGTACCAGCTCTAC | |
| | CGGTTCTCCAGGTACTCCTGGCAGCGGTACTGCATCTTCCTCTCCAGGTGCTTCT | |
| | CCGGGCACCAGCTCTACTGGTTCTCCAGGTGCATCTCCGGGCACTAGCTCTACTG | |
| | GTTCTCCAGGTGCATCCCCTGGCACTAGCTCTACTGGTTCTCCAGGTGCTTCTCC | |
| | TGGTACCAGCTCTACTGGTTCTCCAGGTACCCCTGGTAGCGGTACTGCTTCTTCC | |
| | TCTCCAGGTAGCTCTACTCCGTCTGGTGCTACCGGTTCTCCAGGTACCCCGGGTA | |
| | GCGGTACCGCATCTTCTTCTCCAGGTAGCTCTACCCCGTCTGGTGCTACTGGTTC | |
| | TCCAGGTACTCCGGGCAGCGGTACTGCTTCTTCCTCTCCAGGTAGCTCTACCCCT | |
| | TCTGGTGCTACTGGCTCTCCAGGTAGCTCTACCCCGTCTGGTGCTACTGGCTCCC | |
| | CAGGTTCTAGCCCTTCTGCATCCACCGGTACCGGTCCAGGTTCTAGCCCGTCTGC | |
| | ATCTACTGGTACTGGTCCAGGTGCATCCCCGGGCACTAGCTCTACCGGTTCTCCA | |
| | GGTACTCCTGGTAGCGGTACTGCTTCTTCTTCTCCAGGTAGCTCTACTCCTTCTG | |
| | GTGCTACTGGTTCTCCAGGTTCTAGCCCTTCTGCATCCACCGGTACCGGCCCAGG | |
| | TTCTAGCCCGTCTGCTTCTACCGGTACTGGTCCAGGTGCTTCTCCGGGTACTAGC | |
| | TCTACTGGTTCTCCAGGTGCATCTCCTGGTACTAGCTCTACTGGTTCTCCAGGTA | |
| | GCTCTACTCCGTCTGGTGCAACCGGCTCTCCAGGTTCTAGCCCTTCTGCATCTAC | |
| | CGGTACTGGTCCAGGTGCATCCCCTGGTACCAGCTCTACCGGTTCTCCAGGTTCT | |
| | AGCCCTTCTGCTTCTACCGGTACCGGTCCAGGTACCCCTGGCAGCGGTACCGCA | |
| | TCTTCCTCTCCAGGTAGCTCTACTCCGTCTGGTGCAACCGGTTCCCCAGGTAGCT | |
| | CTACTCCTTCTGGTGCTACTGGCTCCCCAGGTGCATCCCCTGGCACCAGCTCTAC | |
| | CGGTTCTCCA | |
| AM923 | ATGGCTGAACCTGCTGGCTCTCCAACCTCCACTGAGGAAGGTGCATCCCCGGGC | 170 |
| | ACCAGCTCTACCGGTTCTCCAGGTAGCTCTACCCCGTCTGGTGCTACCGGCTCTC | |
| | CAGGTAGCTCTACCCCGTCTGGTGCTACTGGCTCTCCAGGTACTTCTACTGAACC | |
| | GTCTGAAGGCAGCGCACCAGGTAGCGAACCGGCTACTTCCGGTTCTGAAACCCC | |
| | AGGTAGCCCAGCAGGTTCTCCAACTTCTACTGAAGAAGGTTCTACCAGCTCTAC | |
| | CGCAGAATCTCCTGGTCCAGGTACCTCTACTCCGGAAAGCGGCTCTGCATCTCC | |
| | AGGTTCTACTAGCGAATCTCCTTCTGGCACTGCACCAGGTTCTACTAGCGAATCC | |
| | CCGTCTGGTACTGCTCCAGGTACTTCTACTCCTGAAAGCGGTTCCGCTTCTCCAG | |
| | GTACCTCTACTCCGGAAAGCGGTTCTGCATCTCCAGGTAGCGAACCGGCAACCT | |
| | CCGGCTCTGAAACCCCAGGTACCTCTGAAAGCGCTACTCCTGAATCCGGCCCAG | |
| | GTAGCCCGGCAGGTTCTCCGACTTCCACTGAGGAAGGTACCTCTACTGAACCTT | |
| | CTGAGGGCAGCGCTCCAGGTACTTCTGAAAGCGCTACCCCGGAGTCCGGTCCAG | |
| | GTACTTCTACTGAACCGTCCGAAGGTAGCGCACCAGGTACTTCTACCGAACCGT | |
| | CCGAGGGTAGCGCACCAGGTAGCCCAGCAGGTTCTCCTACCTCCACCGAGGAAG | |
| | GTACTTCTACCGAACCGTCCGAGGGTAGCGCACCAGGTACTTCTACCGAACCTT | |
| | CCGAGGGCAGCGCACCAGGTACTTCTGAAAGCGCTACCCCTGAGTCCGGCCCAG | |
| | GTACTTCTGAAAGCGCTACTCCTGAATCCGGTCCAGGTACCTCTACTGAACCTTC | |
| | CGAAGGCAGCGCTCCAGGTACCTCTACCGAACCGTCCGAGGGCAGCGCACCAG | |
| | GTACTTCTGAAAGCGCAACCCCTGAATCCGGTCCAGGTACTTCTACTGAACCTTC | |
| | CGAAGGTAGCGCTCCAGGTAGCGAACCTGCTACTTCTGGTTCTGAAACCCCAGG | |
| | TAGCCCGGCTGGCTCTCCGACCTCCACCGAGGAAGGTAGCTCTACCCCGTCTGG | |
| | TGCTACTGGTTCTCCAGGTACTCCGGGCAGCGGTACTGCTTCTTCCTCTCCAGGT | |
| | AGCTCTACCCCTTCTGGTGCTACTGGCTCTCCAGGTACCTCTACCGAACCGTCCG | |
| | AGGGTAGCGCACCAGGTACCTCTACTGAACCGTCTGAGGGTAGCGCTCCAGGTA | |
| | GCGAACCGGCAACCTCCGGTTCTGAAACTCCAGGTAGCCCTGCTGGCTCTCCGA | |
| | CTTCTACTGAGGAAGGTAGCCCGGCTGGTTCTCCGACTTCTACTGAGGAAGGTA | |
| | CTTCTACCGAACCTTCCGAAGGTAGCGCTCCAGGTGCAAGCGCAAGCGGCGCGC | |
| | CAAGCACGGGAGGTACTTCTGAAAGCGCTACTCCTGAGTCCGGCCCAGGTAGCC | |
| | CGGCTGGCTCTCCGACTTCCACCGAGGAAGGTAGCCCGGCTGGCTCTCCAACTT | |
| | CTACTGAAGAAGGTTCTACCAGCTCTACCGCTGAATCTCCTGGCCCAGGTTCTAC | |
| | TAGCGAATCTCCGTCTGGCACCGCACCAGGTACTTCCCCTAGCGGTGAATCTTCT | |
| | ACTGCACCAGGTACCCCTGGCAGCGGTACCGCTTCTTCCTCTCCAGGTAGCTCTA | |
| | CCCCGTCTGGTGCTACTGGCTCTCCAGGTTCTAGCCCGTCTGCATCTACCGGTAC | |
| | CGGCCCAGGTAGCGAACCGGCAACCTCCGGCTCTGAAACTCCAGGTACTTCTGA | |
| | AAGCGCTACTCCGGAATCCGGCCCAGGTAGCGAACCGGCTACTTCCGGCTCTGA | |
| | AACCCCAGGTTCCACCAGCTCTACTGCAGAATCTCCGGGCCCAGGTTCTACTAG | |
| | CTCTACTGCAGAATCTCCGGGTCCAGGTACTTCTCCTAGCGGCGAATCTTCTACC | |
| | GCTCCAGGTAGCGAACCGGCAACCTCTGGCTCTGAAACTCCAGGTAGCGAACCT | |
| | GCAACCTCCGGCTCTGAAACCCCAGGTACTTCTACTGAACCTTCTGAGGGCAGC | |
| | GCACCAGGTTCTACCAGCTCTACCGCAGAATCTCCTGGTCCAGGTACCTCTACTC | |
| | CGGAAAGCGGCTCTGCATCTCCAGGTTCTACTAGCGAATCTCCTTCTGGCACTGC | |
| | ACCAGGTACTTCTACCGAACCGTCCGAAGGCAGCGCTCCAGGTACCTCTACTGA | |
| | ACCTTCCGAGGGCAGCGCTCCAGGTACCTCTACCGAACCTTCTGAAGGTAGCGC | |
| | ACCAGGTAGCTCTACTCCGTCTGGTGCAACCGGCTCCCCAGGTTCTAGCCCGTCT | |
| | GCTTCCACTGGTACTGGCCCAGGTGCTTCCCCGGGCACCAGCTCTACTGGTTCTC | |
| | CAGGTAGCGAACCTGCTACCTCCGGTTCTGAAACCCCAGGTACCTCTGAAAGCG | |
| | CAACTCCGGAGTCTGGTCCAGGTAGCCCTGCAGGTTCTCCTACCTCCACTGAGG | |
| | AAGGTAGCTCTACTCCGTCTGGTGCAACCGGCTCCCCAGGTTCTAGCCCGTCTGC | |
| | TTCCACTGGTACTGGCCCAGGTGCTTCCCCGGGCACCAGCTCTACTGGTTCTCCA | |
| | GGTACCTCTGAAAGCGCTACTCCGGAGTCTGGCCCAGGTACCTCTACTGAACCG | |
| | TCTGAGGGTAGCGCTCCAGGTACTTCTACTGAACCGTCCGAAGGTAGCGCACCA | |
| AE912 | ATGGCTGAACCTGCTGGCTCTCCAACCTCCACTGAGGAAGGTACCCCGGGTAGC | 171 |
| | GGTACTGCTTCTTCCTCTCCAGGTAGCTCTACCCCTTCTGGTGCAACCGGCTCTC | |
| | CAGGTGCTTCTCCGGGCACCAGCTCTACCGGTTCTCCAGGTAGCCCGGCTGGCT | |
| | CTCCTACCTCTACTGAGGAAGGTACTTCTGAAAGCGCTACTCCTGAGTCTGGTCC | |
| | AGGTACCTCTACTGAACCGTCCGAAGGTAGCGCTCCAGGTAGCCCAGCAGGCTC | |
| | TCCGACTTCCACTGAGGAAGGTACTTCTACTGAACCTTCCGAAGGCAGCGCACC | |
| | AGGTACCTCTACTGAACCTTCTGAGGGCAGCGCTCCAGGTACTTCTGAAAGCGC | |
| | TACCCCGGAATCTGGCCCAGGTAGCGAACCGGCTACTTCTGGTTCTGAAACCCC | |
| | AGGTAGCGAACCGGCTACCTCCGGTTCTGAAACTCCAGGTAGCCCGGCAGGCTC | |
| | TCCGACCTCTACTGAGGAAGGTACTTCTGAAAGCGCAACCCCGGAGTCCGGCCC | |
| | AGGTACCTCTACCGAACCGTCTGAGGGCAGCGCACCAGGTACTTCTACCGAACC | |
| | GTCCGAGGGTAGCGCACCAGGTAGCCCAGCAGGTTCTCCTACCTCCACCGAGGA | |
| | AGGTACTTCTACCGAACCGTCCGAGGGTAGCGCACCAGGTACCTCTACTGAACC | |
| | TTCTGAGGGCAGCGCTCCAGGTACTTCTGAAAGCGCTACCCCGGAGTCCGGTCC | |
| | AGGTACTTCTACTGAACCGTCCGAAGGTAGCGCACCAGGTACTTCTGAAAGCGC | |
| | AACCCCTGAATCCGGTCCAGGTAGCGAACCGGCTACTTCTGGCTCTGAGACTCC | |
| | AGGTACTTCTACCGAACCGTCCGAAGGTAGCGCACCAGGTACTTCTACTGAACC | |
| | GTCTGAAGGTAGCGCACCAGGTACTTCTGAAAGCGCAACCCCGGAATCCGGCCC | |
| | AGGTACCTCTGAAAGCGCAACCCCGGAGTCCGGCCCAGGTAGCCCTGCTGGCTC | |
| | TCCAACCTCCACCGAAGAAGGTACCTCTGAAAGCGCAACCCCTGAATCCGGCCC | |
| | AGGTAGCGAACCGGCAACCTCCGGTTCTGAAACCCCAGGTACCTCTGAAAGCGC | |
| | TACTCCGGAGTCTGGCCCAGGTACCTCTACTGAACCGTCTGAGGGTAGCGCTCC | |
| | AGGTACTTCTACTGAACCGTCCGAAGGTAGCGCACCAGGTACTTCTACCGAACC | |
| | GTCCGAAGGCAGCGCTCCAGGTACCTCTACTGAACCTTCCGAGGGCAGCGCTCC | |
| | AGGTACCTCTACCGAACCTTCTGAAGGTAGCGCACCAGGTACTTCTACCGAACC | |
| | GTCCGAGGGTAGCGCACCAGGTAGCCCAGCAGGTTCTCCTACCTCCACCGAGGA | |
| | AGGTACTTCTACCGAACCGTCCGAGGGTAGCGCACCAGGTACCTCTGAAAGCGC | |
| | AACTCCTGAGTCTGGCCCAGGTAGCGAACCTGCTACCTCCGGCTCTGAGACTCC | |
| | AGGTACCTCTGAAAGCGCAACCCCGGAATCTGGTCCAGGTAGCGAACCTGCAAC | |
| | CTCTGGCTCTGAAACCCCAGGTACCTCTGAAAGCGCTACTCCTGAATCTGGCCC | |
| | AGGTACTTCTACTGAACCGTCCGAGGGCAGCGCACCAGGTACTTCTGAAAGCGC | |
| | TACTCCTGAGTCCGGCCCAGGTAGCCCGGCTGGCTCTCCGACTTCCACCGAGGA | |
| | AGGTAGCCCGGCTGGCTCTCCAACTTCTACTGAAGAAGGTAGCCCGGCAGGCTC | |
| | TCCGACCTCTACTGAGGAAGGTACTTCTGAAAGCGCAACCCCGGAGTCCGGCCC | |
| | AGGTACCTCTACCGAACCGTCTGAGGGCAGCGCACCAGGTACCTCTGAAAGCGC | |
| | AACTCCTGAGTCTGGCCCAGGTAGCGAACCTGCTACCTCCGGCTCTGAGACTCC | |
| | AGGTACCTCTGAAAGCGCAACCCCGGAATCTGGTCCAGGTAGCGAACCTGCAAC | |
| | CTCTGGCTCTGAAACCCCAGGTACCTCTGAAAGCGCTACTCCTGAATCTGGCCC | |
| | AGGTACTTCTACTGAACCGTCCGAGGGCAGCGCACCAGGTAGCCCTGCTGGCTC | |
| | TCCAACCTCCACCGAAGAAGGTACCTCTGAAAGCGCAACCCCTGAATCCGGCCC | |
| | AGGTAGCGAACCGGCAACCTCCGGTTCTGAAACCCCAGGTACTTCTGAAAGCGC | |
| | TACTCCTGAGTCCGGCCCAGGTAGCCCGGCTGGCTCTCCGACTTCCACCGAGGA | |
| | AGGTAGCCCGGCTGGCTCTCCAACTTCTACTGAAGAAGGTACTTCTACCGAACC | |
| | TTCCGAGGGCAGCGCACCAGGTACTTCTGAAAGCGCTACCCCTGAGTCCGGCCC | |
| | AGGTACTTCTGAAAGCGCTACTCCTGAATCCGGTCCAGGTACTTCTGAAAGCGC | |
| | TACCCCGGAATCTGGCCCAGGTAGCGAACCGGCTACTTCTGGTTCTGAAACCCC | |
| | AGGTAGCGAACCGGCTACCTCCGGTTCTGAAACTCCAGGTAGCCCAGCAGGCTC | |
| | TCCGACTTCCACTGAGGAAGGTACTTCTACTGAACCTTCCGAAGGCAGCGCACC | |
| | AGGTACCTCTACTGAACCTTCTGAGGGCAGCGCTCCAGGTAGCGAACCTGCAAC | |
| | CTCTGGCTCTGAAACCCCAGGTACCTCTGAAAGCGCTACTCCTGAATCTGGCCC | |
| | AGGTACTTCTACTGAACCGTCCGAGGGCAGCGCACCA | |
| AM1296 | GGTACTTCTACTGAACCGTCTGAAGGCAGCGCACCAGGTAGCGAACCGGCTACT | 172 |
| | TCCGGTTCTGAAACCCCAGGTAGCCCAGCAGGTTCTCCAACTTCTACTGAAGAA | |
| | GGTTCTACCAGCTCTACCGCAGAATCTCCTGGTCCAGGTACCTCTACTCCGGAA | |
| | AGCGGCTCTGCATCTCCAGGTTCTACTAGCGAATCTCCTTCTGGCACTGCACCAG | |
| | GTTCTACTAGCGAATCCCCGTCTGGTACTGCTCCAGGTACTTCTACTCCTGAAAG | |
| | CGGTTCCGCTTCTCCAGGTACCTCTACTCCGGAAAGCGGTTCTGCATCTCCAGGT | |
| | AGCGAACCGGCAACCTCCGGCTCTGAAACCCCAGGTACCTCTGAAAGCGCTACT | |
| | CCTGAATCCGGCCCAGGTAGCCCGGCAGGTTCTCCGACTTCCACTGAGGAAGGT | |
| | ACCTCTACTGAACCTTCTGAGGGCAGCGCTCCAGGTACTTCTGAAAGCGCTACC | |
| | CCGGAGTCCGGTCCAGGTACTTCTACTGAACCGTCCGAAGGTAGCGCACCAGGT | |
| | ACTTCTACCGAACCGTCCGAGGGTAGCGCACCAGGTAGCCCAGCAGGTTCTCCT | |
| | ACCTCCACCGAGGAAGGTACTTCTACCGAACCGTCCGAGGGTAGCGCACCAGGT | |
| | ACTTCTACCGAACCTTCCGAGGGCAGCGCACCAGGTACTTCTGAAAGCGCTACC | |
| | CCTGAGTCCGGCCCAGGTACTTCTGAAAGCGCTACTCCTGAATCCGGTCCAGGT | |
| | ACCTCTACTGAACCTTCCGAAGGCAGCGCTCCAGGTACCTCTACCGAACCGTCC | |
| | GAGGGCAGCGCACCAGGTACTTCTGAAAGCGCAACCCCTGAATCCGGTCCAGGT | |
| | ACTTCTACTGAACCTTCCGAAGGTAGCGCTCCAGGTAGCGAACCTGCTACTTCT | |
| | GGTTCTGAAACCCCAGGTAGCCCGGCTGGCTCTCCGACCTCCACCGAGGAAGGT | |
| | AGCTCTACCCCGTCTGGTGCTACTGGTTCTCCAGGTACTCCGGGCAGCGGTACTG | |
| | CTTCTTCCTCTCCAGGTAGCTCTACCCCTTCTGGTGCTACTGGCTCTCCAGGTAC | |
| | CTCTACCGAACCGTCCGAGGGTAGCGCACCAGGTACCTCTACTGAACCGTCTGA | |
| | GGGTAGCGCTCCAGGTAGCGAACCGGCAACCTCCGGTTCTGAAACTCCAGGTAG | |
| | CCCTGCTGGCTCTCCGACTTCTACTGAGGAAGGTAGCCCGGCTGGTTCTCCGACT | |
| | TCTACTGAGGAAGGTACTTCTACCGAACCTTCCGAAGGTAGCGCTCCAGGTCCA | |
| | GAACCAACGGGGCCGGCCCCAAGCGGAGGTAGCGAACCGGCAACCTCCGGCTC | |
| | TGAAACCCCAGGTACCTCTGAAAGCGCTACTCCTGAATCCGGCCCAGGTAGCCC | |
| | GGCAGGTTCTCCGACTTCCACTGAGGAAGGTACTTCTGAAAGCGCTACTCCTGA | |
| | GTCCGGCCCAGGTAGCCCGGCTGGCTCTCCGACTTCCACCGAGGAAGGTAGCCC | |
| | GGCTGGCTCTCCAACTTCTACTGAAGAAGGTACTTCTGAAAGCGCTACTCCTGA | |
| | GTCCGGCCCAGGTAGCCCGGCTGGCTCTCCGACTTCCACCGAGGAAGGTAGCCC | |
| | GGCTGGCTCTCCAACTTCTACTGAAGAAGGTTCTACCAGCTCTACCGCTGAATCT | |
| | CCTGGCCCAGGTTCTACTAGCGAATCTCCGTCTGGCACCGCACCAGGTACTTCCC | |
| | CTAGCGGTGAATCTTCTACTGCACCAGGTTCTACCAGCGAATCTCCTTCTGGCAC | |
| | CGCTCCAGGTTCTACTAGCGAATCCCCGTCTGGTACCGCACCAGGTACTTCTCCT | |
| | AGCGGCGAATCTTCTACCGCACCAGGTACTTCTACCGAACCTTCCGAGGGCAGC | |
| | GCACCAGGTACTTCTGAAAGCGCTACCCCTGAGTCCGGCCCAGGTACTTCTGAA | |
| | AGCGCTACTCCTGAATCCGGTCCAGGTAGCGAACCGGCAACCTCTGGCTCTGAA | |
| | ACCCCAGGTACCTCTGAAAGCGCTACTCCGGAATCTGGTCCAGGTACTTCTGAA | |
| | AGCGCTACTCCGGAATCCGGTCCAGGTACCTCTACTGAACCTTCTGAGGGCAGC | |
| | GCTCCAGGTACTTCTGAAAGCGCTACCCCGGAGTCCGGTCCAGGTACTTCTACT | |
| | GAACCGTCCGAAGGTAGCGCACCAGGTACCTCCCCTAGCGGCGAATCTTCTACT | |
| | GCTCCAGGTACCTCTCCTAGCGGCGAATCTTCTACCGCTCCAGGTACCTCCCCTA | |
| | GCGGTGAATCTTCTACCGCACCAGGTACTTCTACCGAACCGTCCGAGGGTAGCG | |
| | CACCAGGTAGCCCAGCAGGTTCTCCTACCTCCACCGAGGAAGGTACTTCTACCG | |
| | AACCGTCCGAGGGTAGCGCACCAGGTTCTAGCCCTTCTGCTTCCACCGGTACCG | |
| | GCCCAGGTAGCTCTACTCCGTCTGGTGCAACTGGCTCTCCAGGTAGCTCTACTCC | |
| | GTCTGGTGCAACCGGCTCCCCAGGTAGCTCTACCCCGTCTGGTGCTACCGGCTCT | |
| | CCAGGTAGCTCTACCCCGTCTGGTGCAACCGGCTCCCCAGGTGCATCCCCGGGT | |
| | ACTAGCTCTACCGGTTCTCCAGGTGCAAGCGCAAGCGGCGCGCCAAGCACGGG | |
| | AGGTACTTCTCCGAGCGGTGAATCTTCTACCGCACCAGGTTCTACTAGCTCTACC | |
| | GCTGAATCTCCGGGCCCAGGTACTTCTCCGAGCGGTGAATCTTCTACTGCTCCAG | |
| | GTACCTCTGAAAGCGCTACTCCGGAGTCTGGCCCAGGTACCTCTACTGAACCGT | |
| | CTGAGGGTAGCGCTCCAGGTACTTCTACTGAACCGTCCGAAGGTAGCGCACCAG | |
| | GTTCTAGCCCTTCTGCATCTACTGGTACTGGCCCAGGTAGCTCTACTCCTTCTGG | |
| | TGCTACCGGCTCTCCAGGTGCTTCTCCGGGTACTAGCTCTACCGGTTCTCCAGGT | |
| | ACTTCTACTCCGGAAAGCGGTTCCGCATCTCCAGGTACTTCTCCTAGCGGTGAAT | |
| | CTTCTACTGCTCCAGGTACCTCTCCTAGCGGCGAATCTTCTACTGCTCCAGGTAC | |
| | TTCTGAAAGCGCAACCCCTGAATCCGGTCCAGGTAGCGAACCGGCTACTTCTGG | |
| | CTCTGAGACTCCAGGTACTTCTACCGAACCGTCCGAAGGTAGCGCACCAGGTTC | |
| | TACCAGCGAATCCCCTTCTGGTACTGCTCCAGGTTCTACCAGCGAATCCCCTTCT | |
| | GGCACCGCACCAGGTACTTCTACCCCTGAAAGCGGCTCCGCTTCTCCAGGTAGC | |
| | CCGGCAGGCTCTCCGACCTCTACTGAGGAAGGTACTTCTGAAAGCGCAACCCCG | |
| | GAGTCCGGCCCAGGTACCTCTACCGAACCGTCTGAGGGCAGCGCACCAGGTAGC | |
| | CCTGCTGGCTCTCCAACCTCCACCGAAGAAGGTACCTCTGAAAGCGCAACCCCT | |
| | GAATCCGGCCCAGGTAGCGAACCGGCAACCTCCGGTTCTGAAACCCCAGGTAGC | |
| | TCTACCCCGTCTGGTGCTACCGGTTCCCCAGGTGCTTCTCCTGGTACTAGCTCTA | |
| | CCGGTTCTCCAGGTAGCTCTACCCCGTCTGGTGCTACTGGCTCTCCAGGTTCTAC | |
| | TAGCGAATCCCCGTCTGGTACTGCTCCAGGTACTTCCCCTAGCGGTGAATCTTCT | |
| | ACTGCTCCAGGTTCTACCAGCTCTACCGCAGAATCTCCGGGTCCAGGTAGCTCT | |
| | ACCCCTTCTGGTGCAACCGGCTCTCCAGGTGCATCCCCGGGTACCAGCTCTACC | |
| | GGTTCTCCAGGTACTCCGGGTAGCGGTACCGCTTCTTCCTCTCCAGGTAGCCCTG | |
| | CTGGCTCTCCGACTTCTACTGAGGAAGGTAGCCCGGCTGGTTCTCCGACTTCTAC | |
| | TGAGGAAGGTACTTCTACCGAACCTTCCGAAGGTAGCGCTCCA | |
| BC864 | GGTACTTCCACCGAACCATCCGAACCAGGTAGCGCAGGTACTTCCACCGAACCA | 173 |
| | TCCGAACCTGGCAGCGCAGGTAGCGAACCGGCAACCTCTGGTACTGAACCATCA | |
| | GGTAGCGGCGCATCCGAGCCTACCTCTACTGAACCAGGTAGCGAACCGGCTACC | |
| | TCCGGTACTGAGCCATCAGGTAGCGAACCGGCAACTTCCGGTACTGAACCATCA | |
| | GGTAGCGAACCGGCAACTTCCGGCACTGAACCATCAGGTAGCGGTGCATCTGAG | |
| | CCGACCTCTACTGAACCAGGTACTTCTACTGAACCATCTGAGCCGGGCAGCGCA | |
| | GGTAGCGAACCAGCTACTTCTGGCACTGAACCATCAGGTACTTCTACTGAACCA | |
| | TCCGAACCAGGTAGCGCAGGTAGCGAACCTGCTACCTCTGGTACTGAGCCATCA | |
| | GGTAGCGAACCGGCTACCTCTGGTACTGAACCATCAGGTACTTCTACCGAACCA | |
| | TCCGAGCCTGGTAGCGCAGGTACTTCTACCGAACCATCCGAGCCAGGCAGCGCA | |
| | GGTAGCGAACCGGCAACCTCTGGCACTGAGCCATCAGGTAGCGAACCAGCAAC | |
| | TTCTGGTACTGAACCATCAGGTACTAGCGAGCCATCTACTTCCGAACCAGGTGC | |
| | AGGTAGCGGCGCATCCGAACCTACTTCCACTGAACCAGGTACTAGCGAGCCATC | |
| | CACCTCTGAACCAGGTGCAGGTAGCGAACCGGCAACTTCCGGCACTGAACCATC | |
| | AGGTAGCGAACCGGCTACCTCTGGTACTGAACCATCAGGTACTTCTACCGAACC | |
| | ATCCGAGCCTGGTAGCGCAGGTACTTCTACCGAACCATCCGAGCCAGGCAGCGC | |
| | AGGTAGCGGTGCATCCGAGCCGACCTCTACTGAACCAGGTAGCGAACCAGCAA | |
| | CTTCTGGCACTGAGCCATCAGGTAGCGAACCAGCTACCTCTGGTACTGAACCAT | |
| | CAGGTAGCGAACCGGCTACTTCCGGCACTGAACCATCAGGTAGCGAACCAGCA | |
| | ACCTCCGGTACTGAACCATCAGGTACTTCCACTGAACCATCCGAACCGGGTAGC | |
| | GCAGGTAGCGAACCGGCAACTTCCGGCACTGAACCATCAGGTAGCGGTGCATCT | |
| | GAGCCGACCTCTACTGAACCAGGTACTTCTACTGAACCATCTGAGCCGGGCAGC | |
| | GCAGGTAGCGAACCTGCAACCTCCGGCACTGAGCCATCAGGTAGCGGCGCATCT | |
| | GAACCAACCTCTACTGAACCAGGTACTTCCACCGAACCATCTGAGCCAGGCAGC | |
| | GCAGGTAGCGGCGCATCTGAACCAACCTCTACTGAACCAGGTAGCGAACCAGC | |
| | AACTTCTGGTACTGAACCATCAGGTAGCGGCGCATCTGAGCCTACTTCCACTGA | |
| | ACCAGGTAGCGAACCGGCAACTTCCGGCACTGAACCATCAGGTAGCGGTGCATC | |
| | TGAGCCGACCTCTACTGAACCAGGTACTTCTACTGAACCATCTGAGCCGGGCAG | |
| | CGCAGGTAGCGAACCGGCAACTTCCGGCACTGAACCATCAGGTAGCGGTGCATC | |
| | TGAGCCGACCTCTACTGAACCAGGTACTTCTACTGAACCATCTGAGCCGGGCAG | |
| | CGCAGGTAGCGAACCAGCTACTTCTGGCACTGAACCATCAGGTACTTCTACTGA | |
| | ACCATCCGAACCAGGTAGCGCAGGTAGCGAACCTGCTACCTCTGGTACTGAGCC | |
| | ATCAGGTACTTCTACTGAACCATCCGAGCCGGGTAGCGCAGGTACTTCCACTGA | |
| | ACCATCTGAACCTGGTAGCGCAGGTACTTCCACTGAACCATCCGAACCAGGTAG | |
| | CGCAGGTACTTCTACTGAACCATCCGAGCCGGGTAGCGCAGGTACTTCCACTGA | |
| | ACCATCTGAACCTGGTAGCGCAGGTACTTCCACTGAACCATCCGAACCAGGTAG | |
| | CGCAGGTACTAGCGAACCATCCACCTCCGAACCAGGCGCAGGTAGCGGTGCATC | |
| | TGAACCGACTTCTACTGAACCAGGTACTTCCACTGAACCATCTGAGCCAGGTAG | |
| | CGCAGGTACTTCCACCGAACCATCCGAACCAGGTAGCGCAGGTACTTCCACCGA | |
| | ACCATCCGAACCTGGCAGCGCAGGTAGCGAACCGGCAACCTCTGGTACTGAACC | |
| | ATCAGGTAGCGGTGCATCCGAGCCGACCTCTACTGAACCAGGTAGCGAACCAGC | |
| | AACTTCTGGCACTGAGCCATCAGGTAGCGAACCAGCTACCTCTGGTACTGAACC | |
| | ATCAGGTAGCGAACCGGCAACCTCTGGCACTGAGCCATCAGGTAGCGAACCAG | |
| | CAACTTCTGGTACTGAACCATCAGGTACTAGCGAGCCATCTACTTCCGAACCAG | |
| | GTGCAGGTAGCGAACCTGCAACCTCCGGCACTGAGCCATCAGGTAGCGGCGCAT | |
| | CTGAACCAACCTCTACTGAACCAGGTACTTCCACCGAACCATCTGAGCCAGGCA | |
| | GCGCAGGTAGCGAACCTGCAACCTCCGGCACTGAGCCATCAGGTAGCGGCGCA | |
| | TCTGAACCAACCTCTACTGAACCAGGTACTTCCACCGAACCATCTGAGCCAGGC | |
| | AGCGCA | |
| BD864 | GGTAGCGAAACTGCTACTTCCGGCTCTGAGACTGCAGGTACTAGTGAATCCGCA | 174 |
| | ACTAGCGAATCTGGCGCAGGTAGCACTGCAGGCTCTGAGACTTCCACTGAAGCA | |
| | GGTACTAGCGAGTCCGCAACCAGCGAATCCGGCGCAGGTAGCGAAACTGCTAC | |
| | CTCTGGCTCCGAGACTGCAGGTAGCGAAACTGCAACCTCTGGCTCTGAAACTGC | |
| | AGGTACTTCCACTGAAGCAAGTGAAGGCTCCGCATCAGGTACTTCCACCGAAGC | |
| | AAGCGAAGGCTCCGCATCAGGTACTAGTGAGTCCGCAACTAGCGAATCCGGTGC | |
| | AGGTAGCGAAACCGCTACCTCTGGTTCCGAAACTGCAGGTACTTCTACCGAGGC | |
| | TAGCGAAGGTTCTGCATCAGGTAGCACTGCTGGTTCCGAGACTTCTACTGAAGC | |
| | AGGTACTAGCGAATCTGCTACTAGCGAATCCGGCGCAGGTACTAGCGAATCCGC | |
| | TACCAGCGAATCCGGCGCAGGTAGCGAAACTGCAACCTCTGGTTCCGAGACTGC | |
| | AGGTACTAGCGAGTCCGCTACTAGCGAATCTGGCGCAGGTACTTCCACTGAAGC | |
| | TAGTGAAGGTTCTGCATCAGGTAGCGAAACTGCTACTTCTGGTTCCGAAACTGC | |
| | AGGTAGCGAAACCGCTACCTCTGGTTCCGAAACTGCAGGTACTTCTACCGAGGC | |
| | TAGCGAAGGTTCTGCATCAGGTAGCACTGCTGGTTCCGAGACTTCTACTGAAGC | |
| | AGGTACTAGCGAGTCCGCTACTAGCGAATCTGGCGCAGGTACTTCCACTGAAGC | |
| | TAGTGAAGGTTCTGCATCAGGTAGCGAAACTGCTACTTCTGGTTCCGAAACTGC | |
| | AGGTAGCACTGCTGGCTCCGAGACTTCTACCGAAGCAGGTAGCACTGCAGGTTC | |
| | CGAAACTTCCACTGAAGCAGGTAGCGAAACTGCTACCTCTGGCTCTGAGACTGC | |
| | AGGTACTAGCGAATCTGCTACTAGCGAATCCGGCGCAGGTACTAGCGAATCCGC | |
| | TACCAGCGAATCCGGCGCAGGTAGCGAAACTGCAACCTCTGGTTCCGAGACTGC | |
| | AGGTACTAGCGAATCTGCTACTAGCGAATCCGGCGCAGGTACTAGCGAATCCGC | |
| | TACCAGCGAATCCGGCGCAGGTAGCGAAACTGCAACCTCTGGTTCCGAGACTGC | |
| | AGGTAGCGAAACCGCTACCTCTGGTTCCGAAACTGCAGGTACTTCTACCGAGGC | |
| | TAGCGAAGGTTCTGCATCAGGTAGCACTGCTGGTTCCGAGACTTCTACTGAAGC | |
| | AGGTAGCGAAACTGCTACTTCCGGCTCTGAGACTGCAGGTACTAGTGAATCCGC | |
| | AACTAGCGAATCTGGCGCAGGTAGCACTGCAGGCTCTGAGACTTCCACTGAAGC | |
| | AGGTAGCACTGCTGGTTCCGAAACCTCTACCGAAGCAGGTAGCACTGCAGGTTC | |
| | TGAAACCTCCACTGAAGCAGGTACTTCCACTGAGGCTAGTGAAGGCTCTGCATC | |
| | AGGTAGCACTGCTGGTTCCGAAACCTCTACCGAAGCAGGTAGCACTGCAGGTTC | |
| | TGAAACCTCCACTGAAGCAGGTACTTCCACTGAGGCTAGTGAAGGCTCTGCATC | |
| | AGGTAGCACTGCAGGTTCTGAGACTTCCACCGAAGCAGGTAGCGAAACTGCTAC | |
| | TTCTGGTTCCGAAACTGCAGGTACTTCCACTGAAGCTAGTGAAGGTTCCGCATC | |
| | AGGTACTAGTGAGTCCGCAACCAGCGAATCCGGCGCAGGTAGCGAAACCGCAA | |
| | CCTCCGGTTCTGAAACTGCAGGTACTAGCGAATCCGCAACCAGCGAATCTGGCG | |
| | CAGGTACTAGTGAGTCCGCAACCAGCGAATCCGGCGCAGGTAGCGAAACCGCA | |
| | ACCTCCGGTTCTGAAACTGCAGGTACTAGCGAATCCGCAACCAGCGAATCTGGC | |
| | GCAGGTAGCGAAACTGCTACTTCCGGCTCTGAGACTGCAGGTACTTCCACCGAA | |
| | GCAAGCGAAGGTTCCGCATCAGGTACTTCCACCGAGGCTAGTGAAGGCTCTGCA | |
| | TCAGGTAGCACTGCTGGCTCCGAGACTTCTACCGAAGCAGGTAGCACTGCAGGT | |
| | TCCGAAACTTCCACTGAAGCAGGTAGCGAAACTGCTACCTCTGGCTCTGAGACT | |
| | GCAGGTACTAGCGAATCTGCTACTAGCGAATCCGGCGCAGGTACTAGCGAATCC | |
| | GCTACCAGCGAATCCGGCGCAGGTAGCGAAACTGCAACCTCTGGTTCCGAGACT | |
| | GCAGGTAGCGAAACTGCTACTTCCGGCTCCGAGACTGCAGGTAGCGAAACTGCT | |
| | ACTTCTGGCTCCGAAACTGCAGGTACTTCTACTGAGGCTAGTGAAGGTTCCGCA | |
| | TCAGGTACTAGCGAGTCCGCAACCAGCGAATCCGGCGCAGGTAGCGAAACTGC | |
| | TACCTCTGGCTCCGAGACTGCAGGTAGCGAAACTGCAACCTCTGGCTCTGAAAC | |
| | TGCAGGTACTAGCGAATCTGCTACTAGCGAATCCGGCGCAGGTACTAGCGAATC | |
| | CGCTACCAGCGAATCCGGCGCAGGTAGCGAAACTGCAACCTCTGGTTCCGAGAC | |
| | TGCA | |

One may clone the library of XTEN-encoding genes into one or more expression vectors known in the art. To facilitate the identification of well-expressing library members, one can construct the library as fusion to a reporter protein. Non-limiting examples of suitable reporter genes are green fluorescent protein, luciferase, alkaline phosphatase, and beta-galactosidase. By screening, one can identify short XTEN sequences that can be expressed in high concentration in the host organism of choice. Subsequently, one can generate a library of random XTEN dimers and repeat the screen for high level of expression. Subsequently, one can screen the resulting constructs for a number of properties such as level of expression, protease stability, or binding to antiserum.

One aspect of the invention is to provide polynucleotide sequences encoding the components of the fusion protein wherein the creation of the sequence has undergone codon optimization. Of particular interest is codon optimization with the goal of improving expression of the polypeptide compositions and to improve the genetic stability of the encoding gene in the production hosts. For example, codon optimization is of particular importance for XTEN sequences that are rich in glycine or that have very repetitive amino acid sequences. Codon optimization can be performed using computer programs (Gustafsson, C., et al. (2004) Trends Biotechnol, 22: 346-53), some of which minimize ribosomal pausing (Coda Genomics Inc.). In one embodiment, one can perform codon optimization by constructing codon libraries where all members of the library encode the same amino acid sequence but where codon usage is varied. Such libraries can be screened for highly expressing and genetically stable members that are particularly suitable for the large-scale production of XTEN-containing products. When designing XTEN sequences one can consider a number of properties. One can minimize the repetitiveness in the encoding DNA sequences. In addition, one can avoid or minimize the use of codons that are rarely used by the production host (e.g. the AGG and AGA arginine codons and one leucine codon in E. coli). In the case of E. coli, two glycine codons, GGA and GGG, are rarely used in highly expressed proteins. Thus codon optimization of the gene encoding XTEN sequences can be very desirable. DNA sequences that have a high level of glycine tend to have a high GC content that can lead to instability or low expression levels. Thus, when possible, it is preferred to choose codons such that the GC-content of XTEN-encoding sequence is suitable for the production organism that will be used to manufacture the XTEN.

Optionally, the full-length XTEN-encoding gene may comprise one or more sequencing islands. In this context, sequencing islands are short-stretch sequences that are distinct from the XTEN library construct sequences and that include a restriction site not present or expected to be present in the full-length XTEN-encoding gene. In one embodiment, a sequencing island is the sequence 5′-AGGTGCAAGCGCAAGCGGCGCGCCAAGCACGGGAGGT-3′ (SEQ ID NO: 175). In another embodiment, a sequencing island is the sequence 5′-AGGTCCAGAACCAACGGGGCCGGCCCCAAGCGGAGGT-3′ (SEQ ID NO: 176).

As an alternative, one can construct codon libraries where all members of the library encode the same amino acid sequence but where codon usage is varied. Such libraries can be screened for highly expressing and genetically stable members that are particularly suitable for the large-scale production of XTEN-containing products.

Optionally, one can sequence clones in the library to eliminate isolates that contain undesirable sequences. The initial library of short XTEN sequences can allow some variation in amino acid sequence. For instance one can randomize some codons such that a number of hydrophilic amino acids can occur in a particular position.

During the process of iterative multimerization one can screen the resulting library members for other characteristics like solubility or protease resistance in addition to a screen for high-level expression.

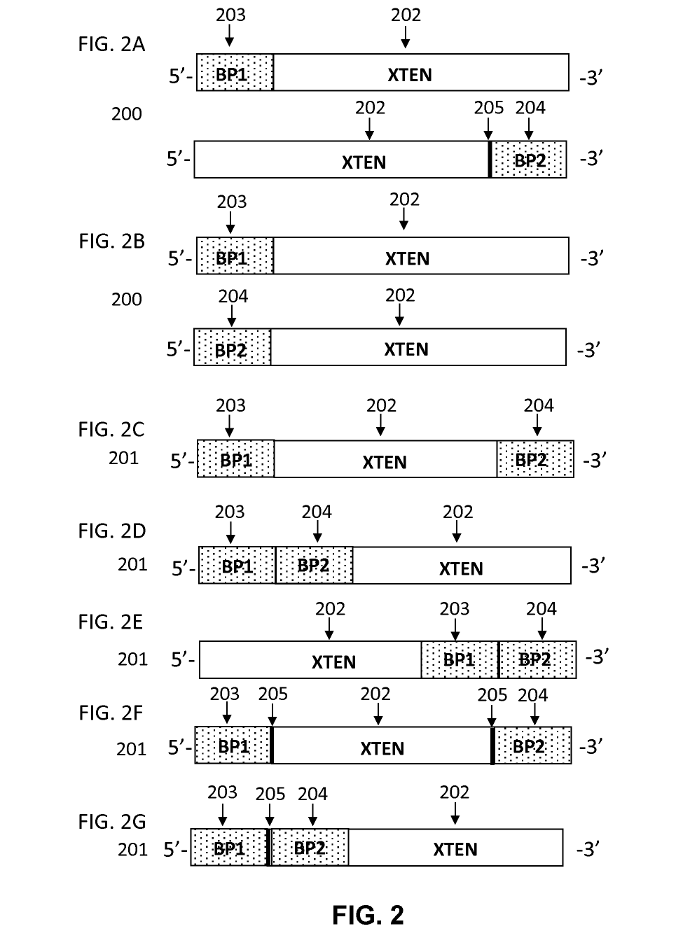

Once the gene that encodes the XTEN of desired length and properties is selected, it is genetically fused to the nucleotides encoding the N- and/or the C-terminus of the BP gene(s) by cloning it into the construct adjacent and in frame with the gene coding for BP or adjacent to a spacer sequence. The invention provides various permutations of the foregoing, depending on the BFXTEN to be encoded. For example, a gene encoding a monomeric fusion protein comprising two BP such as embodied by formula (I) or (II), as depicted above, the gene would have polynucleotides encoding two BP, at least a first XTEN, and optionally a second XTEN and/or spacer sequences. The step of cloning the BP genes into the XTEN construct can occur through a ligation or multimerization step. As shown in FIG. 2, the constructs encoding BFXTEN fusion proteins can be designed in different configurations. In one embodiment, as illustrated in FIG. 2A, the constructs 200 encoding combination (two fusion protein) BFXTEN comprise polynucleotide sequences complementary to, or those that encode a monomeric polypeptide of components XTEN 202, BP1 203, BP2 204 and spacer sequences 205. In another embodiment, as illustrated in FIG. 2B, the construct comprises polynucleotide sequences complementary to, or those that encode a monomeric polypeptide of components in the following order (5′ to 3′) BP1 203 and XTEN 202 and BP2 204. In another embodiment, as illustrated in FIG. 2C, the construct 201 encodes a monomeric BXTEN comprising polynucleotide sequences complementary to, or those that encode components in the following order (5′ to 3′): BP1 203, XTEN 202, and BP2 204. In another embodiment, as illustrated in FIG. 2D, the construct comprises polynucleotide sequences complementary to, or those that encode a monomeric polypeptide of components in the following order (5′ to 3′): BP1 203; BP2 204; and XTEN 202. In another embodiment, as illustrated in FIG. 2E, the construct comprises polynucleotide sequences complementary to, or those that encode a monomeric polypeptide of components in the following order (5′ to 3′): XTEN 202; BP1 203; and BP2 204. In another embodiment, as illustrated in FIG. 2F, the construct comprises polynucleotide sequences complementary to, or those that encode a monomeric polypeptide of components in the following order (5′ to 3′): BP1 203; spacer sequences 205; BP2 204; and XTEN 202. In another embodiment, as illustrated in FIG. 2G, the construct comprises polynucleotide sequences complementary to, or those that encode a monomeric polypeptide of components in the following order (5′ to 3′): BP1 203; spacer sequences 205; BP2 204; and XTEN 202. The spacer polynucleotides can optionally comprise sequences encoding cleavage sequences. The invention also contemplates other permutations of the foregoing. Polynucleotide constructs can also be created that encode a polypeptide with multimers of BP and XTEN linked in alternating units.

The invention also encompasses polynucleotide variants that have a high percentage of sequence identity to (a) a polynucleotide sequence from Table 8, or (b) sequences that are complementary to the polynucleotides of (a). A polynucleotide with a high percentage of sequence identity is one that has at least about an 80% nucleic acid sequence identity, alternatively at least about 81%, alternatively at least about 82%, alternatively at least about 83%, alternatively at least about 84%, alternatively at least about 85%, alternatively at least about 86%, alternatively at least about 87%, alternatively at least about 88%, alternatively at least about 89%, alternatively at least about 90%, alternatively at least about 91%, alternatively at least about 92%, alternatively at least about 93%, alternatively at least about 94%, alternatively at least about 95%, alternatively at least about 96%, alternatively at least about 97%, alternatively at least about 98%, and alternatively at least about 99% nucleic acid sequence identity to (a) or (b) of the foregoing, or that can hybridize with the target polynucleotide or its complement under stringent conditions.

Homology, sequence similarity or sequence identity of nucleotide or amino acid sequences may also be determined conventionally by using known software or computer programs such as the BestFit or Gap pairwise comparison programs (GCG Wisconsin Package, Genetics Computer Group, 575 Science Drive, Madison, Wis. 53711). BestFit uses the local homology algorithm of Smith and Waterman (Advances in Applied Mathematics. 1981. 2: 482-489), to find the best segment of identity or similarity between two sequences. Gap performs global alignments: all of one sequence with all of another similar sequence using the method of Needleman and Wunsch, (Journal of Molecular Biology. 1970. 48:443-453). When using a sequence alignment program such as BestFit, to determine the degree of sequence homology, similarity or identity, the default setting may be used, or an appropriate scoring matrix may be selected to optimize identity, similarity or homology scores.

Nucleic acid sequences that are “complementary” are those that are capable of base-pairing according to the standard Watson-Crick complementarity rules. As used herein, the term “complementary sequences” means nucleic acid sequences that are substantially complementary, as may be assessed by the same nucleotide comparison set forth above, or as defined as being capable of hybridizing to the polynucleotides that encode the BFXTEN sequences under stringent conditions, such as those described herein.

The resulting polynucleotides encoding the BFXTEN chimeric compositions can then be individually cloned into an expression vector. The nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan. Such techniques are well known in the art and well described in the scientific and patent literature.

Various vectors are publicly available. The vector may, for example, be in the form of a plasmid, cosmid, viral particle, or phage. Both expression and cloning vectors contain a nucleic acid sequence that enables the vector to replicate in one or more selected host cells. Such vector sequences are well known for a variety of bacteria, yeast, and viruses. Useful expression vectors that can be used include, for example, segments of chromosomal, non-chromosomal and synthetic DNA sequences. Suitable vectors include, but are not limited to, derivatives of SV40 and pcDNA and known bacterial plasmids such as col EI, pCR1, pBR322, pMal-C2, pET, pGEX as described by Smith, et al., Gene 57:31-40 (1988), pMB9 and derivatives thereof, plasmids such as RP4, phage DNAs such as the numerous derivatives of phage I such as NM98 9, as well as other phage DNA such as M13 and filamentous single stranded phage DNA; yeast plasmids such as the 2 micron plasmid or derivatives of the 2 m plasmid, as well as centromeric and integrative yeast shuttle vectors; vectors useful in eukaryotic cells such as vectors useful in insect or mammalian cells; vectors derived from combinations of plasmids and phage DNAs, such as plasmids that have been modified to employ phage DNA or the expression control sequences; and the like. The requirements are that the vectors are replicable and viable in the host cell of choice. Low- or high-copy number vectors may be used as desired.

Promoters suitable for use in expression vectors with prokaryotic hosts include the β-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)]. Promoters for use in bacterial systems can also contain a Shine-Dalgarno (S.D.) sequence operably linked to the DNA encoding BFXTEN polypeptides.

For example, in a baculovirus expression system, both non-fusion transfer vectors, such as, but not limited to pVL941 (BamHI cloning site, available from Summers, et al., Virology 84:390-402 (1978)), pVL1393 (BamHI, Smal, Xbal, EcoRI, IVotl, Xmalll, BgIII and Pstl cloning sites; Invitrogen), pVL1392 (BgIII, Pstl, NotI, XmaIII, EcoRI, Xball, Smal and BamHI cloning site; Summers, et al., Virology 84:390-402 (1978) and Invitrogen) and pBlueBacIII (BamHI, BgIII, Pstl, Ncol and Hindi II cloning site, with blue/white recombinant screening, Invitrogen), and fusion transfer vectors such as, but not limited to, pAc7 00 (BamHI and Kpn1 cloning sites, in which the BamHI recognition site begins with the initiation codon; Summers, et al., Virology 84:390-402 (1978)), pAc701 and pAc70-2 (same as pAc700, with different reading frames), pAc360 [BamHI cloning site 36 base pairs downstream of a polyhedrin initiation codon; Invitrogen (1995)) and pBlueBacHisA, B, C (three different reading frames with BamH I, BgI II, Pstl, Nco l and Hind III cloning site, an N-terminal peptide for ProBond purification and blue/white recombinant screening of plaques; Invitrogen (220) can be used.

Mammalian expression vectors can comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 splice, and polyadenylation sites may be used to provide the required nontranscribed genetic elements. Mammalian expression vectors contemplated for use in the invention include vectors with inducible promoters, such as the dihydrofolate reductase promoters, any expression vector with a DHFR expression cassette or a DHFR/methotrexate co-amplification vector such as pED (Pstl, Sail, Sbal, Smal and EcoRI cloning sites, with the vector expressing both the cloned gene and DHFR; Randal J. Kaufman, 1991, Randal J. Kaufman, Current Protocols in Molecular Biology, 16, 12 (1991)). Alternatively a glutamine synthetase/methionine sulfoximine co-amplification vector, such as pEE14 (Hindlll, Xball, Smal, Sbal, EcoRI and Sell cloning sites in which the vector expresses glutamine synthetase and the cloned gene; Celltech). A vector that directs episomal expression under the control of the Epstein Barr Virus (EBV) or nuclear antigen (EBNA) can be used such as pREP4 (BamHI r SfH, Xhol, NotI, Nhel, Hindi II, NheI, PvuII and Kpnl cloning sites, constitutive RSV-LTR promoter, hygromycin selectable marker; Invitrogen), pCEP4 (BamHI, SfH, Xhol, NotI, Nhel, Hindlll, Nhel, PvuII and Kpnl cloning sites, constitutive hCMV immediate early gene promoter, hygromycin selectable marker; Invitrogen), pMEP4 (.Kpnl, Pvul, Nhel, Hindlll, NotI, Xhol, Sfil, BamHI cloning sites, inducible metallothionein H a gene promoter, hygromycin selectable marker, Invitrogen), pREP8 (BamHI, XhoI, NotI, Hindlll, Nhel and Kpnl cloning sites, RSV-LTR promoter, histidinol selectable marker; Invitrogen), pREP9 (Kpnl, Nhel, Hind lll, NotI, Xho l, Sfi l, BamH I cloning sites, RSV-LTR promoter, G418 selectable marker; Invitrogen), and pEBVHis (RSV-LTR promoter, hygromycin selectable marker, N-terminal peptide purifiable via ProBond resin and cleaved by enterokinase; Invitrogen).

Selectable mammalian expression vectors for use in the invention include, but are not limited to, pRc/CMV (Hind lll, BstXI, NotI, Sbal and Apal cloning sites, G418 selection, Invitrogen), pRc/RSV (Hind II, Spel, BstXI, NotI, Xbal cloning sites, G418 selection, Invitrogen) and the like. Vaccinia virus mammalian expression vectors (see, for example, Randall J. Kaufman, Current Protocols in Molecular Biology 16.12 (Frederick M. Ausubel, et al., eds. Wiley 1991) that can be used in the present invention include, but are not limited to, pSC11 (Smal cloning site, TK- and beta-gal selection), pMJ601 (Sal l, Sma l, A flI, Narl, BspMlI, BamHI, Apal, Nhel, SacII, Kpnl and Hindlll cloning sites; TK- and -gal selection), pTKgtFlS (EcoRI, Pstl, SaIII, Accl, HindII, Sbal, BamHI and Hpa cloning sites, TK or XPRT selection) and the like.

Yeast expression systems that can also be used in the present invention include, but are not limited to, the non-fusion pYES2 vector (XJbal, Sphl, Shol, NotI, GstXI, EcoRI, BstXI, BamHI, Sad, Kpnl and Hindlll cloning sites, Invitrogen), the fusion pYESHisA, B, C (Xball, Sphl, Shol, NotI, BstXI, EcoRI, BamHI, Sad, Kpnl and Hindi II cloning sites, N-terminal peptide purified with ProBond resin and cleaved with enterokinase; Invitrogen), pRS vectors and the like.

In addition, the expression vector containing the chimeric BFXTEN fusion protein-encoding polynucleotide molecule may include drug selection markers. Such markers aid in cloning and in the selection or identification of vectors containing chimeric DNA molecules. For example, genes that confer resistance to neomycin, puromycin, hygromycin, dihydrofolate reductase (DHFR) inhibitor, guanine phosphoribosyl transferase (GPT), zeocin, and histidinol are useful selectable markers. Alternatively, enzymes such as herpes simplex virus thymidine kinase (tk) or chloramphenicol acetyltransferase (CAT) may be employed. Immunologic markers also can be employed. Any known selectable marker may be employed so long as it is capable of being expressed simultaneously with the nucleic acid encoding a gene product. Further examples of selectable markers are well known to one of skill in the art and include reporters such as enhanced green fluorescent protein (EGFP), beta-galactosidase (β-gal) or chloramphenicol acetyltransferase (CAT).

In one embodiment, the polynucleotide encoding a BFXTEN fusion protein composition can be fused C-terminally to an N-terminal signal sequence appropriate for the expression host system. Signal sequences are typically proteolytically removed from the protein during the translocation and secretion process, generating a defined N-terminus. A wide variety of signal sequences have been described for most expression systems, including bacterial, yeast, insect, and mammalian systems. A non-limiting list of preferred examples for each expression system follows herein. Preferred signal sequences are OmpA, PhoA, and DsbA for E. coli expression. Signal peptides preferred for yeast expression are ppL-alpha, DEX4, invertase signal peptide, acid phosphatase signal peptide, CPY, or INU1. For insect cell expression the preferred signal sequences are sexta adipokinetic hormone precursor, CP1, CP2, CP3, CP4, TPA, PAP, or gp67. For mammalian expression the preferred signal sequences are IL2L, SV40, IgG kappa and IgG lambda.

In another embodiment, a leader sequence, potentially comprising a well-expressed, independent protein domain, can be fused to the N-terminus of the BFXTEN sequence, separated by a protease cleavage site. While any leader peptide sequence which does not inhibit cleavage at the designed proteolytic site can be used, sequences in preferred embodiments will comprise stable, well-expressed sequences such that expression and folding of the overall composition is not significantly adversely affected, and preferably expression, solubility, and/or folding efficiency are significantly improved. A wide variety of suitable leader sequences have been described in the literature. A non-limiting list of suitable sequences includes maltose binding protein, cellulose binding domain, glutathione S-transferase, 6×His tag (SEQ ID NO: 177), FLAG tag, hemaglutinin tag, and green fluorescent protein. The leader sequence can also be further improved by codon optimization, especially in the second codon position following the ATG start codon, by methods well described in the literature and hereinabove.

Various in vitro enzymatic methods for cleaving proteins at specific sites are known. Such methods include use of enterokinase (DDDK) (SEQ ID NO: 178), Factor Xa (IDGR) (SEQ ID NO: 179), thrombin (LVPR/GS) (SEQ ID NO: 180), PreScission™ (LEVLFQ/GP) (SEQ ID NO: 181), TEV protease (EQLYFQ/G) (SEQ ID NO: 182), 3C protease (ETLFQ/GP) (SEQ ID NO: 183), Sortase A (LPET/G) (SEQ ID NO: 2377), Granzyme B (D/X, N/X, M/N or S/X), inteins, SUMO, DAPase (TAGZyme™), Aeromonas aminopeptidase, Aminopeptidase M, and carboxypeptidases A and B. Additional methods are disclosed in Arnau, et al., Protein Expression and Purification 48: 1-13 (2006).

In another embodiment, an optimized polynucleotide sequence encoding at least about 20 to about 60 amino acids with XTEN characteristics can be included at the N-terminus of the XTEN sequence to promote the initiation of translation to allow for expression of XTEN fusions at the N-terminus of proteins without the presence of a helper domain. In the embodiment, the sequence does not require subsequent cleavage, thereby reducing the number of steps to manufacture XTEN-containing compositions. As described in more detail in the Examples, the optimized N-terminal sequence has attributes of an unstructured protein, but may include nucleotide bases encoding amino acids selected for their ability to promote initiation of translation and enhanced expression.

In another embodiment, the protease site of the leader sequence construct is chosen such that it is recognized by an in vivo protease. In this embodiment, the protein is purified from the expression system while retaining the leader by avoiding contact with an appropriate protease. The full-length construct is then injected into a patient. Upon injection, the construct comes into contact with the protease specific for the cleavage site and is cleaved by the protease. In the case where the uncleaved protein is substantially less active than the cleaved form, this method has the beneficial effect of allowing higher initial doses while avoiding toxicity, as the active form is generated slowly in vivo. Some non-limiting examples of in vivo proteases which are useful for this application include tissue kallikrein, plasma kallikrein, trypsin, pepsin, chymotrypsin, thrombin, and matrix metalloproteinases.

In this manner, a chimeric DNA molecule coding for a monomeric BFXTEN fusion protein is generated within the construct. Optionally, this chimeric DNA molecule may be transferred or cloned into another construct that is a more appropriate expression vector. At this point, a host cell capable of expressing the chimeric DNA molecule can be transformed with the chimeric DNA molecule. The vectors containing the DNA segments of interest can be transferred into the host cell by well-known methods, depending on the type of cellular host. For example, calcium chloride transfection is commonly utilized for prokaryotic cells, whereas calcium phosphate treatment, lipofection, or electroporation may be used for other cellular hosts. Other methods used to transform mammalian cells include the use of polybrene, protoplast fusion, liposomes, electroporation, and microinjection. See, generally, Sambrook, et al., supra.

The transformation may occur with or without the utilization of a carrier, such as an expression vector. Then, the transformed host cell is cultured under conditions suitable for expression of the chimeric DNA molecule encoding of BFXTEN.

The present invention also provides a host cell for expressing the monomeric fusion protein compositions disclosed herein. In those cases where the BFXTEN composition comprises two fusion proteins, each comprising a single BP, the invention provides a first host cell comprising the expression vector encoding the first fusion protein and a second host cell comprising the expression vector encoding the second fusion protein. Examples of suitable eukaryotic host cells include, but are not limited to mammalian cells, such as COS-1 (ATCC CRL 1650), COS-7 (ATCC CRL 1651), BHK-21 (ATCC CCL 10)) and BHK-293 (ATCC CRL 1573; Graham et al., J. Gen. Virol. 36:59-72, 1977), BHK-570 cells (ATCC CRL 10314), CHO-K1 (ATCC CCL 61), CHO-S (Invitrogen 11619-012), and 293-F (Invitrogen R790-7). A tk ts13 BHK cell line is also available from the ATCC under accession number CRL 1632. In addition, a number of other cell lines may be used within the present invention, including Rat Hep I (Rat hepatoma; ATCC CRL 1600), Rat Hep II (Rat hepatoma; ATCC CRL 1548), TCMK (ATCC CCL 139), Human lung (ATCC HB 8065), NCTC 1469 (ATCC CCL 9.1), CHO (ATCC CCL 61) and DUKX cells (Urlaub and Chasin, Proc. Natl. Acad. Sci. USA 77:4216-4220, 1980).

Examples of suitable yeasts cells include cells of Saccharomyces spp. or Schizosaccharomyces spp., in particular strains of Saccharomyces cerevisiae or Saccharomyces kluyveri. Methods for transforming yeast cells with heterologous DNA and producing heterologous polypeptides there from are described, e.g. in U.S. Pat. No. 4,599,311, U.S. Pat. No. 4,931,373, U.S. Pat. Nos. 4,870,008, 5,037,743, and U.S. Pat. No. 4,845,075, all of which are hereby incorporated by reference. Transformed cells are selected by a phenotype determined by a selectable marker, commonly drug resistance or the ability to grow in the absence of a particular nutrient, e.g. leucine. A preferred vector for use in yeast is the POT1 vector disclosed in U.S. Pat. No. 4,931,373. The DNA sequences encoding the BFXTEN may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (cf. Gleeson et al., J. Gen. Microbiol. 132, 1986, pp. 3459-3465; U.S. Pat. No. 4,882,279). Examples of other fungal cells are cells of filamentous fungi, e.g. Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277, EP 238 023, EP 184 438 The transformation of F. oxysporum may, for instance, be carried out as described by Malardier et al., 1989, Gene 78: 147-156. The transformation of Trichoderma spp. may be performed for instance as described in EP 244 234.

Other suitable cells that can be used in the present invention include, but are not limited to, prokaryotic host cells strains such as Escherichia coli, (e.g., strain DH5-α), Bacillus subtilis, Salmonella typhimurium, or strains of the genera of Pseudomonas, Streptomyces and Staphylococcus. Non-limiting examples of suitable prokaryotes include those from the genera: Actinoplanes; Archaeoglobus; Bdellovibrio; Borrelia; Chloroflexus; Enterococcus; Escherichia; Lactobacillus; Listeria; Oceanobacillus; Paracoccus; Pseudomonas; Staphylococcus; Streptococcus; Streptomyces; Thermoplasma; and Vibrio. Non-limiting examples of specific strains include: Archaeoglobus fulgidus; Bdellovibrio bacteriovorus; Borrelia burgdorferi; Chloroflexus aurantiacus; Enterococcus faecalis; Enterococcus faecium; Lactobacillus johnsonii; Lactobacillus plantarum; Lactococcus lactis; Listeria innocua; Listeria monocytogenes; Oceanobacillus iheyensis; Paracoccus zeaxanthinifaciens; Pseudomonas mevalonii; Staphylococcus aureus; Staphylococcus epidermidis; Staphylococcus haemolyticus; Streptococcus agalactiae; Streptomyces griseolosporeus; Streptococcus mutans; Streptococcus pneumoniae; Streptococcus pyogenes; Thermoplasma acidophilum; Thermoplasma volcanium; Vibrio cholerae; Vibrio parahaemolyticus; and Vibrio vulnificus.

Host cells containing the polynucleotides of interest can be cultured in conventional nutrient media (e.g., Ham's nutrient mixture) modified as appropriate for activating promoters, selecting transformants or amplifying genes. The culture conditions, such as temperature, pH and the like, are those previously used with the host cell selected for expression, and will be apparent to the ordinarily skilled artisan. Cells are typically harvested by centrifugation, disrupted by physical or chemical means, and the resulting crude extract retained for further purification. For compositions secreted by the host cells, supernatant from centrifugation is separated and retained for further purification. Microbial cells employed in expression of proteins can be disrupted by any convenient method, including freeze-thaw cycling, sonication, mechanical disruption, or use of cell lysing agents, all of which are well known to those skilled in the art. Embodiments that involve cell lysis may entail use of a buffer that contains protease inhibitors that limit degradation after expression of the chimeric DNA molecule. Suitable protease inhibitors include, but are not limited to leupeptin, pepstatin or aprotinin The supernatant then may be precipitated in successively increasing concentrations of saturated ammonium sulfate.

Gene expression may be measured in a sample directly, for example, by conventional Southern blotting, Northern blotting to quantitate the transcription of mRNA [Thomas, Proc. Natl. Acad. Sci. USA, 77:5201-5205 (1980)], dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological of fluorescent methods, such as immunohistochemical staining of cells or tissue sections and assay of cell culture or body fluids or the detection of selectable markers, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence BP polypeptide or against a synthetic peptide based on the DNA sequences provided herein or against exogenous sequence fused to BF and encoding a specific antibody epitope. Examples of selectable markers are well known to one of skill in the art and include reporters such as enhanced green fluorescent protein (EGFP), beta-galactosidase (β-gal) or chloramphenicol acetyltransferase (CAT).

Expressed BFXTEN polypeptide product(s) may be purified via methods known in the art or by methods disclosed herein. Procedures such as gel filtration, affinity purification, salt fractionation, ion exchange chromatography, size exclusion chromatography, hydroxyapatite adsorption chromatography, hydrophobic interaction chromatography and gel electrophoresis may be used; each tailored to recover and purify the fusion protein produced by the respective host cells. Some expressed BFXTEN may require refolding during isolation and purification. Methods of purification are described in Robert K. Scopes, Protein Purification: Principles and Practice, Charles R. Castor (ed.), Springer-Verlag 1994, and Sambrook, et al., supra. Multi-step purification separations are also described in Baron, et al., Crit. Rev. Biotechnol. 10:179-90 (1990) and Below, et al., J. Chromatogr. A. 679:67-83 (1994).

### VII). Pharmaceutical Compositions

The present invention provides pharmaceutical compositions comprising BFXTEN. In one embodiment, the pharmaceutical composition comprises the BFXTEN fusion protein or, in the case of a combination BXTEN, the first and second fusion proteins, and at least one pharmaceutically acceptable carrier. BFXTEN polypeptides of the present invention can be formulated according to known methods to prepare pharmaceutically useful compositions, whereby the polypeptide is combined in admixture with a pharmaceutically acceptable carrier vehicle, such as sterile or aqueous solutions, pharmaceutically acceptable suspensions and emulsions. Examples of non-aqueous solvents include propyl ethylene glycol, polyethylene glycol and vegetable oils. Therapeutic formulations are prepared for storage by mixing the active ingredient having the desired degree of purity with optional physiologically acceptable carriers, excipients or stabilizers, as described in Remington's Pharmaceutical Sciences 16th edition, Osol, A. Ed. (1980), in the form of lyophilized formulations or aqueous solutions.

The pharmaceutical compositions can be administered orally, intranasally, parenterally or by inhalation therapy, and may take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition, the pharmaceutical compositions can also contain other pharmaceutically active compounds or a plurality of compounds of the invention.

More particularly, the present pharmaceutical compositions may be administered for therapy by any suitable route including oral, rectal, nasal, topical (including transdermal, aerosol, buccal and sublingual), vaginal, parenteral (including subcutaneous, subcutaneous by infusion pump, intramuscular, intravenous and intradermal), intravitreal, and pulmonary. It will also be appreciated that the preferred route will vary with the condition and age of the recipient, and the disease being treated.

In one embodiment, the pharmaceutical composition is administered subcutaneously. In this embodiment, the composition may be supplied as a lyophilized powder to be reconstituted prior to administration. The composition may also be supplied in a liquid form, which can be administered directly to a patient. In one embodiment, the composition is supplied as a liquid in a pre-filled syringe such that a patient can easily self-administer the composition.

Extended release formulations useful in the present invention may be oral formulations comprising a matrix and a coating composition. Suitable matrix materials may include waxes (e.g., carnauba, bees wax, paraffin wax, ceresine, shellac wax, fatty acids, and fatty alcohols), oils, hardened oils or fats (e.g., hardened rapeseed oil, castor oil, beef tallow, palm oil, and soya bean oil), and polymers (e.g., hydroxypropyl cellulose, polyvinylpyrrolidone, hydroxypropyl methyl cellulose, and polyethylene glycol). Other suitable matrix tabletting materials are microcrystalline cellulose, powdered cellulose, hydroxypropyl cellulose, ethyl cellulose, with other carriers, and fillers. Tablets may also contain granulates, coated powders, or pellets. Tablets may also be multi-layered. Multi-layered tablets are especially preferred when the active ingredients have markedly different pharmacokinetic profiles. Optionally, the finished tablet may be coated or uncoated.

The coating composition may comprise an insoluble matrix polymer and/or a water soluble material. Water soluble materials can be polymers such as polyethylene glycol, hydroxypropyl cellulose, hydroxypropyl methyl cellulose, polyvinylpyrrolidone, polyvinyl alcohol, or monomeric materials such as sugars (e.g., lactose, sucrose, fructose, mannitol and the like), salts (e.g., sodium chloride, potassium chloride and the like), organic acids (e.g., fumaric acid, succinic acid, lactic acid, and tartaric acid), and mixtures thereof. Optionally, an enteric polymer may be incorporated into the coating composition. Suitable enteric polymers include hydroxypropyl methyl cellulose, acetate succinate, hydroxypropyl methyl cellulose, phthalate, polyvinyl acetate phthalate, cellulose acetate phthalate, cellulose acetate trimellitate, shellac, zein, and polymethacrylates containing carboxyl groups. The coating composition may be plasticised by adding suitable plasticisers such as, for example, diethyl phthalate, citrate esters, polyethylene glycol, glycerol, acetylated glycerides, acetylated citrate esters, dibutylsebacate, and castor oil. The coating composition may also include a filler, which can be an insoluble material such as silicon dioxide, titanium dioxide, talc, kaolin, alumina, starch, powdered cellulose, MCC, or polacrilin potassium. The coating composition may be applied as a solution or latex in organic solvents or aqueous solvents or mixtures thereof. Solvents such as water, lower alcohol, lower chlorinated hydrocarbons, ketones, or mixtures thereof may be used.

The compositions of the invention may be formulated using a variety of excipients. Suitable excipients include microcrystalline cellulose (e.g. Avicel PH102, Avicel PH101), polymethacrylate, poly(ethyl acrylate, methyl methacrylate, trimethylammonioethyl methacrylate chloride) (such as Eudragit RS-30D), hydroxypropyl methylcellulose (Methocel K100M, Premium CR Methocel K100M, Methocel E5, Opadry®), magnesium stearate, talc, triethyl citrate, aqueous ethylcellulose dispersion (Surelease®), and protamine sulfate. The slow release agent may also comprise a carrier, which can comprise, for example, solvents, dispersion media, coatings, antibacterial and antifungal agents, isotonic and absorption delaying agents. Pharmaceutically acceptable salts can also be used in these slow release agents, for example, mineral salts such as hydrochlorides, hydrobromides, phosphates, or sulfates, as well as the salts of organic acids such as acetates, propionates, malonates, or benzoates. The composition may also contain liquids, such as water, saline, glycerol, and ethanol, as well as substances such as wetting agents, emulsifying agents, or pH buffering agents. Liposomes may also be used as a carrier.

In another embodiment, the compositions of the present invention are encapsulated in liposomes, which have demonstrated utility in delivering beneficial active agents in a controlled manner over prolonged periods of time. Liposomes are closed bilayer membranes containing an entrapped aqueous volume. Liposomes may also be unilamellar vesicles possessing a single membrane bilayer or multilamellar vesicles with multiple membrane bilayers, each separated from the next by an aqueous layer. The structure of the resulting membrane bilayer is such that the hydrophobic (non-polar) tails of the lipid are oriented toward the center of the bilayer while the hydrophilic (polar) heads orient towards the aqueous phase. In one embodiment, the liposome may be coated with a flexible water soluble polymer that avoids uptake by the organs of the mononuclear phagocyte system, primarily the liver and spleen. Suitable hydrophilic polymers for surrounding the liposomes include, without limitation, PEG, polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose hydroxyethylcellulose, polyethyleneglycol, polyaspartamide and hydrophilic peptide sequences as described in U.S. Pat. Nos. 6,316,024; 6,126,966; 6,056,973; 6,043,094, the contents of which are incorporated by reference in their entirety.

Liposomes may be comprised of any lipid or lipid combination known in the art. For example, the vesicle-forming lipids may be naturally-occurring or synthetic lipids, including phospholipids, such as phosphatidylcholine, phosphatidylethanolamine, phosphatidic acid, phosphatidylserine, phasphatidylglycerol, phosphatidylinositol, and sphingomyelin as disclosed in U.S. Pat. Nos. 6,056,973 and 5,874,104. The vesicle-forming lipids may also be glycolipids, cerebrosides, or cationic lipids, such as 1,2-dioleyloxy-3-(trimethylamino) propane (DOTAP); N-[1-(2,3,-ditetradecyloxy)propyl]-N,N-dimethyl-N-hydroxyethylammonium bromide (DMRIE); N-[1[(2,3,-dioleyloxy)propyl]-N,N-dimethyl-N-hydroxy ethylammonium bromide (DORIE); N-[1-(2,3-dioleyloxy)propyl]-N,N,N-trimethylammonium chloride (DOTMA); 3 [N—(N′,N′-dimethylaminoethane) carbamoly]cholesterol (DC-Chol); or dimethyldioctadecylammonium (DDAB) also as disclosed in U.S. Pat. No. 6,056,973. Cholesterol may also be present in the proper range to impart stability to the vesicle as disclosed in U.S. Pat. Nos. 5,916,588 and 5,874,104.

Additional liposomal technologies are described in U.S. Pat. Nos. 6,759,057; 6,406,713; 6,352,716; 6,316,024; 6,294,191; 6,126,966; 6,056,973; 6,043,094; 5,965,156; 5,916,588; 5,874,104; 5,215,680; and 4,684,479, the contents of which are incorporated herein by reference. These describe liposomes and lipid-coated microbubbles, and methods for their manufacture. Thus, one skilled in the art, considering both the disclosure of this invention and the disclosures of these other patents could produce a liposome for the extended release of the polypeptides of the present invention.

For liquid formulations, a desired property is that the formulation be supplied in a form that can pass through a 25, 28, 30, 31, 32 gauge needle for intravenous, intramuscular, intraarticular, or subcutaneous administration.

Administration via transdermal formulations can be performed using methods also known in the art, including those described generally in, e.g., U.S. Pat. Nos. 5,186,938 and 6,183,770, 4,861,800, 6,743,211, 6,945,952, 4,284,444, and WO 89/09051, incorporated herein by reference in their entireties. A transdermal patch is a particularly useful embodiment with polypeptides having absorption problems. Patches can be made to control the release of skin-permeable active ingredients over a 12 hour, 24 hour, 3 day, and 7 day period. In one example, a 2-fold daily excess of a polypeptide of the present invention is placed in a non-volatile fluid. The compositions of the invention are provided in the form of a viscous, non-volatile liquid. The penetration through skin of specific formulations may be measures by standard methods in the art (for example, Franz et al., J. Invest. Derm. 64:194-195 (1975)). Examples of suitable patches are passive transfer skin patches, iontophoretic skin patches, or patches with microneedles such as Nicoderm.

In other embodiments, the composition may be delivered via intranasal, buccal, or sublingual routes to the brain to enable transfer of the active agents through the olfactory passages into the CNS and reducing the systemic administration. Devices commonly used for this route of administration are included in U.S. Pat. No. 6,715,485. Compositions delivered via this route may enable increased CNS dosing or reduced total body burden reducing systemic toxicity risks associated with certain drugs. Preparation of a pharmaceutical composition for delivery in a subdermally implantable device can be performed using methods known in the art, such as those described in, e.g., U.S. Pat. Nos. 3,992,518; 5,660,848; and 5,756,115.

Osmotic pumps may be used as slow release agents in the form of tablets, pills, capsules or implantable devices. Osmotic pumps are well known in the art and readily available to one of ordinary skill in the art from companies experienced in providing osmotic pumps for extended release drug delivery. Examples are ALZA's DUROS™; ALZA's OROS™; Osmotica Pharmaceutical's Osmodex™ system; Shire Laboratories' EnSoTrol™ system; and Alzet™. Patents that describe osmotic pump technology are U.S. Pat. Nos. 6,890,918; 6,838,093; 6,814,979; 6,713,086; 6,534,090; 6,514,532; 6,361,796; 6,352,721; 6,294,201; 6,284,276; 6,110,498; 5,573,776; 4,200,0984; and 4,088,864, the contents of which are incorporated herein by reference. One skilled in the art, considering both the disclosure of this invention and the disclosures of these other patents could produce an osmotic pump for the extended release of the polypeptides of the present invention.

Syringe pumps may also be used as slow release agents. Such devices are described in U.S. Pat. Nos. 4,976,696; 4,933,185; 5,017,378; 6,309,370; 6,254,573; 4,435,173; 4,398,908; 6,572,585; 5,298,022; 5,176,502; 5,492,534; 5,318,540; and 4,988,337, the contents of which are incorporated herein by reference. One skilled in the art, considering both the disclosure of this invention and the disclosures of these other patents could produce a syringe pump for the extended release of the compositions of the present invention.

### VII). Pharmaceutical Kits

In another aspect, the invention provides a kit to facilitate the use of the BFXTEN polypeptides. The kit comprises the pharmaceutical composition provided herein, a label identifying the pharmaceutical composition, and an instruction for storage, reconstitution and/or administration of the pharmaceutical compositions to a subject. In some embodiment, the kit comprises, preferably: (a) an amount of a BFXTEN fusion protein composition sufficient to treat a disease, condition or disorder upon administration to a subject in need thereof; and (b) an amount of a pharmaceutically acceptable carrier; together in a formulation ready for injection or for reconstitution with sterile water, buffer, or dextrose; together with a label identifying the BFXTEN drug and storage and handling conditions, and a sheet of the approved indications for the drug, instructions for the reconstitution and/or administration of the BFXTEN drug for the use for the prevention and/or treatment of a approved indication, appropriate dosage and safety information, and information identifying the lot and expiration of the drug. In another embodiment of the foregoing, the kit can comprise a second container that can carry a suitable diluent for the BFXTEN composition, the use of which will provide the user with the appropriate concentration of BFXTEN to be delivered to the subject.

### EXAMPLES

### Example 1

### Construction of XTEN_AD36 Motif Segments

The following example describes the construction of a collection of codon-optimized genes encoding motif sequences of 36 amino acids. As a first step, a stuffer vector pCW0359 was constructed based on a pET vector and that includes a T7 promoter. pCW0359 encodes a cellulose binding domain (CBD) and a TEV protease recognition site followed by a stuffer sequence that is flanked by BsaI, BbsI, and KpnI sites. The BsaI and BbsI sites were inserted such that they generate compatible overhangs after digestion. The stuffer sequence is followed by a truncated version of the GFP gene and a His tag. The stuffer sequence contains stop codons and thus E. coli cells carrying the stuffer plasmid pCW0359 form non-fluorescent colonies. The stuffer vector pCW0359 was digested with BsaI and KpnI to remove the stuffer segment and the resulting vector fragment was isolated by agarose gel purification. The sequences were designated XTEN_AD36, reflecting the AD family of motifs. Its segments have the amino acid sequence [X]₃ where X is a 12mer peptide with the sequences: GESPGGSSGSES (SEQ ID NO: 184), GSEGSSGPGESS (SEQ ID NO: 185), GSSESGSSEGGP (SEQ ID NO: 186), or GSGGEPSESGSS (SEQ ID NO: 187). The insert was obtained by annealing the following pairs of phosphorylated synthetic oligonucleotide pairs:

(SEQ ID NO: 188)
AD1for: AGGTGAATCTCCDGGTGGYTCYAGCGGTTCYGARTC
(SEQ ID NO: 189)
AD1rev: ACCTGAYTCRGAACCGCTRGARCCACCHGGAGATTC
(SEQ ID NO: 190)
AD2for: AGGTAGCGAAGGTTCTTCYGGTCCDGGYGARTCYTC
(SEQ ID NO: 191)
AD2rev: ACCTGARGAYTCRCCHGGACCRGAAGAACCTTCGCT
(SEQ ID NO: 192)
AD3for: AGGTTCYTCYGAAAGCGGTTCTTCYGARGGYGGTCC
(SEQ ID NO: 193)
AD3rev: ACCTGGACCRCCYTCRGAAGAACCGCTTTCRGARGA
(SEQ ID NO: 194)
AD4for: AGGTTCYGGTGGYGAACCDTCYGARTCTGGTAGCTC

We also annealed the phosphorylated oligonucleotide 3KpnIstopperFor: AGGTTCGTCTTCACTCGAGGGTAC (SEQ ID NO: 195) and the non-phosphorylated oligonucleotide pr_{—}3KpnIstopperRev: CCTCGAGTGAAGACGA (SEQ ID NO: 196). The annealed oligonucleotide pairs were ligated, which resulted in a mixture of products with varying length that represents the varying number of 12mer repeats ligated to one BbsI/KpnI segment. The products corresponding to the length of 36 amino acids were isolated from the mixture by preparative agarose gel electrophoresis and ligated into the BsaI/KpnI digested stuffer vector pCW0359. Most of the clones in the resulting library designated LCW0401 showed green fluorescence after induction, which shows that the sequence of XTEN_AD36 had been ligated in frame with the GFP gene and that most sequences of XTEN_AD36 had good expression levels.

We screened 96 isolates from library LCW0401 for high level of fluorescence by stamping them onto agar plate containing IPTG. The same isolates were evaluated by PCR and 48 isolates were identified that contained segments with 36 amino acids as well as strong fluorescence. These isolates were sequenced and 39 clones were identified that contained correct XTEN_AD36 segments. Nucleotide and amino acid sequences for these segments are listed in Table 9.

TABLE 9
DNA and Amino Acid Sequences for 36-mer motifs

| | | SEQ | | SEQ |
|---|---|---|---|---|
| | | ID | | ID |
| File name | Amino acid sequence | NO: | Nucleotide sequence | NO: |
| LCW0401_001_{—} | GSGGEPSESGSSGESPG | 197 | GGTTCTGGTGGCGAACCGTCCGAGTCTGGTAG | 235 |
| GFP-N_A01.ab1 | GSSGSESGESPGGSSGS | | CTCAGGTGAATCTCCGGGTGGCTCTAGCGGTT | |
| | ES | | CCGAGTCAGGTGAATCTCCTGGTGGTTCCAGC | |
| | | | GGTTCCGAGTCA | |
| LCW0401_002_{—} | GSEGSSGPGESSGESPG | 198 | GGTAGCGAAGGTTCTTCTGGTCCTGGCGAGTC | 236 |
| GFP-N_B01.ab1 | GSSGSESGSSESGSSEG | | TTCAGGTGAATCTCCTGGTGGTTCCAGCGGTT | |
| | GP | | CTGAATCAGGTTCCTCCGAAAGCGGTTCTTCC | |
| | | | GAGGGCGGTCCA | |
| LCW0401_003_{—} | GSSESGSSEGGPGSSES | 199 | GGTTCCTCTGAAAGCGGTTCTTCCGAAGGTGG | 237 |
| GFP-N_C01.ab1 | GSSEGGPGESPGGSSGS | | TCCAGGTTCCTCTGAAAGCGGTTCTTCTGAGG | |
| | ES | | GTGGTCCAGGTGAATCTCCGGGTGGCTCCAGC | |
| | | | GGTTCCGAGTCA | |
| LCW0401_004_{—} | GSGGEPSESGSSGSSES | 200 | GGTTCCGGTGGCGAACCGTCTGAATCTGGTAG | 238 |
| GFP-N_D01.ab1 | GSSEGGPGSGGEPSESG | | CTCAGGTTCTTCTGAAAGCGGTTCTTCCGAGG | |
| | SS | | GTGGTCCAGGTTCTGGTGGTGAACCTTCCGAG | |
| | | | TCTGGTAGCTCA | |
| LCW0401_007_{—} | GSSESGSSEGGPGSEGS | 201 | GGTTCTTCCGAAAGCGGTTCTTCTGAGGGTGG | 239 |
| GFP-N_F01.ab1 | SGPGESSGSEGSSGPGE | | TCCAGGTAGCGAAGGTTCTTCCGGTCCAGGTG | |
| | SS | | AGTCTTCAGGTAGCGAAGGTTCTTCTGGTCCT | |
| | | | GGTGAATCTTCA | |
| LCW0401_ 008_{—} | GSSESGSSEGGPGESPG | 202 | GGTTCCTCTGAAAGCGGTTCTTCCGAGGGTGG | 240 |
| GFP-N_G01.ab1 | GSSGSESGSEGSSGPGE | | TCCAGGTGAATCTCCAGGTGGTTCCAGCGGTT | |
| | SS | | CTGAGTCAGGTAGCGAAGGTTCTTCTGGTCCA | |
| | | | GGTGAATCCTCA | |
| LCW0401_012_{—} | GSGGEPSESGSSGSGGE | 203 | GGTTCTGGTGGTGAACCGTCTGAGTCTGGTAG | 241 |
| GFP-N_H01.ab1 | PSESGSSGSEGSSGPGE | | CTCAGGTTCCGGTGGCGAACCATCCGAATCTG | |
| | SS | | GTAGCTCAGGTAGCGAAGGTTCTTCCGGTCCA | |
| | | | GGTGAGTCTTCA | |
| LCW0401_015_{—} | GSSESGSSEGGPGSEGS | 204 | GGTTCTTCCGAAAGCGGTTCTTCCGAAGGCGG | 242 |
| GFP-N_A02.ab1 | SGPGESSGESPGGSSGS | | TCCAGGTAGCGAAGGTTCTTCTGGTCCAGGCG | |
| | ES | | AATCTTCAGGTGAATCTCCTGGTGGCTCCAGC | |
| | | | GGTTCTGAGTCA | |
| LCW0401_016_{—} | GSSESGSSEGGPGSSES | 205 | GGTTCCTCCGAAAGCGGTTCTTCTGAGGGCGG | 243 |
| GFP-N_B02.ab1 | GSSEGGPGSSESGSSEG | | TCCAGGTTCCTCCGAAAGCGGTTCTTCCGAGG | |
| | GP | | GCGGTCCAGGTTCTTCTGAAAGCGGTTCTTCC | |
| | | | GAGGGCGGTCCA | |
| LCW0401_020__ | GSGGEPSESGSSGSEGS | 206 | GGTTCCGGTGGCGAACCGTCCGAATCTGGTAG | 244 |
| GFP-N_E02.ab1 | SGPGESSGSSESGSSEG | | CTCAGGTAGCGAAGGTTCTTCTGGTCCAGGCG | |
| | GP | | AATCTTCAGGTTCCTCTGAAAGCGGTTCTTCT | |
| | | | GAGGGCGGTCCA | |
| LCW0401_022_{—} | GSGGEPSESGSSGSSES | 207 | GGTTCTGGTGGTGAACCGTCCGAATCTGGTAG | 245 |
| GFP-N_F02.ab1 | GSSEGGPGSGGEPSESG | | CTCAGGTTCTTCCGAAAGCGGTTCTTCTGAAG | |
| | SS | | GTGGTCCAGGTTCCGGTGGCGAACCTTCTGAA | |
| | | | TCTGGTAGCTCA | |
| LCW0401_024_{—} | GSGGEPSESGSSGSSES | 208 | GGTTCTGGTGGCGAACCGTCCGAATCTGGTAG | 246 |
| GFP-N_G02.ab1 | GSSEGGPGESPGGSSGS | | CTCAGGTTCCTCCGAAAGCGGTTCTTCTGAAG | |
| | ES | | GTGGTCCAGGTGAATCTCCAGGTGGTTCTAGC | |
| | | | GGTTCTGAATCA | |
| LCW0401_026_{—} | GSGGEPSESGSSGESPG | 209 | GGTTCTGGTGGCGAACCGTCTGAGTCTGGTAG | 247 |
| GFP-N_H02.ab1 | GSSGSESGSEGSSGPGE | | CTCAGGTGAATCTCCTGGTGGCTCCAGCGGTT | |
| | SS | | CTGAATCAGGTAGCGAAGGTTCTTCTGGTCCT | |
| | | | GGTGAATCTTCA | |
| LCW0401_027_{—} | GSGGEPSESGSSGESPG | 210 | GGTTCCGGTGGCGAACCTTCCGAATCTGGTAG | 248 |
| GFP-N_A03.ab1 | GSSGSESGSGGEPSESG | | CTCAGGTGAATCTCCGGGTGGTTCTAGCGGTT | |
| | SS | | CTGAGTCAGGTTCTGGTGGTGAACCTTCCGAG | |
| | | | TCTGGTAGCTCA | |
| LCW0401_028_{—} | GSSESGSSEGGPGSSES | 211 | GGTTCCTCTGAAAGCGGTTCTTCTGAGGGCGG | 249 |
| GFP-N_B03.ab1 | GSSEGGPGSSESGSSEG | | TCCAGGTTCTTCCGAAAGCGGTTCTTCCGAGG | |
| | GP | | GCGGTCCAGGTTCTTCCGAAAGCGGTTCTTCT | |
| | | | GAAGGCGGTCCA | |
| LCW0401_030_{—} | GESPGGSSGSESGSEGS | 212 | GGTGAATCTCCGGGTGGCTCCAGCGGTTCTGA | 250 |
| GFP-N_CO3.ab1 | SGPGESSGSEGSSGPGE | | GTCAGGTAGCGAAGGTTCTTCCGGTCCGGGTG | |
| | SS | | AGTCCTCAGGTAGCGAAGGTTCTTCCGGTCCT | |
| | | | GGTGAGTCTTCA | |
| LCW0401_ 031_{—} | GSGGEPSESGSSGSGGE | 213 | GGTTCTGGTGGCGAACCTTCCGAATCTGGTAG | 251 |
| GFP-N_D03.ab1 | PSESGSSGSSESGSSEG | | CTCAGGTTCCGGTGGTGAACCTTCTGAATCTG | |
| | GP | | GTAGCTCAGGTTCTTCTGAAAGCGGTTCTTCC | |
| | | | GAGGGCGGTCCA | |
| LCW0401_ 033_{—} | GSGGEPSESGSSGSGGE | 214 | GGTTCCGGTGGTGAACCTTCTGAATCTGGTAG | 252 |
| GFP-N_E03.ab1 | PSESGSSGSGGEPSESG | | CTCAGGTTCCGGTGGCGAACCATCCGAGTCTG | |
| | SS | | GTAGCTCAGGTTCCGGTGGTGAACCATCCGAG | |
| | | | TCTGGTAGCTCA | |
| LCW0401_037_{—} | GSGGEPSESGSSGSSES | 215 | GGTTCCGGTGGCGAACCTTCTGAATCTGGTAG | 253 |
| GFP-N_F03.ab1 | GSSEGGPGSEGSSGPGE | | CTCAGGTTCCTCCGAAAGCGGTTCTTCTGAGG | |
| | SS | | GCGGTCCAGGTAGCGAAGGTTCTTCTGGTCCG | |
| | | | GGCGAGTCTTCA | |
| LCW0401_038_{—} | GSGGEPSESGSSGSEGS | 216 | GGTTCCGGTGGTGAACCGTCCGAGTCTGGTAG | 254 |
| GFP-N_G03.ab1 | SGPGESSGSGGEPSESG | | CTCAGGTAGCGAAGGTTCTTCTGGTCCGGGTG | |
| | SS | | AGTCTTCAGGTTCTGGTGGCGAACCGTCCGAA | |
| | | | TCTGGTAGCTCA | |
| LCW0401_039_{—} | GSGGEPSESGSSGESPG | 217 | GGTTCTGGTGGCGAACCGTCCGAATCTGGTAG | 255 |
| GFP-N_H03.ab1 | GSSGSESGSGGEPSESG | | CTCAGGTGAATCTCCTGGTGGTTCCAGCGGTT | |
| | SS | | CCGAGTCAGGTTCTGGTGGCGAACCTTCCGAA | |
| | | | TCTGGTAGCTCA | |
| LCW0401_040_{—} | GSSESGSSEGGPGSGGE | 218 | GGTTCTTCCGAAAGCGGTTCTTCCGAGGGCGG | 256 |
| GFP-N_A04.ab1 | PSESGSSGSSESGSSEG | | TCCAGGTTCCGGTGGTGAACCATCTGAATCTG | |
| | GP | | GTAGCTCAGGTTCTTCTGAAAGCGGTTCTTCT | |
| | | | GAAGGTGGTCCA | |
| LCW0401_042_{—} | GSEGSSGPGESSGESPG | 219 | GGTAGCGAAGGTTCTTCCGGTCCTGGTGAGTC | 257 |
| GFP-N_C04.ab1 | GSSGSESGSEGSSGPGE | | TTCAGGTGAATCTCCAGGTGGCTCTAGCGGTT | |
| | SS | | CCGAGTCAGGTAGCGAAGGTTCTTCTGGTCCT | |
| | | | GGCGAGTCCTCA | |
| LCW0401_046_{—} | GSSESGSSEGGPGSSES | 220 | GGTTCCTCTGAAAGCGGTTCTTCCGAAGGCGG | 258 |
| GFP-N_D04.ab1 | GSSEGGPGSSESGSSEG | | TCCAGGTTCTTCCGAAAGCGGTTCTTCTGAGG | |
| | GP | | GCGGTCCAGGTTCCTCCGAAAGCGGTTCTTCT | |
| | | | GAGGGTGGTCCA | |
| LCW0401_047_{—} | GSGGEPSESGSSGESPG | 221 | GGTTCTGGTGGCGAACCTTCCGAGTCTGGTAG | 259 |
| GFP-N_E04.ab1 | GSSGSESGESPGGSSGS | | CTCAGGTGAATCTCCGGGTGGTTCTAGCGGTT | |
| | ES | | CCGAGTCAGGTGAATCTCCGGGTGGTTCCAGC | |
| | | | GGTTCTGAGTCA | |
| LCW0401_051_{—} | GSGGEPSESGSSGSEGS | 222 | GGTTCTGGTGGCGAACCATCTGAGTCTGGTAG | 260 |
| GFP-N_F04.ab1 | SGPGESSGESPGGSSGS | | CTCAGGTAGCGAAGGTTCTTCCGGTCCAGGCG | |
| | ES | | AGTCTTCAGGTGAATCTCCTGGTGGCTCCAGC | |
| | | | GGTTCTGAGTCA | |
| LCW0401_053_{—} | GESPGGSSGSESGESPG | 223 | GGTGAATCTCCTGGTGGTTCCAGCGGTTCCGA | 261 |
| GFP-N_H04.ab1 | GSSGSESGESPGGSSGS | | GTCAGGTGAATCTCCAGGTGGCTCTAGCGGTT | |
| | ES | | CCGAGTCAGGTGAATCTCCTGGTGGTTCTAGC | |
| | | | GGTTCTGAATCA | |
| LCW0401_054_{—} | GSEGSSGPGESSGSEGS | 224 | GGTAGCGAAGGTTCTTCCGGTCCAGGTGAATC | 262 |
| GFP-N_A05.ab1 | SGPGESSGSGGEPSESG | | TTCAGGTAGCGAAGGTTCTTCTGGTCCTGGTG | |
| | SS | | AATCCTCAGGTTCCGGTGGCGAACCATCTGAA | |
| | | | TCTGGTAGCTCA | |
| LCW0401_059_{—} | GSGGEPSESGSSGSEGS | 225 | GGTTCTGGTGGCGAACCATCCGAATCTGGTAG | 263 |
| GFP-N_D05.ab1 | SGPGESSGESPGGSSGS | | CTCAGGTAGCGAAGGTTCTTCTGGTCCTGGCG | |
| | ES | | AATCTTCAGGTGAATCTCCAGGTGGCTCTAGC | |
| | | | GGTTCCGAATCA | |
| LCW0401_060_{—} | GSGGEPSESGSSGSSES | 226 | GGTTCCGGTGGTGAACCGTCCGAATCTGGTAG | 264 |
| GFP-N_E05.ab1 | GSSEGGPGSGGEPSESG | | CTCAGGTTCCTCTGAAAGCGGTTCTTCCGAGG | |
| | SS | | GTGGTCCAGGTTCCGGTGGTGAACCTTCTGAG | |
| | | | TCTGGTAGCTCA | |
| LCW0401_061_{—} | GSSESGSSEGGPGSGGE | 227 | GGTTCCTCTGAAAGCGGTTCTTCTGAGGGCGG | 265 |
| GFP-N_F05.ab1 | PSESGSSGSEGSSGPGE | | TCCAGGTTCTGGTGGCGAACCATCTGAATCTG | |
| | SS | | GTAGCTCAGGTAGCGAAGGTTCTTCCGGTCCG | |
| | | | GGTGAATCTTCA | |
| LCW0401_063_{—} | GSGGEPSESGSSGSEGS | 228 | GGTTCTGGTGGTGAACCGTCCGAATCTGGTAG | 266 |
| GFP-N_H05.ab1 | SGPGESSGSEGSSGPGE | | CTCAGGTAGCGAAGGTTCTTCTGGTCCTGGCG | |
| | SS | | AGTCTTCAGGTAGCGAAGGTTCTTCTGGTCCT | |
| | | | GGTGAATCTTCA | |
| LCW0401_066_{—} | GSGGEPSESGSSGSSES | 229 | GGTTCTGGTGGCGAACCATCCGAGTCTGGTAG | 267 |
| GFP-N_B06.ab1 | GSSEGGPGSGGEPSESG | | CTCAGGTTCTTCCGAAAGCGGTTCTTCCGAAG | |
| | SS | | GCGGTCCAGGTTCTGGTGGTGAACCGTCCGAA | |
| | | | TCTGGTAGCTCA | |
| LCW0401_067_{—} | GSGGEPSESGSSGESPG | 230 | GGTTCCGGTGGCGAACCTTCCGAATCTGGTAG | 268 |
| GFP-N_C06.ab1 | GSSGSESGESPGGSSGS | | CTCAGGTGAATCTCCGGGTGGTTCTAGCGGTT | |
| | ES | | CCGAATCAGGTGAATCTCCAGGTGGTTCTAGC | |
| | | | GGTTCCGAATCA | |
| LCW0401_069_{—} | GSGGEPSESGSSGSGGE | 231 | GGTTCCGGTGGTGAACCATCTGAGTCTGGTAG | 269 |
| GFP-N_D06.ab1 | PSESGSSGESPGGSSGS | | CTCAGGTTCCGGTGGCGAACCGTCCGAGTCTG | |
| | ES | | GTAGCTCAGGTGAATCTCCGGGTGGTTCCAGC | |
| | | | GGTTCCGAATCA | |
| LCW0401_070_{—} | GSEGSSGPGESSGSSES | 232 | GGTAGCGAAGGTTCTTCTGGTCCGGGCGAATC | 270 |
| GFP-N_E06.ab1 | GSSEGGPGSEGSSGPGE | | CTCAGGTTCCTCCGAAAGCGGTTCTTCCGAAG | |
| | SS | | GTGGTCCAGGTAGCGAAGGTTCTTCCGGTCCT | |
| | | | GGTGAATCTTCA | |
| LCW0401_078_{—} | GSSESGSSEGGPGESPG | 233 | GGTTCCTCTGAAAGCGGTTCTTCTGAAGGCGG | 271 |
| GFP-N_F06.ab1 | GSSGSESGESPGGSSGS | | TCCAGGTGAATCTCCGGGTGGCTCCAGCGGTT | |
| | ES | | CTGAATCAGGTGAATCTCCTGGTGGCTCCAGC | |
| | | | GGTTCCGAGTCA | |
| LCW0401_079_{—} | GSEGSSGPGESSGSEGS | 234 | GGTAGCGAAGGTTCTTCTGGTCCAGGCGAGTC | 272 |
| GFP-N_G06.ab1 | SGPGESSGSGGEPSESG | | TTCAGGTAGCGAAGGTTCTTCCGGTCCTGGCG | |
| | SS | | AGTCTTCAGGTTCCGGTGGCGAACCGTCCGAA | |
| | | | TCTGGTAGCTCA | |

### Example 2

### Construction of XTEN_AE36 Segments

A codon library encoding XTEN sequences of 36 amino acid length was constructed. The XTEN sequence was designated XTEN_AE36. Its segments have the amino acid sequence [X]₃ where X is a 12mer peptide with the sequence: GSPAGSPTSTEE (SEQ ID NO: 273), GSEPATSGSE TP (SEQ ID NO: 274), GTSESA TPESGP (SEQ ID NO: 275), or GTSTEPSEGSAP (SEQ ID NO: 276). The insert was obtained by annealing the following pairs of phosphorylated synthetic oligonucleotide pairs:

(SEQ ID NO: 277)
AE1for: AGGTAGCCCDGCWGGYTCTCCDACYTCYACYGARGA
(SEQ ID NO: 278)
AE1rev: ACCTTCYTCRGTRGARGTHGGAGARCCWGCHGGGCT
(SEQ ID NO: 279)
AE2for: AGGTAGCGAACCKGCWACYTCYGGYTCTGARACYCC
(SEQ ID NO: 280)
AE2rev: ACCTGGRGTYTCAGARCCRGARGTWGCMGGTTCGCT
(SEQ ID NO: 281)
AE3for: AGGTACYTCTGAAAGCGCWACYCCKGARTCYGGYCC
(SEQ ID NO: 282)
AE3rev: ACCTGGRCCRGAYTCMGGRGTWGCGCTTTCAGARGT
(SEQ ID NO: 283)
AE4for: AGGTACYTCTACYGAACCKTCYGARGGYAGCGCWCC
(SEQ ID NO: 284)
AE4rev: ACCTGGWGCGCTRCCYTCRGAMGGTTCRGTAGARGT

We also annealed the phosphorylated oligonucleotide 3KpnIstopperFor: AGGTTCGTCTTCACTCGAGGGTAC (SEQ ID NO: 285) and the non-phosphorylated oligonucleotide pr_{—}3KpnIstopperRev: CCTCGAGTGAAGACGA (SEQ ID NO: 286). The annealed oligonucleotide pairs were ligated, which resulted in a mixture of products with varying length that represents the varying number of 12mer repeats ligated to one BbsI/KpnI segment. The products corresponding to the length of 36 amino acids were isolated from the mixture by preparative agarose gel electrophoresis and ligated into the BsaI/KpnI digested stuffer vector pCW0359. Most of the clones in the resulting library designated LCW0402 showed green fluorescence after induction which shows that the sequence of XTEN_AE36 had been ligated in frame with the GFP gene and most sequences of XTEN_AE36 show good expression.

We screened 96 isolates from library LCW0402 for high level of fluorescence by stamping them onto agar plate containing IPTG. The same isolates were evaluated by PCR and 48 isolates were identified that contained segments with 36 amino acids as well as strong fluorescence. These isolates were sequenced and 37 clones were identified that contained correct XTEN_AE36 segments. Nucleotide and amino acid sequences for these segments are listed in Table 10.

TABLE 10
DNA and Amino Acid Sequences for 36-mer motifs

| | | SEQ | | SEQ |
|---|---|---|---|---|
| | | ID | | ID |
| File name | Amino acid sequence | NO: | Nucleotide sequence | NO: |
| LCW0402_002_{—} | GSPAGSPTSTEEGT | 287 | GGTAGCCCGGCAGGCTCTCCGACCTCTACTGAGGA | 324 |
| GFP-N_A07.ab1 | SESATPESGPGTST | | AGGTACTTCTGAAAGCGCAACCCCGGAGTCCGGCC | |
| | EPSEGSAP | | CAGGTACCTCTACCGAACCGTCTGAGGGCAGCGCA | |
| | | | CCA | |
| LCW0402_003_{—} | GTSTEPSEGSAPGT | 288 | GGTACTTCTACCGAACCGTCCGAAGGCAGCGCTCC | 325 |
| GFP-N_B07.ab1 | STEPSEGSAPGTST | | AGGTACCTCTACTGAACCTTCCGAGGGCAGCGCTC | |
| | EPSEGSAP | | CAGGTACCTCTACCGAACCTTCTGAAGGTAGCGCA | |
| | | | CCA | |
| LCW0402_004_{—} | GTSTEPSEGSAPGT | 289 | GGTACCTCTACCGAACCGTCTGAAGGTAGCGCACC | 326 |
| GFP-N_C07.ab1 | SESATPESGPGTSE | | AGGTACCTCTGAAAGCGCAACTCCTGAGTCCGGTC | |
| | SATPESGP | | CAGGTACTTCTGAAAGCGCAACCCCGGAGTCTGGC | |
| | | | CCA | |
| LCW0402_005_{—} | GTSTEPSEGSAPGTS | 290 | GGTACTTCTACTGAACCGTCTGAAGGTAGCGCACC | 327 |
| GFP-N_D07.ab1 | ESATPESGPGTSESA | | AGGTACTTCTGAAAGCGCAACCCCGGAATCCGGCC | |
| | TPESGP | | CAGGTACCTCTGAAAGCGCAACCCCGGAGTCCGGC | |
| | | | CCA | |
| LCW0402_006_{—} | GSEPATSGSETPGT | 291 | GGTAGCGAACCGGCAACCTCCGGCTCTGAAACCCC | 328 |
| GFP-N_E07.ab1 | SESATPESGPGSPA | | AGGTACCTCTGAAAGCGCTACTCCTGAATCCGGCC | |
| | GSPTSTEE | | CAGGTAGCCCGGCAGGTTCTCCGACTTCCACTGAG | |
| | | | GAA | |
| LCW0402_008_{—} | GTSESATPESGPGS | 292 | GGTACTTCTGAAAGCGCAACCCCTGAATCCGGTCC | 329 |
| GFP-N_F07.ab1 | EPATSGSETPGTST | | AGGTAGCGAACCGGCTACTTCTGGCTCTGAGACTC | |
| | EPSEGSAP | | CAGGTACTTCTACCGAACCGTCCGAAGGTAGCGCA | |
| | | | CCA | |
| LCW0402_009_{—} | GSPAGSPTSTEEGS | 293 | GGTAGCCCGGCTGGCTCTCCAACCTCCACTGAGGA | 330 |
| GFP-N_G07.ab1 | PAGSPTSTEEGSEP | | AGGTAGCCCGGCTGGCTCTCCAACCTCCACTGAAG | |
| | ATSGSETP | | AAGGTAGCGAACCGGCTACCTCCGGCTCTGAAACT | |
| | | | CCA | |
| LCW0402_011_{—} | GSPAGSPTSTEEGT | 294 | GGTAGCCCGGCTGGCTCTCCTACCTCTACTGAGGA | 331 |
| GFP-N_A08.ab1 | SESATPESGPGTST | | AGGTACTTCTGAAAGCGCTACTCCTGAGTCTGGTC | |
| | EPSEGSAP | | CAGGTACCTCTACTGAACCGTCCGAAGGTAGCGCT | |
| | | | CCA | |
| LCW0402_012_{—} | GSPAGSPTSTEEGS | 295 | GGTAGCCCTGCTGGCTCTCCGACTTCTACTGAGGA | 332 |
| GFP-N_B08.ab1 | PAGSPTSTEEGTST | | AGGTAGCCCGGCTGGTTCTCCGACTTCTACTGAGG | |
| | EPSEGSAP | | AAGGTACTTCTACCGAACCTTCCGAAGGTAGCGCT | |
| | | | CCA | |
| LCW0402_013_{—} | GTSESATPESGPGT | 296 | GGTACTTCTGAAAGCGCTACTCCGGAGTCCGGTCC | 333 |
| GFP-N_C08.ab1 | STEPSEGSAPGTST | | AGGTACCTCTACCGAACCGTCCGAAGGCAGCGCTC | |
| | EPSEGSAP | | CAGGTACTTCTACTGAACCTTCTGAGGGTAGCGCT | |
| | | | CCA | |
| LCW0402_014_{—} | GTSTEPSEGSAPGS | 297 | GGTACCTCTACCGAACCTTCCGAAGGTAGCGCTCC | 334 |
| GFP-N_D08.ab1 | PAGSPTSTEEGTST | | AGGTAGCCCGGCAGGTTCTCCTACTTCCACTGAGG | |
| | EPSEGSAP | | AAGGTACTTCTACCGAACCTTCTGAGGGTAGCGCA | |
| | | | CCA | |
| LCW0402_015_{—} | GSEPATSGSETPGS | 298 | GGTAGCGAACCGGCTACTTCCGGCTCTGAGACTCC | 335 |
| GFP-N_E08.ab1 | PAGSPTSTEEGTSE | | AGGTAGCCCTGCTGGCTCTCCGACCTCTACCGAAG | |
| | SATPESGP | | AAGGTACCTCTGAAAGCGCTACCCCTGAGTCTGGC | |
| | | | CCA | |
| LCW0402_016_{—} | GTSTEPSEGSAPGT | 299 | GGTACTTCTACCGAACCTTCCGAGGGCAGCGCACC | 336 |
| GFP-N_F08.ab1 | SESATPESGPGTSE | | AGGTACTTCTGAAAGCGCTACCCCTGAGTCCGGCC | |
| | SATPESGP | | CAGGTACTTCTGAAAGCGCTACTCCTGAATCCGGT | |
| | | | CCA | |
| LCW0402_020_{—} | GTSTEPSEGSAPGS | 300 | GGTACTTCTACTGAACCGTCTGAAGGCAGCGCACC | 337 |
| GFP-N_G08.ab1 | EPATSGSETPGSPA | | AGGTAGCGAACCGGCTACTTCCGGTTCTGAAACCC | |
| | GSPTSTEE | | CAGGTAGCCCAGCAGGTTCTCCAACTTCTACTGAA | |
| | | | GAA | |
| LCW0402_023_{—} | GSPAGSPTSTEEGT | 301 | GGTAGCCCTGCTGGCTCTCCAACCTCCACCGAAGA | 338 |
| GFP-N_A09.ab1 | SESATPESGPGSEP | | AGGTACCTCTGAAAGCGCAACCCCTGAATCCGGCC | |
| | ATSGSETP | | CAGGTAGCGAACCGGCAACCTCCGGTTCTGAAACC | |
| | | | CCA | |
| LCW0402_024_{—} | GTSESATPESGPGS | 302 | GGTACTTCTGAAAGCGCTACTCCTGAGTCCGGCCC | 339 |
| GFP-N_B09.ab1 | PAGSPTSTEEGSPA | | AGGTAGCCCGGCTGGCTCTCCGACTTCCACCGAGG | |
| | GSPTSTEE | | AAGGTAGCCCGGCTGGCTCTCCAACTTCTACTGAA | |
| | | | GAA | |
| LCW0402_025_{—} | GTSTEPSEGSAPGT | 303 | GGTACCTCTACTGAACCTTCTGAGGGCAGCGCTCC | 340 |
| GFP-N_C09.ab1 | SESATPESGPGTST | | AGGTACTTCTGAAAGCGCTACCCCGGAGTCCGGTC | |
| | EPSEGSAP | | CAGGTACTTCTACTGAACCGTCCGAAGGTAGCGCA | |
| | | | CCA | |
| LCW0402_026_{—} | GSPAGSPTSTEEGT | 304 | GGTAGCCCGGCAGGCTCTCCGACTTCCACCGAGGA | 341 |
| GFP-N_D09.ab1 | STEPSEGSAPGSEP | | AGGTACCTCTACTGAACCTTCTGAGGGTAGCGCTC | |
| | ATSGSETP | | CAGGTAGCGAACCGGCAACCTCTGGCTCTGAAACC | |
| | | | CCA | |
| LCW0402_027_{—} | GSPAGSPTSTEEGT | 305 | GGTAGCCCAGCAGGCTCTCCGACTTCCACTGAGGA | 342 |
| GFP-N_E09.ab1 | STEPSEGSAPGTST | | AGGTACTTCTACTGAACCTTCCGAAGGCAGCGCAC | |
| | EPSEGSAP | | CAGGTACCTCTACTGAACCTTCTGAGGGCAGCGCT | |
| | | | CCA | |
| LCW0402_032_{—} | GSEPATSGSETPGT | 306 | GGTAGCGAACCTGCTACCTCCGGTTCTGAAACCCC | 343 |
| GFP-N_H09.ab1 | SESATPESGPGSPA | | AGGTACCTCTGAAAGCGCAACTCCGGAGTCTGGTC | |
| | GSPTSTEE | | CAGGTAGCCCTGCAGGTTCTCCTACCTCCACTGAG | |
| | | | GAA | |
| LCW0402_034_{—} | GTSESATPESGPGT | 307 | GGTACCTCTGAAAGCGCTACTCCGGAGTCTGGCCC | 344 |
| GFP-N_A10.ab1 | STEPSEGSAPGTST | | AGGTACCTCTACTGAACCGTCTGAGGGTAGCGCTC | |
| | EPSEGSAP | | CAGGTACTTCTACTGAACCGTCCGAAGGTAGCGCA | |
| | | | CCA | |
| LCW0402_036_{—} | GSPAGSPTSTEEGT | 308 | GGTAGCCCGGCTGGTTCTCCGACTTCCACCGAGGA | 345 |
| GFP-N_C10.ab1 | STEPSEGSAPGTST | | AGGTACCTCTACTGAACCTTCTGAGGGTAGCGCTC | |
| | EPSEGSAP | | CAGGTACCTCTACTGAACCTTCCGAAGGCAGCGCT | |
| | | | CCA | |
| LCW0402_039_{—} | GTSTEPSEGSAPGT | 309 | GGTACTTCTACCGAACCGTCCGAGGGCAGCGCTCC | 346 |
| GFP-N_E10.ab1 | STEPSEGSAPGTST | | AGGTACTTCTACTGAACCTTCTGAAGGCAGCGCTC | |
| | EPSEGSAP | | CAGGTACTTCTACTGAACCTTCCGAAGGTAGCGCA | |
| | | | CCA | |
| LCW0402_040_{—} | GSEPATSGSETPGT | 310 | GGTAGCGAACCTGCAACCTCTGGCTCTGAAACCCC | 347 |
| GFP-N_F10.ab1 | SESATPESGPGTST | | AGGTACCTCTGAAAGCGCTACTCCTGAATCTGGCC | |
| | EPSEGSAP | | CAGGTACTTCTACTGAACCGTCCGAGGGCAGCGCA | |
| | | | CCA | |
| LCW0402_041_{—} | GTSTEPSEGSAPGS | 311 | GGTACTTCTACCGAACCGTCCGAGGGTAGCGCACC | 348 |
| GFP-N_G10.ab1 | PAGSPTSTEEGTST | | AGGTAGCCCAGCAGGTTCTCCTACCTCCACCGAGG | |
| | EPSEGSAP | | AAGGTACTTCTACCGAACCGTCCGAGGGTAGCGCA | |
| | | | CCA | |
| LCW0402_050_{—} | GSEPATSGSETPGT | 312 | GGTAGCGAACCGGCAACCTCCGGCTCTGAAACTCC | 349 |
| GFP-N_A11.ab1 | SESATPESGPGSEP | | AGGTACTTCTGAAAGCGCTACTCCGGAATCCGGCC | |
| | ATSGSETP | | CAGGTAGCGAACCGGCTACTTCCGGCTCTGAAACC | |
| | | | CCA | |
| LCW0402_051_{—} | GSEPATSGSETPGT | 313 | GGTAGCGAACCGGCAACTTCCGGCTCTGAAACCCC | 350 |
| GFP-N_B11.ab1 | SESATPESGPGSEP | | AGGTACTTCTGAAAGCGCTACTCCTGAGTCTGGCC | |
| | ATSGSETP | | CAGGTAGCGAACCTGCTACCTCTGGCTCTGAAACC | |
| | | | CCA | |
| LCW0402_059__ | GSEPATSGSETPGS | 314 | GGTAGCGAACCGGCAACCTCTGGCTCTGAAACTCC | 351 |
| GFP-N_E11.ab1 | EPATSGSETPGTST | | AGGTAGCGAACCTGCAACCTCCGGCTCTGAAACCC | |
| | EPSEGSAP | | CAGGTACTTCTACTGAACCTTCTGAGGGCAGCGCA | |
| | | | CCA | |
| LCW0402_060_{—} | GTSESATPESGPGS | 315 | GGTACTTCTGAAAGCGCTACCCCGGAATCTGGCCC | 352 |
| GFP-N_F11.ab1 | EPATSGSETPGSEP | | AGGTAGCGAACCGGCTACTTCTGGTTCTGAAACCC | |
| | ATSGSETP | | CAGGTAGCGAACCGGCTACCTCCGGTTCTGAAACT | |
| | | | CCA | |
| LCW0402_061_{—} | GTSTEPSEGSAPGT | 316 | GGTACCTCTACTGAACCTTCCGAAGGCAGCGCTCC | 353 |
| GFP-N_G11.ab1 | STEPSEGSAPGTSE | | AGGTACCTCTACCGAACCGTCCGAGGGCAGCGCAC | |
| | SATPESGP | | CAGGTACTTCTGAAAGCGCAACCCCTGAATCCGGT | |
| | | | CCA | |
| LCW0402_065_{—} | GSEPATSGSETPGT | 317 | GGTAGCGAACCGGCAACCTCTGGCTCTGAAACCCC | 354 |
| GFP-N_A12.ab1 | SESATPESGPGTSE | | AGGTACCTCTGAAAGCGCTACTCCGGAATCTGGTC | |
| | SATPESGP | | CAGGTACTTCTGAAAGCGCTACTCCGGAATCCGGT | |
| | | | CCA | |
| LCW0402_066_{—} | GSEPATSGSETPGS | 318 | GGTAGCGAACCTGCTACCTCCGGCTCTGAAACTCC | 355 |
| GFP-N_B12.ab1 | EPATSGSETPGTST | | AGGTAGCGAACCGGCTACTTCCGGTTCTGAAACTC | |
| | EPSEGSAP | | GCAGTACCTCTACCGAACCTTCCGAAGGCAGCGCA | |
| | | | CCA | |
| LCW0402_067_{—} | GSEPATSGSETPGT | 319 | GGTAGCGAACCTGCTACTTCTGGTTCTGAAACTCC | 356 |
| GFP-N_C12.ab1 | STEPSEGSAPGSEP | | AGGTACTTCTACCGAACCGTCCGAGGGTAGCGCTC | |
| | ATSGSETP | | CAGGTAGCGAACCTGCTACTTCTGGTTCTGAAACT | |
| | | | CCA | |
| LCW0402_069_{—} | GTSTEPSEGSAPGT | 320 | GGTACCTCTACCGAACCGTCCGAGGGTAGCGCACC | 357 |
| GFP-N_D12.ab1 | STEPSEGSAPGSEP | | AGGTACCTCTACTGAACCGTCTGAGGGTAGCGCTC | |
| | ATSGSETP | | ACGGTAGCGAACCGGCAACCTCCGGTTCTGAAACT | |
| | | | CCA | |
| LCW0402_073_{—} | GTSTEPSEGSAPGS | 321 | GGTACTTCTACTGAACCTTCCGAAGGTAGCGCTCC | 358 |
| GFP-N_F12.ab1 | EPATSGSETPGSPA | | AGGTAGCGAACCTGCTACTTCTGGTTCTGAAACCC | |
| | GSPTSTEE | | CAGGTAGCCCGGCTGGCTCTCCGACCTCCACCGAG | |
| | | | GAA | |
| LCW0402_074_{—} | GSEPATSGSETPGS | 322 | GGTAGCGAACCGGCTACTTCCGGCTCTGAGACTCC | 359 |
| GFP-N_G12.ab1 | PAGSPTSTEEGTSE | | AGGTAGCCCAGCTGGTTCTCCAACCTCTACTGAGG | |
| | SATPESGP | | AAGGTACTTCTGAAAGCGCTACCCCTGAATCTGGT | |
| | | | CCA | |
| LCW0402_075_{—} | GTSESATPESGPGS | 323 | GGTACCTCTGAAAGCGCAACTCCTGAGTCTGGCCC | 360 |
| GFP-N_H12.ab1 | EPATSGSETPGTSE | | AGGTAGCGAACCTGCTACCTCCGGCTCTGAGACTC | |
| | SATPESGP | | CAGGTACCTCTGAAAGCGCAACCCCGGAATCTGGT | |
| | | | CCA | |

### Example 3

### Construction of XTEN_AF36 Segments

A codon library encoding sequences of 36 amino acid length was constructed. The sequences were designated XTEN_AF36. Its segments have the amino acid sequence [X]₃ where X is a 12mer peptide with the sequence: GSTSESPSGTAP (SEQ ID NO: 361), GTSTPESGSASP (SEQ ID NO: 362), GTSPSGESSTAP (SEQ ID NO: 363), or GSTSSTAESPGP (SEQ ID NO: 364). The insert was obtained by annealing the following pairs of phosphorylated synthetic oligonucleotide pairs:

(SEQ ID NO: 365)
AF1for: AGGTTCTACYAGCGAATCYCCKTCTGGYACYGCWCC
(SEQ ID NO: 366)
AF1rev: ACCTGGWGCRGTRCCAGAMGGRGATTCGCTRGTAGA
(SEQ ID NO: 367)
AF2for: AGGTACYTCTACYCCKGAAAGCGGYTCYGCWTCTCC
(SEQ ID NO: 368)
AF2rev: ACCTGGAGAWGCRGARCCGCTTTCMGGRGTAGARGT
(SEQ ID NO: 369)
AF3for: AGGTACYTCYCCKAGCGGYGAATCTTCTACYGCWCC
(SEQ ID NO: 370)
AF3rev: ACCTGGWGCRGTAGAAGATTCRCCGCTMGGRGARGT
(SEQ ID NO: 371)
AF4for: AGGTTCYACYAGCTCTACYGCWGAATCTCCKGGYCC
(SEQ ID NO: 372)
AF4rev: ACCTGGRCCMGGAGATTCWGCRGTAGAGCTRGTRGA

We also annealed the phosphorylated oligonucleotide 3KpnIstopperFor: AGGTTCGTCTTCACTCGAGGGTAC (SEQ ID NO: 373) and the non-phosphorylated oligonucleotide pr_{—}3KpnIstopperRev: CCTCGAGTGAAGACGA (SEQ ID NO: 374). The annealed oligonucleotide pairs were ligated, which resulted in a mixture of products with varying length that represents the varying number of 12mer repeats ligated to one BbsI/KpnI segment The products corresponding to the length of 36 amino acids were isolated from the mixture by preparative agarose gel electrophoresis and ligated into the BsaI/KpnI digested stuffer vector pCW0359. Most of the clones in the resulting library designated LCW0403 showed green fluorescence after induction which shows that the sequence of XTEN_AF36 had been ligated in frame with the GFP gene and most sequences of XTEN_AF36 show good expression.

We screened 96 isolates from library LCW0403 for high level of fluorescence by stamping them onto agar plate containing IPTG. The same isolates were evaluated by PCR and 48 isolates were identified that contained segments with 36 amino acids as well as strong fluorescence. These isolates were sequenced and 44 clones were identified that contained correct XTEN_AF36 segments. Nucleotide and amino acid sequences for these segments are listed in Table 11.

TABLE 11
DNA and Amino Acid Sequences for 36-mer motifs

| | | SEQ | | SEQ |
|---|---|---|---|---|
| | | ID | | ID |
| File name | Amino acid sequence | NO: | Nucleotide sequence | NO: |
| LCW0403_004_{—} | GTSTPESGSASPGTSPS | 375 | GGTACTTCTACTCCGGAAAGCGGTTCCGCATCTC | 419 |
| GFP-N_A01.ab1 | GESSTAPGTSPSGESST | | CAGGTACTTCTCCTAGCGGTGAATCTTCTACTGC | |
| | AP | | TCCAGGTACCTCTCCTAGCGGCGAATCTTCTACT | |
| | | | GCTCCA | |
| LCW0403_005_{—} | GTSPSGESSTAPGSTSS | 376 | GGTACTTCTCCGAGCGGTGAATCTTCTACCGCAC | 420 |
| GFP-N_B01.ab1 | TAESPGPGTSPSGESST | | CAGGTTCTACTAGCTCTACCGCTGAATCTCCGGG | |
| | AP | | CCCAGGTACTTCTCCGAGCGGTGAATCTTCTACT | |
| | | | GCTCCA | |
| LCW0403_006_{—} | GSTSSTAESPGPGTSPS | 377 | GGTTCCACCAGCTCTACTGCTGAATCTCCTGGTC | 421 |
| GFP-N_C01.ab1 | GESSTAPGTSTPESGSA | | CAGGTACCTCTCCTAGCGGTGAATCTTCTACTGC | |
| | SP | | TCCAGGTACTTCTACTCCTGAAAGCGGCTCTGCT | |
| | | | TCTCCA | |
| LCW0403_007_{—} | GSTSSTAESPGPGSTSS | 378 | GGTTCTACCAGCTCTACTGCAGAATCTCCTGGCC | 422 |
| GFP-N_DO1.ab1 | TAESPGPGTSPSGESST | | CAGGTTCCACCAGCTCTACCGCAGAATCTCCGGG | |
| | AP | | TCCAGGTACTTCCCCTAGCGGTGAATCTTCTACC | |
| | | | GCACCA | |
| LCW0403_ 08_{—} | GSTSSTAESPGPGTSPS | 379 | GGTTCTACTAGCTCTACTGCTGAATCTCCTGGCC | 423 |
| GFP-N_E01.ab1 | GESSTAPGTSTPESGSA | | CAGGTACTTCTCCTAGCGGTGAATCTTCTACCGC | |
| | SP | | TCCAGGTACCTCTACTCCGGAAAGCGGTTCTGCA | |
| | | | TCTCCA | |
| LCW0403_010_{—} | GSTSSTAESPGPGTSTP | 380 | GGTTCTACCAGCTCTACCGCAGAATCTCCTGGTC | 424 |
| GFP-N_F01.ab1 | ESGSASPGSTSESPSGT | | CAGGTACCTCTACTCCGGAAAGCGGCTCTGCATC | |
| | AP | | TCCAGGTTCTACTAGCGAATCTCCTTCTGGCACT | |
| | | | GCACCA | |
| LCW0403_011_{—} | GSTSSTAESPGPGTSTP | 381 | GGTTCTACTAGCTCTACTGCAGAATCTCCTGGCC | 425 |
| GFP-N_G01.ab1 | ESGSASPGTSTPESGSA | | CAGGTACCTCTACTCCGGAAAGCGGCTCTGCATC | |
| | SP | | TCCAGGTACTTCTACCCCTGAAAGCGGTTCTGCA | |
| | | | TCTCCA | |
| LCW0403_012_{—} | GSTSESPSGTAPGTSPS | 382 | GGTTCTACCAGCGAATCTCCTTCTGGCACCGCTC | 426 |
| GFP-N_H01.ab1 | GESSTAPGSTSESPSGT | | CAGGTACCTCTCCTAGCGGCGAATCTTCTACCGC | |
| | AP | | TCCAGGTTCTACTAGCGAATCTCCTTCTGGCACT | |
| | | | GCACCA | |
| LCW0403_013_{—} | GSTSSTAESPGPGSTSS | 383 | GGTTCCACCAGCTCTACTGCAGAATCTCCGGGCC | 427 |
| GFP-N_A02.ab1 | TAESPGPGTSPSGESST | | CAGGTTCTACTAGCTCTACTGCAGAATCTCCGGG | |
| | AP | | TCCAGGTACTTCTCCTAGCGGCGAATCTTCTACC | |
| | | | GCTCCA | |
| LCW0403_014_{—} | GSTSSTAESPGPGTSTP | 384 | GGTTCCACTAGCTCTACTGCAGAATCTCCTGGCC | 428 |
| GFP-N_B02.ab1 | ESGSASPGSTSESPSGT | | CAGGTACCTCTACCCCTGAAAGCGGCTCTGCATC | |
| | AP | | TCCAGGTTCTACCAGCGAATCCCCGTCTGGCACC | |
| | | | GCACCA | |
| LCW0403_015_{—} | GSTSSTAESPGPGSTSS | 385 | GGTTCTACTAGCTCTACTGCTGAATCTCCGGGTC | 429 |
| GFP-N_C02.ab1 | TAESPGPGTSPSGESST | | CAGGTTCTACCAGCTCTACTGCTGAATCTCCTGG | |
| | AP | | TCCAGGTACCTCCCCGAGCGGTGAATCTTCTACT | |
| | | | GCACCA | |
| LCW0403_017_{—} | GSTSSTAESPGPGSTSE | 386 | GGTTCTACCAGCTCTACCGCTGAATCTCCTGGCC | 430 |
| GFP-N_D02.ab1 | SPSGTAPGSTSSTAESP | | CAGGTTCTACCAGCGAATCCCCGTCTGGCACCGC | |
| | GP | | ACCAGGTTCTACTAGCTCTACCGCTGAATCTCCG | |
| | | | GGTCCA | |
| LCW0403_018_{—} | GSTSSTAESPGPGSTSS | 387 | GGTTCTACCAGCTCTACCGCAGAATCTCCTGGCC | 431 |
| GFP-N_E02.ab1 | TAESPGPGSTSSTAESP | | CAGGTTCCACTAGCTCTACCGCTGAATCTCCTGG | |
| | GP | | TCCAGGTTCTACTAGCTCTACCGCTGAATCTCCT | |
| | | | GGTCCA | |
| LCW0403_019_{—} | GSTSESPSGTAPGSTSS | 388 | GGTTCTACTAGCGAATCCCCTTCTGGTACTGCTC | 432 |
| GFP-N_F02.ab1 | TAESPGPGSTSSTAESP | | CAGGTTCCACTAGCTCTACCGCTGAATCTCCTGG | |
| | GP | | CCCAGGTTCCACTAGCTCTACTGCAGAATCTCCT | |
| | | | GGTCCA | |
| LCW0403_023_{—} | GSTSESPSGTAPGSTSE | 389 | GGTTCTACTAGCGAATCTCCTTCTGGTACCGCTC | 433 |
| GFP-N_H02.ab1 | SPSGTAPGSTSESPSGT | | CAGGTTCTACCAGCGAATCCCCGTCTGGTACTGC | |
| | AP | | TCCAGGTTCTACCAGCGAATCTCCTTCTGGTACT | |
| | | | GCACCA | |
| LCW0403_024_{—} | GSTSSTAESPGPGSTSS | 390 | GGTTCCACCAGCTCTACTGCTGAATCTCCTGGCC | 434 |
| GFP-N_A03.ab1 | TAESPGPGSTSSTAESP | | CAGGTTCTACCAGCTCTACTGCTGAATCTCCGGG | |
| | GP | | CCCAGGTTCCACCAGCTCTACCGCTGAATCTCCG | |
| | | | GGTCCA | |
| LCW0403_025_{—} | GSTSSTAESPGPGSTSS | 391 | GGTTCCACTAGCTCTACCGCAGAATCTCCTGGTC | 435 |
| GFP-N_B03.ab1 | TAESPGPGTSPSGESST | | CAGGTTCTACTAGCTCTACTGCTGAATCTCCGGG | |
| | AP | | TCCAGGTACCTCCCCTAGCGGCGAATCTTCTACC | |
| | | | GCTCCA | |
| LCW0403_028_{—} | GSSPSASTGTGPGSSTP | 392 | GGTTCTAGCCCTTCTGCTTCCACCGGTACCGGCC | 436 |
| GFP-N_D03.ab1 | SGATGSPGSSTPSGAT | | CAGGTAGCTCTACTCCGTCTGGTGCAACTGGCTC | |
| | GSP | | TCCAGGTAGCTCTACTCCGTCTGGTGCAACCGGC | |
| | | | TCCCCA | |
| LCW0403_029_{—} | GTSPSGESSTAPGTSTP | 393 | GGTACTTCCCCTAGCGGTGAATCTTCTACTGCTC | 437 |
| GFP-N_E03.ab1 | ESGSASPGSTSSTAESP | | CAGGTACCTCTACTCCGGAAAGCGGCTCCGCATC | |
| | GP | | TCCAGGTTCTACTAGCTCTACTGCTGAATCTCCT | |
| | | | GGTCCA | |
| LCW0403_030_{—} | GSTSSTAESPGPGSTSS | 394 | GGTTCTACTAGCTCTACCGCTGAATCTCCGGGTC | 438 |
| GFP-N_F03.ab1 | TAESPGPGTSTPESGSA | | CAGGTTCTACCAGCTCTACTGCAGAATCTCCTGG | |
| | SP | | CCCAGGTACTTCTACTCCGGAAAGCGGTTCCGCT | |
| | | | TCTCCA | |
| LCW0403_031_{—} | GTSPSGESSTAPGSTSS | 395 | GGTACTTCTCCTAGCGGTGAATCTTCTACCGCTC | 439 |
| GFP-N_G03.ab1 | TAESPGPGTSTPESGSA | | CAGGTTCTACCAGCTCTACTGCTGAATCTCCTGG | |
| | SP | | CCCAGGTACTTCTACCCCGGAAAGCGGCTCCGCT | |
| | | | TCTCCA | |
| LCW0403_033_{—} | GSTSESPSGTAPGSTSS | 396 | GGTTCTACTAGCGAATCCCCTTCTGGTACTGCAC | 440 |
| GFP-N_H03.ab1 | TAESPGPGSTSSTAESP | | CAGGTTCTACCAGCTCTACTGCTGAATCTCCGGG | |
| | GP | | CCCAGGTTCCACCAGCTCTACCGCAGAATCTCCT | |
| | | | GGTCCA | |
| LCW0403_035_{—} | GSTSSTAESPGPGSTSE | 397 | GGTTCCACCAGCTCTACCGCTGAATCTCCGGGCC | 441 |
| GFP-N_A04.ab1 | SPSGTAPGSTSSTAESP | | CAGGTTCTACCAGCGAATCCCCTTCTGGCACTGC | |
| | GP | | ACCAGGTTCTACTAGCTCTACCGCAGAATCTCCG | |
| | | | GGCCCA | |
| LCW0403_036_{—} | GSTSSTAESPGPGTSPS | 398 | GGTTCTACCAGCTCTACTGCTGAATCTCCGGGTC | 442 |
| GFP-N_B04.ab1 | GESSTAPGTSTPESGSA | | CAGGTACTTCCCCGAGCGGTGAATCTTCTACTGC | |
| | SP | | ACCAGGTACTTCTACTCCGGAAAGCGGTTCCGCT | |
| | | | TCTCCA | |
| LCW0403_039_{—} | GSTSESPSGTAPGSTSE | 399 | GGTTCTACCAGCGAATCTCCTTCTGGCACCGCTC | 443 |
| GFP-N_C04.ab1 | SPSGTAPGTSPSGESST | | CAGGTTCTACTAGCGAATCCCCGTCTGGTACCGC | |
| | AP | | ACCAGGTACTTCTCCTAGCGGCGAATCTTCTACC | |
| | | | GCACCA | |
| LCW0403_041_{—} | GSTSESPSGTAPGSTSE | 400 | GGTTCTACCAGCGAATCCCCTTCTGGTACTGCTC | 444 |
| GFP-N_D04.ab1 | SPSGTAPGTSTPESGSA | | CAGGTTCTACCAGCGAATCCCCTTCTGGCACCGC | |
| | SP | | ACCAGGTACTTCTACCCCTGAAAGCGGCTCCGCT | |
| | | | TCTCCA | |
| LCW0403_044_{—} | GTSTPESGSASPGSTSS | 401 | GGTACCTCTACTCCTGAAAGCGGTTCTGCATCTC | 445 |
| GFP-N_E04.ab1 | TAESPGPGSTSSTAESP | | CAGGTTCCACTAGCTCTACCGCAGAATCTCCGGG | |
| | GP | | CCCAGGTTCTACTAGCTCTACTGCTGAATCTCCT | |
| | | | GGCCCA | |
| LCW0403_046_{—} | GSTSESPSGTAPGSTSE | 402 | GGTTCTACCAGCGAATCCCCTTCTGGCACTGCAC | 446 |
| GFP-N_F04.ab1 | SPSGTAPGTSPSGESST | | CAGGTTCTACTAGCGAATCCCCTTCTGGTACCGC | |
| | AP | | ACCAGGTACTTCTCCGAGCGGCGAATCTTCTACT | |
| | | | GCTCCA | |
| LCW0403_047_{—} | GSTSSTAESPGPGSTSS | 403 | GGTTCTACTAGCTCTACCGCTGAATCTCCTGGCC | 447 |
| GFP-N_G04.ab1 | TAESPGPGSTSESPSGT | | CAGGTTCCACTAGCTCTACCGCAGAATCTCCGGG | |
| | AP | | CCCAGGTTCTACTAGCGAATCCCCTTCTGGTACC | |
| | | | GCTCCA | |
| LCW0403_049_{—} | GSTSSTAESPGPGSTSS | 404 | GGTTCCACCAGCTCTACTGCAGAATCTCCTGGCC | 448 |
| GFP-N_H04.ab1 | TAESPGPGTSTPESGSA | | CAGGTTCTACTAGCTCTACCGCAGAATCTCCTGG | |
| | SP | | TCCAGGTACCTCTACTCCTGAAAGCGGTTCCGCA | |
| | | | TCTCCA | |
| LCW0403_051_{—} | GSTSSTAESPGPGSTSS | 405 | GGTTCTACTAGCTCTACTGCTGAATCTCCGGGCC | 449 |
| GFP-N_A05.ab1 | TAESPGPGSTSESPSGT | | CAGGTTCTACTAGCTCTACCGCTGAATCTCCGGG | |
| | AP | | TCCAGGTTCTACTAGCGAATCTCCTTCTGGTACC | |
| | | | GCTCCA | |
| LCW0403_053_{—} | GTSPSGESSTAPGSTSE | 406 | GGTACCTCCCCGAGCGGTGAATCTTCTACTGCAC | 450 |
| GFP-N_B05.ab1 | SPSGTAPGSTSSTAESP | | CAGGTTCTACTAGCGAATCCCCTTCTGGTACTGC | |
| | GP | | TCCAGGTTCCACCAGCTCTACTGCAGAATCTCCG | |
| | | | GGTCCA | |
| LCW0403_054_{—} | GSTSESPSGTAPGTSPS | 407 | GGTTCTACTAGCGAATCCCCGTCTGGTACTGCTC | 451 |
| GFP-N_C05.ab1 | GESSTAPGSTSSTAESP | | CAGGTACTTCCCCTAGCGGTGAATCTTCTACTGC | |
| | GP | | TCCAGGTTCTACCAGCTCTACCGCAGAATCTCCG | |
| | | | GGTCCA | |
| LCW0403_057_{—} | GSTSSTAESPGPGSTSE | 408 | GGTTCTACCAGCTCTACCGCTGAATCTCCTGGCC | 452 |
| GFP-N_D05.ab1 | SPSGTAPGTSPSGESST | | CAGGTTCTACTAGCGAATCTCCGTCTGGCACCGC | |
| | AP | | ACCAGGTACTTCCCCTAGCGGTGAATCTTCTACT | |
| | | | GCACCA | |
| LCW0403_058_{—} | GSTSESPSGTAPGSTSE | 409 | GGTTCTACTAGCGAATCTCCTTCTGGCACTGCAC | 453 |
| GFP-N_E05.abl | SPSGTAPGTSTPESGSA | | CAGGTTCTACCAGCGAATCTCCGTCTGGCACTGC | |
| | SP | | ACCAGGTACCTCTACCCCTGAAAGCGGTTCCGCT | |
| | | | TCTCCA | |
| LCW0403_060_{—} | GTSTPESGSASPGSTSE | 410 | GGTACCTCTACTCCGGAAAGCGGTTCCGCATCTC | 454 |
| GFP-N_F05.ab1 | SPSGTAPGSTSSTAESP | | CAGGTTCTACCAGCGAATCCCCGTCTGGCACCGC | |
| | GP | | ACCAGGTTCTACTAGCTCTACTGCTGAATCTCCG | |
| | | | GGCCCA | |
| LCW0403_063_{—} | GSTSSTAESPGPGTSPS | 411 | GGTTCTACTAGCTCTACTGCAGAATCTCCGGGCC | 455 |
| GFP-N_G05.ab1 | GESSTAPGTSPSGESST | | CAGGTACCTCTCCTAGCGGTGAATCTTCTACCGC | |
| | AP | | TCCAGGTACTTCTCCGAGCGGTGAATCTTCTACC | |
| | | | GCTCCA | |
| LCW0403_064_{—} | GTSPSGESSTAPGTSPS | 412 | GGTACCTCCCCTAGCGGCGAATCTTCTACTGCTC | 456 |
| GFP-N_H05.ab1 | GESSTAPGTSPSGESST | | CAGGTACCTCTCCTAGCGGCGAATCTTCTACCGC | |
| | AP | | TCCAGGTACCTCCCCTAGCGGTGAATCTTCTACC | |
| | | | GCACCA | |
| LCW0403_065_{—} | GSTSSTAESPGPGTSTP | 413 | GGTTCCACTAGCTCTACTGCTGAATCTCCTGGCC | 457 |
| GFP-N_A06.ab1 | ESGSASPGSTSESPSGT | | CAGGTACTTCTACTCCGGAAAGCGGTTCCGCTTC | |
| | AP | | TCCAGGTTCTACTAGCGAATCTCCGTCTGGCACC | |
| | | | GCACCA | |
| LCW0403_066_{—} | GSTSESPSGTAPGTSPS | 414 | GGTTCTACTAGCGAATCTCCGTCTGGCACTGCTC | 458 |
| GFP-N_B06.ab1 | GESSTAPGTSPSGESST | | CAGGTACTTCTCCTAGCGGTGAATCTTCTACCGC | |
| | AP | | TCCAGGTACTTCCCCTAGCGGCGAATCTTCTACC | |
| | | | GCTCCA | |
| LCW0403_067_{—} | GSTSESPSGTAPGTSTP | 415 | GGTTCTACTAGCGAATCTCCTTCTGGTACCGCTC | 459 |
| GFP-N_C06.ab1 | ESGSASPGSTSSTAESP | | CAGGTACTTCTACCCCTGAAAGCGGCTCCGCTTC | |
| | GP | | TCCAGGTTCCACTAGCTCTACCGCTGAATCTCCG | |
| | | | GGTCCA | |
| LCW0403_068_{—} | GSTSSTAESPGPGSTSS | 416 | GGTTCCACTAGCTCTACTGCTGAATCTCCTGGCC | 460 |
| GFP-N_D06.ab1 | TAESPGPGSTSESPSGT | | CAGGTTCTACCAGCTCTACCGCTGAATCTCCTGG | |
| | AP | | CCCAGGTTCTACCAGCGAATCTCCGTCTGGCACC | |
| | | | GCACCA | |
| LCW0403_069_{—} | GSTSESPSGTAPGTSTP | 417 | GGTTCTACTAGCGAATCCCCGTCTGGTACCGCAC | 461 |
| GFP-N_E06.ab1 | ESGSASPGTSTPESGSA | | CAGGTACTTCTACCCCGGAAAGCGGCTCTGCTTC | |
| | SP | | TCCAGGTACTTCTACCCCGGAAAGCGGCTCCGCA | |
| | | | TCTCCA | |
| LCW0403_070_{—} | GSTSESPSGTAPGTSTP | 418 | GGTTCTACTAGCGAATCCCCGTCTGGTACTGCTC | 462 |
| GFP-N_F06.ab1 | ESGSASPGTSTPESGSA | | CAGGTACTTCTACTCCTGAAAGCGGTTCCGCTTC | |
| | SP | | TCCAGGTACCTCTACTCCGGAAAGCGGTTCTGCA | |
| | | | TCTCCA | |

### Example 4

### Construction of XTEN_AG36 segments

A codon library encoding sequences of 36 amino acid length was constructed. The sequences were designated XTEN_AG36. Its segments have the amino acid sequence [X]₃ where X is a 12mer peptide with the sequence: GTPGSGTASSSP (SEQ ID NO: 463), GSSTPSGATGSP (SEQ ID NO: 464), GSSPSASTGTGP (SEQ ID NO: 465), or GASPGTSSTGSP (SEQ ID NO: 466). The insert was obtained by annealing the following pairs of phosphorylated synthetic oligonucleotide pairs:

(SEQ ID NO: 467)
AG1for: AGGTACYCCKGGYAGCGGTACYGCWTCTTCYTCTCC
(SEQ ID NO: 468)
AG1rev: ACCTGGAGARGAAGAWGCRGTACCGCTRCCMGGRGT
(SEQ ID NO: 469)
AG2for: AGGTAGCTCTACYCCKTCTGGTGCWACYGGYTCYCC
(SEQ ID NO: 470)
AG2rev: ACCTGGRGARCCRGTWGCACCAGAMGGRGTAGAGCT
(SEQ ID NO: 471)
AG3for: AGGTTCTAGCCCKTCTGCWTCYACYGGTACYGGYCC
(SEQ ID NO: 472)
AG3rev: ACCTGGRCCRGTACCRGTRGAWGCAGAMGGGCTAGA
(SEQ ID NO: 473)
AG4for: AGGTGCWTCYCCKGGYACYAGCTCTACYGGTTCTCC
(SEQ ID NO: 474)
AG4rev: ACCTGGAGAACCRGTAGAGCTRGTRCCMGGRGAWGC

We also annealed the phosphorylated oligonucleotide 3KpnIstopperFor: AGGTTCGTCTTCACTCGAGGGTAC (SEQ ID NO: 475) and the non-phosphorylated oligonucleotide pr_{—}3KpnIstopperRev: CCTCGAGTGAAGACGA (SEQ ID NO: 476). The annealed oligonucleotide pairs were ligated, which resulted in a mixture of products with varying length that represents the varying number of 12mer repeats ligated to one BbsI/KpnI segment. The products corresponding to the length of 36 amino acids were isolated from the mixture by preparative agarose gel electrophoresis and ligated into the BsaI/KpnI digested stuffer vector pCW0359. Most of the clones in the resulting library designated LCW0404 showed green fluorescence after induction which shows that the sequence of XTEN_AG36 had been ligated in frame with the GFP gene and most sequences of XTEN_AG36 show good expression.

We screened 96 isolates from library LCW0404 for high level of fluorescence by stamping them onto agar plate containing IPTG. The same isolates were evaluated by PCR and 48 isolates were identified that contained segments with 36 amino acids as well as strong fluorescence. These isolates were sequenced and 44 clones were identified that contained correct XTEN_AG36 segments. Nucleotide and amino acid sequences for these segments are listed in Table 12.

TABLE 12
DNA and Amino Acid Sequences for 36-mer motifs

| | | SEQ | | SEQ |
|---|---|---|---|---|
| | | ID | | ID |
| File name | Amino acid sequence | NO: | Nucleotide sequence | NO: |
| LCW0404_001_{—} | GASPGTSSTGSPGT | 477 | GGTGCATCCCCGGGCACTAGCTCTACCGGTTCTC | 521 |
| GFP-N_A07.ab1 | PGSGTASSSPGSST | | CAGGTACTCCTGGTAGCGGTACTGCTTCTTCTTC | |
| | PSGATGSP | | TCCAGGTAGCTCTACTCCTTCTGGTGCTACTGGT | |
| | | | TCTCCA | |
| LCW0404_003_{—} | GSSTPSGATGSPGS | 478 | GGTAGCTCTACCCCTTCTGGTGCTACCGGCTCTC | 522 |
| GFP-N_B07.ab1 | SPSASTGTGPGSST | | CAGGTTCTAGCCCGTCTGCTTCTACCGGTACCGG | |
| | PSGATGSP | | TCCAGGTAGCTCTACCCCTTCTGGTGCTACTGGT | |
| | | | TCTCCA | |
| LCW0404_006_{—} | GASPGTSSTGSPGS | 479 | GGTGCATCTCCGGGTACTAGCTCTACCGGTTCTC | 523 |
| GFP-N_C07.ab1 | SPSASTGTGPGSST | | CAGGTTCTAGCCCTTCTGCTTCCACTGGTACCGG | |
| | PSGATGSP | | CCCAGGTAGCTCTACCCCGTCTGGTGCTACTGGT | |
| | | | TCCCCA | |
| LCW0404_007_{—} | GTPGSGTASSSPGS | 480 | GGTACTCCGGGCAGCGGTACTGCTTCTTCCTCTC | 524 |
| GFP-N_D07.ab1 | STPSGATGSPGASP | | CAGGTAGCTCTACCCCTTCTGGTGCAACTGGTTC | |
| | GTSSTGSP | | CCCAGGTGCATCCCCTGGTACTAGCTCTACCGGT | |
| | | | TCTCCA | |
| LCW0404_009_{—} | GTPGSGTASSSPGA | 481 | GGTACCCCTGGCAGCGGTACTGCTTCTTCTTCTC | 525 |
| GFP-N_E07.ab1 | SPGTSSTGSPGSRP | | CAGGTGCTTCCCCTGGTACCAGCTCTACCGGTTC | |
| | SASTGTGP | | TCCAGGTTCTAGACCTTCTGCATCCACCGGTACT | |
| | | | GGTCCA | |
| LCW0404_011_{—} | GASPGTSSTGSPGS | 482 | GGTGCATCTCCTGGTACCAGCTCTACCGGTTCTC | 526 |
| GFP-N_F07.ab1 | STPSGATGSPGASP | | CAGGTAGCTCTACTCCTTCTGGTGCTACTGGCTC | |
| | GTSSTGSP | | TCCAGGTGCTTCCCCGGGTACCAGCTCTACCGGT | |
| | | | TCTCCA | |
| LCW0404_012_{—} | GTPGSGTASSSPGS | 483 | GGTACCCCGGGCAGCGGTACCGCATCTTCCTCTC | 527 |
| GFP-N_G07.ab1 | STPSGATGSPGSSTP | | CAGGTAGCTCTACCCCGTCTGGTGCTACCGGTTC | |
| | SGATGSP | | CCCAGGTAGCTCTACCCCGTCTGGTGCAACCGGC | |
| | | | TCCCCA | |
| LCW0404_014_{—} | GASPGTSSTGSPGA | 484 | GGTGCATCTCCGGGCACTAGCTCTACTGGTTCTC | 528 |
| GFP-N_H07.ab1 | SPGTSSTGSPGASP | | CAGGTGCATCCCCTGGCACTAGCTCTACTGGTTC | |
| | GTSSTGSP | | TCCAGGTGCTTCTCCTGGTACCAGCTCTACTGGT | |
| | | | TCTCCA | |
| LCW0404_015_{—} | GSSTPSGATGSPGS | 485 | GGTAGCTCTACTCCGTCTGGTGCAACCGGCTCCC | 529 |
| GFP-N_A08.ab1 | SPSASTGTGPGASP | | CAGGTTCTAGCCCGTCTGCTTCCACTGGTACTGG | |
| | GTSSTGSP | | CCCAGGTGCTTCCCCGGGCACCAGCTCTACTGGT | |
| | | | TCTCCA | |
| LCW0404_016_{—} | GSSTPSGATGSPGS | 486 | GGTAGCTCTACTCCTTCTGGTGCTACCGGTTCCC | 530 |
| GFP-N_B08.ab1 | STPSGATGSPGTPG | | CAGGTAGCTCTACTCCTTCTGGTGCTACTGGTTC | |
| | SGTASSSP | | CCCAGGTACTCCGGGCAGCGGTACTGCTTCTTCC | |
| | | | TCTCCA | |
| LCW0404_017_{—} | GSSTPSGATGSPGS | 487 | GGTAGCTCTACTCCGTCTGGTGCAACCGGTTCCC | 531 |
| GFP-N_C08.ab1 | STPSGATGSPGASP | | CAGGTAGCTCTACTCCTTCTGGTGCTACTGGCTC | |
| | GTSSTGSP | | CCCAGGTGCATCCCCTGGCACCAGCTCTACCGGT | |
| | | | TCTCCA | |
| LCW0404_018_{—} | GTPGSGTASSSPGS | 488 | GGTACTCCTGGTAGCGGTACCGCATCTTCCTCTC | 532 |
| GFP-N_D08.ab1 | SPSASTGTGPGSST | | CAGGTTCTAGCCCTTCTGCATCTACCGGTACCGG | |
| | PSGATGSP | | TCCAGGTAGCTCTACTCCTTCTGGTGCTACTGGC | |
| | | | TCTCCA | |
| LCW0404_023_{—} | GASPGTSSTGSPGS | 489 | GGTGCTTCCCCGGGCACTAGCTCTACCGGTTCTC | 533 |
| GFP-N_F08.ab1 | SPSASTGTGPGTPG | | CAGGTTCTAGCCCTTCTGCATCTACTGGTACTGG | |
| | SGTASSSP | | CCCAGGTACTCCGGGCAGCGGTACTGCTTCTTCC | |
| | | | TCTCCA | |
| LCW0404_025_{—} | GSSTPSGATGSPGS | 490 | GGTAGCTCTACTCCGTCTGGTGCTACCGGCTCTC | 534 |
| GFP-N_G08.ab1 | STPSGATGSPGASP | | CAGGTAGCTCTACCCCTTCTGGTGCAACCGGCTC | |
| | GTSSTGSP | | CCCAGGTGCTTCTCCGGGTACCAGCTCTACTGGT | |
| | | | TCTCCA | |
| LCW0404_029_{—} | GTPGSGTASSSPGS | 491 | GGTACCCCTGGCAGCGGTACCGCTTCTTCCTCTC | 535 |
| GFP-N_A09.ab1 | STPSGATGSPGSSP | | CAGGTAGCTCTACCCCGTCTGGTGCTACTGGCTC | |
| | SASTGTGP | | TCCAGGTTCTAGCCCGTCTGCATCTACCGGTACC | |
| | | | GGCCCA | |
| LCW0404_030_{—} | GSSTPSGATGSPGT | 492 | GGTAGCTCTACTCCTTCTGGTGCAACCGGCTCCC | 536 |
| GFP-N_B09.ab1 | PGSGTASSSPGTPG | | CAGGTACCCCGGGCAGCGGTACCGCATCTTCCTC | |
| | SGTASSSP | | TCCAGGTACTCCGGGTAGCGGTACTGCTTCTTCT | |
| | | | TCTCCA | |
| LCW0404_031_{—} | GTPGSGTASSSPGS | 493 | GGTACCCCGGGTAGCGGTACTGCTTCTTCCTCTC | 537 |
| GFP-N_C09.ab1 | STPSGATGSPGASP | | CAGGTAGCTCTACCCCTTCTGGTGCAACCGGCTC | |
| | GTSSTGSP | | TCCAGGTGCTTCTCCGGGCACCAGCTCTACCGGT | |
| | | | TCTCCA | |
| LCW0404_034_{—} | GSSTPSGATGSPGS | 494 | GGTAGCTCTACCCCGTCTGGTGCTACCGGCTCTC | 538 |
| GFP-N_D09.ab1 | STPSGATGSPGASP | | CAGGTAGCTCTACCCCGTCTGGTGCAACCGGCTC | |
| | GTSSTGSP | | CCCAGGTGCATCCCCGGGTACTAGCTCTACCGGT | |
| | | | TCTCCA | |
| LCW0404_035_{—} | GASPGTSSTGSPGT | 495 | GGTGCTTCTCCGGGCACCAGCTCTACTGGTTCTC | 539 |
| GFP-N_E09.ab1 | PGSGTASSSPGSST | | CAGGTACCCCGGGCAGCGGTACCGCATCTTCTTC | |
| | PSGATGSP | | TCCAGGTAGCTCTACTCCTTCTGGTGCAACTGGT | |
| | | | TCTCCA | |
| LCW0404_036_{—} | GSSPSASTGTGPGS | 496 | GGTTCTAGCCCGTCTGCTTCCACCGGTACTGGCC | 540 |
| GFP-N_F09.ab1 | STPSGATGSPGTPG | | CAGGTAGCTCTACCCCGTCTGGTGCAACTGGTTC | |
| | SGTASSSP | | CCCAGGTACCCCTGGTAGCGGTACCGCTTCTTCT | |
| | | | TCTCCA | |
| LCW0404_037_{—} | GASPGTSSTGSPGS | 497 | GGTGCTTCTCCGGGCACCAGCTCTACTGGTTCTC | 541 |
| GFP-N_G09.ab1 | SPSASTGTGPGSST | | CAGGTTCTAGCCCTTCTGCATCCACCGGTACCGG | |
| | PSGATGSP | | TCCAGGTAGCTCTACCCCTTCTGGTGCAACCGGC | |
| | | | TCTCCA | |
| LCW0404_040_{—} | GASPGTSSTGSPGS | 498 | GGTGCATCCCCGGGCACCAGCTCTACCGGTTCTC | 542 |
| GFP-N_H09.ab1 | STPSGATGSPGSST | | CAGGTAGCTCTACCCCGTCTGGTGCTACCGGCTC | |
| | PSGATGSP | | TCCAGGTAGCTCTACCCCGTCTGGTGCTACTGGC | |
| | | | TCTCCA | |
| LCW0404_041_{—} | GTPGSGTASSSPGS | 499 | GGTACCCCTGGTAGCGGTACTGCTTCTTCCTCTC | 543 |
| GFP-N_A10.ab1 | STPSGATGSPGTPG | | CAGGTAGCTCTACTCCGTCTGGTGCTACCGGTTC | |
| | SGTASSSP | | TCCAGGTACCCCGGGTAGCGGTACCGCATCTTCT | |
| | | | TCTCCA | |
| LCW0404_043_{—} | GSSPSASTGTGPGS | 500 | GGTTCTAGCCCTTCTGCTTCCACCGGTACTGGCC | 544 |
| GFP-N_C10.ab1 | STPSGATGSPGSST | | CAGGTAGCTCTACCCCTTCTGGTGCTACCGGCTC | |
| | PSGATGSP | | CCCAGGTAGCTCTACTCCTTCTGGTGCAACTGGC | |
| | | | TCTCCA | |
| LCW0404_045_{—} | GASPGTSSTGSPGS | 501 | GGTGCTTCTCCTGGCACCAGCTCTACTGGTTCTC | 545 |
| GFP-N_D10.ab1 | SPSASTGTGPGSSP | | CAGGTTCTAGCCCTTCTGCTTCTACCGGTACTGG | |
| | SASTGTGP | | TCCAGGTTCTAGCCCTTCTGCATCCACTGGTACT | |
| | | | GGTCCA | |
| LCW0404_047_{—} | GTPGSGTASSSPGA | 502 | GGTACTCCTGGCAGCGGTACCGCTTCTTCTTCTC | 546 |
| GFP-N_F10.ab1 | SPGTSSTGSPGASP | | CAGGTGCTTCTCCTGGTACTAGCTCTACTGGTTC | |
| | GTSSTGSP | | TCCAGGTGCTTCTCCGGGCACTAGCTCTACTGGT | |
| | | | TCTCCA | |
| LCW0404_048_{—} | GSSTPSGATGSPGA | 503 | GGTAGCTCTACCCCGTCTGGTGCTACCGGTTCCC | 547 |
| GFP-N_G10.ab1 | SPGTSSTGSPGSST | | CAGGTGCTTCTCCTGGTACTAGCTCTACCGGTTC | |
| | PSGATGSP | | TCCAGGTAGCTCTACCCCGTCTGGTGCTACTGGC | |
| | | | TCTCCA | |
| LCW0404_049_{—} | GSSTPSGATGSPGT | 504 | GGTAGCTCTACCCCGTCTGGTGCTACTGGTTCTC | 548 |
| GFP-N_H10.ab1 | PGSGTASSSPGSSTP | | CAGGTACTCCGGGCAGCGGTACTGCTTCTTCCTC | |
| | SGATGSP | | TCCAGGTAGCTCTACCCCTTCTGGTGCTACTGGC | |
| | | | TCTCCA | |
| LCW0404_050_{—} | GASPGTSSTGSPGS | 505 | GGTGCATCTCCTGGTACCAGCTCTACTGGTTCTC | 549 |
| GFP-N_A11.ab1 | SPSASTGTGPGSSTP | | CAGGTTCTAGCCCTTCTGCTTCTACCGGTACCGG | |
| | SGATGSP | | TCCAGGTAGCTCTACTCCTTCTGGTGCTACCGGT | |
| | | | TCTCCA | |
| LCW0404_051_{—} | GSSTPSGATGSPGS | 506 | GGTAGCTCTACCCCGTCTGGTGCTACTGGCTCTC | 550 |
| GFP-N_B11.ab1 | STPSGATGSPGSST | | CAGGTAGCTCTACTCCTTCTGGTGCTACTGGTTC | |
| | PSGATGSP | | CCCAGGTAGCTCTACCCCGTCTGGTGCAACTGGC | |
| | | | TCTCCA | |
| LCW0404_052_{—} | GASPGTSSTGSPGT | 507 | GGTGCATCCCCGGGTACCAGCTCTACCGGTTCTC | 551 |
| GFP-N_C11.ab1 | PGSGTASSSPGASP | | CAGGTACTCCTGGCAGCGGTACTGCATCTTCCTC | |
| | GTSSTGSP | | TCCAGGTGCTTCTCCGGGCACCAGCTCTACTGGT | |
| | | | TCTCCA | |
| LCW0404_053_{—} | GSSTPSGATGSPGS | 508 | GGTAGCTCTACTCCTTCTGGTGCAACTGGTTCTC | 552 |
| GFP-N_D11.ab1 | SPSASTGTGPGASP | | CAGGTTCTAGCCCGTCTGCATCCACTGGTACCGG | |
| | GTSSTGSP | | TCCAGGTGCTTCCCCTGGCACCAGCTCTACCGGT | |
| | | | TCTCCA | |
| LCW0404_057_{—} | GASPGTSSTGSPGS | 509 | GGTGCATCTCCTGGTACTAGCTCTACTGGTTCTC | 553 |
| GFP-N_E11.ab1 | STPSGATGSPGSSP | | CAGGTAGCTCTACTCCGTCTGGTGCAACCGGCTC | |
| | SASTGTGP | | TCCAGGTTCTAGCCCTTCTGCATCTACCGGTACT | |
| | | | GGTCCA | |
| LCW0404_060_{—} | GTPGSGTASSSPGS | 510 | GGTACTCCTGGCAGCGGTACCGCATCTTCCTCTC | 554 |
| GFP-N_F11.ab1 | STPSGATGSPGASP | | CAGGTAGCTCTACTCCGTCTGGTGCAACTGGTTC | |
| | GTSSTGSP | | CCCAGGTGCTTCTCCGGGTACCAGCTCTACCGGT | |
| | | | TCTCCA | |
| LCW0404_062_{—} | GSSTPSGATGSPGT | 511 | GGTAGCTCTACCCCGTCTGGTGCAACCGGCTCCC | 555 |
| GFP-N_G11.ab1 | PGSGTASSSPGSST | | CAGGTACTCCTGGTAGCGGTACCGCTTCTTCTTC | |
| | PSGATGSP | | TCCAGGTAGCTCTACTCCGTCTGGTGCTACCGGC | |
| | | | TCCCCA | |
| LCW0404_066_{—} | GSSPSASTGTGPGS | 512 | GGTTCTAGCCCTTCTGCATCCACCGGTACCGGCC | 556 |
| GFP-N_H11.ab1 | SPSASTGTGPGASP | | CAGGTTCTAGCCCGTCTGCTTCTACCGGTACTGG | |
| | GTSSTGSP | | TCCAGGTGCTTCTCCGGGTACTAGCTCTACTGGT | |
| | | | TCTCCA | |
| LCW0404_067_{—} | GTPGSGTASSSPGS | 513 | GGTACCCCGGGTAGCGGTACCGCTTCTTCTTCTC | 557 |
| GFP-N_A12.ab1 | STPSGATGSPGSNP | | CAGGTAGCTCTACTCCGTCTGGTGCTACCGGCTC | |
| | SASTGTGP | | TCCAGGTTCTAACCCTTCTGCATCCACCGGTACC | |
| | | | GGCCCA | |
| LCW0404_068_{—} | GSSPSASTGTGPGS | 514 | GGTTCTAGCCCTTCTGCATCTACTGGTACTGGCC | 558 |
| GFP-N_B12.ab1 | STPSGATGSPGASP | | CAGGTAGCTCTACTCCTTCTGGTGCTACCGGCTC | |
| | GTSSTGSP | | TCCAGGTGCTTCTCCGGGTACTAGCTCTACCGGT | |
| | | | TCTCCA | |
| LCW0404_069_{—} | GSSTPSGATGSPGA | 515 | GGTAGCTCTACCCCTTCTGGTGCAACCGGCTCTC | 559 |
| GFP-N_C12.ab1 | SPGTSSTGSPGTPG | | CAGGTGCATCCCCGGGTACCAGCTCTACCGGTTC | |
| | SGTASSSP | | TCCAGGTACTCCGGGTAGCGGTACCGCTTCTTCC | |
| | | | TCTCCA | |
| LCW0404_070_{—} | GSSTPSGATGSPGS | 516 | GGTAGCTCTACTCCGTCTGGTGCAACCGGTTCCC | 560 |
| GFP-N_D12.ab1 | STPSGATGSPGSST | | CAGGTAGCTCTACCCCTTCTGGTGCAACCGGCTC | |
| | PSGATGSP | | CCCAGGTAGCTCTACCCCTTCTGGTGCAACTGGC | |
| | | | TCTCCA | |
| LCW0404_073_{—} | GASPGTSSTGSPGT | 517 | GGTGCTTCTCCTGGCACTAGCTCTACCGGTTCTC | 561 |
| GFP-N_E12.ab1 | PGSGTASSSPGSST | | CAGGTACCCCTGGTAGCGGTACCGCATCTTCCTC | |
| | PSGATGSP | | TCCAGGTAGCTCTACTCCTTCTGGTGCTACTGGT | |
| | | | TCCCCA | |
| LCW0404_075_{—} | GSSTPSGATGSPGS | 518 | GGTAGCTCTACCCCGTCTGGTGCTACTGGCTCCC | 562 |
| GFP-N_F12.ab1 | SPSASTGTGPGSSP | | CAGGTTCTAGCCCTTCTGCATCCACCGGTACCGG | |
| | SASTGTGP | | TCCAGGTTCTAGCCCGTCTGCATCTACTGGTACT | |
| | | | GGTCCA | |
| LCW0404_080_{—} | GASPGTSSTGSPGS | 519 | GGTGCTTCCCCGGGCACCAGCTCTACTGGTTCTC | 563 |
| GFP-N_G12.ab1 | SPSASTGTGPGSSP | | CAGGTTCTAGCCCGTCTGCTTCTACTGGTACTGG | |
| | SASTGTGP | | TCCAGGTTCTAGCCCTTCTGCTTCCACTGGTACT | |
| | | | GGTCCA | |
| LCW0404_081_{—} | GASPGTSSTGSPGS | 520 | GGTGCTTCCCCGGGTACCAGCTCTACCGGTTCTC | 564 |
| GFP-N_H12.ab1 | SPSASTGTGPGTPG | | CAGGTTCTAGCCCTTCTGCTTCTACCGGTACCGG | |
| | SGTASSSP | | TCCAGGTACCCCTGGCAGCGGTACCGCATCTTCC | |
| | | | TCTCCA | |

### Example 5

### Construction of XTEN_AE864

XTEN_AE864 was constructed from serial dimerization of XTEN_AE36 to AE72, 144, 288, 576 and 864. A collection of XTEN_AE72 segments was constructed from 37 different segments of XTEN_AE36. Cultures of E. coli harboring all 37 different 36-amino acid segments were mixed and plasmid was isolated. This plasmid pool was digested with BsaI/NcoI to generate the small fragment as the insert. The same plasmid pool was digested with BbsI/NcoI to generate the large fragment as the vector. The insert and vector fragments were ligated resulting in a doubling of the length and the ligation mixture was transformed into BL21Gold(DE3) cells to obtain colonies of XTEN_AE72.

This library of XTEN_AE72 segments was designated LCW0406. All clones from LCW0406 were combined and dimerized again using the same process as described above yielding library LCW0410 of XTEN_AE144. All clones from LCW0410 were combined and dimerized again using the same process as described above yielding library LCW0414 of XTEN_AE288. Two isolates LCW0414.001 and LCW0414.002 were randomly picked from the library and sequenced to verify the identities. All clones from LCW0414 were combined and dimerized again using the same process as described above yielding library LCW0418 of XTEN_AE576. We screened 96 isolates from library LCW0418 for high level of GFP fluorescence. 8 isolates with right sizes of inserts by PCR and strong fluorescence were sequenced and 2 isolates (LCW0418.018 and LCW0418.052) were chosen for future use based on sequencing and expression data.

The specific clone pCW0432 of XTEN_AE864 was constructed by combining LCW0418.018 of XTEN_AE576 and LCW0414.002 of XTEN_AE288 using the same dimerization process as described above.

### Example 6

### Construction of XTEN_AM144

A collection of XTEN_AM144 segments was constructed starting from 37 different segments of XTEN_AE36, 44 segments of XTEN_AF36, and 44 segments of XTEN_AG36.

Cultures of E. coli that harboring all 125 different 36-amino acid segments were mixed and plasmid was isolated. This plasmid pool was digested with BsaI/NcoI to generate the small fragment as the insert. The same plasmid pool was digested with BbsI/NcoI to generate the large fragment as the vector. The insert and vector fragments were ligated resulting in a doubling of the length and the ligation mixture was transformed into BL21Gold(DE3) cells to obtain colonies of XTEN_AM72.

This library of XTEN_AM72 segments was designated LCW0461. All clones from LCW0461 were combined and dimerized again using the same process as described above yielding library LCW0462. 1512 Isolates from library LCW0462 were screened for protein expression. Individual colonies were transferred into 96 well plates and cultured overnight as starter cultures. These starter cultures were diluted into fresh autoinduction medium and cultured for 20-30 h. Expression was measured using a fluorescence plate reader with excitation at 395 nm and emission at 510 nm. 192 isolates showed high level expression and were submitted to DNA sequencing. Most clones in library LCW0462 showed good expression and similar physicochemical properties suggesting that most combinations of XTEN_AM36 segments yield useful XTEN sequences. 30 isolates from LCW0462 were chosen as a preferred collection of XTEN_AM144 segments for the construction of multifunctional proteins that contain multiple XTEN segments. These preferred XTEN_AM144 segments are listed below in Table 13.

TABLE 13
DNA and amino acid sequences for AM144 segments

| | | SEQ | | SEQ |
|---|---|---|---|---|
| | | ID | | ID |
| Clone | Sequence Trimmed | NO: | Protein Sequence | NO: |
| LCW462_{—} | GGTACCCCGGGCAGCGGTACCGCATCTTCCTCTCC | 565 | GTPGSGTASSSPG | 598 |
| r1 | AGGTAGCTCTACCCCGTCTGGTGCTACCGGTTCCC | | SSTPSGATGSPGS | |
| | CAGGTAGCTCTACCCCGTCTGGTGCAACCGGCTC | | STPSGATGSPGSP | |
| | CCCAGGTAGCCCGGCTGGCTCTCCTACCTCTACTG | | AGSPTSTEEGTSE | |
| | AGGAAGGTACTTCTGAAAGCGCTACTCCTGAGTC | | SATPESGPGTSTE | |
| | TGGTCCAGGTACCTCTACTGAACCGTCCGAAGGT | | PSEGSAPGSSPSA | |
| | AGCGCTCCAGGTTCTAGCCCTTCTGCATCCACCGG | | STGTGPGSSPSAS | |
| | TACCGGCCCAGGTTCTAGCCCGTCTGCTTCTACCG | | TGTGPGASPGTSS | |
| | GTACTGGTCCAGGTGCTTCTCCGGGTACTAGCTCT | | TGSPGTSTEPSEG | |
| | ACTGGTTCTCCAGGTACCTCTACCGAACCGTCCGA | | SAPGTSTEPSEGS | |
| | GGGTAGCGCACCAGGTACCTCTACTGAACCGTCT | | APGSEPATSGSET | |
| | GAGGGTAGCGCTCCAGGTAGCGAACCGGCAACCT | | P | |
| | CCGGTTCTGAAACTCCA | | | |
| LCW462_{—} | GGTTCTACCAGCGAATCCCCTTCTGGCACTGCACC | 566 | GSTSESPSGTAPG | 599 |
| r5 | AGGTTCTACTAGCGAATCCCCTTCTGGTACCGCAC | | STSESPSGTAPGT | |
| | CAGGTACTTCTCCGAGCGGCGAATCTTCTACTGCT | | SPSGESSTAPGTS | |
| | CCAGGTACCTCTACTGAACCTTCCGAAGGCAGCG | | TEPSEGSAPGTST | |
| | CTCCAGGTACCTCTACCGAACCGTCCGAGGGCAG | | EPSEGSAPGTSES | |
| | CGCACCAGGTACTTCTGAAAGCGCAACCCCTGAA | | ATPESGPGASPGT | |
| | TCCGGTCCAGGTGCATCTCCTGGTACCAGCTCTAC | | SSTGSPGSSTPSG | |
| | CGGTTCTCCAGGTAGCTCTACTCCTTCTGGTGCTA | | ATGSPGASPGTSS | |
| | CTGGCTCTCCAGGTGCTTCCCCGGGTACCAGCTCT | | TGSPGSTSESPSG | |
| | ACCGGTTCTCCAGGTTCTACTAGCGAATCTCCTTC | | TAPGSTSESPSGT | |
| | TGGCACTGCACCAGGTTCTACCAGCGAATCTCCG | | APGTSTPESGSAS | |
| | TCTGGCACTGCACCAGGTACCTCTACCCCTGAAA | | P | |
| | GCGGTTCCGCTTCTCCA | | | |
| LCW462_{—} | GGTACTTCTACCGAACCTTCCGAGGGCAGCGCAC | 567 | GTSTEPSEGSAPG | 600 |
| r9 | CAGGTACTTCTGAAAGCGCTACCCCTGAGTCCGG | | TSESATPESGPGT | |
| | CCCAGGTACTTCTGAAAGCGCTACTCCTGAATCC | | SESATPESGPGTS | |
| | GGTCCAGGTACCTCTACTGAACCTTCTGAGGGCA | | TEPSEGSAPGTSE | |
| | GCGCTCCAGGTACTTCTGAAAGCGCTACCCCGGA | | SATPESGPGTSTE | |
| | GTCCGGTCCAGGTACTTCTACTGAACCGTCCGAA | | PSEGSAPGTSTEP | |
| | GGTAGCGCACCAGGTACTTCTACTGAACCTTCCG | | SEGSAPGSEPATS | |
| | AAGGTAGCGCTCCAGGTAGCGAACCTGCTACTTC | | GSETPGSPAGSPT | |
| | TGGTTCTGAAACCCCAGGTAGCCCGGCTGGCTCT | | STEEGASPGTSST | |
| | CCGACCTCCACCGAGGAAGGTGCTTCTCCTGGCA | | GSPGSSPSASTGT | |
| | CCAGCTCTACTGGTTCTCCAGGTTCTAGCCCTTCT | | GPGSSPSASTGTG | |
| | GCTTCTACCGGTACTGGTCCAGGTTCTAGCCCTTC | | P | |
| | TGCATCCACTGGTACTGGTCCA | | | |
| LCW462_{—} | GGTAGCGAACCGGCAACCTCTGGCTCTGAAACCC | 568 | GSEPATSGSETPG | 601 |
| r10 | CAGGTACCTCTGAAAGCGCTACTCCGGAATCTGG | | TSESATPESGPGT | |
| | TCCAGGTACTTCTGAAAGCGCTACTCCGGAATCC | | SESATPESGPGST | |
| | GGTCCAGGTTCTACCAGCGAATCTCCTTCTGGCAC | | SESPSGTAPGSTS | |
| | CGCTCCAGGTTCTACTAGCGAATCCCCGTCTGGTA | | ESPSGTAPGTSPS | |
| | CCGCACCAGGTACTTCTCCTAGCGGCGAATCTTCT | | GESSTAPGASPGT | |
| | ACCGCACCAGGTGCATCTCCGGGTACTAGCTCTA | | SSTGSPGSSPSAS | |
| | CCGGTTCTCCAGGTTCTAGCCCTTCTGCTTCCACT | | TGTGPGSSTPSGA | |
| | GGTACCGGCCCAGGTAGCTCTACCCCGTCTGGTG | | TGSPGSSTPSGAT | |
| | CTACTGGTTCCCCAGGTAGCTCTACTCCGTCTGGT | | GSPGSSTPSGATG | |
| | GCAACCGGTTCCCCAGGTAGCTCTACTCCTTCTGG | | SPGASPGTSSTGS | |
| | TGCTACTGGCTCCCCAGGTGCATCCCCTGGCACCA | | P | |
| | GCTCTACCGGTTCTCCA | | | |
| LCW462_{—} | GGTGCTTCTCCGGGCACCAGCTCTACTGGTTCTCC | 569 | GASPGTSSTGSPG | 602 |
| r15 | AGGTTCTAGCCCTTCTGCATCCACCGGTACCGGTC | | SSPSASTGTGPGS | |
| | CAGGTAGCTCTACCCCTTCTGGTGCAACCGGCTCT | | STPSGATGSPGTS | |
| | CCAGGTACTTCTGAAAGCGCTACCCCGGAATCTG | | ESATPESGPGSEP | |
| | GCCCAGGTAGCGAACCGGCTACTTCTGGTTCTGA | | ATSGSETPGSEPA | |
| | AACCCCAGGTAGCGAACCGGCTACCTCCGGTTCT | | TSGSETPGTSESA | |
| | GAAACTCCAGGTACTTCTGAAAGCGCTACTCCGG | | TPESGPGTSTEPS | |
| | AGTCCGGTCCAGGTACCTCTACCGAACCGTCCGA | | EGSAPGTSTEPSE | |
| | AGGCAGCGCTCCAGGTACTTCTACTGAACCTTCTG | | GSAPGTSTEPSEG | |
| | AGGGTAGCGCTCCAGGTACCTCTACCGAACCGTC | | SAPGTSTEPSEGS | |
| | CGAGGGTAGCGCACCAGGTACCTCTACTGAACCG | | APGSEPATSGSET | |
| | TCTGAGGGTAGCGCTCCAGGTAGCGAACCGGCAA | | P | |
| | CCTCCGGTTCTGAAACTCCA | | | |
| LCW462_{—} | GGTACCTCTACCGAACCTTCCGAAGGTAGCGCTC | 570 | GTSTEPSEGSAPG | 603 |
| r16 | CAGGTAGCCCGGCAGGTTCTCCTACTTCCACTGA | | SPAGSPTSTEEGT | |
| | GGAAGGTACTTCTACCGAACCTTCTGAGGGTAGC | | STEPSEGSAPGTS | |
| | GCACCAGGTACCTCTGAAAGCGCAACTCCTGAGT | | ESATPESGPGSEP | |
| | CTGGCCCAGGTAGCGAACCTGCTACCTCCGGCTC | | ATSGSETPGTSES | |
| | TGAGACTCCAGGTACCTCTGAAAGCGCAACCCCG | | ATPESGPGSPAGS | |
| | GAATCTGGTCCAGGTAGCCCGGCTGGCTCTCCTA | | PTSTEEGTSESAT | |
| | CCTCTACTGAGGAAGGTACTTCTGAAAGCGCTAC | | PESGPGTSTEPSE | |
| | TCCTGAGTCTGGTCCAGGTACCTCTACTGAACCGT | | GSAPGSEPATSGS | |
| | CCGAAGGTAGCGCTCCAGGTAGCGAACCTGCTAC | | ETPGTSTEPSEGS | |
| | TTCTGGTTCTGAAACTCCAGGTACTTCTACCGAAC | | APGSEPATSGSET | |
| | CGTCCGAGGGTAGCGCTCCAGGTAGCGAACCTGC | | P | |
| | TACTTCTGGTTCTGAAACTCCA | | | |
| LCW462_{—} | GGTACTTCTACCGAACCGTCCGAAGGCAGCGCTC | 571 | GTSTEPSEGSAPG | 604 |
| r20 | CAGGTACCTCTACTGAACCTTCCGAGGGCAGCGC | | TSTEPSEGSAPGT | |
| | TCCAGGTACCTCTACCGAACCTTCTGAAGGTAGC | | STEPSEGSAPGTS | |
| | GCACCAGGTACTTCTACCGAACCGTCCGAAGGCA | | TEPSEGSAPGTST | |
| | GCGCTCCAGGTACCTCTACTGAACCTTCCGAGGG | | EPSEGSAPGTSTE | |
| | CAGCGCTCCAGGTACCTCTACCGAACCTTCTGAA | | PSEGSAPGTSTEP | |
| | GGTAGCGCACCAGGTACTTCTACCGAACCTTCCG | | SEGSAPGTSESAT | |
| | AGGGCAGCGCACCAGGTACTTCTGAAAGCGCTAC | | PESGPGTSESATP | |
| | CCCTGAGTCCGGCCCAGGTACTTCTGAAAGCGCT | | ESGPGTSTEPSEG | |
| | ACTCCTGAATCCGGTCCAGGTACTTCTACTGAACC | | SAPGSEPATSGSE | |
| | TTCCGAAGGTAGCGCTCCAGGTAGCGAACCTGCT | | TPGSPAGSPTSTE | |
| | ACTTCTGGTTCTGAAACCCCAGGTAGCCCGGCTG | | E | |
| | GCTCTCCGACCTCCACCGAGGAA | | | |
| LCW462_{—} | GGTACTTCTACCGAACCGTCCGAGGGCAGCGCTC | 572 | GTSTEPSEGSAPG | 605 |
| r23 | CAGGTACTTCTACTGAACCTTCTGAAGGCAGCGC | | TSTEPSEGSAPGT | |
| | TCCAGGTACTTCTACTGAACCTTCCGAAGGTAGC | | STEPSEGSAPGST | |
| | GCACCAGGTTCTACCAGCGAATCCCCTTCTGGTAC | | SESPSGTAPGSTS | |
| | TGCTCCAGGTTCTACCAGCGAATCCCCTTCTGGCA | | ESPSGTAPGTSTP | |
| | CCGCACCAGGTACTTCTACCCCTGAAAGCGGCTC | | ESGSASPGSEPAT | |
| | CGCTTCTCCAGGTAGCGAACCTGCAACCTCTGGCT | | SGSETPGTSESAT | |
| | CTGAAACCCCAGGTACCTCTGAAAGCGCTACTCC | | PESGPGTSTEPSE | |
| | TGAATCTGGCCCAGGTACTTCTACTGAACCGTCCG | | GSAPGTSTEPSEG | |
| | AGGGCAGCGCACCAGGTACTTCTACTGAACCGTC | | SAPGTSESATPES | |
| | TGAAGGTAGCGCACCAGGTACTTCTGAAAGCGCA | | GPGTSESATPESG | |
| | ACCCCGGAATCCGGCCCAGGTACCTCTGAAAGCG | | P | |
| | CAACCCCGGAGTCCGGCCCA | | | |
| LCW462_{—} | GGTAGCTCTACCCCTTCTGGTGCTACCGGCTCTCC | 573 | GSSTPSGATGSPG | 606 |
| r24 | AGGTTCTAGCCCGTCTGCTTCTACCGGTACCGGTC | | SSPSASTGTGPGS | |
| | CAGGTAGCTCTACCCCTTCTGGTGCTACTGGTTCT | | STPSGATGSPGSP | |
| | CCAGGTAGCCCTGCTGGCTCTCCGACTTCTACTGA | | AGSPTSTEEGSPA | |
| | GGAAGGTAGCCCGGCTGGTTCTCCGACTTCTACT | | GSPTSTEEGTSTE | |
| | GAGGAAGGTACTTCTACCGAACCTTCCGAAGGTA | | PSEGSAPGASPGT | |
| | GCGCTCCAGGTGCTTCCCCGGGCACTAGCTCTACC | | SSTGSPGSSPSAS | |
| | GGTTCTCCAGGTTCTAGCCCTTCTGCATCTACTGG | | TGTGPGTPGSGT | |
| | TACTGGCCCAGGTACTCCGGGCAGCGGTACTGCT | | ASSSPGSTSSTAE | |
| | TCTTCCTCTCCAGGTTCTACTAGCTCTACTGCTGA | | SPGPGTSPSGESS | |
| | ATCTCCTGGCCCAGGTACTTCTCCTAGCGGTGAAT | | TAPGTSTPESGSA | |
| | CTTCTACCGCTCCAGGTACCTCTACTCCGGAAAGC | | SP | |
| | GGTTCTGCATCTCCA | | | |
| LCW462_{—} | GGTACCTCTACTGAACCTTCTGAGGGCAGCGCTC | 574 | GTSTEPSEGSAPG | 607 |
| r27 | CAGGTACTTCTGAAAGCGCTACCCCGGAGTCCGG | | TSESATPESGPGT | |
| | TCCAGGTACTTCTACTGAACCGTCCGAAGGTAGC | | STEPSEGSAPGTS | |
| | GCACCAGGTACTTCTACTGAACCGTCTGAAGGTA | | TEPSEGSAPGTSE | |
| | GCGCACCAGGTACTTCTGAAAGCGCAACCCCGGA | | SATPESGPGTSES | |
| | ATCCGGCCCAGGTACCTCTGAAAGCGCAACCCCG | | ATPESGPGTPGSG | |
| | GAGTCCGGCCCAGGTACTCCTGGCAGCGGTACCG | | TASSSPGASPGTS | |
| | CTTCTTCTTCTCCAGGTGCTTCTCCTGGTACTAGCT | | STGSPGASPGTSS | |
| | CTACTGGTTCTCCAGGTGCTTCTCCGGGCACTAGC | | TGSPGSPAGSPTS | |
| | TCTACTGGTTCTCCAGGTAGCCCTGCTGGCTCTCC | | TEEGSPAGSPTST | |
| | GACTTCTACTGAGGAAGGTAGCCCGGCTGGTTCT | | EEGTSTEPSEGSA | |
| | CCGACTTCTACTGAGGAAGGTACTTCTACCGAAC | | P | |
| | CTTCCGAAGGTAGCGCTCCA | | | |
| LCW462_{—} | GGTAGCCCAGCAGGCTCTCCGACTTCCACTGAGG | 575 | GSPAGSPTSTEEG | 608 |
| r28 | AAGGTACTTCTACTGAACCTTCCGAAGGCAGCGC | | TSTEPSEGSAPGT | |
| | ACCAGGTACCTCTACTGAACCTTCTGAGGGCAGC | | STEPSEGSAPGTS | |
| | GCTCCAGGTACCTCTACCGAACCGTCTGAAGGTA | | TEPSEGSAPGTSE | |
| | GCGCACCAGGTACCTCTGAAAGCGCAACTCCTGA | | SATPESGPGTSES | |
| | GTCCGGTCCAGGTACTTCTGAAAGCGCAACCCCG | | ATPESGPGTPGSG | |
| | GAGTCTGGCCCAGGTACCCCGGGTAGCGGTACTG | | TASSSPGSSTPSG | |
| | CTTCTTCCTCTCCAGGTAGCTCTACCCCTTCTGGT | | ATGSPGASPGTSS | |
| | GCAACCGGCTCTCCAGGTGCTTCTCCGGGCACCA | | TGSPGTSTEPSEG | |
| | GCTCTACCGGTTCTCCAGGTACCTCTACTGAACCT | | SAPGTSESATPES | |
| | TCTGAGGGCAGCGCTCCAGGTACTTCTGAAAGCG | | GPGTSTEPSEGSA | |
| | CTACCCCGGAGTCCGGTCCAGGTACTTCTACTGA | | P | |
| | ACCGTCCGAAGGTAGCGCACCA | | | |
| LCW462_{—} | GGTAGCGAACCGGCAACCTCCGGCTCTGAAACTC | 576 | GSEPATSGSETPG | 609 |
| r38 | CAGGTACTTCTGAAAGCGCTACTCCGGAATCCGG | | TSESATPESGPGS | |
| | CCCAGGTAGCGAACCGGCTACTTCCGGCTCTGAA | | EPATSGSETPGSS | |
| | ACCCCAGGTAGCTCTACCCCGTCTGGTGCAACCG | | TPSGATGSPGTPG | |
| | GCTCCCCAGGTACTCCTGGTAGCGGTACCGCTTCT | | SGTASSSPGSSTP | |
| | TCTTCTCCAGGTAGCTCTACTCCGTCTGGTGCTAC | | SGATGSPGASPGT | |
| | CGGCTCCCCAGGTGCATCTCCTGGTACCAGCTCTA | | SSTGSPGSSTPSG | |
| | CCGGTTCTCCAGGTAGCTCTACTCCTTCTGGTGCT | | ATGSPGASPGTSS | |
| | ACTGGCTCTCCAGGTGCTTCCCCGGGTACCAGCTC | | TGSPGSEPATSGS | |
| | TACCGGTTCTCCAGGTAGCGAACCTGCTACTTCTG | | ETPGTSTEPSEGS | |
| | GTTCTGAAACTCCAGGTACTTCTACCGAACCGTCC | | APGSEPATSGSET | |
| | GAGGGTAGCGCTCCAGGTAGCGAACCTGCTACTT | | P | |
| | CTGGTTCTGAAACTCCA | | | |
| LCW462_{—} | GGTACCTCTACTGAACCTTCCGAAGGCAGCGCTC | 577 | GTSTEPSEGSAPG | 610 |
| r39 | CAGGTACCTCTACCGAACCGTCCGAGGGCAGCGC | | TSTEPSEGSAPGT | |
| | ACCAGGTACTTCTGAAAGCGCAACCCCTGAATCC | | SESATPESGPGSP | |
| | GGTCCAGGTAGCCCTGCTGGCTCTCCGACTTCTAC | | AGSPTSTEEGSPA | |
| | TGAGGAAGGTAGCCCGGCTGGTTCTCCGACTTCT | | GSPTSTEEGTSTE | |
| | ACTGAGGAAGGTACTTCTACCGAACCTTCCGAAG | | PSEGSAPGSPAGS | |
| | GTAGCGCTCCAGGTAGCCCGGCTGGTTCTCCGAC | | PTSTEEGTSTEPSE | |
| | TTCCACCGAGGAAGGTACCTCTACTGAACCTTCTG | | GSAPGTSTEPSEG | |
| | AGGGTAGCGCTCCAGGTACCTCTACTGAACCTTC | | SAPGASPGTSSTG | |
| | CGAAGGCAGCGCTCCAGGTGCTTCCCCGGGCACC | | SPGSSPSASTGTG | |
| | AGCTCTACTGGTTCTCCAGGTTCTAGCCCGTCTGC | | PGSSPSASTGTGP | |
| | TTCTACTGGTACTGGTCCAGGTTCTAGCCCTTCTG | | | |
| | CTTCCACTGGTACTGGTCCA | | | |
| LCW462_{—} | GGTAGCTCTACCCCGTCTGGTGCTACCGGTTCCCC | 578 | GSSTPSGATGSPG | 611 |
| r41 | AGGTGCTTCTCCTGGTACTAGCTCTACCGGTTCTC | | ASPGTSSTGSPGS | |
| | CAGGTAGCTCTACCCCGTCTGGTGCTACTGGCTCT | | STPSGATGSPGSP | |
| | CCAGGTAGCCCTGCTGGCTCTCCAACCTCCACCG | | AGSPTSTEEGTSE | |
| | AAGAAGGTACCTCTGAAAGCGCAACCCCTGAATC | | SATPESGPGSEPA | |
| | CGGCCCAGGTAGCGAACCGGCAACCTCCGGTTCT | | TSGSETPGASPGT | |
| | GAAACCCCAGGTGCATCTCCTGGTACTAGCTCTA | | SSTGSPGSSTPSG | |
| | CTGGTTCTCCAGGTAGCTCTACTCCGTCTGGTGCA | | ATGSPGSSPSAST | |
| | ACCGGCTCTCCAGGTTCTAGCCCTTCTGCATCTAC | | GTGPGSTSESPSG | |
| | CGGTACTGGTCCAGGTTCTACCAGCGAATCCCCTT | | TAPGSTSESPSGT | |
| | CTGGTACTGCTCCAGGTTCTACCAGCGAATCCCCT | | APGTSTPESGSAS | |
| | TCTGGCACCGCACCAGGTACTTCTACCCCTGAAA | | P | |
| | GCGGCTCCGCTTCTCCA | | | |
| LCW462_{—} | GGTTCTACCAGCGAATCTCCTTCTGGCACCGCTCC | 579 | GSTSESPSGTAPG | 612 |
| r42 | AGGTTCTACTAGCGAATCCCCGTCTGGTACCGCA | | STSESPSGTAPGT | |
| | CCAGGTACTTCTCCTAGCGGCGAATCTTCTACCGC | | SPSGESSTAPGTS | |
| | ACCAGGTACCTCTGAAAGCGCTACTCCGGAGTCT | | ESATPESGPGTST | |
| | GGCCCAGGTACCTCTACTGAACCGTCTGAGGGTA | | EPSEGSAPGTSTE | |
| | GCGCTCCAGGTACTTCTACTGAACCGTCCGAAGG | | PSEGSAPGTSTEP | |
| | TAGCGCACCAGGTACCTCTACTGAACCTTCTGAG | | SEGSAPGTSESAT | |
| | GGCAGCGCTCCAGGTACTTCTGAAAGCGCTACCC | | PESGPGTSTEPSE | |
| | CGGAGTCCGGTCCAGGTACTTCTACTGAACCGTC | | GSAPGSSTPSGAT | |
| | CGAAGGTAGCGCACCAGGTAGCTCTACCCCGTCT | | GSPGASPGTSSTG | |
| | GGTGCTACCGGTTCCCCAGGTGCTTCTCCTGGTAC | | SPGSSTPSGATGS | |
| | TAGCTCTACCGGTTCTCCAGGTAGCTCTACCCCGT | | P | |
| | CTGGTGCTACTGGCTCTCCA | | | |
| LCW462_{—} | GGTTCTACTAGCTCTACTGCAGAATCTCCGGGCCC | 580 | GSTSSTAESPGPG | 613 |
| r43 | AGGTACCTCTCCTAGCGGTGAATCTTCTACCGCTC | | TSPSGESSTAPGT | |
| | CAGGTACTTCTCCGAGCGGTGAATCTTCTACCGCT | | SPSGESSTAPGST | |
| | CCAGGTTCTACTAGCTCTACCGCTGAATCTCCGGG | | SSTAESPGPGSTS | |
| | TCCAGGTTCTACCAGCTCTACTGCAGAATCTCCTG | | STAESPGPGTSTP | |
| | GCCCAGGTACTTCTACTCCGGAAAGCGGTTCCGC | | ESGSASPGTSPSG | |
| | TTCTCCAGGTACTTCTCCTAGCGGTGAATCTTCTA | | ESSTAPGSTSSTA | |
| | CCGCTCCAGGTTCTACCAGCTCTACTGCTGAATCT | | ESPGPGTSTPESG | |
| | CCTGGCCCAGGTACTTCTACCCCGGAAAGCGGCT | | SASPGSTSSTAES | |
| | CCGCTTCTCCAGGTTCTACCAGCTCTACCGCTGAA | | PGPGSTSESPSGT | |
| | TCTCCTGGCCCAGGTTCTACTAGCGAATCTCCGTC | | APGTSPSGESSTA | |
| | TGGCACCGCACCAGGTACTTCCCCTAGCGGTGAA | | P | |
| | TCTTCTACTGCACCA | | | |
| LCW462_{—} | GGTACCTCTACTCCGGAAAGCGGTTCCGCATCTCC | 581 | GTSTPESGSASPG | 614 |
| r45 | AGGTTCTACCAGCGAATCCCCGTCTGGCACCGCA | | STSESPSGTAPGS | |
| | CCAGGTTCTACTAGCTCTACTGCTGAATCTCCGGG | | TSSTAESPGPGTS | |
| | CCCAGGTACCTCTACTGAACCTTCCGAAGGCAGC | | TEPSEGSAPGTST | |
| | GCTCCAGGTACCTCTACCGAACCGTCCGAGGGCA | | EPSEGSAPGTSES | |
| | GCGCACCAGGTACTTCTGAAAGCGCAACCCCTGA | | ATPESGPGTSESA | |
| | ATCCGGTCCAGGTACCTCTGAAAGCGCTACTCCG | | TPESGPGTSTEPS | |
| | GAGTCTGGCCCAGGTACCTCTACTGAACCGTCTG | | EGSAPGTSTEPSE | |
| | AGGGTAGCGCTCCAGGTACTTCTACTGAACCGTC | | GSAPGTSESATPE | |
| | CGAAGGTAGCGCACCAGGTACTTCTGAAAGCGCT | | SGPGTSTEPSEGS | |
| | ACTCCGGAGTCCGGTCCAGGTACCTCTACCGAAC | | APGTSTEPSEGSA | |
| | CGTCCGAAGGCAGCGCTCCAGGTACTTCTACTGA | | P | |
| | ACCTTCTGAGGGTAGCGCTCCC | | | |
| LCW462_{—} | GGTACCTCTACCGAACCGTCCGAGGGTAGCGCAC | 582 | GTSTEPSEGSAPG | 615 |
| r47 | CAGGTACCTCTACTGAACCGTCTGAGGGTAGCGC | | TSTEPSEGSAPGS | |
| | TCCAGGTAGCGAACCGGCAACCTCCGGTTCTGAA | | EPATSGSETPGTS | |
| | ACTCCAGGTACTTCTACTGAACCGTCTGAAGGTA | | TEPSEGSAPGTSE | |
| | GCGCACCAGGTACTTCTGAAAGCGCAACCCCGGA | | SATPESGPGTSES | |
| | ATCCGGCCCAGGTACCTCTGAAAGCGCAACCCCG | | ATPESGPGASPGT | |
| | GAGTCCGGCCCAGGTGCATCTCCGGGTACTAGCT | | SSTGSPGSSPSAS | |
| | CTACCGGTTCTCCAGGTTCTAGCCCTTCTGCTTCC | | TGTGPGSSTPSGA | |
| | ACTGGTACCGGCCCAGGTAGCTCTACCCCGTCTG | | TGSPGSSTPSGAT | |
| | GTGCTACTGGTTCCCCAGGTAGCTCTACTCCGTCT | | GSPGSSTPSGATG | |
| | GGTGCAACCGGTTCCCCAGGTAGCTCTACTCCTTC | | SPGASPGTSSTGS | |
| | TGGTGCTACTGGCTCCCCAGGTGCATCCCCTGGCA | | P | |
| | CCAGCTCTACCGGTTCTCCA | | | |
| LCW462_{—} | GGTAGCGAACCGGCAACCTCTGGCTCTGAAACTC | 583 | GSEPATSGSETPG | 616 |
| r54 | CAGGTAGCGAACCTGCAACCTCCGGCTCTGAAAC | | SEPATSGSETPGT | |
| | CCCAGGTACTTCTACTGAACCTTCTGAGGGCAGC | | STEPSEGSAPGSE | |
| | GCACCAGGTAGCGAACCTGCAACCTCTGGCTCTG | | PATSGSETPGTSE | |
| | AAACCCCAGGTACCTCTGAAAGCGCTACTCCTGA | | SATPESGPGTSTE | |
| | ATCTGGCCCAGGTACTTCTACTGAACCGTCCGAG | | PSEGSAPGSSTPS | |
| | GGCAGCGCACCAGGTAGCTCTACTCCGTCTGGTG | | GATGSPGSSTPSG | |
| | CTACCGGCTCTCCAGGTAGCTCTACCCCTTCTGGT | | ATGSPGASPGTSS | |
| | GCAACCGGCTCCCCAGGTGCTTCTCCGGGTACCA | | TGSPGSSTPSGAT | |
| | GCTCTACTGGTTCTCCAGGTAGCTCTACCCCGTCT | | GSPGASPGTSSTG | |
| | GGTGCTACCGGTTCCCCAGGTGCTTCTCCTGGTAC | | SPGSSTPSGATGS | |
| | TAGCTCTACCGGTTCTCCAGGTAGCTCTACCCCGT | | P | |
| | CTGGTGCTACTGGCTCTCCA | | | |
| LCW462_{—} | GGTACTTCTACCGAACCGTCCGAGGGCAGCGCTC | 584 | GTSTEPSEGSAPG | 617 |
| r55 | CAGGTACTTCTACTGAACCTTCTGAAGGCAGCGC | | TSTEPSEGSAPGT | |
| | TCCAGGTACTTCTACTGAACCTTCCGAAGGTAGC | | STEPSEGSAPGTS | |
| | GCACCAGGTACTTCTGAAAGCGCTACTCCGGAGT | | ESATPESGPGTST | |
| | CCGGTCCAGGTACCTCTACCGAACCGTCCGAAGG | | EPSEGSAPGTSTE | |
| | CAGCGCTCCAGGTACTTCTACTGAACCTTCTGAGG | | PSEGSAPGSTSES | |
| | GTAGCGCTCCAGGTTCTACTAGCGAATCTCCGTCT | | PSGTAPGTSPSGE | |
| | GGCACTGCTCCAGGTACTTCTCCTAGCGGTGAATC | | SSTAPGTSPSGES | |
| | TTCTACCGCTCCAGGTACTTCCCCTAGCGGCGAAT | | STAPGSPAGSPTS | |
| | CTTCTACCGCTCCAGGTAGCCCGGCTGGCTCTCCT | | TEEGTSESATPES | |
| | ACCTCTACTGAGGAAGGTACTTCTGAAAGCGCTA | | GPGTSTEPSEGSA | |
| | CTCCTGAGTCTGGTCCAGGTACCTCTACTGAACCG | | P | |
| | TCCGAAGGTAGCGCTCCA | | | |
| LCW462_{—} | GGTACTTCTACTGAACCTTCCGAAGGTAGCGCTCC | 585 | GTSTEPSEGSAPG | 618 |
| r57 | AGGTAGCGAACCTGCTACTTCTGGTTCTGAAACC | | SEPATSGSETPGS | |
| | CCAGGTAGCCCGGCTGGCTCTCCGACCTCCACCG | | PAGSPTSTEEGSP | |
| | AGGAAGGTAGCCCGGCAGGCTCTCCGACCTCTAC | | AGSPTSTEEGTSE | |
| | TGAGGAAGGTACTTCTGAAAGCGCAACCCCGGAG | | SATPESGPGTSTE | |
| | TCCGGCCCAGGTACCTCTACCGAACCGTCTGAGG | | PSEGSAPGTSTEP | |
| | GCAGCGCACCAGGTACCTCTACTGAACCTTCCGA | | SEGSAPGTSTEPS | |
| | AGGCAGCGCTCCAGGTACCTCTACCGAACCGTCC | | EGSAPGTSESATP | |
| | GAGGGCAGCGCACCAGGTACTTCTGAAAGCGCAA | | ESGPGSSTPSGAT | |
| | CCCCTGAATCCGGTCCAGGTAGCTCTACTCCGTCT | | GSPGSSPSASTGT | |
| | GGTGCAACCGGCTCCCCAGGTTCTAGCCCGTCTG | | GPGASPGTSSTGS | |
| | CTTCCACTGGTACTGGCCCAGGTGCTTCCCCGGGC | | P | |
| | ACCAGCTCTACTGGTTCTCCA | | | |
| LCW462_{—} | GGTAGCGAACCGGCTACTTCCGGCTCTGAGACTC | 586 | GSEPATSGSETPG | 619 |
| r61 | CAGGTAGCCCTGCTGGCTCTCCGACCTCTACCGA | | SPAGSPTSTEEGT | |
| | AGAAGGTACCTCTGAAAGCGCTACCCCTGAGTCT | | SESATPESGPGTS | |
| | GGCCCAGGTACCTCTACTGAACCTTCCGAAGGCA | | TEPSEGSAPGTST | |
| | GCGCTCCAGGTACCTCTACCGAACCGTCCGAGGG | | EPSEGSAPGTSES | |
| | CAGCGCACCAGGTACTTCTGAAAGCGCAACCCCT | | ATPESGPGTSTPE | |
| | GAATCCGGTCCAGGTACCTCTACTCCGGAAAGCG | | SGSASPGSTSESP | |
| | GTTCCGCATCTCCAGGTTCTACCAGCGAATCCCCG | | SGTAPGSTSSTAE | |
| | TCTGGCACCGCACCAGGTTCTACTAGCTCTACTGC | | SPGPGTSESATPE | |
| | TGAATCTCCGGGCCCAGGTACTTCTGAAAGCGCT | | SGPGTSTEPSEGS | |
| | ACTCCGGAGTCCGGTCCAGGTACCTCTACCGAAC | | APGTSTEPSEGSA | |
| | CGTCCGAAGGCAGCGCTCCAGGTACTTCTACTGA | | P | |
| | ACCTTCTGAGGGTAGCGCTCCA | | | |
| LCW462_{—} | GGTACTTCTACCGAACCGTCCGAGGGCAGCGCTC | 587 | GTSTEPSEGSAPG | 620 |
| r64 | CAGGTACTTCTACTGAACCTTCTGAAGGCAGCGC | | TSTEPSEGSAPGT | |
| | TCCAGGTACTTCTACTGAACCTTCCGAAGGTAGC | | STEPSEGSAPGTS | |
| | GCACCAGGTACCTCTACCGAACCGTCTGAAGGTA | | TEPSEGSAPGTSE | |
| | GCGCACCAGGTACCTCTGAAAGCGCAACTCCTGA | | SATPESGPGTSES | |
| | GTCCGGTCCAGGTACTTCTGAAAGCGCAACCCCG | | ATPESGPGTPGSG | |
| | GAGTCTGGCCCAGGTACTCCTGGCAGCGGTACCG | | TASSSPGSSTPSG | |
| | CATCTTCCTCTCCAGGTAGCTCTACTCCGTCTGGT | | ATGSPGASPGTSS | |
| | GCAACTGGTTCCCCAGGTGCTTCTCCGGGTACCA | | TGSPGSTSSTAES | |
| | GCTCTACCGGTTCTCCAGGTTCCACCAGCTCTACT | | PGPGTSPSGESST | |
| | GCTGAATCTCCTGGTCCAGGTACCTCTCCTAGCGG | | APGTSTPESGSAS | |
| | TGAATCTTCTACTGCTCCAGGTACTTCTACTCCTG | | P | |
| | AAAGCGGCTCTGCTTCTCCA | | | |
| LCW462_{—} | GGTAGCCCGGCAGGCTCTCCGACCTCTACTGAGG | 588 | GSPAGSPTSTEEG | 621 |
| r67 | AAGGTACTTCTGAAAGCGCAACCCCGGAGTCCGG | | TSESATPESGPGT | |
| | CCCAGGTACCTCTACCGAACCGTCTGAGGGCAGC | | STEPSEGSAPGTS | |
| | GCACCAGGTACTTCTGAAAGCGCAACCCCTGAAT | | ESATPESGPGSEP | |
| | CCGGTCCAGGTAGCGAACCGGCTACTTCTGGCTC | | ATSGSETPGTSTE | |
| | TGAGACTCCAGGTACTTCTACCGAACCGTCCGAA | | PSEGSAPGSPAGS | |
| | GGTAGCGCACCAGGTAGCCCGGCTGGTTCTCCGA | | PTSTEEGTSTEPSE | |
| | CTTCCACCGAGGAAGGTACCTCTACTGAACCTTCT | | GSAPGTSTEPSEG | |
| | GAGGGTAGCGCTCCAGGTACCTCTACTGAACCTT | | SAPGTSTEPSEGS | |
| | CCGAAGGCAGCGCTCCAGGTACTTCTACCGAACC | | APGTSTEPSEGSA | |
| | GTCCGAGGGCAGCGCTCCAGGTACTTCTACTGAA | | PGTSTEPSEGSAP | |
| | CCTTCTGAAGGCAGCGCTCCAGGTACTTCTACTGA | | | |
| | ACCTTCCGAAGGTAGCGCACCA | | | |
| LCW462_{—} | GGTACTTCTCCGAGCGGTGAATCTTCTACCGCACC | 589 | GTSPSGESSTAPG | 622 |
| r69 | AGGTTCTACTAGCTCTACCGCTGAATCTCCGGGCC | | STSSTAESPGPGT | |
| | CAGGTACTTCTCCGAGCGGTGAATCTTCTACTGCT | | SPSGESSTAPGTS | |
| | CCAGGTACCTCTGAAAGCGCTACTCCGGAGTCTG | | ESATPESGPGTST | |
| | GCCCAGGTACCTCTACTGAACCGTCTGAGGGTAG | | EPSEGSAPGTSTE | |
| | CGCTCCAGGTACTTCTACTGAACCGTCCGAAGGT | | PSEGSAPGSSPSA | |
| | AGCGCACCAGGTTCTAGCCCTTCTGCATCTACTGG | | STGTGPGSSTPSG | |
| | TACTGGCCCAGGTAGCTCTACTCCTTCTGGTGCTA | | ATGSPGASPGTSS | |
| | CCGGCTCTCCAGGTGCTTCTCCGGGTACTAGCTCT | | TGSPGTSTPESGS | |
| | ACCGGTTCTCCAGGTACTTCTACTCCGGAAAGCG | | ASPGTSPSGESST | |
| | GTTCCGCATCTCCAGGTACTTCTCCTAGCGGTGAA | | APGTSPSGESSTA | |
| | TCTTCTACTGCTCCAGGTACCTCTCCTAGCGGCGA | | P | |
| | ATCTTCTACTGCTCCA | | | |
| LCW462_{—} | GGTACCTCTGAAAGCGCTACTCCGGAGTCTGGCC | 590 | GTSESATPESGPG | 623 |
| r70 | CAGGTACCTCTACTGAACCGTCTGAGGGTAGCGC | | TSTEPSEGSAPGT | |
| | TCCAGGTACTTCTACTGAACCGTCCGAAGGTAGC | | STEPSEGSAPGSP | |
| | GCACCAGGTAGCCCTGCTGGCTCTCCGACTTCTAC | | AGSPTSTEEGSPA | |
| | TGAGGAAGGTAGCCCGGCTGGTTCTCCGACTTCT | | GSPTSTEEGTSTE | |
| | ACTGAGGAAGGTACTTCTACCGAACCTTCCGAAG | | PSEGSAPGSSPSA | |
| | GTAGCGCTCCAGGTTCTAGCCCTTCTGCTTCCACC | | STGTGPGSSTPSG | |
| | GGTACTGGCCCAGGTAGCTCTACCCCTTCTGGTGC | | ATGSPGSSTPSGA | |
| | TACCGGCTCCCCAGGTAGCTCTACTCCTTCTGGTG | | TGSPGSEPATSGS | |
| | CAACTGGCTCTCCAGGTAGCGAACCGGCAACTTC | | ETPGTSESATPES | |
| | CGGCTCTGAAACCCCAGGTACTTCTGAAAGCGCT | | GPGSEPATSGSET | |
| | ACTCCTGAGTCTGGCCCAGGTAGCGAACCTGCTA | | P | |
| | CCTCTGGCTCTGAAACCCCA | | | |
| LCW462_{—} | GGTACTTCTACCGAACCGTCCGAAGGCAGCGCTC | 591 | GTSTEPSEGSAPG | 624 |
| r72 | CAGGTACCTCTACTGAACCTTCCGAGGGCAGCGC | | TSTEPSEGSAPGT | |
| | TCCAGGTACCTCTACCGAACCTTCTGAAGGTAGC | | STEPSEGSAPGSS | |
| | GCACCAGGTAGCTCTACCCCGTCTGGTGCTACCG | | TPSGATGSPGASP | |
| | GTTCCCCAGGTGCTTCTCCTGGTACTAGCTCTACC | | GTSSTGSPGSSTP | |
| | GGTTCTCCAGGTAGCTCTACCCCGTCTGGTGCTAC | | SGATGSPGTSESA | |
| | TGGCTCTCCAGGTACTTCTGAAAGCGCAACCCCT | | TPESGPGSEPATS | |
| | GAATCCGGTCCAGGTAGCGAACCGGCTACTTCTG | | GSETPGTSTEPSE | |
| | GCTCTGAGACTCCAGGTACTTCTACCGAACCGTCC | | GSAPGSTSESPSG | |
| | GAAGGTAGCGCACCAGGTTCTACTAGCGAATCTC | | TAPGSTSESPSGT | |
| | CTTCTGGCACTGCACCAGGTTCTACCAGCGAATCT | | APGTSTPESGSAS | |
| | CCGTCTGGCACTGCACCAGGTACCTCTACCCCTGA | | P | |
| | AAGCGGTTCCGCTTCTCCA | | | |
| LCW462_{—} | GGTACCTCTACTCCTGAAAGCGGTTCTGCATCTCC | 592 | GTSTPESGSASPG | 625 |
| r73 | AGGTTCCACTAGCTCTACCGCAGAATCTCCGGGC | | STSSTAESPGPGS | |
| | CCAGGTTCTACTAGCTCTACTGCTGAATCTCCTGG | | TSSTAESPGPGSS | |
| | CCCAGGTTCTAGCCCTTCTGCATCTACTGGTACTG | | PSASTGTGPGSST | |
| | GCCCAGGTAGCTCTACTCCTTCTGGTGCTACCGGC | | PSGATGSPGASPG | |
| | TCTCCAGGTGCTTCTCCGGGTACTAGCTCTACCGG | | TSSTGSPGSEPAT | |
| | TTCTCCAGGTAGCGAACCGGCAACCTCCGGCTCT | | SGSETPGTSESAT | |
| | GAAACCCCAGGTACCTCTGAAAGCGCTACTCCTG | | PESGPGSPAGSPT | |
| | AATCCGGCCCAGGTAGCCCGGCAGGTTCTCCGAC | | STEEGSTSESPSG | |
| | TTCCACTGAGGAAGGTTCTACTAGCGAATCTCCTT | | TAPGSTSESPSGT | |
| | CTGGCACTGCACCAGGTTCTACCAGCGAATCTCC | | APGTSTPESGSAS | |
| | GTCTGGCACTGCACCAGGTACCTCTACCCCTGAA | | P | |
| | AGCGGTTCCGCTTCTCCC | | | |
| LCW462_{—} | GGTAGCCCGGCTGGCTCTCCTACCTCTACTGAGG | 593 | GSPAGSPTSTEEG | 626 |
| r78 | AAGGTACTTCTGAAAGCGCTACTCCTGAGTCTGG | | TSESATPESGPGT | |
| | TCCAGGTACCTCTACTGAACCGTCCGAAGGTAGC | | STEPSEGSAPGST | |
| | GCTCCAGGTTCTACCAGCGAATCTCCTTCTGGCAC | | SESPSGTAPGSTS | |
| | CGCTCCAGGTTCTACTAGCGAATCCCCGTCTGGTA | | ESPSGTAPGTSPS | |
| | CCGCACCAGGTACTTCTCCTAGCGGCGAATCTTCT | | GESSTAPGTSTEP | |
| | ACCGCACCAGGTACCTCTACCGAACCTTCCGAAG | | SEGSAPGSPAGSP | |
| | GTAGCGCTCCAGGTAGCCCGGCAGGTTCTCCTAC | | TSTEEGTSTEPSE | |
| | TTCCACTGAGGAAGGTACTTCTACCGAACCTTCTG | | GSAPGSEPATSGS | |
| | AGGGTAGCGCACCAGGTAGCGAACCTGCAACCTC | | ETPGTSESATPES | |
| | TGGCTCTGAAACCCCAGGTACCTCTGAAAGCGCT | | GPGTSTEPSEGSA | |
| | ACTCCTGAATCTGGCCCAGGTACTTCTACTGAACC | | P | |
| | GTCCGAGGGCAGCGCACCA | | | |
| LCW462_{—} | GGTACCTCTACCGAACCTTCCGAAGGTAGCGCTC | 594 | GTSTEPSEGSAPG | 627 |
| r79 | CAGGTAGCCCGGCAGGTTCTCCTACTTCCACTGA | | SPAGSPTSTEEGT | |
| | GGAAGGTACTTCTACCGAACCTTCTGAGGGTAGC | | STEPSEGSAPGTS | |
| | GCACCAGGTACCTCCCCTAGCGGCGAATCTTCTA | | PSGESSTAPGTSP | |
| | CTGCTCCAGGTACCTCTCCTAGCGGCGAATCTTCT | | SGESSTAPGTSPS | |
| | ACCGCTCCAGGTACCTCCCCTAGCGGTGAATCTTC | | GESSTAPGSTSES | |
| | TACCGCACCAGGTTCTACCAGCGAATCCCCTTCTG | | PSGTAPGSTSESP | |
| | GTACTGCTCCAGGTTCTACCAGCGAATCCCCTTCT | | SGTAPGTSTPESG | |
| | GGCACCGCACCAGGTACTTCTACCCCTGAAAGCG | | SASPGSEPATSGS | |
| | GCTCCGCTTCTCCAGGTAGCGAACCTGCAACCTCT | | ETPGTSESATPES | |
| | GGCTCTGAAACCCCAGGTACCTCTGAAAGCGCTA | | GPGTSTEPSEGSA | |
| | CTCCTGAATCTGGCCCAGGTACTTCTACTGAACCG | | P | |
| | TCCGAGGGCAGCGCACCA | | | |
| LCW462_{—} | GGTAGCGAACCGGCAACCTCTGGCTCTGAAACCC | 595 | GSEPATSGSETPG | 628 |
| r87 | CAGGTACCTCTGAAAGCGCTACTCCGGAATCTGG | | TSESATPESGPGT | |
| | TCCAGGTACTTCTGAAAGCGCTACTCCGGAATCC | | SESATPESGPGTS | |
| | GGTCCAGGTACTTCTCCGAGCGGTGAATCTTCTAC | | PSGESSTAPGSTS | |
| | CGCACCAGGTTCTACTAGCTCTACCGCTGAATCTC | | STAESPGPGTSPS | |
| | CGGGCCCAGGTACTTCTCCGAGCGGTGAATCTTCT | | GESSTAPGSTSES | |
| | ACTGCTCCAGGTTCTACTAGCGAATCCCCGTCTGG | | PSGTAPGTSPSGE | |
| | TACTGCTCCAGGTACTTCCCCTAGCGGTGAATCTT | | SSTAPGSTSSTAE | |
| | CTACTGCTCCAGGTTCTACCAGCTCTACCGCAGAA | | SPGPGSSTPSGAT | |
| | TCTCCGGGTCCAGGTAGCTCTACTCCGTCTGGTGC | | GSPGSSTPSGATG | |
| | AACCGGTTCCCCAGGTAGCTCTACCCCTTCTGGTG | | SPGSSTPSGANW | |
| | CAACCGGCTCCCCAGGTAGCTCTACCCCTTCTGGT | | LS | |
| | GCAAACTGGCTCTCC | | | |
| LCW462_{—} | GGTAGCCCTGCTGGCTCTCCGACTTCTACTGAGGA | 596 | GSPAGSPTSTEEG | 629 |
| r88 | AGGTAGCCCGGCTGGTTCTCCGACTTCTACTGAG | | SPAGSPTSTEEGT | |
| | GAAGGTACTTCTACCGAACCTTCCGAAGGTAGCG | | STEPSEGSAPGTS | |
| | CTCCAGGTACCTCTACTGAACCTTCCGAAGGCAG | | TEPSEGSAPGTST | |
| | CGCTCCAGGTACCTCTACCGAACCGTCCGAGGGC | | EPSEGSAPGTSES | |
| | AGCGCACCAGGTACTTCTGAAAGCGCAACCCCTG | | ATPESGPGASPGT | |
| | AATCCGGTCCAGGTGCATCTCCTGGTACCAGCTCT | | SSTGSPGSSTPSG | |
| | ACCGGTTCTCCAGGTAGCTCTACTCCTTCTGGTGC | | ATGSPGASPGTSS | |
| | TACTGGCTCTCCAGGTGCTTCCCCGGGTACCAGCT | | TGSPGSSTPSGAT | |
| | CTACCGGTTCTCCAGGTAGCTCTACCCCGTCTGGT | | GSPGTPGSGTASS | |
| | GCTACTGGTTCTCCAGGTACTCCGGGCAGCGGTA | | SPGSSTPSGATGS | |
| | CTGCTTCTTCCTCTCCAGGTAGCTCTACCCCTTCT | | P | |
| | GGTGCTACTGGCTCTCCA | | | |
| LCW462_{—} | GGTAGCTCTACCCCGTCTGGTGCTACTGGTTCTCC | 597 | GSSTPSGATGSPG | 630 |
| r89 | AGGTACTCCGGGCAGCGGTACTGCTTCTTCCTCTC | | TPGSGTASSSPGS | |
| | CAGGTAGCTCTACCCCTTCTGGTGCTACTGGCTCT | | STPSGATGSPGSP | |
| | CCAGGTAGCCCGGCTGGCTCTCCTACCTCTACTGA | | AGSPTSTEEGTSE | |
| | GGAAGGTACTTCTGAAAGCGCTACTCCTGAGTCT | | SATPESGPGTSTE | |
| | GGTCCAGGTACCTCTACTGAACCGTCCGAAGGTA | | PSEGSAPGTSESA | |
| | GCGCTCCAGGTACCTCTGAAAGCGCAACTCCTGA | | TPESGPGSEPATS | |
| | GTCTGGCCCAGGTAGCGAACCTGCTACCTCCGGC | | GSETPGTSESATP | |
| | TCTGAGACTCCAGGTACCTCTGAAAGCGCAACCC | | ESGPGTSTEPSEG | |
| | CGGAATCTGGTCCAGGTACTTCTACTGAACCGTCT | | SAPGTSESATPES | |
| | GAAGGTAGCGCACCAGGTACTTCTGAAAGCGCAA | | GPGTSESATPESG | |
| | CCCCGGAATCCGGCCCAGGTACCTCTGAAAGCGC | | P | |
| | AACCCCGGAGTCCGGCCCA | | | |

### Example 7

### Construction of XTEN_AM288

The entire library LCW0462 was dimerized as described in Example 6 resulting in a library of XTEN_AM288 clones designated LCW0463. 1512 isolates from library LCW0463 were screened using the protocol described in Example 6. 176 highly expressing clones were sequenced and 40 preferred XTEN_AM288 segments were chosen for the construction of multifunctional proteins that contain multiple XTEN segments.

### Example 8

### Construction of XTEN_AM432

We generated a library of XTEN_AM432 segments by recombining segments from library LCW0462 of XTEN_AM144 segments and segments from library LCW0463 of XTEN_AM288 segments. This new library of XTEN_AM432 segment was designated LCW0464. Plasmid was isolated from cultures of E. coli harboring LCW0462 and LCW0463, respectively. 1512 isolates from library LCW0464 were screened using the protocol described in Example 6. 176 highly expressing clones were sequenced and 39 preferred XTEN_AM432 segment were chosen for the construction of longer XTENs and for the construction of multifunctional proteins that contain multiple XTEN segments.

In parallel we constructed library LMS0100 of XTEN_AM432 segments using preferred segments of XTEN_AM144 and XTEN_AM288. Screening this library yielded 4 isolates that were selected for further construction

### Example 9

### Construction of XTEN_AM875

The stuffer vector pCW0359 was digested with BsaI and KpnI to remove the stuffer segment and the resulting vector fragment was isolated by agarose gel purification.

We annealed the phosphorylated oligonucleotide BsaI-AscI-KpnIforP: AGGTGCAAGCGCAAGCGGCGCGCCAAGCACGGGAGGTTCGTCTTCACTCGAGGGTAC (SEQ ID NO: 631) and the non-phosphorylated oligonucleotide BsaI-AscI-KpnIrev: CCTCGAGTGAAGACGAACCTCCCGTGCTTGGCGCGCCGCTTGCGCTTGC (SEQ ID NO: 632) for introducing the sequencing island A (SI-A) which encodes amino acids GASASGAPSTG (SEQ ID NO: 633) and has the restriction enzyme AscI recognition nucleotide sequence GGCGCGCC inside. The annealed oligonucleotide pairs were ligated with BsaI and KpnI digested stuffer vector pCW0359 prepared above to yield pCW0466 containing SI-A. We then generated a library of XTEN_AM443 segments by recombining 43 preferred XTEN_AM432 segments from Example 8 and SI-A segments from pCW0466 at C-terminus using the same dimerization process described in Example 5. This new library of XTEN_AM443 segments was designated LCW0479.

We generated a library of XTEN_AM875 segments by recombining segments from library LCW0479 of XTEN_AM443 segments and 43 preferred XTEN_AM432 segments from Example 8 using the same dimerization process described in example 5. This new library of XTEN_AM875 segment was designated LCW0481.

### Example 10

### Construction of XTEN_AM1318

We annealed the phosphorylated oligonucleotide BsaI-FseI-KpnIforP:

(SEQ ID NO: 634)
AGGTCCAGAACCAACGGGGCCGGCCCCAAGCGGAGGTTCGTCTTCACTC
GAGGGTAC

and the non-phosphorylated oligonucleotide BsaI-FseI-KpnIrev:

(SEQ ID NO: 635)
CCTCGAGTGAAGACGAACCTCCGCTTGGGGCCGGCCCCGTTGGTTCTGG

the sequencing island B (SI-B) which encodes amino acids GPEPTGPAPSG (SEQ ID NO: 636) and has the restriction enzyme FseI recognition nucleotide sequence GGCCGGCC inside. The annealed oligonucleotide pairs were ligated with BsaI and KpnI digested stuffier vector pCW0359 as used in Example 9 to yield pCW0467 containing SI-B. We then generated a library of XTEN_AM443 segments by recombining 43 preferred XTEN_AM432 segments from Example 8 and SI-B segments from pCW0467 at C-terminus using the same dimerization process described in example 5. This new library of XTEN_AM443 segments was designated LCW0480.

We generated a library of XTEN_AM1318 segments by recombining segments from library LCW0480 of XTEN_AM443 segments and segments from library LCW0481 of XTEN_AM875 segments using the same dimerization process as in example 5. This new library of XTEN_AM1318 segment was designated LCW0487.

### Example 11

### Construction of XTEN_AD864

Using the several consecutive rounds of dimerization, we assembled a collection of XTEN_AD864 sequences starting from segments of XTEN_AD36 listed in Example 1. These sequences were assembled as described in Example 5. Several isolates from XTEN_AD864 were evaluated and found to show good expression and excellent solubility under physiological conditions. One intermediate construct of XTEN_AD576 was sequenced. This clone was evaluated in a PK experiment in cynomolgus monkeys and a half-life of about 20 h was measured.

### Example 12

### Construction of XTEN_AF864

Using the several consecutive rounds of dimerization, we assembled a collection of XTEN_AF864 sequences starting from segments of XTEN_AF36 listed in Example 3. These sequences were assembled as described in Example 5. Several isolates from XTEN_AF864 were evaluated and found to show good expression and excellent solubility under physiological conditions. One intermediate construct of XTEN_AF540 was sequenced. This clone was evaluated in a PK experiment in cynomolgus monkeys and a half-life of about 20 h was measured. A full length clone of XTEN_AF864 had excellent solubility and showed half-life exceeding 60 h in cynomolgus monkeys. A second set of XTEN_AF sequences was assembled including a sequencing island as described in Example 9.

### Example 13

### Construction of XTEN_AG864

Using the several consecutive rounds of dimerization, we assembled a collection of XTEN_AG864 sequences starting from segments of XTEN_AG36 listed in Example 4. These sequences were assembled as described in Example 5. Several isolates from XTEN_AG864 were evaluated and found to show good expression and excellent solubility under physiological conditions. A full length clone of XTEN_AG864 had excellent solubility and showed half-life exceeding 60 h in cynomolgus monkeys.

### Example 14

### Construction of N-Terminal Extensions of XTEN-Construction and Screening of 12Mer Addition Libraries

This example details a step in the optimization of the N-terminus of the XTEN protein to promote the initiation of translation to allow for expression of XTEN fusions at the N-terminus of fusion proteins without the presence of a helper domain. Historically expression of proteins with XTEN at the N-terminus was poor, yielding values that would essentially undetectable in the GFP fluorescence assay (<25% of the expression with the N-terminal CBD helper domain). To create diversity at the codon level, seven amino acid sequences were selected and prepared with a diversity of codons. Seven pairs of oligonucleotides encoding 12 amino acids with codon diversities were designed, annealed and ligated into the NdeI/BsaI restriction enzyme digested stuffer vector pCW0551 (Stuffer-XTEN_AM875-GFP), and transformed into E. coli BL21Gold(DE3) competent cells to obtain colonies of seven libraries. The resulting clones have N-terminal XTEN 12mers fused in-frame to XTEN_AM875-GFP to allow use of GFP fluorescence for screening the expression. Individual colonies from the seven created libraries were picked and grown overnight to saturation in 500 μl of super broth media in a 96 deep well plate. The number of colonies picked ranged from approximately half to a third of the theoretical diversity of the library (see Table 14).

TABLE 14
Theoretical Diversity and Sampling Numbers for 12mer Addition Libraries.
The amino acid residues with randomized codons are underlined.

| | | | SEQ ID | Theoretical | Number |
|---|---|---|---|---|---|
| Library | Motif Family | Amino Acid Sequence | NO: | Diversity | screened |
| LCW546 | AE12 | MASPAGSPTSTEE | 637 | 572 | 2 plates (168) |
| LCW547 | AE12 | MATSESATPESGP | 638 | 1536 | 5 plates (420) |
| LCW548 | AF12 | MATSPSGESSTAP | 639 | 192 | 2 plates (168) |
| LCW549 | AF12 | MESTSSTAESPGP | 640 | 384 | 2 plates (168) |
| LCW552 | AG12 | MASSTPSGATGSP | 641 | 384 | 2 plates (168) |
| LCW553 | AG12 | MEASPGTSSTGSP | 642 | 384 | 2 plates (168) |
| LCW554 | (CBD-like) | MASTPESGSSG | 643 | 32 | 1 plate (84) |

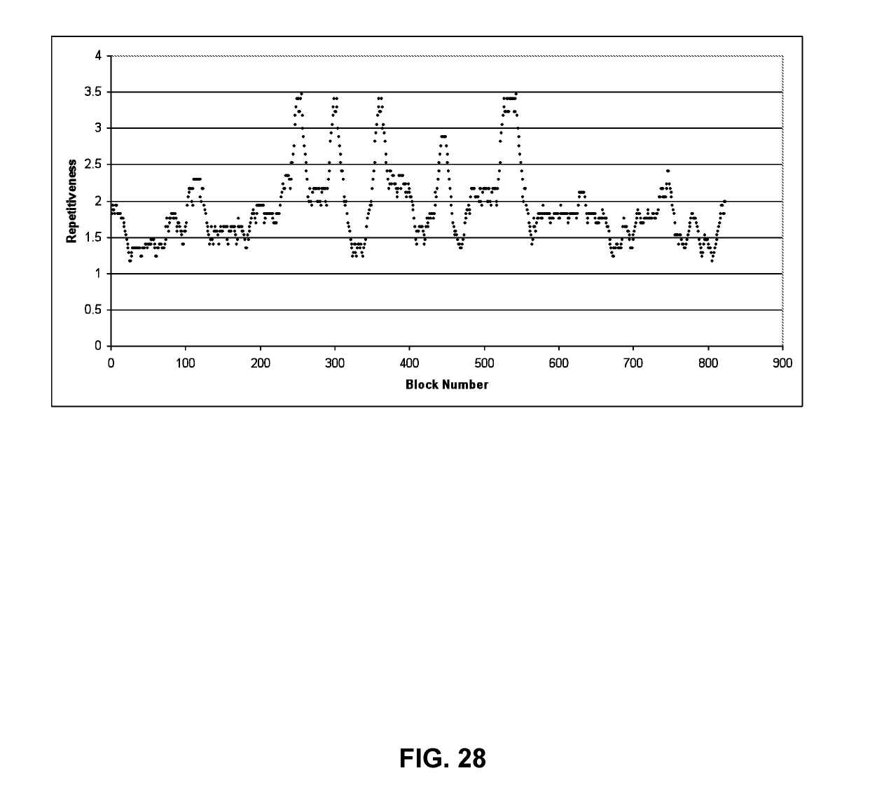

The saturated overnight cultures were used to inoculate fresh 500 μl cultures in auto-induction media in which they were grown overnight at 26° C. These expression cultures were then assayed using a fluorescence plate reader (excitation 395 nm, emission 510 nm) to determine the amount of GFP reporter present (see FIG. 28 for results of expression assays). The results indicated that while median expression levels were approximately half of the expression levels compared to the “benchmark” CBD N-terminal helper domain, the best clones from the libraries were much closer to the benchmarks, indicating that further optimization around those sequences was warranted. This is in contrast to previous XTEN versions that were <25% of the expression levels of the CBD N-terminal benchmark. The results also show that the libraries starting with amino acids MA had better expression levels than those beginning with ME. This was most apparent when looking at the best clones, which were closer to the benchmarks as they mostly start with MA. Of the 176 clones within 33% of the CBD-AM875 benchmark, 87% begin with MA, where as only 75% of the sequences in the libraries beginning with MA, a clear over representation of the clones beginning with MA at the highest level of expression. 96 of the best clones were sequenced to confirm identity and twelve sequences (see Table 15), 4 from LCW546, 4 from LCW547 and 4 from LCW552 were selected for further optimization.

TABLE 15
Advanced 12mer DNA Nucleotide Sequences

| | | SEQ ID |
|---|---|---|
| Clone | DNA NucleotideSequence | NO: |
| LCW546_02 | ATGGCTAGTCCGGCTGGCTCTCCGACCTCCACTGAGGAAGGTACTTCTACT | 644 |
| LCW546_06 | ATGGCTAGTCCTGCTGGCTCTCCAACCTCCACTGAGGAAGGTACTTCTACT | 645 |
| LCW546_07 | ATGGCTAGTCCAGCAGGCTCTCCTACCTCCACCGAGGAAGGTACTTCTACT | 646 |
| LCW546_09 | ATGGCTAGTCCTGCTGGCTCTCCGACCTCTACTGAGGAAGGTACTTCTACT | 647 |
| LCW547_03 | ATGGCTACATCCGAAAGCGCAACCCCTGAGTCCGGTCCAGGTACTTCTACT | 648 |
| LCW547_06 | ATGGCTACATCCGAAAGCGCAACCCCTGAATCTGGTCCAGGTACTTCTACT | 649 |
| LCW547_10 | ATGGCTACGTCTGAAAGCGCTACTCCGGAATCTGGTCCAGGTACTTCTACT | 650 |
| LCW547_17 | ATGGCTACGTCCGAAAGCGCTACCCCTGAATCCGGTCCAGGTACTTCTACT | 651 |
| LCW552_03 | ATGGCTAGTTCTACCCCGTCTGGTGCAACCGGTTCCCCAGGTACTTCTACT | 652 |
| LCW552_05 | ATGGCTAGCTCCACTCCGTCTGGTGCTACCGGTTCCCCAGGTACTTCTACT | 653 |
| LCW552_10 | ATGGCTAGCTCTACTCCGTCTGGTGCTACTGGTTCCCCAGGTACTTCTACT | 654 |
| LCW552_11 | ATGGCTAGTTCTACCCCTTCTGGTGCTACTGGTTCTCCAGGTACTTCTACT | 655 |

### Example 15

### Construction of N-Terminal Extensions of XTEN-Construction and Screening of Libraries Optimizing Codons 3 and 4

This example details a step in the optimization of the N-terminus of the XTEN protein to promote the initiation of translation to allow for expression of XTEN fusions at the N-terminus of proteins without the presence of a helper domain. With preferences for the first two codons established (see Example supra), the third and fourth codons were randomized to determine preferences. Three libraries, based upon best clones from LCW546, LCW547 and LCW552, were designed with the third and fourth residues modified such that all combinations of allowable XTEN codons were present at these positions. In order to include all the allowable XTEN codons for each library, nine pairs of oligonucleotides encoding 12 amino acids with codon diversities of third and fourth residues were designed, annealed and ligated into the NdeI/BsaI restriction enzyme digested stuffer vector pCW0551 (Stuffer-XTEN_AM875-GFP), and transformed into E. coli BL21Gold(DE3) competent cells to obtain colonies of three libraries LCW0569-571. With 24 XTEN codons the theoretical diversity of each library is 576 unique clones. A total of 504 individual colonies from the three created libraries were picked and grown overnight to saturation in 500 μl of super broth media in a 96 deep well plate. This provided sufficient coverage to understand relative library performance and sequence preferences. The saturated overnight cultures were used to inoculate new 500 μl cultures in auto-induction media in which were grown overnight at 26° C. These expression cultures were then assayed using a fluorescence plate reader (excitation 395 nm, emission 510 nm) to determine the amount of GFP reporter present. The top 75 clones from the screen were sequenced and retested for GFP reporter expression versus the benchmark samples. 52 clones yielded usable sequencing data and were used for subsequent analysis. The results were broken down by library and indicate that LCW546 was the superior library. The results are presented in Table 16.

TABLE 16
Third and Fourth Codon Optimization Library Comparison

| | LCW569 | LCW570 | LCW571 |
|---|---|---|---|
| N | 21 | 15 | 16 |
| Mean Fluorescence (AU) | 628 | 491 | 537 |
| SD | 173 | 71 | 232 |
| CV | 28% | 15% | 43% |

Further trends were seen in the data showing preferences for particular codons at the third and fourth position. Within the LCW569 library the glutamate codon GAA at the third position and the threonine codon ACT were associated with higher expression as seen in Table 17.

TABLE 17
Preferred Third and Fourth Codons in LCW569

| | 3 = GAA | Rest | 4 = ACT | Rest |
|---|---|---|---|---|
| N | 8 | 13 | 4 | 17 |
| Mean Fluorescence (AU) | 749 | 554 | 744 | 601 |
| SD | 234 | 47 | 197 | 162 |
| CV | 31% | 9% | 26% | 27% |

Additionally, the retest of the top 75 clones indicated that several were now superior to the benchmark clones.

### Example 16

### Construction of N-Terminal Extensions of XTEN-Construction and Screening of Combinatorial 12mer and 36mer Libraries

This example details a step in the optimization of the N-terminus of the XTEN protein to promote the initiation of translation to allow for expression of XTEN fusions at the N-terminus of proteins without the presence of a helper domain. With preferences for the first two codons established (see Example supra), the N-terminus was examined in a broader context by combining the 12 selected 12mer sequences (see Example supra) at the very N-terminus followed by 125 previously constructed 36mer segments (see example supra) in a combinatorial manner. This created novel 48mers at the N-terminus of the XTEN protein and enabled the assessment of the impact of longer-range interactions at the N-terminus on expression of the longer sequences (FIG. 29) Similar to the dimerization procedures used to assemble 36mers (see Example infra), the plasmids containing the 125 selected 36mer segments were digested with restriction enzymes BbsI/NcoI and the appropriate fragment was gel-purified. The plasmid from clone AC94 (CBD-XTEN_AM875-GFP) was also digested with BsaI/NcoI and the appropriate fragments were gel-purified. These fragments were ligated together and transformed into E. coli BL21Gold(DE3) competent cells to obtain colonies of the library LCW0579, which also served as the vector for further cloning 12 selected 12mers at the very N-terminus. The plasmids of LCW0579 were digested with NdeI/EcoRI/BsaI and the appropriate fragments were gel-purified. 12 pairs of oligonucleotides encoding 12 selected 12mer sequences were designed, annealed and ligated with the NdeI/EcoRI/BsaI digested LCW0579 vector, and transformed into E. coli BL21Gold(DE3) competent cells to obtain colonies of the library LCW0580. With a theoretical diversity of 1500 unique clones, a total of 1512 individual colonies from the created library were picked and grown overnight to saturation in 500 μl of super broth media in a 96 deep well plate. This provided sufficient coverage to understand relative library performance and sequence preferences. The saturated overnight cultures were used to inoculate new 500 μl cultures in auto-induction media that were grown overnight at 26° C. These expression cultures were then assayed using a fluorescence plate reader (excitation 395 nm, emission 510 nm) to determine the amount of GFP reporter present. The top 90 clones were sequenced and retested for GFP reporter expression. 83 clones yielded usable sequencing data and were used for subsequent analysis. The sequencing data was used to determine the lead 12mer that was present in each clone and the impact of each 12mer on expression was assessed. Clones LCW546_{—}06 and LCW546_{—}09 stood out as being the superior N-terminus (see Table 18).

TABLE 18
Relative Performance of Clones Starting with LCW546_06 and LCW459_09

| | LCW546_06 | All Others | LCW546_09 | All Others |
|---|---|---|---|---|
| N | 11 | 72 | 9 | 74 |
| Mean | 1100 | 752 | 988 | 775 |
| Fluorescence (AU) | | | | |
| SD | 275 | 154 | 179 | 202 |
| CV | 25% | 20% | 18% | 26% |

The sequencing and retest also revealed several instances of independent replicates of the same sequence in the data producing similar results, thus increasing confidence in the assay. Additionally, 10 clones with 6 unique sequences were superior to the benchmark clone. They are presented in Table 19. It was noted that these were the only occurrences of these sequences and in no case did one of these sequences occur and fail to beat the bench-mark clone. These six sequences were advanced for further optimization.

TABLE 19
Combinatorial 12mer and 36mer Clones Superior to Benchmark Clone

| | | SEQ | | |
|---|---|---|---|---|
| | | ID | | |
| Clone Name | First 60 codons | NO: | 12mer Name | 36mer Name |
| LCW580_51 | ATGGCTAGTCCTGCTGGCTCTCCAACCTCCACTGA | 656 | LCW546_06 | LCW0404_040 |
| | GGAAGGTGCATCCCCGGGCACCAGCTCTACCGGTT | | | |
| | CTCCAGGTAGCTCTACCCCGTCTGGTGCTACCGGC | | | |
| | TCTCCAGGTAGCTCTACCCCGTCTGGTGCTACTGG | | | |
| | CTCTCCAGGTACTTCTACTGAACCGTCTGAAGGCA | | | |
| | GCGCA | | | |
| LCW580_81 | ATGGCTAGTCCTGCTGGCTCTCCAACCTCCACTGA | 657 | LCW546_06 | LCW0404_040 |
| | GGAAGGTGCATCCCCGGGCACCAGCTCTACCGGTT | | | |
| | CTCCAGGTAGCTCTACCCCGTCTGGTGCTACCGGC | | | |
| | TCTCCAGGTAGCTCTACCCCGTCTGGTGCTACTGG | | | |
| | CTCTCCAGGTACTTCTACTGAACCGTCTGAAGGCA | | | |
| | GCGCA | | | |
| LCW580_38 | ATGGCTAGTCCTGCTGGCTCTCCAACCTCCACTGA | 658 | LCW546_06 | LCW0402_041 |
| | GGAAGGTACTTCTACCGAACCGTCCGAGGGTAGCG | | | |
| | CACCAGGTAGCCCAGCAGGTTCTCCTACCTCCACC | | | |
| | GAGGAAGGTACTTCTACCGAACCGTCCGAGGGTA | | | |
| | GCGCACCAGGTACTTCTACTGAACCGTCTGAAGGC | | | |
| | AGCGCA | | | |
| LCW580_63 | ATGGCTAGTCCTGCTGGCTCTCCGACCTCTACTGA | 659 | LCW546_09 | LCW0402_020 |
| | GGAAGGTACTTCTACTGAACCGTCTGAAGGCAGCG | | | |
| | CACCAGGTAGCGAACCGGCTACTTCCGGTTCTGAA | | | |
| | ACCCCAGGTAGCCCAGCAGGTTCTCCAACTTCTAC | | | |
| | TGAAGAAGGTACTTCTACTGAACCGTCTGAAGGCA | | | |
| | GCGCA | | | |
| LCW580_06 | ATGGCTAGTCCTGCTGGCTCTCCAACCTCCACTGA | 660 | LCW546_06 | LCW0404_031 |
| | GGAAGGTACCCCGGGTAGCGGTACTGCTTCTTCCT | | | |
| | CTCCAGGTAGCTCTACCCCTTCTGGTGCAACCGGC | | | |
| | TCTCCAGGTGCTTCTCCGGGCACCAGCTCTACCGG | | | |
| | TTCTCCAGGTACTTCTACTGAACCGTCTGAAGGCA | | | |
| | GCGCA | | | |
| LCW580_35 | ATGGCTAGTCCTGCTGGCTCTCCGACCTCTACTGA | 661 | LCW546_09 | LCW0402_020 |
| | GGAAGGTACTTCTACTGAACCGTCTGAAGGCAGCG | | | |
| | CACCAGGTAGCGAACCGGCTACTTCCGGTTCTGAA | | | |
| | ACCCCAGGTAGCCCAGCAGGTTCTCCAACTTCTAC | | | |
| | TGAAGAAGGTACTTCTACTGAACCGTCTGAAGGCA | | | |
| | GCGCA | | | |
| LCW580_67 | ATGGCTAGTCCTGCTGGCTCTCCGACCTCTACTGA | 662 | LCW546_09 | LCW0403_064 |
| | GGAAGGTACCTCCCCTAGCGGCGAATCTTCTACTG | | | |
| | CTCCAGGTACCTCTCCTAGCGGCGAATCTTCTACC | | | |
| | GCTCCAGGTACCTCCCCTAGCGGTGAATCTTCTAC | | | |
| | CGCACCAGGTACTTCTACTGAACCGTCTGAAGGCA | | | |
| | GCGCA | | | |
| LCW580_13 | ATGGCTAGTCCTGCTGGCTCTCCGACCTCTACTGA | 663 | LCW546_09 | LCW0403_060 |
| | GGAAGGTACCTCTACTCCGGAAAGCGGTTCCGCAT | | | |
| | CTCCAGGTTCTACCAGCGAATCCCCGTCTGGCACC | | | |
| | GCACCAGGTTCTACTAGCTCTACTGCTGAATCTCC | | | |
| | GGGCCCAGGTACTTCTACTGAACCGTCTGAAGGCA | | | |
| | GCGCA | | | |
| LCW580_88 | ATGGCTAGTCCTGCTGGCTCTCCGACCTCTACTGA | 664 | LCW546_09 | LCW0403_064 |
| | GGAAGGTACCTCCCCTAGCGGCGAATCTTCTACTG | | | |
| | CTCCAGGTACCTCTCCTAGCGGCGAATCTTCTACC | | | |
| | GCTCCAGGTACCTCCCCTAGCGGTGAATCTTCTAC | | | |
| | CGCACCAGGTACTTCTACTGAACCGTCTGAAGGCA | | | |
| | GCGCA | | | |
| LCW580_11 | ATGGCTAGTCCTGCTGGCTCTCCGACCTCTACTGA | 665 | LCW546_09 | LCW0403_060 |
| | GGAAGGTACCTCTACTCCGGAAAGCGGTTCCGCAT | | | |
| | CTCCAGGTTCTACCAGCGAATCCCCGTCTGGCACC | | | |
| | GCACCAGGTTCTACTAGCTCTACTGCTGAATCTCC | | | |
| | GGGCCCAGGTACTTCTACTGAACCGTCTGAAGGCA | | | |
| | GCGCA | | | |

### Example 17

### Construction of N-Terminal Extensions of XTEN-Construction and Screening of Combinatorial 12mer and 36mer Libraries for XTEN-AM875 and XTEN-AE864

This example details a step in the optimization of the N-terminus of the XTEN protein to promote the initiation of translation to allow for expression of XTEN fusions at the N-terminus of proteins without the presence of a helper domain. With preferences for the first four codons (see Examples supra, and for the best pairing of N-terminal 12mers and 36mers (see Example supra) established, a combinatorial approach was undertaken to examine the union of these preferences. This created novel 48mers at the N-terminus of the XTEN protein and enabled the testing of the confluence of previous conclusions. Additionally, the ability of these leader sequences to be a universal solution for all XTEN proteins was assessed by placing the new 48mers in front of both XTEN-AE864 and XTEN-AM875. Instead of using all 125 clones of 36mer segment, the plasmids from 6 selected clones of 36mer segment with best GFP expression in the combinatorial library were digested with NdeI/EcoRI/BsaI and the appropriate fragments were gel-purified. The plasmids from clones AC94 (CBD-XTEN_AM875-GFP) and AC104 (CBD-XTEN_AE864-GFP) were digested with digested with NdeI/EcoRI/BsaI and the appropriate fragments were gel-purified. These fragments were ligated together and transformed into E. coli BL21Gold(DE3) competent cells to obtain colonies of the libraries LCW0585 (—XTEN_AM875-GFP) and LCW0586 (—XTEN_AE864-GFP), which could also serve as the vectors for further cloning 8 selected 12mers at the very N-terminus. The plasmids of LCW0585 and LCW0586 were digested with NdeI/EcoRI/BsaI and the appropriate fragments were gel-purified. 8 pairs of oligonucleotides encoding 8 selected 12mer sequences with best GFP expression in the previous (Generation 2) screening were designed, annealed and ligated with the NdeI/EcoRI/BsaI digested LCW0585 and LCW0586 vectors, and transformed into E. coli BL21Gold(DE3) competent cells to obtain colonies of the final libraries LCW0587 (XTEN_AM923-GFP) and LCW0588 (XTEN_AE912-GFP). With a theoretical diversity of 48 unique clones, a total of 252 individual colonies from the created libraries were picked and grown overnight to saturation in 500 μl of super broth media in a 96 deep well plate. This provided sufficient coverage to understand relative library performance and sequence preferences. The saturated overnight cultures were used to inoculate new 500 μl cultures in auto-induction media in which were grown overnight at 26° C. These expression cultures were then assayed using a fluorescence plate reader (excitation 395 nm, emission 510 nm) to determine the amount of GFP reporter present. The top 36 clones were sequenced and retested for GFP reporter expression. 36 clones yielded usable sequencing data and these 36 were used for the subsequent analysis. The sequencing data determined the 12mer, the third codon, the fourth codon and the 36mer present in the clone and revealed that many of the clones were independent replicates of the same sequence. Additionally, the retest results for these clones are close in value, indicating the screening process was robust. Preferences for certain combinations at the N-terminus were seen and were consistently yielding higher fluorescence values approximately 50% greater than the benchmark controls (see Tables 20 and 21). These date support the conclusion that the inclusion of the sequences encoding the optimized N-terminal XTEN into the fusion protein genes conferred a marked enhancement on the expression of the fusion proteins.

TABLE 20
Preferred N-terminal Combinations for XTEN-AM875

| Clone Name | Number of Replicates | 12 mer | 36 mer | Mean | SD | CV |
|---|---|---|---|---|---|---|
| CBD-AM875 | NA | NA | NA | 1715 | 418 | 16% |
| LCW587_08 | 7 | LCW546_06_3=GAA | LCW404_40 | 2333 | 572 | 18% |
| LCW587_17 | 5 | LCW546_09_3=GAA | LCW403_64 | 2172 | 293 | 10% |

TABLE 21
Preferred N-terminal Combinations for XTEN-AE864

| | Num- | | | | | |
|---|---|---|---|---|---|---|
| | ber of | | | | | |
| | Repli- | | | | | |
| Clone Name | cates | 12 mer | 36 mer | Mean | SD | CV |
| AC82 | NA | NA | NA | 1979 | 679 | 24% |
| LCW588_14 | 8 | LCW546_06_opt3 | LCW404_31 | 2801 | 240 | 6% |
| LCW588_27 | 2 | LCW546_06_opt34 | LCW404_40 | 2839 | 556 | 15% |

Notably, the preferred combination of the N-terminal for the XTEN-AM875 and the preferred combination for the XTEN-AE864 are not the same, indicating more complex interactions further than 150 bases from the initiation site influence expression levels. The sequences for the preferred nucleotide sequences are listed in Table 22 and the preferred clones were analyzed by SDS-PAGE to independently confirm expression (see FIG. 30). The complete sequences of XTEN_AM923 and XTEN_AE912 were selected for further analysis.

TABLE 22
Preferred DNA Nucleotide Sequences for first 48 Amino
Acid Residues of N-terminal XTEN-AM875 and XTEN-AE864

| | | | SEQ |
|---|---|---|---|
| | XTEN | | ID |
| Clone Name | Modified | DNA Nucleotide Sequence | NO: |
| LCW587_08 | AM875 | ATGGCTGAACCTGCTGGCTCTCCAACCTCCACTGAGGAAGGTGCATC | 666 |
| | | CCCGGGCACCAGCTCTACCGGTTCTCCAGGTAGCTCTACCCCGTCTG | |
| | | GTGCTACCGGCTCTCCAGGTAGCTCTACCCCGTCTGGTGCTACTGGC | |
| | | TCTCCAGGTACTTCTACTGAACCGTCTGAAGGCAGCGCA | |
| LCW587_17 | AM875 | ATGGCTGAACCTGCTGGCTCTCCGACCTCTACTGAGGAAGGTACCTC | 667 |
| | | CCCTAGCGGCGAATCTTCTACTGCTCCAGGTACCTCTCCTAGCGGCG | |
| | | AATCTTCTACCGCTCCAGGTACCTCCCCTAGCGGTGAATCTTCTACC | |
| | | GCACCAGGTACTTCTACTGAACCGTCTGAAGGCAGCGCA | |
| LCW588_14 | AE864 | ATGGCTGAACCTGCTGGCTCTCCAACCTCCACTGAGGAAGGTACCCC | 668 |
| | | GGGTAGCGGTACTGCTTCTTCCTCTCCAGGTAGCTCTACCCCTTCTGG | |
| | | TGCAACCGGCTCTCCAGGTGCTTCTCCGGGCACCAGCTCTACCGGTT | |
| | | CTCCAGGTAGCCCGGCTGGCTCTCCTACCTCTACTGAG | |
| LCW588_27 | AE864 | ATGGCTGAAACTGCTGGCTCTCCAACCTCCACTGAGGAAGGTGCATC | 669 |
| | | CCCGGGCACCAGCTCTACCGGTTCTCCAGGTAGCTCTACCCCGTCTG | |
| | | GTGCTACCGGCTCTCCAGGTAGCTCTACCCCGTCTGGTGCTACTGGC | |
| | | TCTCCAGGTAGCCCGGCTGGCTCTCCTACCTCTACTGAG | |

### Example 18

### Methods of Producing and Evaluating BFXTEN

A general schema for producing and evaluating BFXTEN compositions is presented in FIG. 6, and forms the basis for the general description of this Example. Using the disclosed methods and those known to one of ordinary skill in the art, together with guidance provided in the illustrative examples, a skilled artesian can create and evaluate BFXTEN fusion proteins comprising, XTENs, BP and variants of BP known in the art. The Example is, therefore, to be construed as merely illustrative, and not limitative of the methods in any way whatsoever; numerous variations will be apparent to the ordinarily skilled artisan.

The general schema for producing polynucleotides encoding XTEN is presented in FIGS. 4 and 5. FIG. 5 is a schematic flowchart of representative steps in the assembly of a XTEN polynucleotide construct in one of the embodiments of the invention. Individual oligonucleotides 501 are annealed into sequence motifs 502 such as a 12 amino acid motif (“12-mer”), which is subsequently ligated with an oligo containing BbsI, and KpnI restriction sites 503. The motif libraries can be limited to the specific sequence families; e.g., the AD, AE, AF, AG, AM, AQ, BC or BD sequences of Table 1. Additional sequence motifs from a library are annealed to the 12-mer to create a “building block” length; e.g., a segment that encodes 36 amino acids. The gene encoding the XTEN sequence is assembled by ligation and multimerization of the “building blocks” until the desired length of the XTEN gene 504 is achieved. For example, multimerization can be performed by ligation, overlap extension, PCR assembly or similar cloning techniques known in the art. The XTEN gene is then cloned into a stuffer vector. In one example, the vector can encode a Flag sequence 506 followed by a stuffer sequence that is flanked by BsaI, BbsI, and KpnI sites 507 and a BP gene 508, resulting in the gene encoding BFXTEN 500.

DNA sequences encoding a candidate BP are conveniently obtained by standard procedures known in the art from a cDNA library prepared from an appropriate cellular source, from a genomic library, or may be created synthetically (e.g., automated nucleic acid synthesis) using DNA sequences obtained from publicly available databases, patents, or literature references. A gene or polynucleotide encoding each of the BP portions of the protein is then be cloned into a construct, such as those described herein, which can be a plasmid or other vector under control of appropriate transcription and translation sequences for high level protein expression in a biological system. A second gene or polynucleotide coding for each XTEN is genetically fused to the nucleotides encoding the N- and/or C-terminus of the BP gene, depending on the desired N- to C-terminus configuration desired, by cloning it into the construct adjacent and in frame with the gene coding for the BP through a ligation or multimerization step. In this manner, a chimeric DNA molecule coding for (or complementary to) a BFXTEN fusion protein is generated within the construct. The construct is designed in different configurations to encode the various permutations of the fusion partners as described herein. For example, the gene can be created to encode the fusion protein in the order (N- to C-terminus): BP-XTEN; XTEN-BP; BP-XTEN-BP; XTEN-BP-XTEN (FIG. 1); as well a configuration of formula I-VI. Optionally, this chimeric DNA molecule may be transferred or cloned into another construct that is a more appropriate expression vector. At this point, a host cell capable of expressing the chimeric DNA molecule is transformed with the chimeric DNA molecule. The vectors containing the DNA segments of interest are transferred into an appropriate host cell by well-known methods, depending on the type of cellular host, as described supra.

Host cells containing the polynucleotides of interest are cultured in conventional nutrient media modified as appropriate for activating promoters, selecting transformants or amplifying genes. The culture conditions, such as temperature, pH and the like, are those previously used with the host cell selected for expression, and will be apparent to the ordinarily skilled artisan. Cells are typically harvested by centrifugation, disrupted by physical or chemical means, and the resulting crude extract retained for further purification. For compositions secreted by the host cells, supernatant from centrifugation is separated and retained for further purification.

Gene expression may be measured in a sample directly, for example, by conventional Southern blotting, Northern blotting to quantitate the transcription of mRNA [Thomas, Proc. Natl. Acad. Sci. USA, 77:5201-5205 (1980)], dot blotting (DNA analysis), or in situ hybridization, using an appropriately labeled probe, based on the sequences provided herein. Gene expression, alternatively, may be measured by immunological of fluorescent methods, such as immunohistochemical staining of cells or tissue sections and assay of cell culture or body fluids or the detection of selectable markers, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence BP polypeptide or against a synthetic peptide based on the DNA sequences provided herein or against exogenous sequence fused to BP and encoding a specific antibody epitope. Examples of selectable markers are well known to one of skill in the art and include reporters such as enhanced green fluorescent protein (EGFP), beta-galactosidase (β-gal) or chloramphenicol acetyltransferase (CAT).

BFXTEN polypeptide product may be purified via methods known in the art. Procedures such as gel filtration, affinity purification, salt fractionation, ion exchange chromatography, size exclusion chromatography, hydroxyapatite adsorption chromatography, hydrophobic interaction chromatography and gel electrophoresis may be used. Some expressed BFXTEN may require refolding during isolation and purification. Methods of purification are described in Robert K. Scopes, Protein Purification: Principles and Practice, Charles R. Castor, ed., Springer-Verlag 1994, and Sambrook, et al., supra. Multi-step purification separations are also described in Baron, et al., Crit. Rev. Biotechnol. 10:179-90 (1990) and Below, et al., J. Chromatogr. A. 679:67-83 (1994).

As illustrated in FIG. 6, the isolated BFXTEN fusion proteins are characterized for their chemical and biological activity properties. Isolated BFXTEN may be characterized, e.g., for sequence, purity, apparent molecular weight, solubility and stability using standard methods known in the art. BFXTEN meeting expected standards can then be evaluated for biological activity, which can be measured using in vitro or in vivo assays, such as the assays of Table 32. For example, one or more assays known in the art for evaluating BP is performed and used as the endpoint for which therapeutic activity is measured. One such assay is receptor binding, to verify that the configuration of the BFXTEN permits binding to the target receptor, relative to BP not linked to XTEN. To evaluate the receptor binding activity of the fusion proteins an ELISA based receptor binding assay is used. The wells of an assay plate are coated with 50 ng per well of the target receptor fused to Fc domain of human IgG. Subsequently the wells are blocked with 3% BSA to prevent nonspecific interactions with the solid phase. After thoroughly washing the wells, a dilution series of different configurations of BFXTEN fusion proteins are applied to the wells. The binding reaction is allowed to proceed for 2 hr at room temperature. Unbound fusion protein or free BP is removed by repeated washing. The bound fusion proteins (or BP positive control) are detected with a biotinylated anti-BP antibody and a horseradish peroxidase-conjugated streptavidin. The reaction is developed with TMB substrate for 20 minutes at room temperature. Color development is stopped with the addition of 0.2 N sulfuric acid. The absorbance of each well at 450 nm and 570 nm is recorded on a SpectrMax 384Plus spectrophotometer. The corrected absorbance signal (Abs_{corr}=Abs₄₅₀ₙₘ−Abs₅₇₀ₙₘ) is plotted as a function of reactant concentration to produce a binding isotherm. To estimate the binding affinity of each fusion protein for the receptor, the binding data are fit to a sigmoidal dose-response curve. From the fit of the data an EC50 (the concentration of BP or fusion protein at which the signal is half maximal) for each construct is determined. BFXTEN fusion proteins with the desired degree of binding affinity are considered candidates for further evaluation. Other in vitro or ex vivo assays, such as the assays of Table 32, are performed, depending on the biological activity to be confirmed.

In addition, BFXTEN fusion proteins (either singly in the case of BMXTEN or in combination in the case of BCXTEN), are administered to one or more animal species to determine standard pharmacokinetic parameters, using methods described in Example 24. BFXTEN with enhanced pharmacokinetics compared to BP not bound to XTEN are considered candidates for further evaluation.

By the iterative process of producing, expressing, and recovering BFXTEN constructs of the invention, followed by their characterization using methods disclosed herein or others known in the art, BFXTEN compositions comprising any BP and any XTEN as contemplated by the invention are produced and evaluated by one of ordinary skill in the art to confirm the expected properties such as enhanced solubility, enhanced stability, retention of biological activity, improved pharmacokinetics and reduced immunogenicity, leading to an overall enhanced therapeutic activity compared to the corresponding unfused BP. For those fusion proteins not possessing the desired properties, a different sequence or configuration, or a different combination of BPs is constructed, expressed, isolated and evaluated by these methods in order to obtain a BFXTEN composition with the desired properties.

### Example 19

### Analytical Size Exclusion Chromatography of XTEN Fusion Proteins with Diverse Payloads

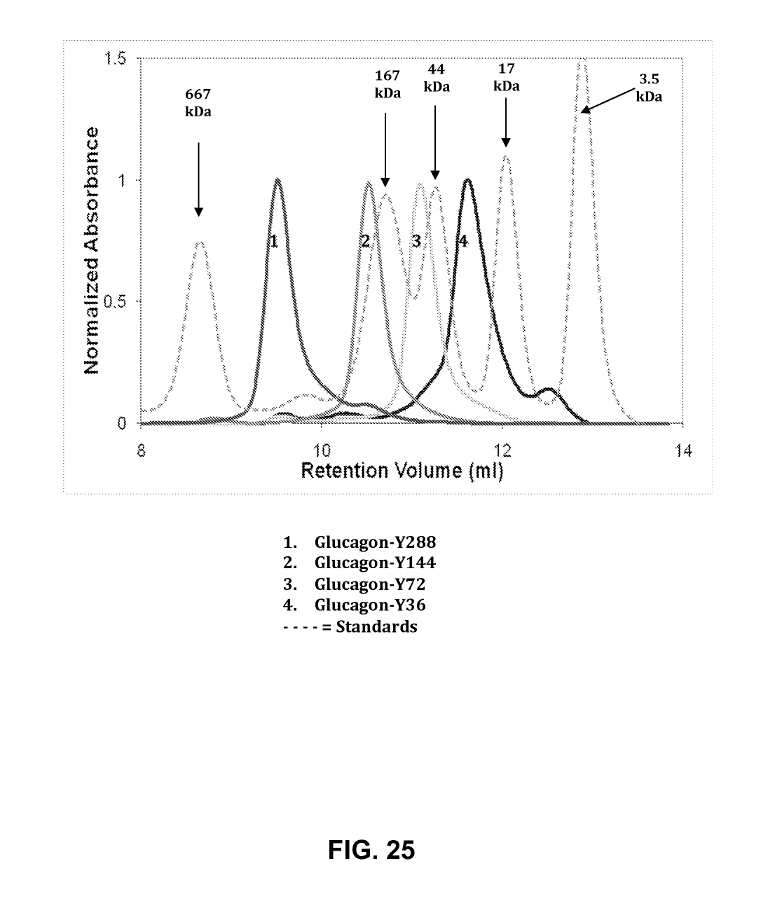

Size exclusion chromatography analyses were performed on fusion proteins containing various therapeutic proteins and unstructured recombinant proteins of increasing length. An exemplary assay used a TSKGel-G4000 SWXL (7.8 mm×30 cm) column in which 40 μg of purified glucagon fusion protein at a concentration of 1 mg/ml was separated at a flow rate of 0.6 ml/min in 20 mM phosphate pH 6.8, 114 mM NaCl. Chromatogram profiles were monitored using OD214 nm and OD280 nm. Column calibration for all assays were performed using a size exclusion calibration standard from BioRad; the markers include thyroglobulin (670 kDa), bovine gamma-globulin (158 kDa), chicken ovalbumin (44 kDa), equine myoglobuin (17 kDa) and vitamin B12 (1.35 kDa). Representative chromatographic profiles of Glucagon-Y288, Glucagon-Y144, Glucagon-Y72, Glucagon-Y36 are shown as an overlay in FIG. 25. The data show that the apparent molecular weight of each compound is proportional to the length of the attached unstructured sequence. However, the data also show that the apparent molecular weight of each construct is significantly larger than that expected for a globular protein (as shown by comparison to the standard proteins run in the same assay). Based on the SEC analyses for all constructs evaluated, the apparent molecular weights, the apparent molecular weight factor (expressed as the ratio of apparent molecular weight to the calculated molecular weight) and the hydrodynamic radius (R_{H} in nM) are shown in Table 23. The results indicate that incorporation of different XTENs of 576 amino acids or greater confers an apparent molecular weight for the fusion protein of approximately 339 kDa to 760, and that XTEN of 864 amino acids or greater confers an apparent molecular weight greater than approximately 800 kDA. The results of proportional increases in apparent molecular weight to actual molecular weight were consistent for fusion proteins created with XTEN from several different motif families; i.e., AD, AE, AF, AG, and AM, with increases of at least four-fold and ratios as high as about 17-fold. Additionally, the incorporation of XTEN fusion partners with 576 amino acids or more into fusion proteins with biologically active proteins resulted with a hydrodynamic radius of 7 nm or greater; well beyond the glomerular pore size of approximately 3-5 nm. Accordingly, it is concluded that fusion proteins comprising biologically active proteins and XTEN would have reduced renal clearance, contributing to increased terminal half-life and improving the therapeutic or biologic effect relative to a corresponding un-fused biologically active protein.

TABLE 23
SEC analysis of various polypeptides

| | XTEN | | | Appar- | Apparent | |
|---|---|---|---|---|---|---|
| Con- | or | Thera- | Actual | ent | Molecular | |
| struct | fusion | peutic | MW | MW | Weight | R_{H} |
| Name | partner | Protein | (kDa) | (kDa) | Factor | (nm) |
| AC14 | Y288 | Glucagon | 28.7 | 370 | 12.9 | 7.0 |
| AC28 | Y144 | Glucagon | 16.1 | 117 | 7.3 | 5.0 |
| AC34 | Y72 | Glucagon | 9.9 | 58.6 | 5.9 | 3.8 |
| AC33 | Y36 | Glucagon | 6.8 | 29.4 | 4.3 | 2.6 |
| AC89 | AF120 | Glucagon | 14.1 | 76.4 | 5.4 | 4.3 |
| AC88 | AF108 | Glucagon | 13.1 | 61.2 | 4.7 | 3.9 |
| AC73 | AF144 | Glucagon | 16.3 | 95.2 | 5.8 | 4.7 |
| AC53 | AG576 | GFP | 74.9 | 339 | 4.5 | 7.0 |
| AC39 | AD576 | GFP | 76.4 | 546 | 7.1 | 7.7 |
| AC41 | AE576 | GFP | 80.4 | 760 | 9.5 | 8.3 |
| AC52 | AF576 | GFP | 78.3 | 526 | 6.7 | 7.6 |
| AC85 | AE864 | Exendin-4 | 83.6 | 938 | 11.2 | 8.9 |
| AC114 | AM875 | Exendin-4 | 82.4 | 1344 | 16.3 | 9.4 |
| AC143 | AM875 | hGH | 100.6 | 846 | 8.4 | 8.7 |
| AC227 | AM875 | IL-1ra | 95.4 | 1103 | 11.6 | 9.2 |
| AC228 | AM1296 | IL-1ra | 134.8 | 2286 | 17.0 | 10.5 |

### Example 20

### Pharmacokinetics of Extended Polypeptides Fused to GFP in Cynomolgus Monkeys

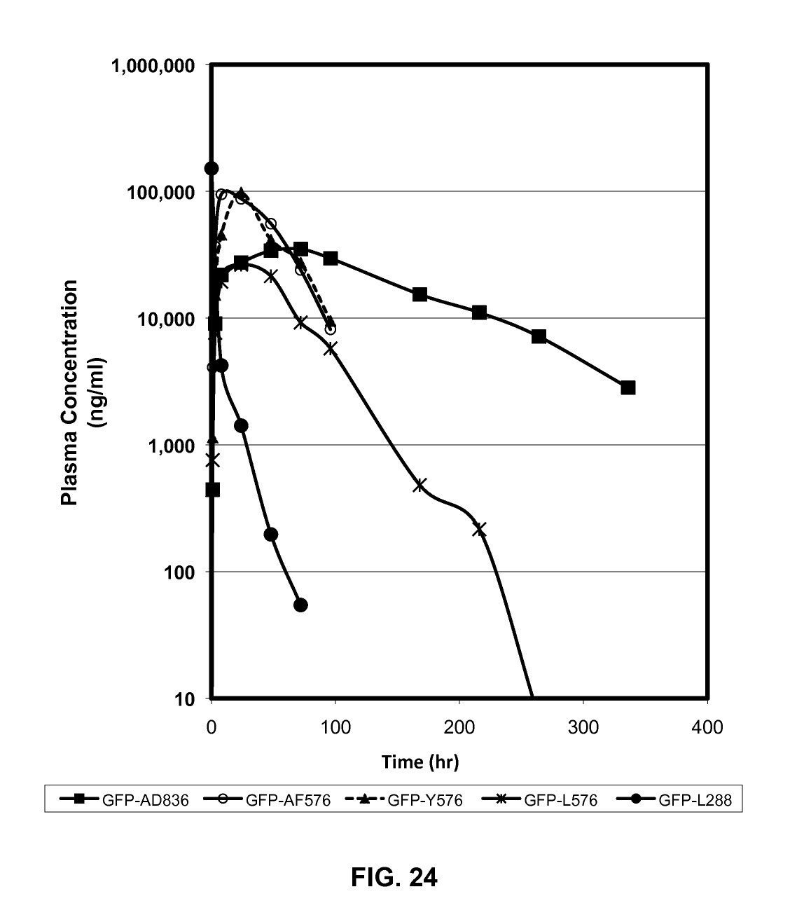

The pharmacokinetics of GFP-L288, GFP-L576, GPF-XTEN_AF576, GFP-XTEN_Y576 and XTEN_AD836-GFP were tested in cynomolgus monkeys to determine the effect of composition and length of the unstructured polypeptides on PK parameters. Blood samples were analyzed at various times after injection and the concentration of GFP in plasma was measured by ELISA using a polyclonal antibody against GFP for capture and a biotinylated preparation of the same polyclonal antibody for detection. Results are summarized in FIG. 24. They show a surprising increase of half-life with increasing length of the XTEN sequence. For example, a half-life of 10 h was determined for GFP-XTEN_L288 (with 288 amino acid residues in the XTEN). Doubling the length of the unstructured polypeptide fusion partner to 576 amino acids increased the half-life to 20-22 h for multiple fusion protein constructs; i.e., GFP-XTEN_L576, GPF-XTEN_AF576, GFP-XTEN_Y576. A further increase of the unstructured polypeptide fusion partner length to 836 residues resulted in a half-life of 72-75 h for XTEN_AD836-GFP. Thus, increasing the polymer length by 288 residues from 288 to 576 residues increased in vivo half-life by about 10 h. However, increasing the polypeptide length by 260 residues from 576 residues to 836 residues increased half-life by more than 50 h. These results show that there is a surprising threshold of unstructured polypeptide length that results in a greater than proportional gain in in vivo half-life. Thus, fusion proteins comprising extended, unstructured polypeptides are expected to have the property of enhanced pharmacokinetics compared to polypeptides of shorter lengths.

### Example 21

### Serum Stability of XTEN

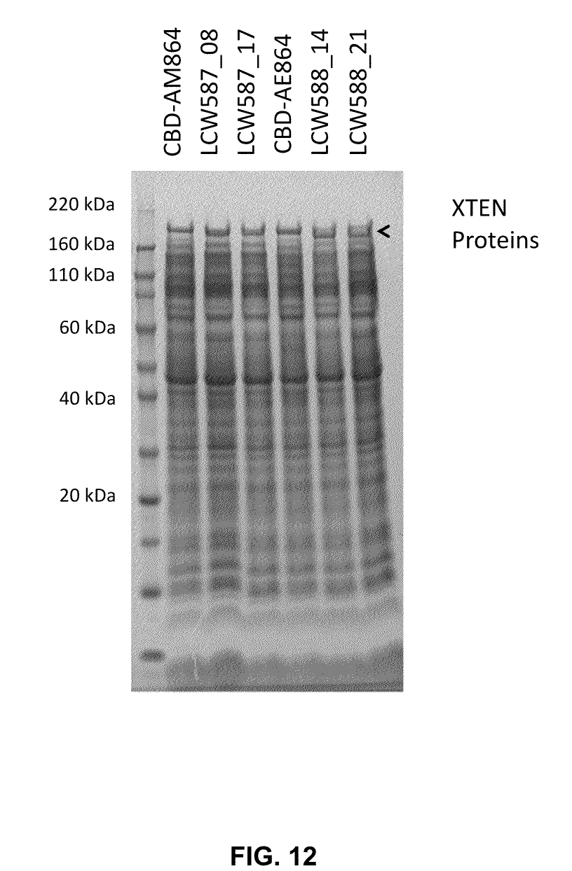

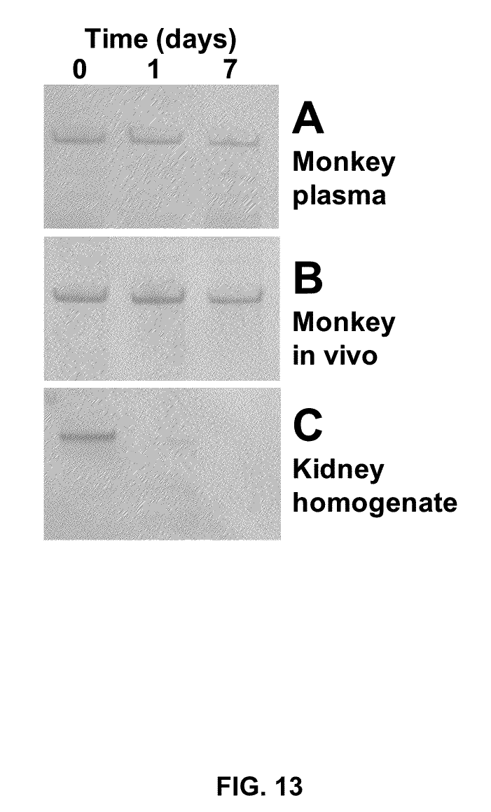

A fusion protein containing XTEN_AE864 fused to the N-terminus of GFP was incubated in monkey plasma and rat kidney lysate for up to 7 days at 37° C. Samples were withdrawn at time 0, Day 1 and Day 7 and analyzed by SDS PAGE followed by detection using Western analysis and detection with antibodies against GFP as shown in FIG. 13. The sequence of XTEN_AE864 showed negligible signs of degradation over 7 days in plasma. However, XTEN_AE864 was rapidly degraded in rat kidney lysate over 3 days. The in vivo stability of the fusion protein was tested in plasma samples wherein the GFP_AE864 was immunoprecipitated and analyzed by SDS PAGE as described above. Samples that were withdrawn up to 7 days after injection showed very few signs of degradation. The results demonstrate the resistance of BPXTEN to degradation due to serum proteases; a factor in the enhancement of pharmacokinetic properties of the BPXTEN fusion proteins.

### Example 22

### Construction of BFXTEN Component XTEN_IL-1ra Genes and Vectors

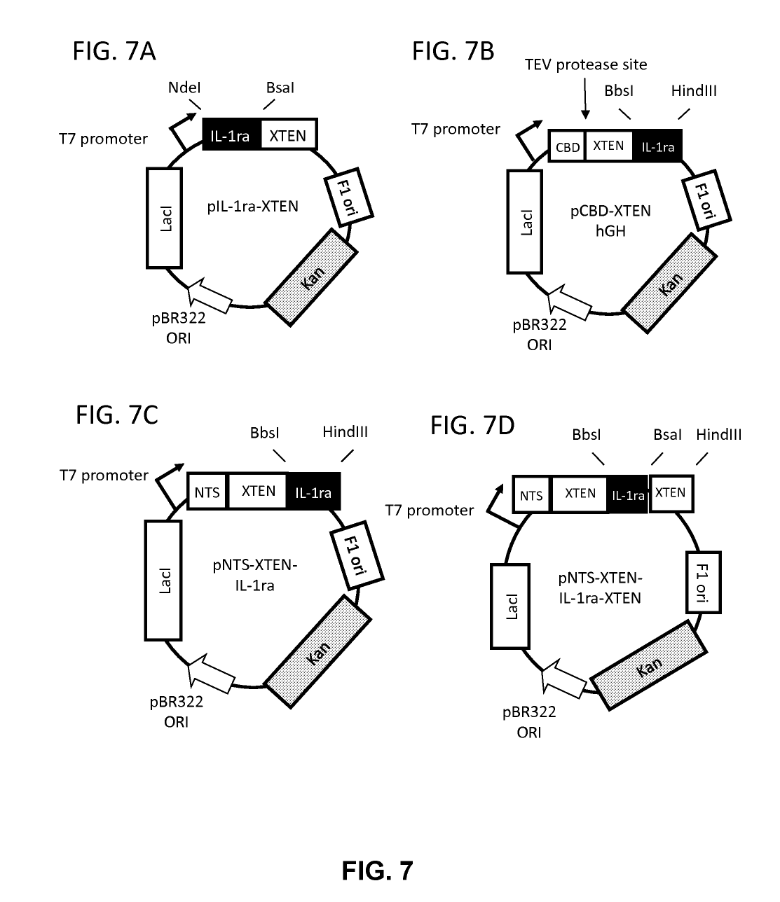

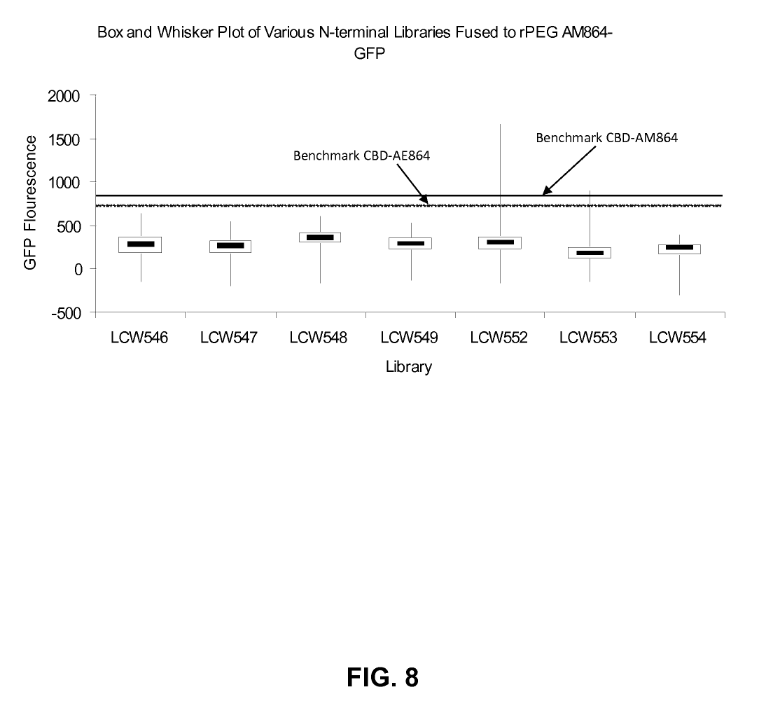

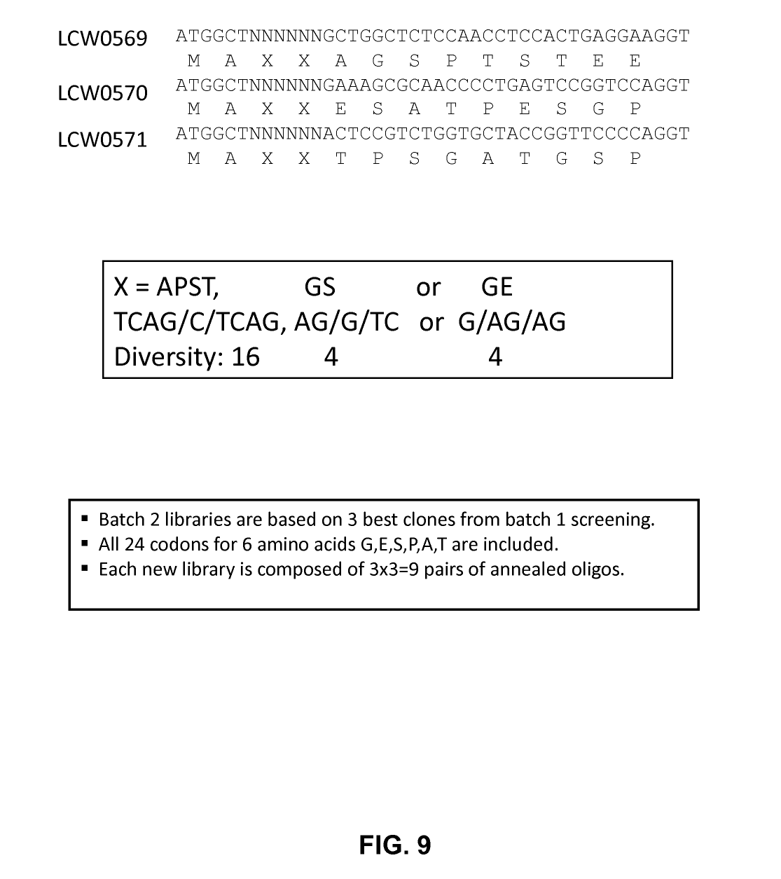

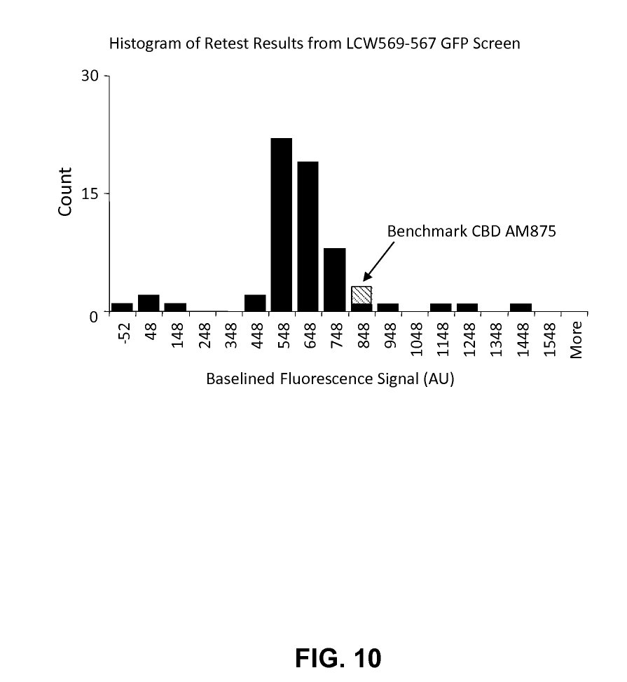

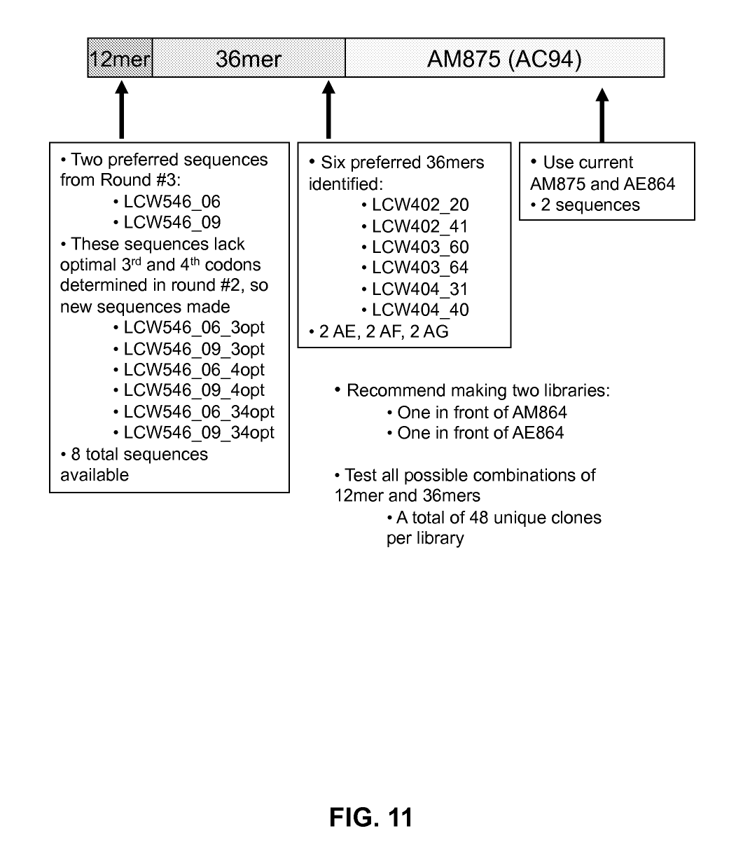

The gene encoding human IL-1ra of 153aa was amplified by polymerase chain reaction (PCR) with primers 5′-ATAAAGGGTCTCCAGGTCGTCCGTCCGGTCGTAAATC (SEQ ID NO: 670) and 5′-AACTCGaagcttTTATTCGTCCTCCTGGAAGTAAAA (SEQ ID NO: 671), which introduced flanking BsaI and HindIII (underlined) restriction sites that are compatible with the BbsI and HindIII sites that flank the stuffer in the XTEN destination vector (FIG. 7C). The XTEN destination vectors contain the kanamycin-resistance gene and are pET30 derivatives from Novagen in the format of Cellulose Binding Domain (CBD)-XTEN-Green Fluorescent Protein (GFP), where GFP is the stuffer for cloning payloads at C-terminus. Constructs were generated by replacing GFP in the XTEN destination vectors with the IL-1ra encoding fragment (FIG. 7). The XTEN destination vector features a T7 promoter upstream of CBD followed by an XTEN sequence fused in-frame upstream of the stuffer GFP sequence. The XTEN sequences employed are XTEN_AM875, XTEN_AM1318, AF875 and AE864 which have lengths of 875, 1318, 875 and 864 amino acids, respectively. The stuffer GFP fragment was removed by restriction digestion using BbsI and HindIII endonucleases. BsaI and HindIII restriction digested IL-1ra DNA fragment was ligated into the BbsI and HindIII digested XTEN destination vector using T4 DNA ligase and the ligation mixture was transformed into E. coli strain BL21 (DE3) Gold (Stratagene) by electroporation. Transformants were identified by the ability to grow on LB plates containing the antibiotic kanamycin. Plasmid DNAs were isolated from selected clones and confirmed by restriction analysis and DNA sequencing. The final vector yields the CBD_XTEN_IL-1ra gene under the control of a T7 promoter and CBD is cleaved by engineered TEV cleavage site at the end to generate XTEN_IL1-ra. Various constructs with IL-1ra fused at C-terminus to different XTENs include AC1723 (CBD-XTEN_AM875-IL-1ra), AC175 (CBD-XTEN_AM1318-IL-1ra), AC180 (CBD-XTEN_AF875-IL-1ra), and AC182 (CBD-XTEN_AE864-IL-1ra).

### Example 23

### Expression, Purification, and Characterization of Human Interleukin-1 Receptor Agonist (IL-1ra) Fused to XTEN_AM875 and XTEN_AE864

Cell Culture Production

A starter culture was prepared by inoculating glycerol stocks of E. coli carrying a plasmid encoding for IL-1ra fused to AE864, AM875, or AM1296 [SEQ ID No. 54, 56, or 60] into 100 mL 2×YT media containing 40 ug/mL kanamycin. The culture was then shaken overnight at 37° C. 100 mL of the starter culture was used to inoculate 25 liters of 2×YT containing 40 μg/mL kanamycin and shaken until the OD600 reached about 1.0 (for 5 hours) at 37° C. The temperature was then reduced to 26° C. and protein expression was induced with IPTG at 1.0 mM final concentration. The culture was then shaken overnight at 26° C. Cells were harvested by centrifugation yielding a total of 200 grams cell paste. The paste was stored frozen at −80° C. until use.

Purification of BFXTEN Comprising IL-1ra-XTEN AE864 or IL-1ra-AM875

Cell paste was suspended in 20 mM Tris pH 6.8, 50 mM NaCl at a ratio of 4 ml of buffer per gram of cell paste. The cell paste was then homogenized using a top-stirrer. Cell lysis was achieved by passing the sample once through a microfluidizer at 20000 psi. The lysate was clarified to by centrifugation at 12000 rpm in a Sorvall G3A rotor for 20 minutes.

Clarified lysate was directly applied to 800 ml of Macrocap Q anion exchange resin (GE Life Sciences) that had been equilibrated with 20 mM Tris pH 6.8, 50 mM NaCl. The column was sequentially washed with Tris pH 6.8 buffer containing 50 mM, 100 mM, and 150 mM NaCl. The product was eluted with 20 mM Tris pH 6.8, 250 mM NaCl.

A 250 mL Octyl Sepharose FF column was equilibrated with equilibration buffer (20 mM Tris pH 6.8, 1.0 M Na₂SO₄). Solid Na₂SO₄ was added to the Macrocap Q eluate pool to achieve a final concentration of 1.0 M. The resultant solution was filtered (0.22 micron) and loaded onto the HIC column. The column was then washed with equilibration buffer for 10 CV to remove unbound protein and host cell DNA. The product was then eluted with 20 mM Tris pH 6.8, 0.5 M Na₂SO₄.

The pooled HIC eluate fractions were then diluted with 20 mM Tris pH 7.5 to achieve a conductivity of less than 5.0 mOhms. The dilute product was loaded onto a 300 ml Q Sepharose FF anion exchange column that had been equilibrated with 20 mM Tris pH 7.5, 50 mM NaCl.

The buffer exchanged proteins were then concentrated by ultrafiltration/diafiltration (UF/DF), using a Pellicon XL Biomax 30000 mwco cartridge, to greater than 30 mg/ml. The concentrate was sterile filtered using a 0.22 micron syringe filter. The final solution was aliquoted and stored at −80° C., and was used for the experiments that follow, infra.

SDS-PAGE Analysis

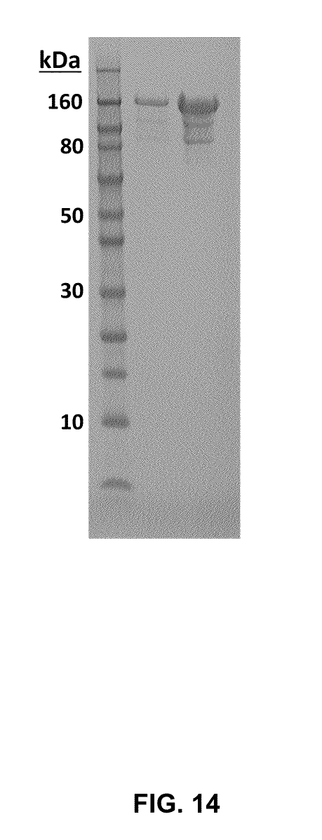

2 and 10 mcg of final purified protein were subjected to non-reducing SDS-PAGE using NuPAGE 4-12% Bis-Tris gel from Invitrogen according to manufacturer's specifications. The results (FIG. 14) show that the IL-1ra-XTEN_AE864 composition was recovered by the process detailed above, with an approximate MW of about 160 kDa.

Analytical Size Exclusion Chromatography

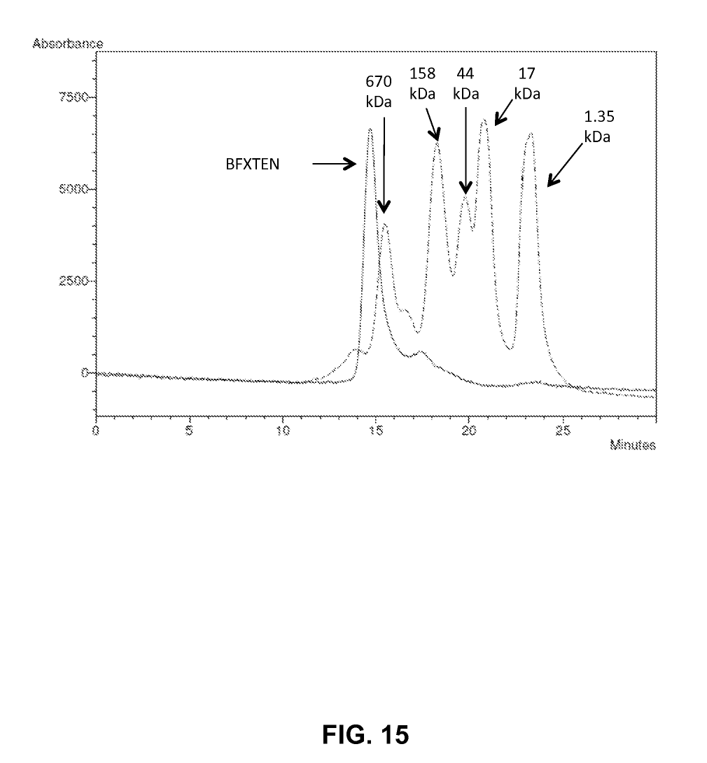

Size exclusion chromatography analysis was performed using a Phenomenex BioSEP SEC 54000 (7.8×300 mm) column. 20 μg of the purified protein at a concentration of 1 mg/ml was separated at a flow rate of 0.5 ml/min in 20 mM Tris-Cl pH 7.5, 300 mM NaCl. Chromatogram profiles were monitored by absorbance at 214 and 280 nm. Column calibration was performed using a size exclusion calibration standard from BioRad, the markers include thyroglobulin (670 kDa), bovine gamma-globulin (158 kDa), chicken ovalbumin (44 kDa), equine myoglobuin (17 kDa) and vitamin B12 (1.35 kDa). A representative chromatographic profile of IL-1ra-XTEN_AM875 is shown in FIG. 15, where the calibration standards are shown in the dashed line and IL-1ra-XTEN_AM875 is shown as the solid line. The data show that the apparent molecular weight of each construct is significantly larger than that expected for a globular protein (as shown by comparison to the standard proteins run in the same assay), and has an apparent molecular weight significantly greater than that determined by SDS-PAGE, describe above.

### Analytical RP-HPLC

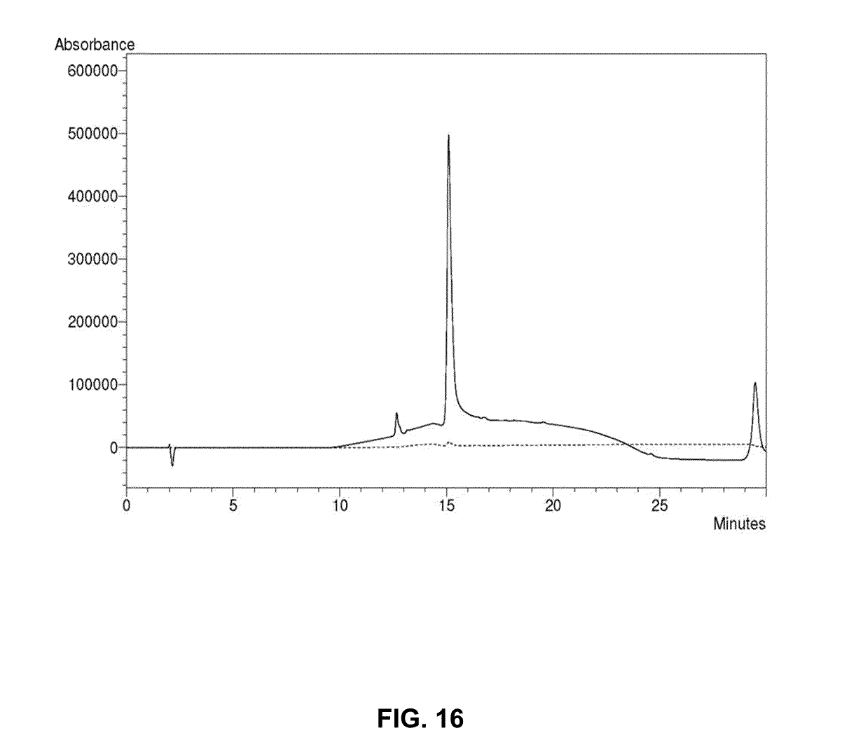

Analytical RP-HPLC chromatography analysis was performed using a Vydac Protein C4 (4.6×150 mm) column. The column was equilibrated with 0.1% trifluoroacetic acid in HPLC grade water at a flow rate of 1 ml/min Ten micrograms of the purified protein at a concentration of 0.2 mg/ml was injected separately. The protein was eluted with a linear gradient from 5% to 90% acetonitrile in 0.1% TFA. Chromatogram profiles were monitored using OD214 nm and OD280 nm. A chromatogram of a representative batch of IL-1ra-XTEN_AM875 is shown in FIG. 16.

IL-1 Receptor Binding

To evaluate the activity of the IL-1ra-containing XTEN fusion proteins, an ELISA based receptor binding assay was used. Here the wells of a Costar 3690 assay plate were coated overnight with 50 ng per well of mouse IL-1 receptor fused to Fc domain of human IgG (IL-1R/Fc, R&D Systems). Subsequently the wells were blocked with 3% BSA to prevent nonspecific interactions with the solid phase. After thoroughly washing the wells, a dilution series of either IL-1ra-XTEN_AM875, XTEN_AM875-IL-1ra, or IL-1ra (anakinra) was applied to the wells. The binding reaction was allowed to proceed for 2 hr at room temperature. Unbound Il-1ra was removed by repeated washing. The bound IL-1ra ad IL-1ra-XTEn fusions were detected with a biotinylated anti-human II-1ra antibody and a horseradish peroxidase-conjugated streptavidin. The reaction was developed with TMB substrate for 20 minutes at room temperature. Color development was stopped with the addition of 0.2 N sulfuric acid. The absorbance of each well at 450 nm and 570 nm was recorded on a SpectrMax 384Plus spectrophotometer. The corrected absorbance signal (Abs_{corr}=Abs₄₅₀ₙₘ−Abs₅₇₀ₙₘ) was plotted as a function of IL-1ra-XTEN or IL-1ra concentration to produce a binding isotherm as shown in FIG. 17.

To estimate the binding affinity of each fusion protein for the IL-1 receptor, the binding data was fit to a sigmoidal dose-response curve. From the fit of the data an EC50 (the concentration of IL-1ra or IL-1ra-XTEN at which the signal is half maximal) for each construct was determined. As shown in FIG. 17, the EC50 of IL-1ra-XTEN_AM875, where the payload was attached to the N-terminus of the XTEN, was comparable to unmodified IL-1ra (anakinra EC50=0.013 nM, IL-1ra-XTEN_AM875 EC50=0.019 nM). XTEN_AM875-IL-1ra, where the payload was attached to the C-terminus of the XTEN, exhibited weaker binding with an EC50 (0.204 nM) that was approximately 15-fold higher that IL-1ra. The negative control XTEN_hGH construct showed no binding under the experimental conditions. The results indicate that the configuration of the fusion protein has an effect on binding affinity, and that, in this case, attaching the IL-1ra BP to the C-terminus of the XTEN significantly reduced the binding affinity to the receptor, compared to the alternative configuration.

Thermal Stabilization of IL-1ra by XTEN

In addition to extending the serum half-life of protein therapeutics, XTEN polypeptides have the property improving the thermal stability of a payload to which it is fused. For example, the hydrophilic nature of the XTEN polypeptide may reduce or prevent aggregation and thus favor refolding of the payload protein. This feature of XTEN may aid in the development of room temperature stable formulations for a variety of protein therapeutics.

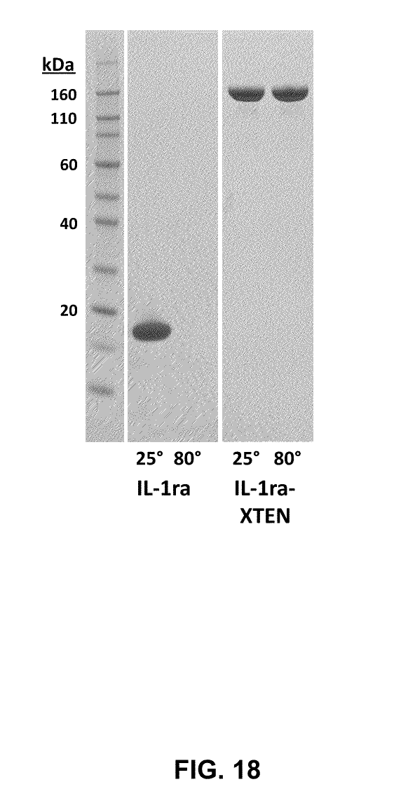

In order to demonstrate thermal stabilization of IL-1ra conferred by XTEN conjugation, IL-1ra-XTEN and recombinant IL-1ra, 200 micromoles per liter, were incubated at 25° C. and 85° C. for 15 min, at which time any insoluble protein was rapidly removed by centrifugation. The soluble fraction was then analyzed by SDS-PAGE as shown in FIG. 18. Note that only IL-1ra-XTEN remained soluble after heating, while, in contrast, recombinant IL-1ra (without XTEN as a fusion partner) was completely precipitated after heating.

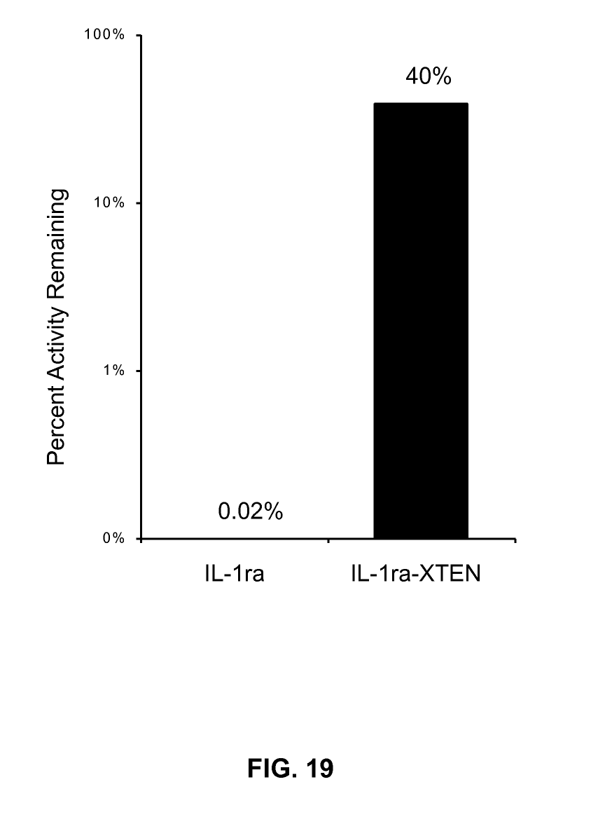

The IL-1 receptor binding activity of IL-1ra-XTEN was evaluated following the heat treatment described above. Receptor binding was performed as described above. Recombinant IL-1ra, which was fully denatured by heat treatment, retained less than 0.1% of its receptor activity following heat treatment. However, IL-1ra-XTEN retained approximately 40% of its receptor binding activity (FIG. 19). Together these data demonstrate that the XTEN polypeptide can prevent thermal-induced denaturation of its payload fusion partner and support the conclusion that XTEN have stabilizing properties.

### Example 24

### PK Analysis of Fusion Proteins Comprising IL-1ra and XTEN

The BFXTEN fusion proteins IL-1ra_AE864, IL-1ra_AM875, and IL-1ra_AM1296 were evaluated in cynomolgus monkeys in order to determine in vivo pharmacokinetic parameters of the respective fusion proteins. All compositions were provided in an aqueous buffer and were administered by subcutaneous (SC) route into separate animals using 1 mg/kg and/or 10 mg/kg single doses. Plasma samples were collected at various time points following administration and analyzed for concentrations of the test articles. Analysis was performed using a sandwich ELISA format. Rabbit polyclonal anti-XTEN antibodies were coated onto wells of an ELISA plate. The wells were blocked, washed and plasma samples were then incubated in the wells at varying dilutions to allow capture of the compound by the coated antibodies. Wells were washed extensively, and bound protein was detected using a biotinylated preparation of the polyclonal anti IL-1ra antibody and streptavidin HRP. Concentrations of test article were calculated at each time point by comparing the colorimetric response at each serum dilution to a standard curve. Pharmacokinetic parameters were calculated using the WinNonLin software package.

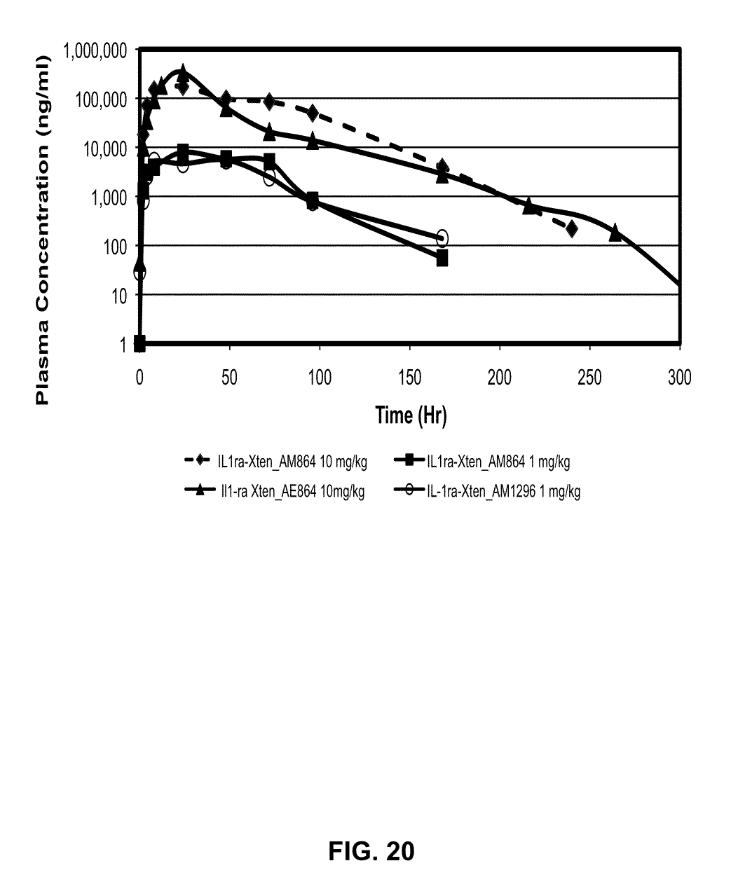

FIG. 20 shows the concentration profiles of the four IL-1ra-containing constructs, and calculated PK parameters are shown in Table 24. Following subcutaneous administration, the terminal half-life was calculated to be approximately 15-28 hours for the various preparations over the 336 h period. For reference, the published half-life of unmodified IL-1ra is well described in the literature as 4-6 h in adult humans.

Conclusions: The incorporation of different XTEN sequences into fusion proteins comprising IL-1ra results in significant enhancement of pharmacokinetic parameters for all three compositions, as demonstrated in the primate model, demonstrating the utility of such fusion protein compositions.

TABLE 24
PK parameters of BFXTEN compositions comprising IL-1ra and XTEN

| | IL-1ra | IL-1ra- | L-1ra- | IL-1ra- | |
|---|---|---|---|---|---|
| | XTEN_AE864 | XTEN_AM1296 | XTEN_AM875 | XTEN_AM875 | |
| Dose | 10 mg/kg | 1 mg/kg | 1 mg/kg | 10 mg/kg | Units |
| Tmax | 24 | 48 | 24 | 24 | Hr |
| Cmax | 334,571.5 | 5,493.3 | 7,894.7 | 172,220.5 | ng/ml |
| t1/2 | 28.0 | 24.2 | 15.5 | 19.3 | Hr |
| AUCall | 9,830,115.9 | 372,519.3 | 485,233.9 | 11,410,136.2 | (ng*Hr)/ml |
| Vz(observed)/F | 165.7 | 337.1 | 149.2 | 88.4 | ml |
| Cl(observed)/F | 4.1 | 9.7 | 6.7 | 3.2 | ml/hr |

### Example 25

### Use of BFXTEN in Diet-Induced Obese Mouse Model

The effects of combination therapy of biologically active proteins linked to XTEN were evaluated in a mouse model of diet-induced obesity to confirm the utility of fixed combinations of monomeric fusion proteins as a single BFXTEN composition.

Methods: The effects of combination therapy of glucagon linked to Y-288-XTEN (“Gcg-XTEN”) and exenatide linked to AE576-XTEN (“Ex4-XTEN”) or exenatide singly were tested in male C57BL/6J Diet-Induced Obese (DIO) Mice, age 10 weeks old. Mice raised on a 60% high fat diet were randomized into the treatment groups (n=10 per group) Ex4-XTEN864 (10 mg/kg IP Q2D), Ex4-XTEN864 (20 mg/kg IP Q4D), Ex4-XTEN864 (10 mg/kg IP Q2D) plus Gcg-XTEN288 (20 mg/kg IP BID), and Ex4-XTEN864 (20 mg/kg IP Q4D) plus Gcg-XTEN288 (40 mg/kg IP Q1D). A placebo group (n=10) treated with 20 mM Tris pH 7.5, 135 mM NaCl IP Q1D was tested in parallel. All groups were dosed continuously for 28 days. Body weight was monitored at regular intervals throughout the study and fasting blood glucose was measured before and after the treatment period. Groups were dosed continuously for a 28 day treatment period. Body weight was monitored continuously throughout the study and fasting blood glucose was measured before and after the treatment period, and lipid levels were determined after the treatment period.

Results: The results are shown in FIGS. 21-23. The data indicate that continuous dosing for one month yielded a significant reduction in weight gain in the animals treated with Gcg-XTEN alone and Ex4-XTEN alone, relative to placebo over the course of the study. In addition, animals dosed with Ex4-XTEN or Gcg-XTEN and Ex4-XTEN concurrently showed a statistically significantly greater weight loss compared to Glg-XTEN administered alone and compared to placebo. The toxic effects of glucagon administration are well documented. The maximum no-effect dose for glucagon in rats and beagle dogs has recently been reported as 1 mg/kg/day was regarded as a clear no-toxic-effect-level in both species (Eistrup C, Glucagon produced by recombinant DNA technology: repeated dose toxicity studies, intravenous administration to CD rats and beagle dogs for four weeks. Pharmacol Toxicol. 1993 August; 73(2):103-108).

The data also show that continuous dosing for one month yielded a significant reduction in fasting blood glucose for the animals treated with Ex4-XTEN alone relative to placebo, but not for animals treated with Gcg-XTEN alone. However, animals dosed with both Gcg-XTEN and exenatide concurrently showed a statistically significantly greater reduction in fasting blood glucose levels compared to either biologically active protein administered alone. Of note, the doses of Gcg-XTEN composition that resulted in the beneficial effects in combination with Ex4-XTEN were 20 and 40 μg/kg (complete fusion protein composition weight); at least 25-fold lower than the no-effect dose reported for glucagon alone in a rodent species.

Conclusions: The data support the conclusion that combination therapy with two fusion proteins of biologically active proteins linked to XTEN can result in a synergistic beneficial effect over that seen with a single biologically active protein such that administration of a combination composition can be tailored to reduce frequency of dosing or dosage compared to administration of a single biologic in order to reduce the threat of toxicity or unacceptable side effects.

### Example 26

### Human Clinical Trial Designs for Evaluating BFXTEN

Clinical trials are designed such that the efficacy and advantages of the BFXTEN compositions, relative to single biologics, can be verified in humans. For example, the BFXTEN fusion constructs comprising both glucagon and exenatide, as described in Example 25 above, would be used in clinical trials for characterizing the efficacy of the compositions. The trials would be conducted in one or more metabolic and/or cardiovascular diseases, disorders, or conditions that is improved, ameliorated, or inhibited by the administration of glucagon and exenatide. Such studies in adult patients would comprise three phases. First, a Phase I safety and pharmacokinetics study in adult patients would be conducted to determine the maximum tolerated dose and pharmacokinetics and pharmacodynamics in humans (either normal subjects or patients with a metabolic and/or cardiovascular disease or condition), as well as to define potential toxicities and adverse events to be tracked in future studies. The study is conducted in which single rising doses of compositions of fusion proteins of XTEN linked to glucagon and exenatide are administered and biochemical, PK, and clinical parameters are measured. This permits the determination of the maximum tolerated dose and establish the threshold and maximum concentrations in dosage and circulating drug that constitute the therapeutic window for the respective components. Thereafter, clinical trials of the BFXTEN compositions would be conducted in patients with the disease, disorder or condition.

Clinical Trial in Diabetes

A phase II dosing study would be conducted in diabetic patients where serum glucose pharmacodynamics and other physiologic, PK, safety and clinical parameters (such as listed below) appropriate for diabetes, insulin resistance and obesity conditions are measured as a function of the dosing of the fusion proteins comprising XTEN linked to glucagon and exenatide, yielding dose-ranging information on doses appropriate for a Phase III trial, in addition to collecting safety data related to adverse events. The PK parameters are correlated to the physiologic, clinical and safety parameter data to establish the therapeutic window for each component of the BFXTEN composition, permitting the clinician to establish either the appropriate ratio of the two component fusion proteins each comprising one biologically active protein, or to determine the single dose for a monomeric BFXTEN comprising two biologically active proteins. Finally, a phase III efficacy study would be conducted wherein diabetic patients are administered either the BFXTEN composition, a positive control, or a placebo daily, bi-weekly, or weekly (or other dosing schedule deemed appropriate given the pharmacokinetic and pharmacodynamic properties of the BFXTEN composition) for an extended period of time. Primary outcome measures of efficacy could include HbA1c concentrations, while secondary outcome measures include insulin requirements during the study, stimulated C peptide and insulin concentrations, fasting plasma glucose (FPG), serum cytokine levels, CRP levels, and insulin secretion and Insulin-sensitivity index derived from an OGTT with insulin and glucose measurements, as well as body weight, food consumption, and other accepted diabetic markers that are tracked relative to the placebo or positive control group. Efficacy outcomes are determined using standard statistical methods. Toxicity and adverse event markers would also be followed in this study to verify that the compound is safe when used in the manner described.

Clinical Trial in Arthritis

A phase II clinical study of human patients would be conducted in arthritis patients administered BFXTEN comprising XTEN linked to IL-1ra and/or anti-IL-2, anti-CD3 or a suitable anti-inflammatory protein to determine an appropriate dose to relieve at least one symptom associated with rheumatoid arthritis, including reducing joint swelling, joint tenderness, inflammation, morning stiffness, and pain, or at least one biological surrogate marker associated with rheumatoid arthritis, including reducing erythrocyte sedimentation rates, and serum levels of C-reactive protein and/or IL2 receptor. In addition, safety data related to adverse events would be collected. A phase III efficacy study would be conducted wherein arthritis patients are administered either the BFXTEN, a positive control, or a placebo daily, bi-weekly, or weekly (or other dosing schedule deemed appropriate given the pharmacokinetic and pharmacodynamic properties of the compound) for an extended period of time. Patients are evaluated for baseline symptoms of disease activity prior to receiving any treatments, including joint swelling, joint tenderness, inflammation, morning stiffness, disease activity evaluated by patient and physician as well as disability evaluated by, for example, a standardized Health Questionnaire Assessment (HAQ), and pain. Additional baseline evaluations include erythrocyte sedimentation rates (ESR), serum levels of C-reactive protein (CRP) and soluble IL-2 receptor (IL-2r). The clinical response to treatment is assessed using the criteria established by the American College of Rheumatology (ACR), such as the ACR20 criterion; i.e., if there was a 20 percent improvement in tender and swollen joint counts and 20 percent improvement in three of the five remaining symptoms measured, such as patient and physician global disease changes, pain, disability, and an acute phase reactant (Felson, D. T., et al., 1993 Arthritis and Rheumatism 36:729-740; Felson, D. T., et al., 1995 Arthritis and Rheumatism 38:1-9) Similarly, a subject would satisfy the ACR50 or ACR70 criterion if there was a 50 or 70 percent improvement, respectively, in tender and swollen joint counts and 50 or 70 percent improvement, respectively, in three of the five remaining symptoms measured, such as patient and physician global disease changes, pain, physical disability, and an acute phase reactant such as CRP or ESR. In addition, potential biomarkers of disease activity are measured, including rheumatoid factor, CRP, ESR, soluble IL-2R, soluble ICAM-1, soluble E-selectin, and MMP-3. Efficacy outcomes would be determined using standard statistical methods. Toxicity and adverse event markers would also be followed in this study to verify that the compound is safe when used in the manner described.

Clinical Trial in Acute Coronary Syndrome and Acute Myocardial Infarction.

A phase III trial in acute coronary syndrome (ACS) and/or acute myocardial infarction (AMI) would be conducted wherein patients diagnosed with ACS and/or AMI are administered either a BFXTEN fusion protein comprising, for example, IL-1ra and BNP, a positive control, the combination of the BFXTEN fusion protein plus a positive control substance, or a placebo daily, bi-weekly, or weekly (or other dosing schedule deemed appropriate given the pharmacokinetic and pharmacodynamic properties of the compound) for an extended period of time. The study is conducted to determine whether the BFXTEN is superior to the other treatment regimens for preventing cardiovascular death, non-fatal myocardial infarction, or ischemic stroke in subjects with a recent acute coronary syndrome. Patients are evaluated for baseline symptoms of disease activity prior to receiving any treatments, including signs or symptoms of unstable angina, chest pain experienced as tightness around the chest radiating to the left arm and the left angle of the jaw, diaphoresis (sweating), nausea and vomiting, shortness of breath, as well as electrocardiogram (ECG) evidence of non-Q-wave myocardial infarction and Q-wave myocardial infarction. Additional baseline evaluations include measurement of biomarkers, including ischemia-modified albumin (IMA), myeloperoxidase (MPO), glycogen phosphorylase isoenzyme BB-(GPBB), troponin, natriuretic peptide (both B-type natriuretic peptide (BNP) and N-terminal Pro BNP), and monocyte chemo attractive protein (MCP)-1. The clinical response to treatment is assessed using time to first occurrence of cardiovascular death, myocardial infarction, or ischemic stroke as primary outcome measures, while occurrences of or time to first unstable angina, hemorrhagic stroke, or fatal bleeding could serve as secondary outcome measures. Efficacy outcomes would be determined using standard statistical methods. Toxicity and adverse event markers are followed in this study to verify that the compound is safe when used in the manner described.

### Example 27

### Characterization of BP-XTEN Secondary Structure

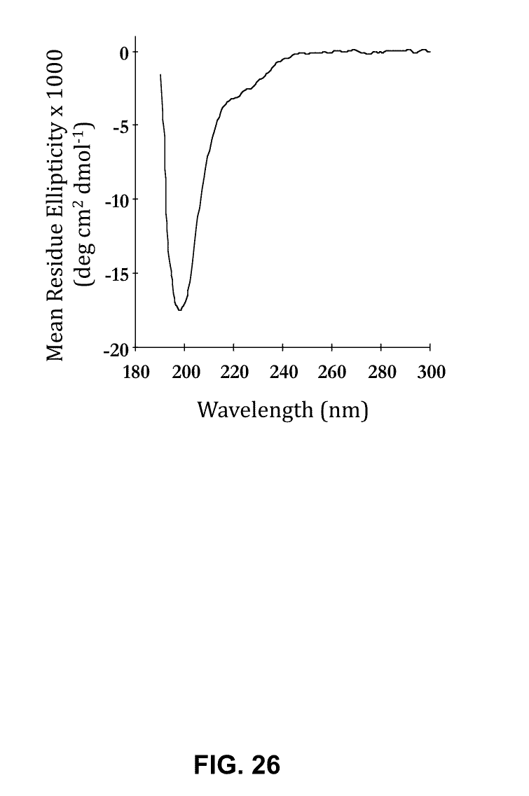

The fusion protein Ex4-XTEN_AE864 was evaluated for degree of secondary structure by circular dichroism spectroscopy. CD spectroscopy was performed on a Jasco J-715 (Jasco Corporation, Tokyo, Japan) spectropolarimeter equipped with Jasco Peltier temperature controller (TPC-348WI). The concentration of protein was adjusted to 0.2 mg/mL in 20 mM sodium phosphate pH 7.0, 50 mM NaCl. The experiments were carried out using HELLMA quartz cells with an optical path-length of 0.1 cm. The CD spectra were acquired at 5°, 25°, 45°, and 65° C. and processed using the J-700 version 1.08.01 (Build 1) Jasco software for Windows. The samples were equilibrated at each temperature for 5 min before performing CD measurements. All spectra were recorded in duplicate from 300 nm to 185 nm using a bandwidth of 1 nm and a time constant of 2 sec, at a scan speed of 100 nm/min The CD spectrum shown in FIG. 26 shows no evidence of stable secondary structure and is consistent with an unstructured polypeptide.

### Example 28

### C-terminal XTEN releasable by Elastase-2

A fusion protein consisting of an XTEN protein fused to the C-terminus of a BP, such as exendin-4 (Ex4) can be created with a XTEN release site cleavage sequence placed in between the BP and XTEN components. In this case, the release site contains an amino acid sequence that is recognized and cleaved by the elastase-2 protease (EC 3.4.21.37, Uniprot P08246). Specifically the sequence LGPVSGVP (SEQ ID NO: 672) [Rawlings N. D., et al. (2008) Nucleic Acids Res., 36: D320], would be cut after position 4 in the sequence. Elastase is constitutively expressed by neutrophils and is present at all times in the circulation. Its activity is tightly controlled by serpins and is therefore minimally active most of the time. Therefore as the long-lived Ex4-XTEN circulates, a fraction of it would be cleaved, creating a pool of shorter-lived exendin-4 to be used in glucose homeostasis. In a desirable feature of the inventive composition, this creates a circulating pro-drug depot that constantly releases an amount of free, fully active exendin-4.

### Example 29

### C-Terminal XTEN Releasable by MMP-12

An amylin-XTEN fusion protein consisting of an XTEN protein fused to the C-terminus of amylin can be created with a XTEN release site cleavage sequence placed in between the amylin and XTEN components. In this case, the XTEN release site contains an amino acid sequence that is recognized and cleaved by the MMP-12 protease (EC 3.4.24.65, Uniprot P39900). Specifically the sequence GPAGLGGA (SEQ ID NO: 673) [Rawlings N. D., et al. (2008) Nucleic Acids Res., 36: D320] would be cut after position 4 of the sequence. MMP-12 is constitutively expressed in whole blood. Therefore as the long-lived amylin-XTEN circulates, a fraction of it would be cleaved, creating a pool of shorter-lived amylin to be used in glucose homeostasis. In a desirable feature of the inventive composition, this creates a circulating pro-drug depot that constantly releases an amount of free, fully active amylin.

### Example 30

### C-Terminal XTEN Releasable by FXIa

A glucagon fusion protein consisting of an XTEN protein fused to the N-terminus of glucagon can be created with a XTEN release site cleavage sequence placed in between the glucagon and XTEN components. In this case, the release site cleavage sequence can be incorporated into the XTEN-glucagon that contains an amino acid sequence that is recognized and cleaved by the FXIa protease (EC 3.4.21.27, Uniprot PO₃₉₅₁). Specifically the amino acid sequence KLTRAET (SEQ ID NO: 674) is cut after the arginine of the sequence by FXIa protease. FXI is the pro-coagulant protease located immediately before FVIII in the intrinsic or contact activated coagulation pathway. Active FXIa is produced from FXI by proteolytic cleavage of the zymogen by FXIIa. Production of FXIa is tightly controlled and only occurs when coagulation is necessary for proper hemostasis. Therefore, by incorporation of the KLTRAET (SEQ ID NO: 674) cleavage sequence, the XTEN domain would only be removed from glucagon concurrent with activation of the intrinsic coagulation pathway. This creates a situation where the XTEN-glucagon fusion protein is processed in one additional manner during the activation of the intrinsic pathway.

### Example 31

### Analysis of Sequences for Secondary Structure by Prediction Algorithms

Amino acid sequences can be assessed for secondary structure via certain computer programs or algorithms, such as the well-known Chou-Fasman algorithm (Chou, P. Y., et al. (1974) Biochemistry, 13: 222-45) and the Garnier-Osguthorpe-Robson, or “GOR” method (Garnier J, Gibrat J F, Robson B. (1996). GOR method for predicting protein secondary structure from amino acid sequence. Methods Enzymol 266:540-553). For a given sequence, the algorithms can predict whether there exists some or no secondary structure at all, expressed as total and/or percentage of residues of the sequence that form, for example, alpha-helices or beta-sheets or the percentage of residues of the sequence predicted to result in random coil formation.

Several representative sequences from XTEN “families” have been assessed using two algorithm tools for the Chou-Fasman and GOR methods to assess the degree of secondary structure in these sequences. The Chou-Fasman tool was provided by William R. Pearson and the University of Virginia, at the “Biosupport” internet site, URL located on the World Wide Web at fasta.bioch.virginia.edu/fasta_www2/fasta_www.cgi?rm=misc1 as it existed on Jun. 19, 2009. The GOR tool was provided by Pole Informatique Lyonnais at the Network Protein Sequence Analysis internet site, URL located on the World Wide Web at .npsa-pbil.ibcp.fr/cgi-bin/secpred_gor4.pl as it existed on Jun. 19, 2008.

As a first step in the analyses, a single XTEN sequence was analyzed by the two algorithms. The AE864 composition is a XTEN with 864 amino acid residues created from multiple copies of four 12 amino acid sequence motifs consisting of the amino acids G, S, T, E, P, and A. The sequence motifs are characterized by the fact that there is limited repetitiveness within the motifs and within the overall sequence in that the sequence of any two consecutive amino acids is not repeated more than twice in any one 12 amino acid motif, and that no three contiguous amino acids of full-length the XTEN are identical. Successively longer portions of the AF 864 sequence from the N-terminus were analyzed by the Chou-Fasman and GOR algorithms (the latter requires a minimum length of 17 amino acids). The sequences were analyzed by entering the FASTA format sequences into the prediction tools and running the analysis. The results from the analyses are presented in Table 25.

The results indicate that, by the Chou-Fasman calculations, short XTEN of the AE and AG families, up to at least 288 amino acid residues, have no alpha-helices or beta sheets, but amounts of predicted percentage of random coil by the GOR algorithm vary from 78-99%. With increasing XTEN lengths of 504 residues to greater than 1300, the XTEN analyzed by the Chou-Fasman algorithm had predicted percentages of alpha-helices or beta sheets of 0 to about 2%, while the calculated percentages of random coil increased to from 94-99%. Those XTEN with alpha-helices or beta sheets were those sequences with one or more instances of three contiguous serine residues, which resulted in predicted beta-sheet formation. However, even these sequences still had approximately 99% random coil formation.

The analysis supports the conclusion that: 1) XTEN created from multiple sequence motifs of G, S, T, E, P, and A that have limited repetitiveness as to contiguous amino acids are predicted to have very low amounts of alpha-helices and beta-sheets; 2) that increasing the length of the XTEN does not appreciably increase the probability of alpha-helix or beta-sheet formation; and 3) that progressively increasing the length of the XTEN sequence by addition of non-repetitive 12-mers consisting of the amino acids G, S, T, E, P, and A results in increased percentage of random coil formation. Based on the numerous sequences evaluated by these methods, it is concluded that XTEN created from sequence motifs of G, S, T, E, P, and A that have limited repetitiveness (defined as no more than two identical contiguous amino acids in any one motif) are expected to have very limited secondary structure. With the exception of motifs containing three contiguous serines, it is believed that any order or combination of sequence motifs from Table 3 can be used to create an XTEN polypeptide that will result in an XTEN sequence that is substantially devoid of secondary structure, and that the effects of three contiguous serines is ameliorated by increasing the length of the XTEN. Such sequences are expected to have the characteristics described in the BFXTEN embodiments of the invention disclosed herein.

TABLE 25
CHOU-FASMAN and GOR prediction calculations of polypeptide sequences

| | | SEQ | | | |
|---|---|---|---|---|---|
| SEQ | | ID | No. | Chou-Fasman | GOR |
| NAME | Sequence | NO: | Residues | Calculation | Calculation |
| AE36: | GSPAGSPTSTEEGTSESATPESGPGT | 675 | 36 | Residue totals: H: 0 E: 0 | 94.44% |
| LCW0402_{—} | STEPSEGSAP | | | percent: H: 0.0 E: 0.0 | |
| 002 | | | | | |
| AE36: | GTSTEPSEGSAPGTSTEPSEGSAPGT | 676 | 36 | Residue totals: H: 0 E: 0 | 94.44% |
| LCW0402_{—} | STEPSEGSAP | | | percent: H: 0.0 E: 0.0 | |
| 003 | | | | | |
| AG36: | GASPGTSSTGSPGTPGSGTASSSPGS | 677 | 36 | Residue totals: H: 0 E: 0 | 77.78% |
| LCW0404_{—} | STPSGATGSP | | | percent: H: 0.0 E: 0.0 | |
| 001 | | | | | |
| AG36: | GSSTPSGATGSPGSSPSASTGTGPGS | 678 | 36 | Residue totals: H: 0 E: 0 | 83.33% |
| LCW0404_{—} | STPSGATGSP | | | percent: H: 0.0 E: 0.0 | |
| 003 | | | | | |
| AE42_1 | TEPSEGSAPGSPAGSPTSTEEGTSES | 679 | 42 | Residue totals: H: 0 E: 0 | 90.48% |
| | ATPESGPGSEPATSGS | | | percent: H: 0.0 E: 0.0 | |
| AE42_1 | TEPSEGSAPGSPAGSPTSTEEGTSES | 680 | 42 | Residue totals: H: 0 E: 0 | 90.48% |
| | ATPESGPGSEPATSGS | | | percent: H: 0.0 E: 0.0 | |
| AG42_1 | GAPSPSASTGTGPGTPGSGTASSSPG | 681 | 42 | Residue totals: H: 0 E: 0 | 88.10% |
| | SSTPSGATGSPGPSGP | | | percent: H: 0.0 E: 0.0 | |
| AG42_2 | GPGTPGSGTASSSPGSSTPSGATGSP | 682 | 42 | Residue totals: H: 0 E: 0 | 88.10% |
| | GSSPSASTGTGPGASP | | | percent: H: 0.0 E: 0.0 | |
| AE144 | GSEPATSGSETPGTSESATPESGPGS | 683 | 144 | Residue totals: H: 0 E: 0 | 98.61% |
| | EPATSGSETPGSPAGSPTSTEEGTST | | | percent: H: 0.0 E: 0.0 | |
| | EPSEGSAPGSEPATSGSETPGSEPAT | | | | |
| | SGSETPGSEPATSGSETPGTSTEPSE | | | | |
| | GSAPGTSESATPESGPGSEPATSGSE | | | | |
| | TPGTSTEPSEGSAP | | | | |
| AG144_1 | PGSSPSASTGTGPGSSPSASTGTGPG | 684 | 144 | Residue totals: H: 0 E: 0 | 91.67% |
| | TPGSGTASSSPGSSTPSGATGSPGSS | | | percent: H: 0.0 E: 0.0 | |
| | PSASTGTGPGASPGTSSTGSPGTPGS | | | | |
| | GTASSSPGSSTPSGATGSPGTPGSGT | | | | |
| | ASSSPGASPGTSSTGSPGASPGTSST | | | | |
| | GSPGTPGSGTASSS | | | | |
| AE288 | GTSESATPESGPGSEPATSGSETPGT | 685 | 288 | Residue totals: H: 0 E: 0 | 99.31% |
| | SESATPESGPGSEPATSGSETPGTSE | | | percent: H: 0.0 E: 0.0 | |
| | SATPESGPGTSTEPSEGSAPGSPAGS | | | | |
| | PTSTEEGTSESATPESGPGSEPATSG | | | | |
| | SETPGTSESATPESGPGSPAGSPTST | | | | |
| | EEGSPAGSPTSTEEGTSTEPSEGSAP | | | | |
| | GTSESATPESGPGTSESATPESGPGT | | | | |
| | SESATPESGPGSEPATSGSETPGSEP | | | | |
| | ATSGSETPGSPAGSPTSTEEGTSTEP | | | | |
| | SEGSAPGTSTEPSEGSAPGSEPATSG | | | | |
| | SETPGTSESATPESGPGTSTEPSEGS | | | | |
| | AP | | | | |
| AG288_2 | GSSPSASTGTGPGSSPSASTGTGPGT | 686 | 288 | Residue totals: H: 0 E: 0 | 92.71 |
| | PGSGTASSSPGSSTPSGATGSPGSSP | | | percent: H: 0.0 E: 0.0 | |
| | SASTGTGPGASPGTSSTGSPGTPGSG | | | | |
| | TASSSPGSSTPSGATGSPGTPGSGTA | | | | |
| | SSSPGASPGTSSTGSPGASPGTSSTG | | | | |
| | SPGTPGSGTASSSPGSSTPSGATGSP | | | | |
| | GASPGTSSTGSPGTPGSGTASSSPGS | | | | |
| | STPSGATGSPGSSPSASTGTGPGSSP | | | | |
| | SASTGTGPGSSTPSGATGSPGSSTPS | | | | |
| | GATGSPGASPGTSSTGSPGASPGTSS | | | | |
| | TGSPGASPGTSSTGSPGTPGSGTASS | | | | |
| | SP | | | | |
| AF504 | GASPGTSSTGSPGSSPSASTGTGPGS | 687 | 504 | Residue totals: H: 0 E: 0 | 94.44% |
| | SPSASTGTGPGTPGSGTASSSPGSST | | | percent: H: 0.0 E: 0.0 | |
| | PSGATGSPGSNPSASTGTGPGASPG | | | | |
| | TSSTGSPGTPGSGTASSSPGSSTPSG | | | | |
| | ATGSPGTPGSGTASSSPGASPGTSST | | | | |
| | GSPGASPGTSSTGSPGTPGSGTASSS | | | | |
| | PGSSTPSGATGSPGASPGTSSTGSPG | | | | |
| | TPGSGTASSSPGSSTPSGATGSPGSN | | | | |
| | PSASTGTGPGSSPSASTGTGPGSSTP | | | | |
| | SGATGSPGSSTPSGATGSPGASPGTS | | | | |
| | STGSPGASPGTSSTGSPGASPGTSST | | | | |
| | GSPGTPGSGTASSSPGASPGTSSTGS | | | | |
| | PGASPGTSSTGSPGASPGTSSTGSPG | | | | |
| | SSPSASTGTGPGTPGSGTASSSPGAS | | | | |
| | PGTSSTGSPGASPGTSSTGSPGASPG | | | | |
| | TSSTGSPGSSTPSGATGSPGSSTPSG | | | | |
| | ATGSPGASPGTSSTGSPGTPGSGTAS | | | | |
| | SSPGSSTPSGATGSPGSSTPSGATGS | | | | |
| | PGSSTPSGATGSPGSSPSASTGTGPG | | | | |
| | ASPGTSSTGSP | | | | |
| AD 576 | GSSESGSSEGGPGSGGEPSESGSSGS | 688 | 576 | Residue totals: H: 7 E: 0 | 99.65% |
| | SESGSSEGGPGSSESGSSEGGPGSSE | | | percent: H: 1.2 E: 0.0 | |
| | SGSSEGGPGSSESGSSEGGPGSSESG | | | | |
| | SSEGGPGESPGGSSGSESGSEGSSGP | | | | |
| | GESSGSSESGSSEGGPGSSESGSSEG | | | | |
| | GPGSSESGSSEGGPGSGGEPSESGSS | | | | |
| | GESPGGSSGSESGESPGGSSGSESGS | | | | |
| | GGEPSESGSSGSSESGSSEGGPGSGG | | | | |
| | EPSESGSSGSGGEPSESGSSGSEGSS | | | | |
| | GPGESSGESPGGSSGSESGSGGEPSE | | | | |
| | SGSSGSGGEPSESGSSGSGGEPSESG | | | | |
| | SSGSSESGSSEGGPGESPGGSSGSES | | | | |
| | GESPGGSSGSESGESPGGSSGSESGE | | | | |
| | SPGGSSGSESGESPGGSSGSESGSSE | | | | |
| | SGSSEGGPGSGGEPSESGSSGSEGSS | | | | |
| | GPGESSGSSESGSSEGGPGSGGEPSE | | | | |
| | SGSSGSSESGSSEGGPGSGGEPSESG | | | | |
| | SSGESPGGSSGSESGESPGGSSGSES | | | | |
| | GSSESGSSEGGPGSGGEPSESGSSGS | | | | |
| | SESGSSEGGPGSGGEPSESGSSGSGG | | | | |
| | EPSESGSSGESPGGSSGSESGSEGSS | | | | |
| | GPGESSGSSESGSSEGGPGSEGSSGP | | | | |
| | GESS | | | | |
| AE576 | GSPAGSPTSTEEGTSESATPESGPGT | 689 | 576 | Residue totals: H: 2 E: 0 | 99.65% |
| | STEPSEGSAPGSPAGSPTSTEEGTST | | | percent: H: 0.4 E: 0.0 | |
| | EPSEGSAPGTSTEPSEGSAPGTSESA | | | | |
| | TPESGPGSEPATSGSETPGSEPATSG | | | | |
| | SETPGSPAGSPTSTEEGTSESATPES | | | | |
| | GPGTSTEPSEGSAPGTSTEPSEGSAP | | | | |
| | GSPAGSPTSTEEGTSTEPSEGSAPGT | | | | |
| | STEPSEGSAPGTSESATPESGPGTST | | | | |
| | EPSEGSAPGTSESATPESGPGSEPAT | | | | |
| | SGSETPGTSTEPSEGSAPGTSTEPSE | | | | |
| | GSAPGTSESATPESGPGTSESATPES | | | | |
| | GPGSPAGSPTSTEEGTSESATPESGP | | | | |
| | GSEPATSGSETPGTSESATPESGPGT | | | | |
| | STEPSEGSAPGTSTEPSEGSAPGTST | | | | |
| | EPSEGSAPGTSTEPSEGSAPGTSTEP | | | | |
| | SEGSAPGTSTEPSEGSAPGSPAGSPT | | | | |
| | STEEGTSTEPSEGSAPGTSESATPES | | | | |
| | GPGSEPATSGSETPGTSESATPESGP | | | | |
| | GSEPATSGSETPGTSESATPESGPGT | | | | |
| | STEPSEGSAPGTSESATPESGPGSPA | | | | |
| | GSPTSTEEGSPAGSPTSTEEGSPAGS | | | | |
| | PTSTEEGTSESATPESGPGTSTEPSE | | | | |
| | GSAP | | | | |
| AG576 | PGTPGSGTASSSPGSSTPSGATGSPG | 690 | 576 | Residue totals: H: 0 E: 3 | 99.31% |
| | SSPSASTGTGPGSSPSASTGTGPGSS | | | percent: H: 0.4 E: 0.5 | |
| | TPSGATGSPGSSTPSGATGSPGASPG | | | | |
| | TSSTGSPGASPGTSSTGSPGASPGTS | | | | |
| | STGSPGTPGSGTASSSPGASPGTSST | | | | |
| | GSPGASPGTSSTGSPGASPGTSSTGS | | | | |
| | PGSSPSASTGTGPGTPGSGTASSSPG | | | | |
| | ASPGTSSTGSPGASPGTSSTGSPGAS | | | | |
| | PGTSSTGSPGSSTPSGATGSPGSSTPS | | | | |
| | GATGSPGASPGTSSTGSPGTPGSGT | | | | |
| | ASSSPGSSTPSGATGSPGSSTPSGAT | | | | |
| | GSPGSSTPSGATGSPGSSPSASTGTG | | | | |
| | PGASPGTSSTGSPGASPGTSSTGSPG | | | | |
| | TPGSGTASSSPGASPGTSSTGSPGAS | | | | |
| | PGTSSTGSPGASPGTSSTGSPGASPG | | | | |
| | TSSTGSPGTPGSGTASSSPGSSTPSG | | | | |
| | ATGSPGTPGSGTASSSPGSSTPSGAT | | | | |
| | GSPGTPGSGTASSSPGSSTPSGATGS | | | | |
| | PGSSTPSGATGSPGSSPSASTGTGPG | | | | |
| | SSPSASTGTGPGASPGTSSTGSPGTP | | | | |
| | GSGTASSSPGSSTPSGATGSPGSSPS | | | | |
| | ASTGTGPGSSPSASTGTGPGASPGTS | | | | |
| | STGS | | | | |
| AF540 | GSTSSTAESPGPGSTSSTAESPGPGS | 691 | 540 | Residue totals: H: 2 E: 0 | 99.65 |
| | TSESPSGTAPGSTSSTAESPGPGSTSS | | | percent: H: 0.4 E: 0.0 | |
| | TAESPGPGTSTPESGSASPGSTSESPS | | | | |
| | GTAPGTSPSGESSTAPGSTSESPSGT | | | | |
| | APGSTSESPSGTAPGTSPSGESSTAP | | | | |
| | GSTSESPSGTAPGSTSESPSGTAPGT | | | | |
| | SPSGESSTAPGSTSESPSGTAPGSTSE | | | | |
| | SPSGTAPGSTSESPSGTAPGTSTPES | | | | |
| | GSASPGSTSESPSGTAPGTSTPESGS | | | | |
| | ASPGSTSSTAESPGPGSTSSTAESPG | | | | |
| | PGTSTPESGSASPGTSTPESGSASPG | | | | |
| | STSESPSGTAPGTSTPESGSASPGTST | | | | |
| | PESGSASPGSTSESPSGTAPGSTSESP | | | | |
| | SGTAPGSTSESPSGTAPGSTSSTAES | | | | |
| | PGPGTSTPESGSASPGTSTPESGSAS | | | | |
| | PGSTSESPSGTAPGSTSESPSGTAPG | | | | |
| | TSTPESGSASPGSTSESPSGTAPGSTS | | | | |
| | ESPSGTAPGTSTPESGSASPGTSPSG | | | | |
| | ESSTAPGSTSSTAESPGPGTSPSGESS | | | | |
| | TAPGSTSSTAESPGPGTSTPESGSAS | | | | |
| | PGSTSESPSGTAP | | | | |
| AD836 | GSSESGSSEGGPGSSESGSSEGGPGE | 692 | 836 | Residue totals: H: 0 E: 0 | 98.44% |
| | SPGGSSGSESGSGGEPSESGSSGESP | | | percent: H: 0.0 E: 0.0 | |
| | GGSSGSESGESPGGSSGSESGSSESG | | | | |
| | SSEGGPGSSESGSSEGGPGSSESGSS | | | | |
| | EGGPGESPGGSSGSESGESPGGSSGS | | | | |
| | ESGESPGGSSGSESGSSESGSSEGGP | | | | |
| | GSSESGSSEGGPGSSESGSSEGGPGS | | | | |
| | SESGSSEGGPGSSESGSSEGGPGSSE | | | | |
| | SGSSEGGPGSGGEPSESGSSGESPGG | | | | |
| | SSGSESGESPGGSSGSESGSGGEPSE | | | | |
| | SGSSGSEGSSGPGESSGSSESGSSEG | | | | |
| | GPGSGGEPSESGSSGSEGSSGPGESS | | | | |
| | GSSESGSSEGGPGSGGEPSESGSSGE | | | | |
| | SPGGSSGSESGSGGEPSESGSSGSGG | | | | |
| | EPSESGSSGSSESGSSEGGPGSGGEP | | | | |
| | SESGSSGSGGEPSESGSSGSEGSSGP | | | | |
| | GESSGESPGGSSGSESGSEGSSGPGE | | | | |
| | SSGSEGSSGPGESSGSGGEPSESGSS | | | | |
| | GSSESGSSEGGPGSSESGSSEGGPGE | | | | |
| | SPGGSSGSESGSGGEPSESGSSGSEG | | | | |
| | SSGPGESSGESPGGSSGSESGSEGSS | | | | |
| | GPGSSESGSSEGGPGSGGEPSESGSS | | | | |
| | GSEGSSGPGESSGSEGSSGPGESSGS | | | | |
| | EGSSGPGESSGSGGEPSESGSSGSGG | | | | |
| | EPSESGSSGESPGGSSGSESGESPGG | | | | |
| | SSGSESGSGGEPSESGSSGSEGSSGP | | | | |
| | GESSGESPGGSSGSESGSSESGSSEG | | | | |
| | GPGSSESGSSEGGPGSSESGSSEGGP | | | | |
| | GSGGEPSESGSSGSSESGSSEGGPGE | | | | |
| | SPGGSSGSESGSGGEPSESGSSGSSE | | | | |
| | SGSSEGGPGESPGGSSGSESGSGGEP | | | | |
| | SESGSSGESPGGSSGSESGSGGEPSE | | | | |
| | SGSS | | | | |
| AE864 | GSPAGSPTSTEEGTSESATPESGPGT | 693 | 864 | Residue totals: H: 2 E: 3 | 99.77% |
| | STEPSEGSAPGSPAGSPTSTEEGTST | | | percent: H: 0.2 E: 0.4 | |
| | EPSEGSAPGTSTEPSEGSAPGTSESA | | | | |
| | TPESGPGSEPATSGSETPGSEPATSG | | | | |
| | SETPGSPAGSPTSTEEGTSESATPES | | | | |
| | GPGTSTEPSEGSAPGTSTEPSEGSAP | | | | |
| | GSPAGSPTSTEEGTSTEPSEGSAPGT | | | | |
| | STEPSEGSAPGTSESATPESGPGTST | | | | |
| | EPSEGSAPGTSESATPESGPGSEPAT | | | | |
| | SGSETPGTSTEPSEGSAPGTSTEPSE | | | | |
| | GSAPGTSESATPESGPGTSESATPES | | | | |
| | GPGSPAGSPTSTEEGTSESATPESGP | | | | |
| | GSEPATSGSETPGTSESATPESGPGT | | | | |
| | STEPSEGSAPGTSTEPSEGSAPGTST | | | | |
| | EPSEGSAPGTSTEPSEGSAPGTSTEP | | | | |
| | SEGSAPGTSTEPSEGSAPGSPAGSPT | | | | |
| | STEEGTSTEPSEGSAPGTSESATPES | | | | |
| | GPGSEPATSGSETPGTSESATPESGP | | | | |
| | GSEPATSGSETPGTSESATPESGPGT | | | | |
| | STEPSEGSAPGTSESATPESGPGSPA | | | | |
| | GSPTSTEEGSPAGSPTSTEEGSPAGS | | | | |
| | PTSTEEGTSESATPESGPGTSTEPSE | | | | |
| | GSAPGTSESATPESGPGSEPATSGSE | | | | |
| | TPGTSESATPESGPGSEPATSGSETP | | | | |
| | GTSESATPESGPGTSTEPSEGSAPGS | | | | |
| | PAGSPTSTEEGTSESATPESGPGSEP | | | | |
| | ATSGSETPGTSESATPESGPGSPAGS | | | | |
| | PTSTEEGSPAGSPTSTEEGTSTEPSE | | | | |
| | GSAPGTSESATPESGPGTSESATPES | | | | |
| | GPGTSESATPESGPGSEPATSGSETP | | | | |
| | GSEPATSGSETPGSPAGSPTSTEEGT | | | | |
| | STEPSEGSAPGTSTEPSEGSAPGSEP | | | | |
| | ATSGSETPGTSESATPESGPGTSTEP | | | | |
| | SEGSAP | | | | |
| AF864 | GSTSESPSGTAPGTSPSGESSTAPGS | 694 | 875 | Residue totals: H: 2 E: 0 | 95.20% |
| | TSESPSGTAPGSTSESPSGTAPGTSTP | | | percent: H: 0.2 E: 0.0 | |
| | ESGSASPGTSTPESGSASPGSTSESPS | | | | |
| | GTAPGSTSESPSGTAPGTSPSGESST | | | | |
| | APGSTSESPSGTAPGTSPSGESSTAP | | | | |
| | GTSPSGESSTAPGSTSSTAESPGPGT | | | | |
| | SPSGESSTAPGTSPSGESSTAPGSTSS | | | | |
| | TAESPGPGTSTPESGSASPGTSTPES | | | | |
| | GSASPGSTSESPSGTAPGSTSESPSG | | | | |
| | TAPGTSTPESGSASPGSTSSTAESPG | | | | |
| | PGTSTPESGSASPGSTSESPSGTAPG | | | | |
| | TSPSGESSTAPGSTSSTAESPGPGTSP | | | | |
| | SGESSTAPGTSTPESGSASPGSTSST | | | | |
| | AESPGPGSTSSTAESPGPGSTSSTAE | | | | |
| | SPGPGSTSSTAESPGPGTSPSGESST | | | | |
| | APGSTSESPSGTAPGSTSESPSGTAP | | | | |
| | GTSTPESGPXXXGASASGAPSTXXX | | | | |
| | XSESPSGTAPGSTSESPSGTAPGSTS | | | | |
| | ESPSGTAPGSTSESPSGTAPGSTSESP | | | | |
| | SGTAPGSTSESPSGTAPGTSTPESGS | | | | |
| | ASPGTSPSGESSTAPGTSPSGESSTA | | | | |
| | PGSTSSTAESPGPGTSPSGESSTAPG | | | | |
| | TSTPESGSASPGSTSESPSGTAPGSTS | | | | |
| | ESPSGTAPGTSPSGESSTAPGSTSESP | | | | |
| | SGTAPGTSTPESGSASPGTSTPESGS | | | | |
| | ASPGSTSESPSGTAPGTSTPESGSAS | | | | |
| | PGSTSSTAESPGPGSTSESPSGTAPG | | | | |
| | STSESPSGTAPGTSPSGESSTAPGSTS | | | | |
| | STAESPGPGTSPSGESSTAPGTSTPES | | | | |
| | GSASPGTSPSGESSTAPGTSPSGESS | | | | |
| | TAPGTSPSGESSTAPGSTSSTAESPG | | | | |
| | PGSTSSTAESPGPGTSPSGESSTAPG | | | | |
| | SSPSASTGTGPGSSTPSGATGSPGSS | | | | |
| | TPSGATGSP | | | | |
| AG864 | GASPGTSSTGSPGSSPSASTGTGPGS | 695 | 864 | Residue totals: H: 0 E: 0 | 94.91% |
| | SPSASTGTGPGTPGSGTASSSPGSST | | | percent: H: 0.0 E: 0.0 | |
| | PSGATGSPGSSPSASTGTGPGASPGT | | | | |
| | SSTGSPGTPGSGTASSSPGSSTPSGA | | | | |
| | TGSPGTPGSGTASSSPGASPGTSSTG | | | | |
| | SPGASPGTSSTGSPGTPGSGTASSSP | | | | |
| | GSSTPSGATGSPGASPGTSSTGSPGT | | | | |
| | PGSGTASSSPGSSTPSGATGSPGSSP | | | | |
| | SASTGTGPGSSPSASTGTGPGSSTPS | | | | |
| | GATGSPGSSTPSGATGSPGASPGTSS | | | | |
| | TGSPGASPGTSSTGSPGASPGTSSTG | | | | |
| | SPGTPGSGTASSSPGASPGTSSTGSP | | | | |
| | GASPGTSSTGSPGASPGTSSTGSPGS | | | | |
| | SPSASTGTGPGTPGSGTASSSPGASP | | | | |
| | GTSSTGSPGASPGTSSTGSPGASPGT | | | | |
| | SSTGSPGSSTPSGATGSPGSSTPSGA | | | | |
| | TGSPGASPGTSSTGSPGTPGSGTASS | | | | |
| | SPGSSTPSGATGSPGSSTPSGATGSP | | | | |
| | GSSTPSGATGSPGSSPSASTGTGPGA | | | | |
| | SPGTSSTGSPGASPGTSSTGSPGTPG | | | | |
| | SGTASSSPGASPGTSSTGSPGASPGT | | | | |
| | SSTGSPGASPGTSSTGSPGASPGTSS | | | | |
| | TGSPGTPGSGTASSSPGSSTPSGATG | | | | |
| | SPGTPGSGTASSSPGSSTPSGATGSP | | | | |
| | GTPGSGTASSSPGSSTPSGATGSPGS | | | | |
| | STPSGATGSPGSSPSASTGTGPGSSP | | | | |
| | SASTGTGPGASPGTSSTGSPGTPGSG | | | | |
| | TASSSPGSSTPSGATGSPGSSPSAST | | | | |
| | GTGPGSSPSASTGTGPGASPGTSSTG | | | | |
| | SPGASPGTSSTGSPGSSTPSGATGSP | | | | |
| | GSSPSASTGTGPGASPGTSSTGSPGS | | | | |
| | SPSASTGTGPGTPGSGTASSSPGSST | | | | |
| | PSGATGSPGSSTPSGATGSPGASPGT | | | | |
| | SSTGSP | | | | |
| AM875 | GTSTEPSEGSAPGSEPATSGSETPGS | 696 | 875 | Residue totals: H: 7 E: 3 | 98.63% |
| | PAGSPTSTEEGSTSSTAESPGPGTST | | | percent: H: 0.8 E: 0.3 | |
| | PESGSASPGSTSESPSGTAPGSTSESP | | | | |
| | SGTAPGTSTPESGSASPGTSTPESGS | | | | |
| | ASPGSEPATSGSETPGTSESATPESG | | | | |
| | PGSPAGSPTSTEEGTSTEPSEGSAPG | | | | |
| | TSESATPESGPGTSTEPSEGSAPGTS | | | | |
| | TEPSEGSAPGSPAGSPTSTEEGTSTE | | | | |
| | PSEGSAPGTSTEPSEGSAPGTSESAT | | | | |
| | PESGPGTSESATPESGPGTSTEPSEG | | | | |
| | SAPGTSTEPSEGSAPGTSESATPESG | | | | |
| | PGTSTEPSEGSAPGSEPATSGSETPG | | | | |
| | SPAGSPTSTEEGSSTPSGATGSPGTP | | | | |
| | GSGTASSSPGSSTPSGATGSPGTSTE | | | | |
| | PSEGSAPGTSTEPSEGSAPGSEPATS | | | | |
| | GSETPGSPAGSPTSTEEGSPAGSPTS | | | | |
| | TEEGTSTEPSEGSAPGASASGAPSTG | | | | |
| | GTSESATPESGPGSPAGSPTSTEEGS | | | | |
| | PAGSPTSTEEGSTSSTAESPGPGSTS | | | | |
| | ESPSGTAPGTSPSGESSTAPGTPGSG | | | | |
| | TASSSPGSSTPSGATGSPGSSPSAST | | | | |
| | GTGPGSEPATSGSETPGTSESATPES | | | | |
| | GPGSEPATSGSETPGSTSSTAESPGP | | | | |
| | GSTSSTAESPGPGTSPSGESSTAPGS | | | | |
| | EPATSGSETPGSEPATSGSETPGTST | | | | |
| | EPSEGSAPGSTSSTAESPGPGTSTPES | | | | |
| | GSASPGSTSESPSGTAPGTSTEPSEG | | | | |
| | SAPGTSTEPSEGSAPGTSTEPSEGSA | | | | |
| | PGSSTPSGATGSPGSSPSASTGTGPG | | | | |
| | ASPGTSSTGSPGSEPATSGSETPGTS | | | | |
| | ESATPESGPGSPAGSPTSTEEGSSTPS | | | | |
| | GATGSPGSSPSASTGTGPGASPGTSS | | | | |
| | TGSPGTSESATPESGPGTSTEPSEGS | | | | |
| | APGTSTEPSEGSAP | | | | |
| AM1318 | GTSTEPSEGSAPGSEPATSGSETPGS | 697 | 1318 | Residue totals: H: 7 E: 0 | 99.17% |
| | PAGSPTSTEEGSTSSTAESPGPGTST | | | percent: H: 0.7 E: 0.0 | |
| | PESGSASPGSTSESPSGTAPGSTSESP | | | | |
| | SGTAPGTSTPESGSASPGTSTPESGS | | | | |
| | ASPGSEPATSGSETPGTSESATPESG | | | | |
| | PGSPAGSPTSTEEGTSTEPSEGSAPG | | | | |
| | TSESATPESGPGTSTEPSEGSAPGTS | | | | |
| | TEPSEGSAPGSPAGSPTSTEEGTSTE | | | | |
| | PSEGSAPGTSTEPSEGSAPGTSESAT | | | | |
| | PESGPGTSESATPESGPGTSTEPSEG | | | | |
| | SAPGTSTEPSEGSAPGTSESATPESG | | | | |
| | PGTSTEPSEGSAPGSEPATSGSETPG | | | | |
| | SPAGSPTSTEEGSSTPSGATGSPGTP | | | | |
| | GSGTASSSPGSSTPSGATGSPGTSTE | | | | |
| | PSEGSAPGTSTEPSEGSAPGSEPATS | | | | |
| | GSETPGSPAGSPTSTEEGSPAGSPTS | | | | |
| | TEEGTSTEPSEGSAPGPEPTGPAPSG | | | | |
| | GSEPATSGSETPGTSESATPESGPGS | | | | |
| | PAGSPTSTEEGTSESATPESGPGSPA | | | | |
| | GSPTSTEEGSPAGSPTSTEEGTSESA | | | | |
| | TPESGPGSPAGSPTSTEEGSPAGSPT | | | | |
| | STEEGSTSSTAESPGPGSTSESPSGT | | | | |
| | APGTSPSGESSTAPGSTSESPSGTAP | | | | |
| | GSTSESPSGTAPGTSPSGESSTAPGT | | | | |
| | STEPSEGSAPGTSESATPESGPGTSE | | | | |
| | SATPESGPGSEPATSGSETPGTSESA | | | | |
| | TPESGPGTSESATPESGPGTSTEPSE | | | | |
| | GSAPGTSESATPESGPGTSTEPSEGS | | | | |
| | APGTSPSGESSTAPGTSPSGESSTAP | | | | |
| | GTSPSGESSTAPGTSTEPSEGSAPGS | | | | |
| | PAGSPTSTEEGTSTEPSEGSAPGSSPS | | | | |
| | ASTGTGPGSSTPSGATGSPGSSTPSG | | | | |
| | ATGSPGSSTPSGATGSPGSSTPSGAT | | | | |
| | GSPGASPGTSSTGSPGASASGAPSTG | | | | |
| | GTSPSGESSTAPGSTSSTAESPGPGT | | | | |
| | SPSGESSTAPGTSESATPESGPGTST | | | | |
| | EPSEGSAPGTSTEPSEGSAPGSSPSA | | | | |
| | STGTGPGSSTPSGATGSPGASPGTSS | | | | |
| | TGSPGTSTPESGSASPGTSPSGESST | | | | |
| | APGTSPSGESSTAPGTSESATPESGP | | | | |
| | GSEPATSGSETPGTSTEPSEGSAPGS | | | | |
| | TSESPSGTAPGSTSESPSGTAPGTSTP | | | | |
| | ESGSASPGSPAGSPTSTEEGTSESAT | | | | |
| | PESGPGTSTEPSEGSAPGSPAGSPTS | | | | |
| | TEEGTSESATPESGPGSEPATSGSET | | | | |
| | PGSSTPSGATGSPGASPGTSSTGSPG | | | | |
| | SSTPSGATGSPGSTSESPSGTAPGTS | | | | |
| | PSGESSTAPGSTSSTAESPGPGSSTPS | | | | |
| | GATGSPGASPGTSSTGSPGTPGSGT | | | | |
| | ASSSPGSPAGSPTSTEEGSPAGSPTS | | | | |
| | TEEGTSTEPSEGSAP | | | | |
| AM923 | MAEPAGSPTSTEEGASPGTSSTGSP | 698 | 924 | Residue totals: H: 4 E: 3 | 98.70% |
| | GSSTPSGATGSPGSSTPSGATGSPGT | | | percent: H: 0.4 E: 0.3 | |
| | STEPSEGSAPGSEPATSGSETPGSPA | | | | |
| | GSPTSTEEGSTSSTAESPGPGTSTPES | | | | |
| | GSASPGSTSESPSGTAPGSTSESPSG | | | | |
| | TAPGTSTPESGSASPGTSTPESGSAS | | | | |
| | PGSEPATSGSETPGTSESATPESGPG | | | | |
| | SPAGSPTSTEEGTSTEPSEGSAPGTS | | | | |
| | ESATPESGPGTSTEPSEGSAPGTSTE | | | | |
| | PSEGSAPGSPAGSPTSTEEGTSTEPS | | | | |
| | EGSAPGTSTEPSEGSAPGTSESATPE | | | | |
| | SGPGTSESATPESGPGTSTEPSEGSA | | | | |
| | PGTSTEPSEGSAPGTSESATPESGPG | | | | |
| | TSTEPSEGSAPGSEPATSGSETPGSP | | | | |
| | AGSPTSTEEGSSTPSGATGSPGTPGS | | | | |
| | GTASSSPGSSTPSGATGSPGTSTEPS | | | | |
| | EGSAPGTSTEPSEGSAPGSEPATSGS | | | | |
| | ETPGSPAGSPTSTEEGSPAGSPTSTE | | | | |
| | EGTSTEPSEGSAPGASASGAPSTGG | | | | |
| | TSESATPESGPGSPAGSPTSTEEGSP | | | | |
| | AGSPTSTEEGSTSSTAESPGPGSTSE | | | | |
| | SPSGTAPGTSPSGESSTAPGTPGSGT | | | | |
| | ASSSPGSSTPSGATGSPGSSPSASTG | | | | |
| | TGPGSEPATSGSETPGTSESATPESG | | | | |
| | PGSEPATSGSETPGSTSSTAESPGPG | | | | |
| | STSSTAESPGPGTSPSGESSTAPGSEP | | | | |
| | ATSGSETPGSEPATSGSETPGTSTEP | | | | |
| | SEGSAPGSTSSTAESPGPGTSTPESG | | | | |
| | SASPGSTSESPSGTAPGTSTEPSEGS | | | | |
| | APGTSTEPSEGSAPGTSTEPSEGSAP | | | | |
| | GSSTPSGATGSPGSSPSASTGTGPGA | | | | |
| | SPGTSSTGSPGSEPATSGSETPGTSES | | | | |
| | ATPESGPGSPAGSPTSTEEGSSTPSG | | | | |
| | ATGSPGSSPSASTGTGPGASPGTSST | | | | |
| | GSPGTSESATPESGPGTSTEPSEGSA | | | | |
| | PGTSTEPSEGSAP | | | | |
| AE912 | MAEPAGSPTSTEEGTPGSGTASSSP | 699 | 913 | Residue totals: H: 8 E: 3 | 99.45% |
| | GSSTPSGATGSPGASPGTSSTGSPGS | | | percent: H: 0.9 E: 0.3 | |
| | PAGSPTSTEEGTSESATPESGPGTST | | | | |
| | EPSEGSAPGSPAGSPTSTEEGTSTEP | | | | |
| | SEGSAPGTSTEPSEGSAPGTSESATP | | | | |
| | ESGPGSEPATSGSETPGSEPATSGSE | | | | |
| | TPGSPAGSPTSTEEGTSESATPESGP | | | | |
| | GTSTEPSEGSAPGTSTEPSEGSAPGS | | | | |
| | PAGSPTSTEEGTSTEPSEGSAPGTST | | | | |
| | EPSEGSAPGTSESATPESGPGTSTEP | | | | |
| | SEGSAPGTSESATPESGPGSEPATSG | | | | |
| | SETPGTSTEPSEGSAPGTSTEPSEGS | | | | |
| | APGTSESATPESGPGTSESATPESGP | | | | |
| | GSPAGSPTSTEEGTSESATPESGPGS | | | | |
| | EPATSGSETPGTSESATPESGPGTST | | | | |
| | EPSEGSAPGTSTEPSEGSAPGTSTEP | | | | |
| | SEGSAPGTSTEPSEGSAPGTSTEPSE | | | | |
| | GSAPGTSTEPSEGSAPGSPAGSPTST | | | | |
| | EEGTSTEPSEGSAPGTSESATPESGP | | | | |
| | GSEPATSGSETPGTSESATPESGPGS | | | | |
| | EPATSGSETPGTSESATPESGPGTST | | | | |
| | EPSEGSAPGTSESATPESGPGSPAGS | | | | |
| | PTSTEEGSPAGSPTSTEEGSPAGSPT | | | | |
| | STEEGTSESATPESGPGTSTEPSEGS | | | | |
| | APGTSESATPESGPGSEPATSGSETP | | | | |
| | GTSESATPESGPGSEPATSGSETPGT | | | | |
| | SESATPESGPGTSTEPSEGSAPGSPA | | | | |
| | GSPTSTEEGTSESATPESGPGSEPAT | | | | |
| | SGSETPGTSESATPESGPGSPAGSPT | | | | |
| | STEEGSPAGSPTSTEEGTSTEPSEGS | | | | |
| | APGTSESATPESGPGTSESATPESGP | | | | |
| | GTSESATPESGPGSEPATSGSETPGS | | | | |
| | EPATSGSETPGSPAGSPTSTEEGTST | | | | |
| | EPSEGSAPGTSTEPSEGSAPGSEPAT | | | | |
| | SGSETPGTSESATPESGPGTSTEPSE | | | | |
| | GSAP | | | | |
| BC 864 | GTSTEPSEPGSAGTSTEPSEPGSAGS | 700 | | Residue totals: H: 0 E:0 | 99.77% |
| | EPATSGTEPSGSGASEPTSTEPGSEP | | | percent: H: 0 E: 0 | |
| | ATSGTEPSGSEPATSGTEPSGSEPAT | | | | |
| | SGTEPSGSGASEPTSTEPGTSTEPSEP | | | | |
| | GSAGSEPATSGTEPSGTSTEPSEPGS | | | | |
| | AGSEPATSGTEPSGSEPATSGTEPSG | | | | |
| | TSTEPSEPGSAGTSTEPSEPGSAGSE | | | | |
| | PATSGTEPSGSEPATSGTEPSGTSEP | | | | |
| | STSEPGAGSGASEPTSTEPGTSEPST | | | | |
| | SEPGAGSEPATSGTEPSGSEPATSGT | | | | |
| | EPSGTSTEPSEPGSAGTSTEPSEPGS | | | | |
| | AGSGASEPTSTEPGSEPATSGTEPSG | | | | |
| | SEPATSGTEPSGSEPATSGTEPSGSE | | | | |
| | PATSGTEPSGTSTEPSEPGSAGSEPA | | | | |
| | TSGTEPSGSGASEPTSTEPGTSTEPSE | | | | |
| | PGSAGSEPATSGTEPSGSGASEPTST | | | | |
| | EPGTSTEPSEPGSAGSGASEPTSTEP | | | | |
| | GSEPATSGTEPSGSGASEPTSTEPGS | | | | |
| | EPATSGTEPSGSGASEPTSTEPGTST | | | | |
| | EPSEPGSAGSEPATSGTEPSGSGASE | | | | |
| | PTSTEPGTSTEPSEPGSAGSEPATSG | | | | |
| | TEPSGTSTEPSEPGSAGSEPATSGTE | | | | |
| | PSGTSTEPSEPGSAGTSTEPSEPGSA | | | | |
| | GTSTEPSEPGSAGTSTEPSEPGSAGT | | | | |
| | STEPSEPGSAGTSTEPSEPGSAGTSE | | | | |
| | PSTSEPGAGSGASEPTSTEPGTSTEP | | | | |
| | SEPGSAGTSTEPSEPGSAGTSTEPSE | | | | |
| | PGSAGSEPATSGTEPSGSGASEPTST | | | | |
| | EPGSEPATSGTEPSGSEPATSGTEPS | | | | |
| | GSEPATSGTEPSGSEPATSGTEPSGT | | | | |
| | SEPSTSEPGAGSEPATSGTEPSGSGA | | | | |
| | SEPTSTEPGTSTEPSEPGSAGSEPATS | | | | |
| | GTEPSGSGASEPTSTEPGTSTEPSEP | | | | |
| | GSA | | | | |

* H: alpha-helix E: beta-sheet

### Example 28

In this Example, different polypeptides, including several XTEN sequences, were assessed for repetitiveness in the amino acid sequence. Polypeptide amino acid sequences can be assessed for repetitiveness by quantifying the number of times a shorter subsequence appears within the overall polypeptide. For example, a polypeptide of 200 amino acid residues length has a total of 165 overlapping 36-amino acid “blocks” (or “36-mers”) and 198 3-mer “subsequences”, but the number of unique 3-mer subsequences will depend on the amount of repetitiveness within the sequence. For the analyses, different polypeptide sequences were assessed for repetitiveness by determining the subsequence score obtained by application of the following equation:

$$Subsequence score = \frac{{\sum }_{i = 1}^{n} ( \frac{{Count}_{i}}{m} )}{n}$$

where: n=(amino acid length of polypeptide)−(amino acid length of block)+1;
   m=(amino acid length of block)−(amino acid length of subsequence)+1; and
   Countᵢ=cumulative number of occurrences of each unique subsequence within blockᵢ
      In the analyses of the present Example, the subsequence score for the polypeptides of Table 26 were determined using the foregoing equation in a computer program wherein the block length was set at 36 amino acids and the subsequence length was set at 3 amino acids. The resulting subsequence score is a reflection of the degree of repetitiveness within the polypeptide.

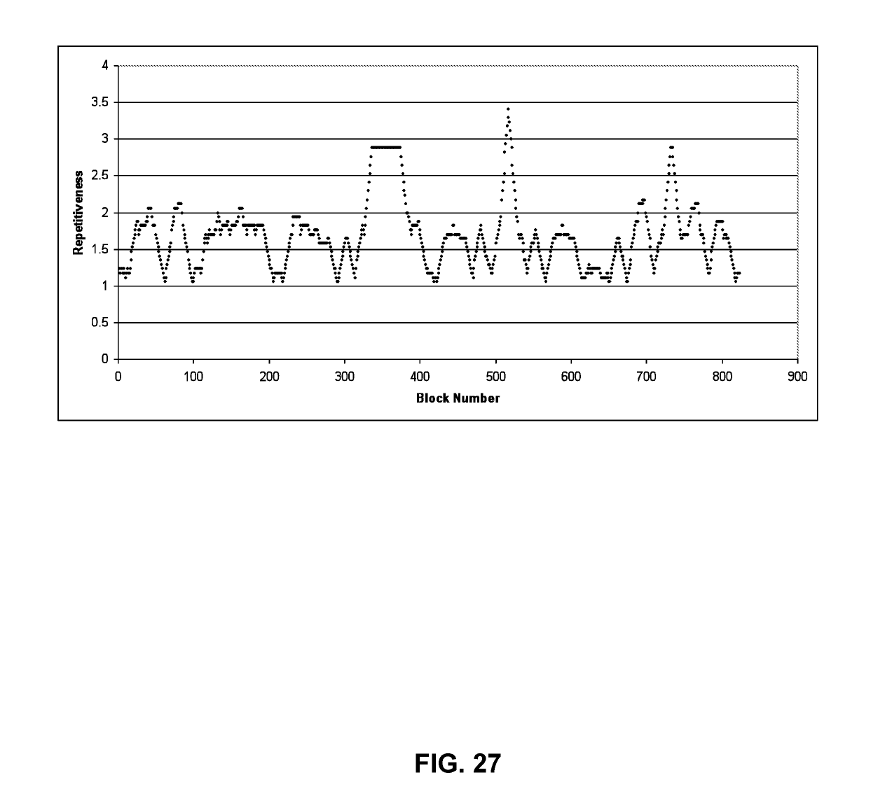

The results, shown in Table 26, indicate that the polypeptides consisting of 2 or 3 amino acid types have high subsequence scores and, hence, a high degree of repetitiveness, while XTEN designed with only four types of 12 amino acids motifs (e.g., motifs from a family of Table 3), each consisting of four to six amino acids (i.e., G, S, T, E, P, and A) in a non-repetitive sequence, have subsequence scores of less than 3 and, in many cases, less than 2, reflecting a low degree of repetitiveness across the entire sequence. For example, the L288 sequence has two amino acid types and has short, highly repetitive block sequences, resulting in a subsequence score of 8.5. The polypeptide J288 has three amino acid types but also has short, repetitive block sequences, resulting in a subsequence score of 5.7. Y576 also has three amino acid types, but is not made of internal repeats, reflected in the subsequence score of 4.7. W576 consists of four types of amino acids, but has a higher degree of internal repetitiveness with the blocks, e.g., “GGSG” (SEQ ID NO: 701), resulting in a subsequence score of 4.3. The XTEN AD576 consists of four types of 12 amino acid motifs, each consisting of four types of amino acids. Because of the low degree of internal repetitiveness of the individual motifs, the overall subsequence score amino acids is 2.5. In contrast, the XTEN's consisting of four motifs containing six types of amino acids, each with a low degree of internal repetitiveness, have subsequence scores less than 2. For the XTEN sequences AE864 and AG864, the output of the program was graphed to show the variation in repetitiveness over the length of the sequence. FIG. 27, for AE864 and FIG. 28 for Ag864, show the output, in which the individual subsequence score for the sequential 36-mer blocks are plotted as individual points corresponding to the start of each block as the amino acid number in the sequence in the X axis versus the subsequence scores for the corresponding blocks in the Y-axis. Examination of the graph for AE864 shows that the sequence, which has an overall subsequence score of 1.7, varies between scores of 1 and 2 for much of the sequence, but has areas of higher repetitiveness starting around amino acid 330, 505, and 725. Conversely, there are approximately 10 blocks where the subsequence score approaches 1, a score that represents a complete lack of repetitiveness. Similarly, examination of the graph for AG864 shows that the sequence, which has an overall subsequence score of 1.9, varies between scores of 1.2 and 2 for much of the sequence, but has four areas of higher repetitiveness where the subsequence scores are above 3.

Conclusions: The results indicate that the combination of 12 amino acid subsequence motifs, each consisting of four to six amino acid types that are essentially non-repetitive, into a longer XTEN polypeptide results in an overall sequence that is substantially non-repetitive, as indicated by overall subsequence scores less than 3 and, in many cases, less than 2. This is despite the fact that each subsequence motif may be used multiple times across the sequence. In contrast, polymers created from smaller numbers of amino acid types resulted in higher subsequence scores, with polypeptides consisting of two amino acid type having higher scores that those consisting of three amino acid types.

TABLE 26
Subsequence score calculations of polypeptide sequences

| | | SEQ | |
|---|---|---|---|
| | | ID | |
| Seq Name | Amino Acid Sequence | NO: | Score |
| US | NNTNNTNNTNNTNNTNNTNNTNNTNNTNNTNNTNNTNNTNNTNNTNNTN | 702 | 11.4 |
| 20090298762 | NTNNTNNTNNTNNTNNTNNTNNTNNTNNTNNTNNTNNTNNTNNTNNTNN | | |
| SEQ ID NO: 1 | TNNTNNTNNTNNTNNTNNTNNTNNTNNTNNTNNTNNTNNTNNTNNTNNT | | |
| | NNTNNTNNTNNTNNTNNTNNTNNTNNTNNTNNTNNTNNTNNTNNTNNT | | |
| H288 | GSGGEGGSGGSGGSGGEGGSGGSGGSGGEGGSGGSGGSGGEGGSGGSGGS | 703 | 7.1 |
| | GGEGGSGGSGGSGGEGGSGGSGGSGGEGGSGGSGGSGGEGGSGGSGGSGG | | |
| | EGGSGGSGGSGGEGGSGGSGGSGGEGGSGGSGGSGGEGGSGGSGGSGGEG | | |
| | GSGGSGGSGGEGGSGGSGGSGGEGGSGGSGGSGGEGGSGGSGGSGGEGGS | | |
| | GGSGGSGGEGGSGGSGGSGGEGGSGGSGGSGGEGGSGGSGGSGGEGGSGG | | |
| | SGGSGGEGGSGGSGGSGGEGGSGGSGGSGGEGGSGGSG | | |
| J288 | GSGGEGGSGGEGGSGGEGGSGGEGGSGGEGGSGGEGGSGGEGGSGGEGGS | 704 | 5.7 |
| | GGEGGSGGEGGSGGEGGSGGEGGSGGEGGSGGEGGSGGEGGSGGEGGSGG | | |
| | EGGSGGEGGSGGEGGSGGEGGSGGEGGSGGEGGSGGEGGSGGEGGSGGEG | | |
| | GSGGEGGSGGEGGSGGEGGSGGEGGSGGEGGSGGEGGSGGEGGSGGEGGS | | |
| | GGEGGSGGEGGSGGEGGSGGEGGSGGEGGSGGEGGSGGEGGSGGEGGSGG | | |
| | EGGSGGEGGSGGEGGSGGEGGSGGEGGSGGEGGSGGEG | | |
| K288 | GEGEGGGEGGEGEGGGEGGEGEGGGEGGEGEGGGEGGEGEGGGEGGEGE | 705 | 8.0 |
| | GGGEGGEGEGGGEGGEGEGGGEGGEGEGGGEGGEGEGGGEGGEGEGGGE | | |
| | GGEGEGGGEGGEGEGGGEGGEGEGGGEGGEGEGGGEGGEGEGGGEGGEG | | |
| | EGGGEGGEGEGGGEGGEGEGGGEGGEGEGGGEGGEGEGGGEGGEGEGGG | | |
| | EGGEGEGGGEGGEGEGGGEGGEGEGGGEGGEGEGGGEGGEGEGGGEGGE | | |
| | GEGGGEGGEGEGGGEGGEGEGGGEGGEGEGGGEGGEGEGGGEG | | |
| L288 | SSESSESSSSESSSESSESSSSESSSESSESSSSESSSESSESSSSESSSESSESSSSE | 706 | 8.5 |
| | SSSESSESSSSESSSESSESSSSESSSESSESSSSESSSESSESSSSESSSESSESSSS | | |
| | ESSSESSESSSSESSSESSESSSSESSSESSESSSSESSSESSESSSSESSSESSESSS | | |
| | SESSSESSESSSSESSSESSESSSSESSSESSESSSSESSSESSESSSSESSSESSESS | | |
| | SSESSSESSESSSSESSSESSESSSSESSSESSESSSSESSSESSESSSSES | | |
| Y288 | GEGSGEGSEGEGSEGSGEGEGSEGSGEGEGGSEGSEGEGGSEGSEGEGGSEG | 707 | 4.7 |
| | SEGEGSGEGSEGEGGSEGSEGEGSGEGSEGEGSEGGSEGEGGSEGSEGEGSG | | |
| | EGSEGEGGEGGSEGEGSEGSGEGEGSGEGSEGEGSEGSGEGEGSGEGSEGEG | | |
| | SEGSGEGEGSEGSGEGEGGSEGSEGEGSEGSGEGEGGEGSGEGEGSGEGSEG | | |
| | EGGGEGSEGEGSGEGGEGEGSEGGSEGEGGSEGGEGEGSEGSGEGEGSEGG | | |
| | SEGEGSEGGSEGEGSEGSGEGEGSEGSGE | | |
| Q576 | GGKPGEGGKPEGGGGKPGGKPEGEGEGKPGGKPEGGGKPGGGEGGKPEGG | 708 | 3.4 |
| | KPEGEGKPGGGEGKPGGKPEGGGGKPEGEGKPGGGGGKPGGKPEGEGKPG | | |
| | GGEGGKPEGKPGEGGEGKPGGKPEGGGEGKPGGGKPGEGGKPGEGKPGGG | | |
| | EGGKPEGGKPEGEGKPGGGEGKPGGKPGEGGKPEGGGEGKPGGKPGEGGE | | |
| | GKPGGGKPEGEGKPGGGKPGGGEGGKPEGEGKPGGKPEGGGEGKPGGKPE | | |
| | GGGKPEGGGEGKPGGGKPGEGGKPGEGEGKPGGKPEGEGKPGGEGGGKPE | | |
| | GKPGGGEGGKPEGGKPGEGGKPEGGKPGEGGEGKPGGGKPGEGGKPEGGG | | |
| | KPEGEGKPGGGGKPGEGGKPEGGKPEGGGEGKPGGGKPEGEGKPGGGEGK | | |
| | PGGKPEGGGGKPGEGGKPEGGKPGGEGGGKPEGEGKPGGKPGEGGGGKPG | | |
| | GKPEGEGKPGEGGEGKPGGKPEGGGEGKPGGKPEGGGEGKPGGGKPGEGG | | |
| | KPEGGGKPGEGGKPGEGGKPEGEGKPGGGEGKPGGKPGEGGKPEGGGEGK | | |
| | PGGKPGGEGGGKPEGGKPGEGGKPEG | | |
| U576 | GEGKPGGKPGSGGGKPGEGGKPGSGEGKPGGKPGSGGSGKPGGKPGEGGK | 709 | 3.4 |
| | PEGGSGGKPGGGGKPGGKPGGEGSGKPGGKPEGGGKPEGGSGGKPGGKPE | | |
| | GGSGGKPGGKPGSGEGGKPGGGKPGGEGKPGSGKPGGEGSGKPGGKPEGG | | |
| | SGGKPGGKPEGGSGGKPGGSGKPGGKPGEGGKPEGGSGGKPGGSGKPGGK | | |
| | PEGGGSGKPGGKPGEGGKPGSGEGGKPGGGKPGGEGKPGSGKPGGEGSGK | | |
| | PGGKPGSGGEGKPGGKPEGGSGGKPGGGKPGGEGKPGSGGKPGEGGKPGS | | |
| | GGGKPGGKPGGEGEGKPGGKPGEGGKPGGEGSGKPGGGGKPGGKPGGEGG | | |
| | KPEGSGKPGGGSGKPGGKPEGGGGKPEGSGKPGGGGKPEGSGKPGGGKPE | | |
| | GGSGGKPGGSGKPGGKPGEGGGKPEGSGKPGGGSGKPGGKPEGGGKPEGG | | |
| | SGGKPGGKPEGGSGGKPGGKPGGEGSGKPGGKPGSGEGGKPGGKPGEGSG | | |
| | GKPGGKPEGGSGGKPGGSGKPGGKPEGGGSGKPGGKPGEGGKPGGEGSGK | | |
| | PGGSGKPG | | |
| W576 | GGSGKPGKPGGSGSGKPGSGKPGGGSGKPGSGKPGGGSGKPGSGKPGGGSG | 710 | 4.3 |
| | KPGSGKPGGGGKPGSGSGKPGGGKPGGSGGKPGGGSGKPGKPGSGGSGKP | | |
| | GSGKPGGGSGGKPGKPGSGGSGGKPGKPGSGGGSGKPGKPGSGGSGGKPG | | |
| | KPGSGGSGGKPGKPGSGGSGKPGSGKPGGGSGKPGSGKPGSGGSGKPGKPG | | |
| | SGGSGKPGSGKPGSGSGKPGSGKPGGGSGKPGSGKPGSGGSGKPGKPGSGG | | |
| | GKPGSGSGKPGGGKPGSGSGKPGGGKPGGSGGKPGGSGGKPGKPGSGGGS | | |
| | GKPGKPGSGGGSGKPGKPGGSGSGKPGSGKPGGGSGKPGSGKPGSGGSGKP | | |
| | GKPGSGGSGGKPGKPGSGGGKPGSGSGKPGGGKPGSGSGKPGGGKPGSGSG | | |
| | KPGGGKPGSGSGKPGGSGKPGSGKPGGGSGGKPGKPGSGGSGKPGSGKPGS | | |
| | GGSGKPGKPGGSGSGKPGSGKPGGGSGKPGSGKPGGGSGKPGSGKPGGGSG | | |
| | KPGSGKPGGGGKPGSGSGKPGGSGGKPGKPGSGGSGGKPGKPGSGGSGKPG | | |
| | SGKPGGGSGGKPGKPGSGG | | |
| Y576 | GEGSGEGSEGEGSEGSGEGEGSEGSGEGEGGSEGSEGEGSEGSGEGEGGEGS | 711 | 4.7 |
| | GEGEGSGEGSEGEGGGEGSEGEGSGEGGEGEGSEGGSEGEGGSEGGEGEGS | | |
| | EGSGEGEGSEGGSEGEGSEGGSEGEGSEGSGEGEGSEGSGEGEGSEGSGEGE | | |
| | GSEGSGEGEGSEGGSEGEGGSEGSEGEGSGEGSEGEGGSEGSEGEGGGEGSE | | |
| | GEGSGEGSEGEGGSEGSEGEGGSEGSEGEGGEGSGEGEGSEGSGEGEGSGEG | | |
| | SEGEGSEGSGEGEGSEGSGEGEGGSEGSEGEGSGEGSEGEGSEGSGEGEGSE | | |
| | GSGEGEGGSEGSEGEGGSEGSEGEGGSEGSEGEGGEGSGEGEGSEGSGEGEG | | |
| | SGEGSEGEGSEGSGEGEGSEGSGEGEGGSEGSEGEGSEGSGEGEGGEGSGEG | | |
| | EGSGEGSEGEGGGEGSEGEGSEGSGEGEGSEGSGEGEGSEGGSEGEGGSEGS | | |
| | EGEGSEGGSEGEGSEGGSEGEGSEGSGEGEGSEGSGEGEGSGEGSEGEGGSE | | |
| | GGEGEGSEGGSEGEGSEGGSEGEGGEGSGEGEGGGEGSEGEGSEGSGEGEG | | |
| | SGEGSE | | |
| AE42_1 | TEPSEGSAPGSPAGSPTSTEEGTSESATPESGPGSEPATSGS | 712 | 1.2 |
| AE42_2 | PAGSPTSTEEGTSTEPSEGSAPGTSESATPESGPGSEPATSG | 713 | 1.2 |
| AE42_3 | SEPATSGSETPGTSESATPESGPGTSTEPSEGSAPGSPAGSP | 714 | 1.1 |
| AG42_1 | GAPSPSASTGTGPGTPGSGTASSSPGSSTPSGATGSPGPSGP | 715 | 1.1 |
| AG42_2 | GPGTPGSGTASSSPGSSTPSGATGSPGSSPSASTGTGPGASP | 716 | 1.3 |
| AG42_3 | SPSASTGTGPGASPGTSSTGSPGTPGSGTASSSPGSSTPSGA | 717 | 1.4 |
| AG42_4 | SASTGTGPGASPGTSSTGSPGTPGSGTASSSPGSSTPSGATG | 718 | 1.4 |
| AE48 | MAEPAGSPTSTEEGTPGSGTASSSPGSSTPSGATGSPGASPGTSSTGS | 719 | 1.2 |
| AM48 | MAEPAGSPTSTEEGASPGTSSTGSPGSSTPSGATGSPGSSTPSGATGS | 720 | 1.7 |
| AE144 | GSEPATSGSETPGTSESATPESGPGSEPATSGSETPGSPAGSPTSTEEGTSTEPS | 721 | 1.6 |
| | EGSAPGSEPATSGSETPGSEPATSGSETPGSEPATSGSETPGTSTEPSEGSAPGT | | |
| | SESATPESGPGSEPATSGSETPGTSTEPSEGSAP | | |
| AF144 | GTSTPESGSASPGTSPSGESSTAPGTSPSGESSTAPGSTSSTAESPGPGSTSESPS | 722 | 1.7 |
| | GTAPGSTSSTAESPGPGTSPSGESSTAPGTSTPESGSASPGSTSSTAESPGPGTS | | |
| | PSGESSTAPGTSPSGESSTAPGTSPSGESSTAP | | |
| AG144_1 | PGSSPSASTGTGPGSSPSASTGTGPGTPGSGTASSSPGSSTPSGATGSPGSSPSA | 723 | 1.6 |
| | STGTGPGASPGTSSTGSPGTPGSGTASSSPGSSTPSGATGSPGTPGSGTASSSP | | |
| | GASPGTSSTGSPGASPGTSSTGSPGTPGSGTASSS | | |
| AG144_2 | SGTASSSPGSSTPSGATGSPGTPGSGTASSSPGSSTPSGATGSPGSSTPSGATGS | 724 | 1.7 |
| | PGSSPSASTGTGPGSSPSASTGTGPGASPGTSSTGSPGTPGSGTASSSPGSSTPS | | |
| | GATGSPGSSPSASTGTGPGSSPSASTGTGPGASP | | |
| AG144_3 | GTPGSGTASSSPGSSTPSGATGSPGSSTPSGATGSPGSSPSASTGTGPGSSPSAS | 725 | 1.7 |
| | TGTGPGASPGTSSTGSPGTPGSGTASSSPGSSTPSGATGSPGSSPSASTGTGPG | | |
| | SSPSASTGTGPGASPGTSSTGSPGASPGTSSTGSP | | |
| AG144_4 | GTPGSGTASSSPGSSTPSGATGSPGSSPSASTGTGPGSSPSASTGTGPGASPGT | 726 | 1.7 |
| | SSTGSPGASPGTSSTGSPGSSTPSGATGSPGSSPSASTGTGPGASPGTSSTGSPG | | |
| | SSPSASTGTGPGTPGSGTASSSPGSSTPSGATGSP | | |
| AE288 | GTSESATPESGPGSEPATSGSETPGTSESATPESGPGSEPATSGSETPGTSESAT | 727 | 1.6 |
| | PESGPGTSTEPSEGSAPGSPAGSPTSTEEGTSESATPESGPGSEPATSGSETPGT | | |
| | SESATPESGPGSPAGSPTSTEEGSPAGSPTSTEEGTSTEPSEGSAPGTSESATPE | | |
| | SGPGTSESATPESGPGTSESATPESGPGSEPATSGSETPGSEPATSGSETPGSPA | | |
| | GSPTSTEEGTSTEPSEGSAPGTSTEPSEGSAPGSEPATSGSETPGTSESATPESG | | |
| | PGTSTEPSEGSAP | | |
| AG288_1 | PGASPGTSSTGSPGASPGTSSTGSPGTPGSGTASSSPGSSTPSGATGSPGTPGS | 728 | 1.8 |
| | GTASSSPGSSTPSGATGSPGTPGSGTASSSPGS STPSGATGSPGSSTPSGATGSP | | |
| | GSSPSASTGTGPGSSPSASTGTGPGASPGTSSTGSPGTPGSGTASSSPGSSTPSG | | |
| | ATGSPGSSPSASTGTGPGSSPSASTGTGPGASPGTSSTGSPGASPGTSSTGSPG | | |
| | SSTPSGATGSPGSSPSASTGTGPGASPGTSSTGSPGSSPSASTGTGPGTPGSGT | | |
| | ASSSPGSSTPSGATGS | | |
| AG288_2 | GSSPSASTGTGPGSSPSASTGTGPGTPGSGTASSSPGSSTPSGATGSPGSSPSAS | 729 | 1.8 |
| | TGTGPGASPGTSSTGSPGTPGSGTASSSPGSSTPSGATGSPGTPGSGTASSSPG | | |
| | ASPGTSSTGSPGASPGTSSTGSPGTPGSGTASSSPGSSTPSGATGSPGASPGTSS | | |
| | TGSPGTPGSGTASSSPGSSTPSGATGSPGSSPSASTGTGPGSSPSASTGTGPGSS | | |
| | TPSGATGSPGSSTPSGATGSPGASPGTSSTGSPGASPGTSSTGSPGASPGTSST | | |
| | GSPGTPGSGTASSSP | | |
| AD576 | GSSESGSSEGGPGSGGEPSESGSSGSSESGSSEGGPGSSESGSSEGGPGSSESGS | 730 | 2.5 |
| | SEGGPGSSESGSSEGGPGSSESGSSEGGPGESPGGSSGSESGSEGSSGPGESSG | | |
| | SSESGSSEGGPGSSESGSSEGGPGSSESGSSEGGPGSGGEPSESGSSGESPGGSS | | |
| | GSESGESPGGSSGSESGSGGEPSESGSSGSSESGSSEGGPGSGGEPSESGSSGS | | |
| | GGEPSESGSSGSEGSSGPGESSGESPGGSSGSESGSGGEPSESGSSGSGGEPSE | | |
| | SGSSGSGGEPSESGSSGSSESGSSEGGPGESPGGSSGSESGESPGGSSGSESGES | | |
| | PGGSSGSESGESPGGSSGSESGESPGGSSGSESGSSESGSSEGGPGSGGEPSES | | |
| | GSSGSEGSSGPGESSGSSESGSSEGGPGSGGEPSESGSSGSSESGSSEGGPGSG | | |
| | GEPSESGSSGESPGGSSGSESGESPGGSSGSESGSSESGSSEGGPGSGGEPSES | | |
| | GSSGSSESGSSEGGPGSGGEPSESGSSGSGGEPSESGSSGESPGGSSGSESGSE | | |
| | GSSGPGESSGSSESGSSEGGPGSEGSSGPGESS | | |
| AE576 | AGSPAGSPTSTEEGTSESATPESGPGTSTEPSEGSAPGSPAGSPTSTEEGTSTEP | 731 | 1.7 |
| | SEGSAPGTSTEPSEGSAPGTSESATPESGPGSEPATSGSETPGSEPATSGSETPG | | |
| | SPAGSPTSTEEGTSESATPESGPGTSTEPSEGSAPGTSTEPSEGSAPGSPAGSPT | | |
| | STEEGTSTEPSEGSAPGTSTEPSEGSAPGTSESATPESGPGTSTEPSEGSAPGTS | | |
| | ESATPESGPGSEPATSGSETPGTSTEPSEGSAPGTSTEPSEGSAPGTSESATPES | | |
| | GPGTSESATPESGPGSPAGSPTSTEEGTSESATPESGPGSEPATSGSETPGTSES | | |
| | ATPESGPGTSTEPSEGSAPGTSTEPSEGSAPGTSTEPSEGSAPGTSTEPSEGSAP | | |
| | GTSTEPSEGSAPGTSTEPSEGSAPGSPAGSPTSTEEGTSTEPSEGSAPGTSESAT | | |
| | PESGPGSEPATSGSETPGTSESATPESGPGSEPATSGSETPGTSESATPESGPGT | | |
| | STEPSEGSAPGTSESATPESGPGSPAGSPTSTEEGSPAGSPTSTEEGSPAGSPTS | | |
| | TEEGTSESATPESGPGTSTEPSEGSAP | | |
| AF540 | GSTSSTAESPGPGSTSSTAESPGPGSTSESPSGTAPGSTSSTAESPGPGSTSSTA | 732 | 1.8 |
| | ESPGPGTSTPESGSASPGSTSESPSGTAPGTSPSGESSTAPGSTSESPSGTAPGS | | |
| | TSESPSGTAPGTSPSGESSTAPGSTSESPSGTAPGSTSESPSGTAPGTSPSGESS | | |
| | TAPGSTSESPSGTAPGSTSESPSGTAPGSTSESPSGTAPGTSTPESGSASPGSTS | | |
| | ESPSGTAPGTSTPESGSASPGSTSSTAESPGPGSTSSTAESPGPGTSTPESGSAS | | |
| | PGTSTPESGSASPGSTSESPSGTAPGTSTPESGSASPGTSTPESGSASPGSTSESP | | |
| | SGTAPGSTSESPSGTAPGSTSESPSGTAPGSTSSTAESPGPGTSTPESGSASPGT | | |
| | STPESGSASPGSTSESPSGTAPGSTSESPSGTAPGTSTPESGSASPGSTSESPSGT | | |
| | APGSTSESPSGTAPGTSTPESGSASPGTSPSGESSTAPGSTSSTAESPGPGTSPS | | |
| | GESSTAPGSTSSTAESPGPGTSTPESGSASPGSTSESPSGTAP | | |
| AF504 | GASPGTSSTGSPGSSPSASTGTGPGSSPSASTGTGPGTPGSGTASSSPGSSTPSG | 733 | 1.9 |
| | ATGSPGSNPSASTGTGPGASPGTSSTGSPGTPGSGTASSSPGSSTPSGATGSPG | | |
| | TPGSGTASSSPGASPGTSSTGSPGASPGTSSTGSPGTPGSGTASSSPGSSTPSGA | | |
| | TGSPGASPGTSSTGSPGTPGSGTASSSPGSSTPSGATGSPGSNPSASTGTGPGS | | |
| | SPSASTGTGPGSSTPSGATGSPGSSTPSGATGSPGASPGTSSTGSPGASPGTSS | | |
| | TGSPGASPGTSSTGSPGTPGSGTASSSPGASPGTSSTGSPGASPGTSSTGSPGA | | |
| | SPGTSSTGSPGSSPSASTGTGPGTPGSGTASSSPGASPGTSSTGSPGASPGTSST | | |
| | GSPGASPGTSSTGSPGSSTPSGATGSPGSSTPSGATGSPGASPGTSSTGSPGTP | | |
| | GSGTASSSPGSSTPSGATGSPGSSTPSGATGSPGSSTPSGATGSPGSSPSASTGT | | |
| | GPGASPGTSSTGSP | | |
| AG576 | PGTPGSGTASSSPGSSTPSGATGSPGSSPSASTGTGPGSSPSASTGTGPGSSTPS | 734 | 2.1 |
| | GATGSPGSSTPSGATGSPGASPGTSSTGSPGASPGTSSTGSPGASPGTSSTGSP | | |
| | GTPGSGTASSSPGASPGTSSTGSPGASPGTSSTGSPGASPGTSSTGSPGSSPSAS | | |
| | TGTGPGTPGSGTASSSPGASPGTSSTGSPGASPGTSSTGSPGASPGTSSTGSPG | | |
| | SSTPSGATGSPGSSTPSGATGSPGASPGTSSTGSPGTPGSGTASSSPGSSTPSGA | | |
| | TGSPGSSTPSGATGSPGSSTPSGATGSPGSSPSASTGTGPGASPGTSSTGSPGA | | |
| | SPGTSSTGSPGTPGSGTASSSPGASPGTSSTGSPGASPGTSSTGSPGASPGTSST | | |
| | GSPGASPGTSSTGSPGTPGSGTASSSPGSSTPSGATGSPGTPGSGTASS SPGSST | | |
| | PSGATGSPGTPGSGTASSSPGSSTPSGATGSPGSSTPSGATGSPGSSPSASTGT | | |
| | GPGSSPSASTGTGPGASPGTSSTGSPGTPGSGTASSSPGSSTPSGATGSPGSSPS | | |
| | ASTGTGPGSSPSASTGTGPGASPGTSSTGS | | |
| AD836 | GSSESGSSEGGPGSSESGSSEGGPGESPGGSSGSESGSGGEPSESGSSGESPGG | 735 | 2.5 |
| | SSGSESGESPGGSSGSESGSSESGSSEGGPGSSESGSSEGGPGSSESGSSEGGPG | | |
| | ESPGGSSGSESGESPGGSSGSESGESPGGSSGSESGSSESGSSEGGPGSSESGSS | | |
| | EGGPGSSESGSSEGGPGSSESGSSEGGPGSSESGSSEGGPGSSESGSSEGGPGS | | |
| | GGEPSESGSSGESPGGSSGSESGESPGGSSGSESGSGGEPSESGSSGSEGSSGP | | |
| | GESSGSSESGSSEGGPGSGGEPSESGSSGSEGSSGPGESSGSSESGSSEGGPGS | | |
| | GGEPSESGSSGESPGGSSGSESGSGGEPSESGSSGSGGEPSESGSSGSSESGSSE | | |
| | GGPGSGGEPSESGSSGSGGEPSESGSSGSEGSSGPGESSGESPGGSSGSESGSE | | |
| | GSSGPGESSGSEGSSGPGESSGSGGEPSESGSSGSSESGSSEGGPGSSESGSSEG | | |
| | GPGESPGGSSGSESGSGGEPSESGSSGSEGSSGPGESSGESPGGSSGSESGSEG | | |
| | SSGPGSSESGSSEGGPGSGGEPSESGSSGSEGSSGPGESSGSEGSSGPGESSGSE | | |
| | GSSGPGESSGSGGEPSESGSSGSGGEPSESGSSGESPGGSSGSESGESPGGSSG | | |
| | SESGSGGEPSESGSSGSEGSSGPGESSGESPGGSSGSESGSSESGSSEGGPGSSE | | |
| | SGSSEGGPGSSESGSSEGGPGSGGEPSESGSSGSSESGSSEGGPGESPGGSSGS | | |
| | ESGSGGEPSESGSSGSSESGSSEGGPGESPGGSSGSESGSGGEPSESGSSGESP | | |
| | GGSSGSESGSGGEPSESGSS | | |
| AE864 | GSPAGSPTSTEEGTSESATPESGPGTSTEPSEGSAPGSPAGSPTSTEEGTSTEPS | 736 | 1.7 |
| | EGSAPGTSTEPSEGSAPGTSESATPESGPGSEPATSGSETPGSEPATSGSETPGS | | |
| | PAGSPTSTEEGTSESATPESGPGTSTEPSEGSAPGTSTEPSEGSAPGSPAGSPTS | | |
| | TEEGTSTEPSEGSAPGTSTEPSEGSAPGTSESATPESGPGTSTEPSEGSAPGTSE | | |
| | SATPESGPGSEPATSGSETPGTSTEPSEGSAPGTSTEPSEGSAPGTSESATPESG | | |
| | PGTSESATPESGPGSPAGSPTSTEEGTSESATPESGPGSEPATSGSETPGTSESA | | |
| | TPESGPGTSTEPSEGSAPGTSTEPSEGSAPGTSTEPSEGSAPGTSTEPSEGSAPG | | |
| | TSTEPSEGSAPGTSTEPSEGSAPGSPAGSPTSTEEGTSTEPSEGSAPGTSESATP | | |
| | ESGPGSEPATSGSETPGTSESATPESGPGSEPATSGSETPGTSESATPESGPGTS | | |
| | TEPSEGSAPGTSESATPESGPGSPAGSPTSTEEGSPAGSPTSTEEGSPAGSPTST | | |
| | EEGTSESATPESGPGTSTEPSEGSAPGTSESATPESGPGSEPATSGSETPGTSES | | |
| | ATPESGPGSEPATSGSETPGTSESATPESGPGTSTEPSEGSAPGSPAGSPTSTEE | | |
| | GTSESATPESGPGSEPATSGSETPGTSESATPESGPGSPAGSPTSTEEGSPAGSP | | |
| | TSTEEGTSTEPSEGSAPGTSESATPESGPGTSESATPESGPGTSESATPESGPGS | | |
| | EPATSGSETPGSEPATSGSETPGSPAGSPTSTEEGTSTEPSEGSAP | | |
| AF864 | GSTSESPSGTAPGTSPSGESSTAPGSTSESPSGTAPGSTSESPSGTAPGTSTPES | 737 | 1.8 |
| | GSASPGTSTPESGSASPGSTSESPSGTAPGSTSESPSGTAPGTSPSGESSTAPGS | | |
| | TSESPSGTAPGTSPSGESSTAPGTSPSGESSTAPGSTSSTAESPGPGTSPSGESS | | |
| | TAPGTSPSGESSTAPGSTSSTAESPGPGTSTPESGSASPGTSTPESGSASPGSTS | | |
| | ESPSGTAPGSTSESPSGTAPGTSTPESGSASPGSTSSTAESPGPGTSTPESGSAS | | |
| | PGSTSESPSGTAPGTSPSGESSTAPGSTSSTAESPGPGTSPSGESSTAPGTSTPE | | |
| | SGSASPGSTSSTAESPGPGSTSSTAESPGPGSTSSTAESPGPGSTSSTAESPGPG | | |
| | TSPSGESSTAPGSTSESPSGTAPGSTSESPSGTAPGTSTPESGPXXXGASASGA | | |
| | PSTXXXXSESPSGTAPGSTSESPSGTAPGSTSESPSGTAPGSTSESPSGTAPGST | | |
| | SESPSGTAPGSTSESPSGTAPGTSTPESGSASPGTSPSGESSTAPGTSPSGESST | | |
| | APGSTSSTAESPGPGTSPSGESSTAPGTSTPESGSASPGSTSESPSGTAPGSTSE | | |
| | SPSGTAPGTSPSGESSTAPGSTSESPSGTAPGTSTPESGSASPGTSTPESGSASP | | |
| | GSTSESPSGTAPGTSTPESGSASPGSTSSTAESPGPGSTSESPSGTAPGSTSESPS | | |
| | GTAPGTSPSGESSTAPGSTSSTAESPGPGTSPSGESSTAPGTSTPESGSASPGTS | | |
| | PSGESSTAPGTSPSGESSTAPGTSPSGESSTAPGSTSSTAESPGPGSTSSTAESP | | |
| | GPGTSPSGESSTAPGSSPSASTGTGPGSSTPSGATGSPGSSTPSGATGSP | | |
| AG864 | GASPGTSSTGSPGSSPSASTGTGPGSSPSASTGTGPGTPGSGTASSSPGSSTPSG | 738 | 1.9 |
| | ATGSPGSSPSASTGTGPGASPGTSSTGSPGTPGSGTASSSPGSSTPSGATGSPG | | |
| | TPGSGTASSSPGASPGTSSTGSPGASPGTSSTGSPGTPGSGTASSSPGSSTPSGA | | |
| | TGSPGASPGTSSTGSPGTPGSGTASSSPGSSTPSGATGSPGSSPSASTGTGPGSS | | |
| | PSASTGTGPGSSTPSGATGSPGSSTPSGATGSPGASPGTSSTGSPGASPGTSST | | |
| | GSPGASPGTSSTGSPGTPGSGTASSSPGASPGTSSTGSPGASPGTSSTGSPGAS | | |
| | PGTSSTGSPGSSPSASTGTGPGTPGSGTASSSPGASPGTSSTGSPGASPGTSST | | |
| | GSPGASPGTSSTGSPGSSTPSGATGSPGSSTPSGATGSPGASPGTSSTGSPGTP | | |
| | GSGTASSSPGSSTPSGATGSPGSSTPSGATGSPGSSTPSGATGSPGSSPSASTGT | | |
| | GPGASPGTSSTGSPGASPGTSSTGSPGTPGSGTASSSPGASPGTSSTGSPGASP | | |
| | GTSSTGSPGASPGTSSTGSPGASPGTSSTGSPGTPGSGTASSSPGSSTPSGATG | | |
| | SPGTPGSGTASSSPGSSTPSGATGSPGTPGSGTASSSPGSSTPSGATGSPGSSTP | | |
| | SGATGSPGSSPSASTGTGPGSSPSASTGTGPGASPGTSSTGSPGTPGSGTASSS | | |
| | PGSSTPSGATGSPGSSPSASTGTGPGSSPSASTGTGPGASPGTSSTGSPGASPG | | |
| | TSSTGSPGSSTPSGATGSPGSSPSASTGTGPGASPGTSSTGSPGSSPSASTGTGP | | |
| | GTPGSGTASSSPGSSTPSGATGSPGSSTPSGATGSPGASPGTSSTGSP | | |
| AM875 | GTSTEPSEGSAPGSEPATSGSETPGSPAGSPTSTEEGSTSSTAESPGPGTSTPES | 739 | 1.5 |
| | GSASPGSTSESPSGTAPGSTSESPSGTAPGTSTPESGSASPGTSTPESGSASPGS | | |
| | EPATSGSETPGTSESATPESGPGSPAGSPTSTEEGTSTEPSEGSAPGTSESATPE | | |
| | SGPGTSTEPSEGSAPGTSTEPSEGSAPGSPAGSPTSTEEGTSTEPSEGSAPGTST | | |
| | EPSEGSAPGTSESATPESGPGTSESATPESGPGTSTEPSEGSAPGTSTEPSEGSA | | |
| | PGTSESATPESGPGTSTEPSEGSAPGSEPATSGSETPGSPAGSPTSTEEGSSTPS | | |
| | GATGSPGTPGSGTASSSPGSSTPSGATGSPGTSTEPSEGSAPGTSTEPSEGSAP | | |
| | GSEPATSGSETPGSPAGSPTSTEEGSPAGSPTSTEEGTSTEPSEGSAPGASASG | | |
| | APSTGGTSESATPESGPGSPAGSPTSTEEGSPAGSPTSTEEGSTSSTAESPGPGS | | |
| | TSESPSGTAPGTSPSGESSTAPGTPGSGTASSSPGSSTPSGATGSPGSSPSASTG | | |
| | TGPGSEPATSGSETPGTSESATPESGPGSEPATSGSETPGSTSSTAESPGPGSTS | | |
| | STAESPGPGTSPSGESSTAPGSEPATSGSETPGSEPATSGSETPGTSTEPSEGSA | | |
| | PGSTSSTAESPGPGTSTPESGSASPGSTSESPSGTAPGTSTEPSEGSAPGTSTEP | | |
| | SEGSAPGTSTEPSEGSAPGSSTPSGATGSPGSSPSASTGTGPGASPGTSSTGSP | | |
| | GSEPATSGSETPGTSESATPESGPGSPAGSPTSTEEGSSTPSGATGSPGSSPSAS | | |
| | TGTGPGASPGTSSTGSPGTSESATPESGPGTSTEPSEGSAPGTSTEPSEGSAP | | |
| AM1296 | GTSTEPSEGSAPGSEPATSGSETPGSPAGSPTSTEEGSTSSTAESPGPGTSTPES | 740 | 1.6 |
| | GSASPGSTSESPSGTAPGSTSESPSGTAPGTSTPESGSASPGTSTPESGSASPGS | | |
| | EPATSGSETPGTSESATPESGPGSPAGSPTSTEEGTSTEPSEGSAPGTSESATPE | | |
| | SGPGTSTEPSEGSAPGTSTEPSEGSAPGSPAGSPTSTEEGTSTEPSEGSAPGTST | | |
| | EPSEGSAPGTSESATPESGPGTSESATPESGPGTSTEPSEGSAPGTSTEPSEGSA | | |
| | PGTSESATPESGPGTSTEPSEGSAPGSEPATSGSETPGSPAGSPTSTEEGSSTPS | | |
| | GATGSPGTPGSGTASSSPGSSTPSGATGSPGTSTEPSEGSAPGTSTEPSEGSAP | | |
| | GSEPATSGSETPGSPAGSPTSTEEGSPAGSPTSTEEGTSTEPSEGSAPGPEPTGP | | |
| | APSGGSEPATSGSETPGTSESATPESGPGSPAGSPTSTEEGTSESATPESGPGSP | | |
| | AGSPTSTEEGSPAGSPTSTEEGTSESATPESGPGSPAGSPTSTEEGSPAGSPTST | | |
| | EEGSTSSTAESPGPGSTSESPSGTAPGTSPSGESSTAPGSTSESPSGTAPGSTSE | | |
| | SPSGTAPGTSPSGESSTAPGTSTEPSEGSAPGTSESATPESGPGTSESATPESGP | | |
| | GSEPATSGSETPGTSESATPESGPGTSESATPESGPGTSTEPSEGSAPGTSESAT | | |
| | PESGPGTSTEPSEGSAPGTSPSGESSTAPGTSPSGESSTAPGTSPSGESSTAPGT | | |
| | STEPSEGSAPGSPAGSPTSTEEGTSTEPSEGSAPGSSPSASTGTGPGSSTPSGAT | | |
| | GSPGSSTPSGATGSPGSSTPSGATGSPGSSTPSGATGSPGASPGTSSTGSPGAS | | |
| | ASGAPSTGGTSPSGESSTAPGSTSSTAESPGPGTSPSGESSTAPGTSESATPESG | | |
| | PGTSTEPSEGSAPGTSTEPSEGSAPGSSPSASTGTGPGSSTPSGATGSPGASPG | | |
| | TSSTGSPGTSTPESGSASPGTSPSGESSTAPGTSPSGESSTAPGTSESATPESGP | | |
| | GSEPATSGSETPGTSTEPSEGSAPGSTSESPSGTAPGSTSESPSGTAPGTSTPES | | |
| | GSASPGSPAGSPTSTEEGTSESATPESGPGTSTEPSEGSAPGSPAGSPTSTEEGT | | |
| | SESATPESGPGSEPATSGSETPGSSTPSGATGSPGASPGTSSTGSPGSSTPSGAT | | |
| | GSPGSTSESPSGTAPGTSPSGESSTAPGSTSSTAESPGPGSSTPSGATGSPGASP | | |
| | GTSSTGSPGTPGSGTASSSPGSPAGSPTSTEEGSPAGSPTSTEEGTSTEPSEGSA | | |
| | P | | |
| AM923 | MAEPAGSPTSTEEGASPGTSSTGSPGSSTPSGATGSPGSSTPSGATGSPGTSTE | 741 | 1.5 |
| | PSEGSAPGSEPATSGSETPGSPAGSPTSTEEGSTSSTAESPGPGTSTPESGSASP | | |
| | GSTSESPSGTAPGSTSESPSGTAPGTSTPESGSASPGTSTPESGSASPGSEPATS | | |
| | GSETPGTSESATPESGPGSPAGSPTSTEEGTSTEPSEGSAPGTSESATPESGPGT | | |
| | STEPSEGSAPGTSTEPSEGSAPGSPAGSPTSTEEGTSTEPSEGSAPGTSTEPSEG | | |
| | SAPGTSESATPESGPGTSESATPESGPGTSTEPSEGSAPGTSTEPSEGSAPGTSE | | |
| | SATPESGPGTSTEPSEGSAPGSEPATSGSETPGSPAGSPTSTEEGSSTPSGATGS | | |
| | PGTPGSGTASSSPGSSTPSGATGSPGTSTEPSEGSAPGTSTEPSEGSAPGSEPAT | | |
| | SGSETPGSPAGSPTSTEEGSPAGSPTSTEEGTSTEPSEGSAPGASASGAPSTGG | | |
| | TSESATPESGPGSPAGSPTSTEEGSPAGSPTSTEEGSTSSTAESPGPGSTSESPS | | |
| | GTAPGTSPSGESSTAPGTPGSGTASSSPGSSTPSGATGSPGSSPSASTGTGPGS | | |
| | EPATSGSETPGTSESATPESGPGSEPATSGSETPGSTSSTAESPGPGSTSSTAES | | |
| | PGPGTSPSGESSTAPGSEPATSGSETPGSEPATSGSETPGTSTEPSEGSAPGSTS | | |
| | STAESPGPGTSTPESGSASPGSTSESPSGTAPGTSTEPSEGSAPGTSTEPSEGSA | | |
| | PGTSTEPSEGSAPGSSTPSGATGSPGSSPSASTGTGPGASPGTSSTGSPGSEPAT | | |
| | SGSETPGTSESATPESGPGSPAGSPTSTEEGSSTPSGATGSPGSSPSASTGTGPG | | |
| | ASPGTSSTGSPGTSESATPESGPGTSTEPSEGSAPGTSTEPSEGSAP | | |
| AE912 | MAEPAGSPTSTEEGTPGSGTASSSPGSSTPSGATGSPGASPGTSSTGSPGSPAG | 742 | 1.7 |
| | SPTSTEEGTSESATPESGPGTSTEPSEGSAPGSPAGSPTSTEEGTSTEPSEGSAP | | |
| | GTSTEPSEGSAPGTSESATPESGPGSEPATSGSETPGSEPATSGSETPGSPAGSP | | |
| | TSTEEGTSESATPESGPGTSTEPSEGSAPGTSTEPSEGSAPGSPAGSPTSTEEGT | | |
| | STEPSEGSAPGTSTEPSEGSAPGTSESATPESGPGTSTEPSEGSAPGTSESATPE | | |
| | SGPGSEPATSGSETPGTSTEPSEGSAPGTSTEPSEGSAPGTSESATPESGPGTSE | | |
| | SATPESGPGSPAGSPTSTEEGTSESATPESGPGSEPATSGSETPGTSESATPESG | | |
| | PGTSTEPSEGSAPGTSTEPSEGSAPGTSTEPSEGSAPGTSTEPSEGSAPGTSTEP | | |
| | SEGSAPGTSTEPSEGSAPGSPAGSPTSTEEGTSTEPSEGSAPGTSESATPESGPG | | |
| | SEPATSGSETPGTSESATPESGPGSEPATSGSETPGTSESATPESGPGTSTEPSE | | |
| | GSAPGTSESATPESGPGSPAGSPTSTEEGSPAGSPTSTEEGSPAGSPTSTEEGTS | | |
| | ESATPESGPGTSTEPSEGSAPGTSESATPESGPGSEPATSGSETPGTSESATPES | | |
| | GPGSEPATSGSETPGTSESATPESGPGTSTEPSEGSAPGSPAGSPTSTEEGTSES | | |
| | ATPESGPGSEPATSGSETPGTSESATPESGPGSPAGSPTSTEEGSPAGSPTSTEE | | |
| | GTSTEPSEGSAPGTSESATPESGPGTSESATPESGPGTSESATPESGPGSEPATS | | |
| | GSETPGSEPATSGSETPGSPAGSPTSTEEGTSTEPSEGSAPGTSTEPSEGSAPGS | | |
| | EPATSGSETPGTSESATPESGPGTSTEPSEGSAP | | |

### Example 29

### Calculation of TEPITOPE Scores

TEPITOPE scores of 9mer peptide sequence can be calculated by adding pocket potentials as described by Sturniolo [Sturniolo, T., et al. (1999) Nat Biotechnol, 17: 555]. In the present Example, separate Tepitope scores were calculated for individual HLA alleles. Table 27 shows as an example the pocket potentials for HLA*0101B, which occurs in high frequency in the Caucasian population. To calculate the TEPITOPE score of a peptide with sequence P1-P2-P3-P4-P5-P6-P7-P8-P9, the corresponding individual pocket potentials in Table 27 were added. The HLA*0101B score of a 9mer peptide with the sequence FDKLPRTSG (SEQ ID NO: 743) would be the sum of 0, −1.3, 0, 0.9, 0, −1.8, 0.09, 0, 0.

To evaluate the TEPITOPE scores for long peptides one can repeat the process for all 9mer subsequences of the sequences. This process can be repeated for the proteins encoded by other HLA alleles. Tables 28-31 give pocket potentials for the protein products of HLA alleles that occur with high frequency in the Caucasian population.

TEPITOPE scores calculated by this method range from approximately −10 to +10. However, 9mer peptides that lack a hydrophobic amino acid (FKLMVWY) (SEQ ID NO: 744) in P1 position have calculated TEPITOPE scores in the range of −1009 to −989. This value is biologically meaningless and reflects the fact that a hydrophobic amino acid serves as an anchor residue for HLA binding and peptides lacking a hydrophobic residue in P1 are considered non binders to HLA. Because most XTEN sequences lack hydrophobic residues, all combinations of 9mer subsequences will have TEPITOPEs in the range in the range of −1009 to −989. This method confirms that XTEN polypeptides may have few or no predicted T-cell epitopes.

TABLE 27
Pocket potential for HLA*0101B allele.

| Amino Acid | P1 | P2 | P3 | P4 | P5 | P6 | P7 | P8 | P9 |
|---|---|---|---|---|---|---|---|---|---|
| A | −999 | 0 | 0 | 0 | — | 0 | 0 | — | 0 |
| C | −999 | 0 | 0 | 0 | — | 0 | 0 | — | 0 |
| D | −999 | −1.3 | −1.3 | −2.4 | — | −2.7 | −2 | — | −1.9 |
| E | −999 | 0.1 | −1.2 | −0.4 | — | −2.4 | −0.6 | — | −1.9 |
| F | 0 | 0.8 | 0.8 | 0.08 | — | −2.1 | 0.3 | — | −0.4 |
| G | −999 | 0.5 | 0.2 | −0.7 | — | −0.3 | −1.1 | — | −0.8 |
| H | −999 | 0.8 | 0.2 | −0.7 | — | −2.2 | 0.1 | — | −1.1 |
| I | −1 | 1.1 | 1.5 | 0.5 | — | −1.9 | 0.6 | — | 0.7 |
| K | −999 | 1.1 | 0 | −2.1 | — | −2 | −0.2 | — | −1.7 |
| L | −1 | 1 | 1 | 0.9 | — | −2 | 0.3 | — | 0.5 |
| M | −1 | 1.1 | 1.4 | 0.8 | — | −1.8 | 0.09 | — | 0.08 |
| N | −999 | 0.8 | 0.5 | 0.04 | — | −1.1 | 0.1 | — | −1.2 |
| P | −999 | −0.5 | 0.3 | −1.9 | — | −0.2 | 0.07 | — | −1.1 |
| Q | −999 | 1.2 | 0 | 0.1 | — | −1.8 | 0.2 | — | −1.6 |
| R | −999 | 2.2 | 0.7 | −2.1 | — | −1.8 | 0.09 | — | −1 |
| S | −999 | −0.3 | 0.2 | −0.7 | — | −0.6 | −0.2 | — | −0.3 |
| T | −999 | 0 | 0 | −1 | — | −1.2 | 0.09 | — | −0.2 |
| V | −1 | 2.1 | 0.5 | −0.1 | — | −1.1 | 0.7 | — | 0.3 |
| W | 0 | −0.1 | 0 | −1.8 | — | −2.4 | −0.1 | — | −1.4 |
| Y | 0 | 0.9 | 0.8 | −1.1 | — | −2 | 0.5 | — | −0.9 |

TABLE 28
Pocket potential for HLA*0301B allele.

| Amino | | | | | | | | | |
|---|---|---|---|---|---|---|---|---|---|
| acid | P1 | P2 | P3 | P4 | P5 | P6 | P7 | P8 | P9 |
| A | −999 | 0 | 0 | 0 | — | 0 | 0 | — | 0 |
| C | −999 | 0 | 0 | 0 | — | 0 | 0 | — | 0 |
| D | −999 | −1.3 | −1.3 | 2.3 | — | −2.4 | −0.6 | — | −0.6 |
| E | −999 | 0.1 | −1.2 | −1 | — | −1.4 | −0.2 | — | −0.3 |
| F | −1 | 0.8 | 0.8 | −1 | — | −1.4 | 0.5 | — | 0.9 |
| G | −999 | 0.5 | 0.2 | 0.5 | — | −0.7 | 0.1 | — | 0.4 |
| H | −999 | 0.8 | 0.2 | 0 | — | −0.1 | −0.8 | — | −0.5 |
| I | 0 | 1.1 | 1.5 | 0.5 | — | 0.7 | 0.4 | — | 0.6 |
| K | −999 | 1.1 | 0 | −1 | — | 1.3 | −0.9 | — | −0.2 |
| L | 0 | 1 | 1 | 0 | — | 0.2 | 0.2 | — | −0 |
| M | 0 | 1.1 | 1.4 | 0 | — | −0.9 | 1.1 | — | 1.1 |
| N | −999 | 0.8 | 0.5 | 0.2 | — | −0.6 | −0.1 | — | −0.6 |
| P | −999 | −0.5 | 0.3 | −1 | — | 0.5 | 0.7 | — | −0.3 |
| Q | −999 | 1.2 | 0 | 0 | — | −0.3 | −0.1 | — | −0.2 |
| R | −999 | 2.2 | 0.7 | −1 | — | 1 | −0.9 | — | 0.5 |
| S | −999 | −0.3 | 0.2 | 0.7 | — | −0.1 | 0.07 | — | 1.1 |
| T | −999 | 0 | 0 | −1 | — | 0.8 | −0.1 | — | −0.5 |
| V | 0 | 2.1 | 0.5 | 0 | — | 1.2 | 0.2 | — | 0.3 |
| W | −1 | −0.1 | 0 | −1 | — | −1.4 | −0.6 | — | −1 |
| Y | −1 | 0.9 | 0.8 | −1 | — | −1.4 | −0.1 | — | 0.3 |

TABLE 29
Pocket potential for HLA*0401B allele.

| Amino | | | | | | | | | |
|---|---|---|---|---|---|---|---|---|---|
| acid | P1 | P2 | P3 | P4 | P5 | P6 | P7 | P8 | P9 |
| A | −999 | 0 | 0 | 0 | — | 0 | 0 | — | 0 |
| C | −999 | 0 | 0 | 0 | — | 0 | 0 | — | 0 |
| D | −999 | −1.3 | −1.3 | 1.4 | — | −1.1 | −0.3 | — | −1.7 |
| E | −999 | 0.1 | −1.2 | 1.5 | — | −2.4 | 0.2 | — | −1.7 |
| F | 0 | 0.8 | 0.8 | −0.9 | — | −1.1 | −1 | — | −1 |
| G | −999 | 0.5 | 0.2 | −1.6 | — | −1.5 | −1.3 | — | −1 |
| H | −999 | 0.8 | 0.2 | 1.1 | — | −1.4 | 0 | — | 0.08 |
| I | −1 | 1.1 | 1.5 | 0.8 | — | −0.1 | 0.08 | — | −0.3 |
| K | −999 | 1.1 | 0 | −1.7 | — | −2.4 | −0.3 | — | −0.3 |
| L | −1 | 1 | 1 | 0.8 | — | −1.1 | 0.7 | — | −1 |
| M | −1 | 1.1 | 1.4 | 0.9 | — | −1.1 | 0.8 | — | −0.4 |
| N | −999 | 0.8 | 0.5 | 0.9 | — | 1.3 | 0.6 | — | −1.4 |
| P | −999 | −0.5 | 0.3 | −1.6 | — | 0 | −0.7 | — | −1.3 |
| Q | −999 | 1.2 | 0 | 0.8 | — | −1.5 | 0 | — | 0.5 |
| R | −999 | 2.2 | 0.7 | −1.9 | — | −2.4 | −1.2 | — | −1 |
| S | −999 | −0.3 | 0.2 | 0.8 | — | 1 | −0.2 | — | 0.7 |
| T | −999 | 0 | 0 | 0.7 | — | 1.9 | −0.1 | — | −1.2 |
| V | −1 | 2.1 | 0.5 | −0.9 | — | 0.9 | 0.08 | — | −0.7 |
| W | 0 | −0.1 | 0 | −1.2 | — | −1 | −1.4 | — | −1 |
| Y | 0 | 0.9 | 0.8 | −1.6 | — | −1.5 | −1.2 | — | −1 |

TABLE 30
Pocket potential for HLA*0701B allele.

| Amino | | | | | | | | | |
|---|---|---|---|---|---|---|---|---|---|
| acid | P1 | P2 | P3 | P4 | P5 | P6 | P7 | P8 | P9 |
| A | −999 | 0 | 0 | 0 | — | 0 | 0 | — | 0 |
| C | −999 | 0 | 0 | 0 | — | 0 | 0 | — | 0 |
| D | −999 | −1.3 | −1.3 | −1.6 | — | −2.5 | −1.3 | — | −1.2 |
| E | −999 | 0.1 | −1.2 | −1.4 | — | −2.5 | 0.9 | — | −0.3 |
| F | 0 | 0.8 | 0.8 | 0.2 | — | −0.8 | 2.1 | — | 2.1 |
| G | −999 | 0.5 | 0.2 | −1.1 | — | −0.6 | 0 | — | −0.6 |
| H | −999 | 0.8 | 0.2 | 0.1 | — | −0.8 | 0.9 | — | −0.2 |
| I | −1 | 1.1 | 1.5 | 1.1 | — | −0.5 | 2.4 | — | 3.4 |
| K | −999 | 1.1 | 0 | −1.3 | — | −1.1 | 0.5 | — | −1.1 |
| L | −1 | 1 | 1 | −0.8 | — | −0.9 | 2.2 | — | 3.4 |
| M | −1 | 1.1 | 1.4 | −0.4 | — | −0.8 | 1.8 | — | 2 |
| N | −999 | 0.8 | 0.5 | −1.1 | — | −0.6 | 1.4 | — | −0.5 |
| P | −999 | −0.5 | 0.3 | −1.2 | — | −0.5 | −0.2 | — | −0.6 |
| Q | −999 | 1.2 | 0 | −1.5 | — | −1.1 | 1.1 | — | −0.9 |
| R | −999 | 2.2 | 0.7 | −1.1 | — | −1.1 | 0.7 | — | −0.8 |
| S | −999 | −0.3 | 0.2 | 1.5 | — | 0.6 | 0.4 | — | −0.3 |
| T | −999 | 0 | 0 | 1.4 | — | −0.1 | 0.9 | — | 0.4 |
| V | −1 | 2.1 | 0.5 | 0.9 | — | 0.1 | 1.6 | — | 2 |
| W | 0 | −0.1 | 0 | −1.1 | — | −0.9 | 1.4 | — | 0.8 |
| Y | 0 | 0.9 | 0.8 | −0.9 | — | −1 | 1.7 | — | 1.1 |

TABLE 31
Pocket potential for HLA*1501B allele.

| Amino | | | | | | | | | |
|---|---|---|---|---|---|---|---|---|---|
| acid | P1 | P2 | P3 | P4 | P5 | P6 | P7 | P8 | P9 |
| A | −999 | 0 | 0 | 0 | — | 0 | 0 | — | 0 |
| C | −999 | 0 | 0 | 0 | — | 0 | 0 | — | 0 |
| D | −999 | −1.3 | −1.3 | −0.4 | — | −0.4 | −0.7 | — | −1.9 |
| E | −999 | 0.1 | −1.2 | −0.6 | — | −1 | −0.7 | — | −1.9 |
| F | −1 | 0.8 | 0.8 | 2.4 | — | −0.3 | 1.4 | — | −0.4 |
| G | −999 | 0.5 | 0.2 | 0 | — | 0.5 | 0 | — | −0.8 |
| H | −999 | 0.8 | 0.2 | 1.1 | — | −0.5 | 0.6 | — | −1.1 |
| I | 0 | 1.1 | 1.5 | 0.6 | — | 0.05 | 1.5 | — | 0.7 |
| K | −999 | 1.1 | 0 | −0.7 | — | −0.3 | −0.3 | — | −1.7 |
| L | 0 | 1 | 1 | 0.5 | — | 0.2 | 1.9 | — | 0.5 |
| M | 0 | 1.1 | 1.4 | 1 | — | 0.1 | 1.7 | — | 0.08 |
| N | −999 | 0.8 | 0.5 | −0.2 | — | 0.7 | 0.7 | — | −1.2 |
| P | −999 | −0.5 | 0.3 | −0.3 | — | −0.2 | 0.3 | — | −1.1 |
| Q | −999 | 1.2 | 0 | −0.8 | — | −0.8 | −0.3 | — | −1.6 |
| R | −999 | 2.2 | 0.7 | 0.2 | — | 1 | −0.5 | — | −1 |
| S | −999 | −0.3 | 0.2 | −0.3 | — | 0.6 | 0.3 | — | −0.3 |
| T | −999 | 0 | 0 | −0.3 | — | −0 | 0.2 | — | −0.2 |
| V | 0 | 2.1 | 0.5 | 0.2 | — | −0.3 | 0.3 | — | 0.3 |
| W | −1 | −0.1 | 0 | 0.4 | — | −0.4 | 0.6 | — | −1.4 |
| Y | −1 | 0.9 | 0.8 | 2.5 | — | 0.4 | 0.7 | — | −0.9 |

### Example 30

### Assay for Effects of BFXTEN on Cardiac Remodeling

BFXTEN comprising GLP-1 and an exendin-4 would be evaluated for biologic activity in a rat model of cardiac remodeling. Male Sprague-Dawley rats (250-300 g) are anesthetized by using 5% isoflurane and a left thoracotomy performed. The left main anterior descending artery (LAD) are ligated to induce myocardial infarction. In addition, sham animals (n=10) would be subjected to the same surgical procedure without ligation of the LAD.

After two weeks recovery, rats are treated with graded doses of the BFXTEN comprising GLP-1 and exendin-4, or GLP-1 not linked to XTEN as a positive control, or vehicle, delivered via subcutaneous infusion for 11 weeks. Echocardiography is performed at the 3rd, 5th, 9th, and 13th week of myocardial infarction. Left ventricular (LV) end systolic dimension (ESD) and diastolic dimension (EDD), LV systolic volume and diastolic volume, left atrial volume parameters would be recorded. At the 13th week of MI, the hearts would be excised, the LV mass weighed and LV mass/body weight ratio determined.

The vehicle control group would be expected to show an increased E/A ratio (peak velocity of early diastolic filling/peak velocity of atrial contraction), compared to sham controls. BFXTEN demonstrating GLP-1 and exendin agonist activity would be expected to have a lower, or no increase in the ratio over the measured time points during CHF progression.

Administration of BFXTEN with GLP-1 exendin-4 would be expected to eliminate the LV end diastolic pressure (LVEDP) elevation, and cardiac output and +dp/dtmax would be reduced, as compared to the sham group, and may be normalized. Administration of GLP-1 and exendin-4 BFXTEN would also be expected to reduce LV mass, LV end diastolic dimension and systolic dimension in comparison to vehicle during the progression of CHF. The administration of bioactive BFXTEN is expected to significantly reduce cardiac remodeling, as assessed histologically, including reduction in infarct size compared to the control group. Further, administration of bioactive GLP-1 and exendin-4 BFXTEN would improve exercise capacity (EC) and exercise efficiency (EC/VO2) during a treadmill test in animals, compared to vehicle control treated animals. Results of bioactive, BFXTEN would be expected to demonstrate cardioprotective effects in the MI-induced rat model that include slowed enlargement of LV chamber, improved cardiac diastolic and systolic function, improved exercise capacity and efficiency, attenuated baseline plasma lactate, improved exercise capacity/peak lactate ratio, reduced infarction sire attenuated LV weight, and improved insulin sensitivity. BFXTEN providing such results would be expected to have utility in the treatment or prevention of cardiovascular disease.

Lengthy table referenced here
US20110312881A1-20111222-T00001
Please refer to the end of the specification for access instructions.

Lengthy table referenced here
US20110312881A1-20111222-T00002
Please refer to the end of the specification for access instructions.

Lengthy table referenced here
US20110312881A1-20111222-T00003
Please refer to the end of the specification for access instructions.

Lengthy table referenced here
US20110312881A1-20111222-T00004
Please refer to the end of the specification for access instructions.

Lengthy table referenced here
US20110312881A1-20111222-T00005
Please refer to the end of the specification for access instructions.

Lengthy table referenced here
US20110312881A1-20111222-T00006
Please refer to the end of the specification for access instructions.

Lengthy table referenced here
US20110312881A1-20111222-T00007
Please refer to the end of the specification for access instructions.

Lengthy table referenced here
US20110312881A1-20111222-T00008
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20110312881A1). An electronic copy of the table will also be available from the USPTO upon request and payment of the fee set forth in 37 CFR 1.19(b)(3).

What is claimed is:

1. An isolated monomeric fusion protein of formula V:
(XTEN)ᵤ-(S)ᵥ-(BP1)-(S)_{w}-(XTEN)-(S)ₓ-(BP2)-(S)_{y}-(XTEN)_{z} V
wherein independently for each occurrence:
(a) BP1 is a is a biologically active protein comprising a sequence that exhibits at least 90% sequence identity to a sequence from Table 1;
(b) BP2 is a is a biologically active protein comprising a sequence that exhibits at least 90% sequence identity to a sequence from Table 1 that is different from the BP1 of (a);
(c) S is a spacer sequence having between 1 to about 50 amino acid residues that can optionally comprise a cleavage sequence from Table 6;
(d) u is either 0 or 1;
(e) v is either 0 or 1;
(f) w is either 0 or 1;
(g) x is either 0 or 1;
(h) y is either 0 or 1;
(i) z is either 0 or 1, with the proviso that u+v+w+x+y+z≧1; and
(j) XTEN is an extended recombinant polypeptide comprising greater than about 100 to about 3000 amino acids wherein the XTEN is characterized in that:
(i) the sequence is substantially non-repetitive sequence such that: (1) the XTEN sequence contains no three contiguous amino acids that are identical unless the amino acids are serine residues; or (2) at least about 80% of the XTEN sequence consists of non-overlapping sequence motifs, each of the sequence motifs comprising about 9 to about 14 amino acid residues, wherein any two contiguous amino acid residues does not occur more than twice in each of the sequence motifs;
(ii) the sum of glycine (G), alanine (A), serine (S), threonine (T), glutamate (E) and proline (P) residues constitutes more than about 80% of the total amino acid sequence of the XTEN;
(iii) the sequence lacks a predicted T-cell epitope when analyzed by TEPITOPE algorithm, wherein the TEPITOPE algorithm prediction for epitopes within the XTEN sequence is based on a score of −9.
(iv) the sequence has greater than 90% random coil formation as determined by GOR algorithm;
(v) the sequence has less than 2% alpha helices and 2% beta-sheets as determined by Chou-Fasman algorithm; and
(k) the fusion protein, when administered to a subject, exhibits a terminal half-life at least about three-fold longer compared to the corresponding BP1 of (a) not linked to the XTEN and administered at a comparable dose to a subject and/or three-fold longer compared to the corresponding BP2 of (b) not linked to the XTEN and administered at a comparable dose to a subject.

2. An isolated monomeric fusion protein of formula VI:
(XTEN)ᵥ-(S)_{w}-(BP1)-(S)ₓ-(BP2)-(S)_{y}-(XTEN)_{z} VI
wherein independently for each occurrence:
(a) BP1 is a is a biologically active protein comprising a sequence that exhibits at least 90% sequence identity to a sequence from Table 1;
(b) BP2 is a is a biologically active protein comprising a sequence that exhibits at least 90% sequence identity to a sequence from Table 1 that is different from the BP1 of (a);
(c) S is a spacer sequence having between 1 to about 50 amino acid residues that can optionally comprise a cleavage sequence from Table 6;
(d) v is either 0 or 1;
(e) w is either 0 or 1;
(f) x is either 0 or 1;
(g) y is either 0 or 1;
(h) z is either 0 or 1, with the proviso that v+w+x+y+z≧1;
(i) XTEN is an extended recombinant polypeptide comprising greater than about 100 to about 3000 amino acids wherein the XTEN is characterized in that:
(i) the sequence is substantially non-repetitive sequence such that: (1) the XTEN sequence contains no three contiguous amino acids that are identical unless the amino acids are serine residues; or (2) at least about 80% of the XTEN sequence consists of non-overlapping sequence motifs, each of the sequence motifs comprising about 9 to about 14 amino acid residues, wherein any two contiguous amino acid residues does not occur more than twice in each of the sequence motifs;
(ii) the sum of glycine (G), alanine (A), serine (S), threonine (T), glutamate (E) and proline (P) residues constitutes more than about 80% of the total amino acid sequence of the XTEN;
(iii) the sequence lacks a predicted T-cell epitope when analyzed by TEPITOPE algorithm, wherein the TEPITOPE algorithm prediction for epitopes within the XTEN sequence is based on a score of −9.
(iv) the sequence has greater than 90% random coil formation as determined by GOR algorithm; and
(v) the sequence has less than 2% alpha helices and 2% beta-sheets as determined by Chou-Fasman algorithm; and
(j) the fusion protein, when administered to a subject, exhibits a terminal half-life at least about three-fold longer compared to the corresponding BP1 of (a) not linked to the XTEN and administered at a comparable dose to a subject and/or three-fold longer compared to the corresponding BP2 of (b) not linked to the XTEN and administered at a comparable dose to a subject.

3. The isolated fusion protein of claim 1 or 2, wherein the XTEN exhibit at least 90% sequence identity to one or more sequences from Table 4.

4. The isolated fusion protein of claim 1 or 2, wherein administration of multiple consecutive doses using a therapeutically effective dose regimen of the fusion protein to a subject in need thereof results in a gain in time of at least three-fold between consecutive Cₘₐₓ peaks and/or Cₘᵢₙ troughs for blood levels of the fusion protein compared to the corresponding BP1 of (a) and/or the BP2 of (b) not linked to the XTEN and administered to a subject at a therapeutically effective dose regimen for the BP1 or BP2.

5. The isolated fusion protein of claim 1 or 2, wherein administration of multiple consecutive doses using a therapeutically effective dose regimen of the fusion protein to a subject in need thereof results in an improvement in at least one measured parameter using an accumulatively smaller amount in moles of the fusion protein compared to the corresponding BP1 and/or BP2 not linked to the XTEN and administered at a therapeutically effective dose regimen for the BP1 and/or BP2 to a subject.

6. The isolated fusion protein of claim 5, wherein the one measured parameter is selected from fasting glucose level, response to oral glucose tolerance test, peak change of postprandial glucose from baseline glucose level, HA_{1c} level, daily caloric intake, satiety, rate of gastric emptying, insulin secretion in response to glucose challenge, peripheral insulin sensitivity, glucose level in response to insulin challenge, beta cell mass, and body weight reduction.

7. A composition comprising a first fusion protein and a second fusion protein, wherein:
(a) the first fusion protein comprises a first biologically active protein (BP1) comprising a sequence that exhibits at least 90% sequence identity to a sequence from Table 1, wherein the BP1 is linked to one or more extended recombinant polypeptides (XTEN) each comprising greater than about 100 to about 3000 amino acid residues;
(b) the second fusion protein comprises a second biologically active protein (BP2) comprising a sequence that exhibits at least 90% sequence identity to a sequence from Table 1 and that is different from the BP1 of (a), wherein the BP2 is linked to one or more extended recombinant polypeptides (XTEN) each comprising greater than about 100 to about 3000 amino acid residues;
(c) the XTEN of (a) and (b) is characterized in that:
(i) the sequence is substantially non-repetitive sequence such that (1) the XTEN sequence contains no three contiguous amino acids that are identical unless the amino acids are serine residues, or (2) at least about 80% of the XTEN sequence consists of non-overlapping sequence motifs, each of the sequence motifs comprising about 9 to about 14 amino acid residues, wherein any two contiguous amino acid residues does not occur more than twice in each of the sequence motifs;
(ii) the sum of glycine (G), alanine (A), serine (S), threonine (T), glutamate (E) and proline (P) residues constitutes more than about 80% of the total amino acid sequence of the XTEN;
(iii) the sequence lacks a predicted T-cell epitope when analyzed by TEPITOPE algorithm, wherein the TEPITOPE algorithm prediction for epitopes within the XTEN sequence is based on a score of −9.
(iv) the sequence has greater than 90% random coil formation as determined by GOR algorithm; and
(v) the sequence has less than 2% alpha helices and 2% beta-sheets as determined by Chou-Fasman algorithm;
(d) the first and the second fusion protein are at a fixed ratio in the composition of about 1:1 to about 1:1500; and
(e) the composition, when administered to a subject, exhibits a terminal half-life for the first and the second fusion protein in the subject at least about three-fold longer compared to the corresponding BP1 of (a) not linked to the XTEN and administered at a comparable dose to a subject and/or the BP2 of (b) not linked to the XTEN and administered at a comparable dose to a subject.

8. The composition of claim 7, wherein the first and/or the second fusion protein further comprises a spacer sequence between the biologically active protein and the XTEN having between 1 to about 50 amino acid residues that can optionally include a cleavage sequence from Table 6.

9. The composition of claim 7, wherein each of the XTEN has a subsequence score less than 3.

10. The composition of claim 7, wherein each of the XTEN is further characterized in that:
(a) the sum of asparagine and glutamine residues is less than 10% of the total amino acid sequence of the XTEN;
(b) the sum of methionine and tryptophan residues is less than 2% of the total amino acid sequence of the XTEN; and/or
(c) no one type of amino acid constitutes more than 30% of the XTEN sequence.

11. The composition of claim 7, wherein:
(a) the first fusion protein is of formula I
(BP1)-(S)ₓ-(XTEN) I
or formula III
(XTEN)-(S)ₓ-(BP1) III
(b) the second fusion protein is of formula II
(BP2)-(S)_{y}-(XTEN) II
or formula IV
(XTEN)-(S)_{y}-(BP2) IV
wherein independently for each occurrence:
(i) BP1 is a is a biologically active protein comprising a sequence that exhibits at least 90% sequence identity to a sequence from Table 1;
(ii) BP2 is a is a biologically active protein comprising a sequence that exhibits at least 90% sequence identity to a sequence from Table 1 that is different from the BP1 of (i);
(iii) S is a spacer sequence having between 1 to about 50 amino acid residues that can optionally include a cleavage sequence from Table 6;
(iv) x is either 0 or 1; and
(v) y is either 0 or 1.

12. The composition of claim 7, wherein administration of a therapeutically effective amount of the composition to a subject in need thereof results in a gain in time of at least three-fold spent within a therapeutic window for the first fusion protein of (a) and the second fusion protein of (b) compared to the corresponding BP1 of (a) not linked to the XTEN and administered at a comparable dose to a subject and/or the BP2 of (b) not linked to the XTEN and administered at a comparable dose to a subject.

13. A pharmaceutical composition comprising the fusion protein of claim 1 or 2, and at least one pharmaceutically acceptable carrier.

14. A method of treating a metabolic or cardiovascular condition, comprising administering a therapeutically effective amount of the pharmaceutical composition of claim 13 to a subject in need thereof.

15. The method of claim 14, wherein the condition is selected from the group consisting of type 1 diabetes, type 2 diabetes, obesity, hyperglycemia, hyperinsulinemia, decreased insulin production, insulin resistance, syndrome X, excessive appetite, insufficient satiety, glucagonomas, dyslipidemia, retinal neurodegenerative processes, myocardial infarction, cardiac valve disease, stroke, post-surgical catabolic changes, hibernating myocardium or diabetic cardiomyopathy, hypertrophic cardiomyopathy, heart insufficiency, aortic stenosis, valvular regurgitation, and intermittent claudication.

16. An isolated nucleic acid comprising a polynucleotide sequence selected from (a) a polynucleotide encoding the fusion protein of claim 1 or claim 2, or (b) the complement of the polynucleotide of (a).

17. An expression vector comprising the polynucleotide sequence of claim 16.

18. The expression vector of claim 17, further comprising a recombinant regulatory sequence operably linked to the polynucleotide sequence, wherein the regulatory sequence is a promoter.

19. A host cell, comprising the expression vector of claim 17.

20. An isolated fusion protein comprising a sequence that has at least 90% sequence identity to a sequence selected from Table 33, Table 34, Table 35, Table 36, Table 37, and Table 38.
